(19) United States
(12) Patent Application Publication
(10) Pub. No.: US 2026/0015409 A1
(43) Pub. Date: Jan. 15, 2026

(54) RECEPTOR-MEDIATED ENDOCYTOSIS FOR TARGETED INTERNALIZATION AND DEGRADATION OF MEMBRANE PROTEINS AND CARGOS

(71) Applicant: Dana-Farber Cancer Institute, Inc., Boston, MA (US)

(72) Inventors: Xin Zhou, Boston, MA (US); DingPeng Zhang, Boston, MA (US)

(21) Appl. No.: 19/113,760

(22) PCT Filed: Sep. 20, 2023
(86) PCT No.: PCT/US2023/074695

§ 371 Date: Mar. 20, 2025

Related U.S. Application Data
(60) Provisional application No. 63/376,389, filed on Sep. 20, 2022.
(60) Provisional application No. 63/462,828, filed on Apr. 28, 2023.

(51) Int. Cl. C07K 14/79; A61P 35/00; C07K 14/705; C07K 16/28

(52) U.S. Cl. CPC C07K 14/79; A61P 35/00; C07K 14/70503; C07K 16/2863; C07K 16/2887; C07K 2317/52; C07K 2317/77; C07K 2319/30; C07K 2319/50

(57) ABSTRACT

Disclosed are bispecific modulators. Bispecific modulators can bind to a protein of interest and to an internalizing receptor on a cell surface. Once bound, the protein of interest can be internalized and/or degraded inside a cell.

This application claims priority to U.S. Provisional Application No. 63/376,389, filed on Sep. 20, 2022, and U.S. Provisional Application No. 63/462,828, filed on Apr. 28, 2023, the entire contents of which are incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD

Aspects of the invention are drawn to compositions and methods for modulating molecules on cell surfaces, including chimeric antigen receptors (CAR) on the surface of CAR-T cells to reversibly control CAR receptors and receptors on cancer cells to inhibit cancer cell signaling and growth.

### SEQUENCE LISTING

The instant application contains a Sequence Listing which has been submitted electronically in ASCII format and is hereby incorporated by reference in its entirety. Said ASCII copy, created on [ ], is named [ ] and is [ ] bytes in size.

### BACKGROUND

Traditional small molecule inhibitor-based therapies have certain disadvantages, such as toxicity and limited efficacy. Examples of inhibitor features that contribute to their disadvantages include a binding affinity that is the determinant of inhibitor potency; a therapeutic response that depends on sustained target binding; a limited functionality on the target protein; and subverted efficacy through target protein overexpression, native ligand competition, and development of target protein mutations that limit binding or facilitate resistance. Thus, a desirable therapeutic modality exhibits low toxicity and high efficacy. For example, a desirable therapeutic modality includes a variety of features that contribute to overall potency; a therapeutic response that does not entirely or mostly depend on sustained target binding; the ability to completely or substantially limit functionality of the target protein; and limited or no ability of a target cell to subvert efficacy, such as through target protein overexpression, native ligand competition, and/or development of target protein mutations that limit binding or facilitate resistance.

Membrane proteins are central to a myriad of cellular functions and serve as targets for over half of all drugs. Therefore, developing strategies to degrade membrane proteins is of exceptional interest for both basic research and therapeutic intervention purposes.

Targeted protein degradation (TPD) is a rapidly growing field in drug discovery and pharmacology. Complementing traditional drug modalities, TPD molecules offer a novel therapeutic mechanism to tackle challenging targets or increase the therapeutic potential of currently used drugs.

Proof-of-concept strategies for membrane receptor degradation have been described. These strategies use heterobifunctional biologics that recruit a specific “effector” protein such as a membrane E3 ligase (Cotton, A. D., Nguyen, D. P., Gramespacher, J. A., Seiple, I. B. & Wells, J. A. Development of Antibody-Based PROTACs for the Degradation of the Cell-Surface Immune Checkpoint Protein PD-L1. J. Am Chem Soc 143, 593-598 (2021)) or a lysosome shuttling receptor (Ahn, G. et al. LYTACs that engage the asialoglycoprotein receptor for targeted protein degradation. Nat Chem Biol 17, 937-946 (2021)) to the protein of interest (POI) to induce lysosome-mediated protein degradation. However, the effectiveness of these biological effectors is often limited by their tissue-specific expression patterns. GalNAc-LYTAC, for instance, targets the hepatocyte-specific receptor asialoglycoprotein receptor (ASGPR), making it only suitable for treating liver disease or clearing circulating targets (Ahn, G. et al. Nat Chem Biol 17, 937-946 (2021); Zhou, Y., Teng, P., Montgomery, N. T., Li, X. & Tang, W. Development of Triantennary N-Acetylgalactosamine Conjugates as Degraders for Extracellular Proteins. ACS Cent Sci 7, 499-506 (2021)), while RNF43- or ZNRF3-based methods are more effective for treating Wnt-signaling upregulated disorders where RNF43 and ZNRF3 are expressed at high levels. Current technologies are therefore not able to cover the full spectrum of diseases, and developing alternative effectors overexpressed in different diseases and tissues would greatly expand the range of cell surface targets that can be regulated and also increase the targeting specificity. Accordingly, there is a need to improve targeted protein degradation, particularly of membrane proteins to treat an array of diseases, most notably cancer.

### SUMMARY

Disclosed here are new reagents and methods for regulating molecules on the surface of cells (e.g., molecules of interest, such as proteins of interest). In some embodiments, the reagents and methods are used to control the levels of CAR molecules on CAR-T cells. In some embodiments, the reagents and methods are used to reversibly control CAR-T cell activation. In some embodiments, CAR-T cell activation is controlled in vivo. In some embodiments, the reagents and methods are used to control the levels of receptors on the surface of cancer cells (e.g., epidermal growth factor receptor or EGFR, programmed death-ligand 1 or PDL1) to inhibit cancer cell growth or modulate immune responses. In some embodiments, the reagents and methods can be used on cells that are not cancer cells.

In some embodiments, disclosed are bispecific modulators as described herein. In some embodiments, an antigen to which a cell-surface molecule can bind or an antibody or antibody fragment that can bind to the cell-surface molecule is fused to a ligand for an internalizing receptor or an antibody or antibody fragment that can bind to the internalizing receptor or membrane protein. In embodiments, after binding, the bispecific modulator can cause the cell-surface molecule (e.g., molecules of interest, such as proteins of interest) to be internalized by the cell and, in some embodiments, the internalized cell-surface molecule can be degraded. In various embodiments, the bispecific modulators can target single-pass or multi-pass membrane proteins.

In some embodiments, disclosed are reagents and methods for improving degradation of an internalized cell-surface protein using the bispecific modulators disclosed herein. In some embodiments, peptide linkers sensitive to certain proteases are inserted into the bispecific modulators. In some embodiments, the peptide linkers are sensitive to cathepsin proteases.

Disclosed are nucleic acids encoding these molecules, vectors that contain the nucleic acids, and cells that contain the vectors and/or express the bispecific modulator molecules as disclosed herein.

In some embodiments, disclosed are methods of administering the bispecific modulators to a subject.

### BRIEF DESCRIPTION OF THE DRAWINGS

Certain illustrations, charts, or flow charts are provided to allow for a better understanding for the present invention. It is to be noted, however, that the drawings illustrate only selected embodiments of the inventions and are therefore not to be considered limiting of scope. Additional and equally effective embodiments and applications of the present invention exist.

FIG. 1 is a schematic illustrating CAR-T cell therapy in a patient.

FIG. 2 illustrates an example approach for treating toxicities observed after CAR-T-cell therapy using immunosuppressive agents.

FIG. 3 illustrates an example approach for treating toxicities observed after CAR-T-cell therapy using suicide genes or elimination markers.

FIG. 4 illustrates an example approach for treating toxicities observed after CAR-T-cell therapy using reversible genetic switches.

FIG. 5 illustrates reversible CAR-T regulatory mechanisms as strategies to enhance CAR-T cell efficacy.

FIG. 6 illustrates an example approach for controlling CAR-T cell activation using targeted CAR internalization and/or degradation (bispecific modulators, including TransTAC molecules)(TransTAC is Transferrin receptor-mediated TArgeting Chimera), as disclosed herein.

FIG. 7 illustrates an example schematic of TransTAC technology (e.g., a type of bispecific modulator). Extracellular proteins (e.g., membrane proteins with extracellular domains) can be selectively internalized and degraded by tethering, for example, an antibody to (or a ligand of) the target membrane protein to a transferrin receptor with a bispecific modulator, shown as a membrane protein-specific antibody-transferrin fusion protein.

FIG. 8 illustrates another example schematic of bispecific modulator/TransTAC technology. Not discussed in text.

FIG. 9 illustrates another example schematic of bispecific modulator/TransTAC technology. Not discussed in text.

FIG. 10 illustrates results showing expression of an anti-EGFR affibody-Fc-Tr TransTAC molecule (Left) and the effect on EGFR levels of incubating the TransTAC molecule with a MCF10A EGFR overexpression cell line (Right).

FIG. 11A-B illustrates results showing the effect on EGFR levels of incubating a TransTAC molecule with A549 cells (A) and killing of MCF10A EGFR cells (B).

FIG. 12 illustrates results showing that TransTAC targeting effectively internalized the receptor.

FIG. 13 illustrates example results showing expression of various TransTAC proteins in cultured cells.

FIG. 14 illustrates results showing TransTAC targeting of a CAR specific for CD19 effectively decreased levels of the CAR (e.g., internalized the CAR).

FIG. 15 illustrates example results showing that TransTAC targeting of a CAR specific for CD19 effectively internalized the CAR.

FIG. 16 illustrates example results showing that TransTAC targeting of a CAR specific for CD19 on Jurkat cells, in the presence of K562 cells, inhibited activation of the Jurkat cells.

FIG. 17 illustrates example results showing TransTAC targeting of a CAR specific for CD19 internalized the CAR/inhibited CAR-T activation.

FIG. 18 illustrates results showing that a TransTAC molecule specific for CD19 blocks CAR-T cell activation in presence of K562 cells, and also shows that the TransTAC molecule had minimal effect on the Jurkat cells in absence of the K562 cells.

FIG. 19A-B illustrates a schematic diagram of a CARTrap molecule (domain to which CAR can bind fused to an Fc region), a TransTAC molecule that can bind a CAR and an internalizing receptor, and a dimer of a TransTAC molecule that can bind a CAR and an internalizing receptor (A), and results of using the molecules on cells expressing the CAR on the levels of the CAR (B).

FIG. 19C illustrates fluorescence microscopy of the targeted CAR on the cells in FIG. 19B.

FIGS. 20A-B, 20C, 20D-E, 20F-G, 20H and 201 illustrate an embodiment where an internalized CAR is not degraded, but linker engineering (here, incorporating a cathepsin-sensitive linker) resulted in degradation of the CAR. (A) shows schematic diagrams of various molecules used in this study. GFLG indicates a Gly-Phe-Leu-Gly peptide linker which is sensitive to lysosomal cathepsin proteases. (B) shows Western blots of the targeted CAR (Anti-CD3z) and actin control (β-Actin) when various of the molecules in (A) were used. (C) shows a graph of the data from (B), where CAR levels are normalized to 3-Actin levels. (D) shows Western blots as above, using other of the molecules in (A). (E) shows a graph of the normalized data from (D). (F) shows a schematic of a dimer of a TransTAC molecule that can bind a CAR and an internalizing receptor, which also contains GFLG linkers. (G) shows Western blots using the molecule shown in (F). (H) shows a graph of the normalized data from (F). (I) shows results from screening additional cathepsin-sensitive TransTAC molecules that have improved inhibition potency.

FIG. 21A-B shows results demonstrating TransTAC inhibition of T cell activity is more potent than inhibition by CARTrap (domain to which CAR can bind fused to an Fc region) in Jurkat cells (A) and in primary T cells (B).

FIG. 22A and FIG. 22B show results illustrating that CAR-TransTAC turns off tumor cell killing of the CAR-T cell, and that tumor cell killing of the CAR-T resumes when the TransTAC molecule is removed.

FIG. 23A-B shows schematic diagrams of molecules used in the study, including affibody-based EGFR TransTAC molecules (A). (B) shows results of removing EGFR from the surface of A549 cells using the molecules shown in (A). The data show that use of the affibody-based TransTAC molecule produces good results (approximately 10-50-fold improvement in IC₅₀).

FIG. 23C-D shows results of inhibition of cell proliferation by the molecules shown in (A), as measured using the MTT cellular proliferation assay. The data show that use of the affibody-based TransTAC molecule produces good results (approximately 10-50-fold improvement in IC₅₀).

FIG. 24A-B shows a schematic of molecules used in this study (B) and western blot results measuring internalization and degradation of the molecules (C).

FIG. 24C shows a graph of the normalized data from FIG. 54(C).

FIG. 25A-B illustrates example data demonstrating protein internalization by TransTAC and reversibility of the internalization.

FIG. 26 shows example data demonstrating TransTAC can interfere with IFNγ production.

FIGS. 27A and 27B show example data of transferrin receptor expression on various cells.

FIG. 28 shows some examples of of cell-surface molecules that can be regulated by TransTAC.

FIGS. 29A and 29B show example data demonstrating that TransTAC can degrade EGFR in cells and the underlying cellular machinery that mediates the degradation.

FIGS. 30A, 30B and 30C-D shows example approaches for treating lung cancer with TransTAC.

FIG. 31 shows example data demonstrating that TransTAC with the linker variants can degrade CAR in CAR-Jurkat cells.

FIGS. 32A and 32B shows example data demonstrating that TransTAC can degrade PD-L1 in breast cancer cells.

FIG. 33 shows example data demonstrating that TransTAC can degrade CD20 in lymphoma cells.

FIGS. 34A-C and 34D show example TransTAC molecules that include protease-sensitive linkers and example data obtained with the molecules.

FIG. 35 shows example data obtained with TransTAC molecules containing various protease-sensitive linkers.

FIG. 36A-C show example TransTAC molecules that include an antibody fragment specific for transferrin binding and example data obtained with the molecules.

FIG. 37A-B show examples of TransTAC molecules and example data obtained with the molecules.

FIGS. 38A-E and 38F-H show an example overview of the TransTAC technology and TfR expression analysis. (A) Schematic of the example TransTAC technology. TransTAC induces close proximity of TfR and POI at the cell surface, leading to co-internalization of the complex to early endosomes (EE), where a cathepsin enzyme cleaves TransTAC and separates the POI from the TfR. The POI then traffics to late endosomes (LE)/lysosomes for degradation, while TfR is recycled back to the cell surface. (B) Illustration of an example TransTAC protein. Some example designs to make TransTACs efficient degraders include: (1) containing two anti-TfR binders for binding and priming a TfR dimer for endocytosis, (2) having a cathepsin B-sensitive linker between the anti-POI binder and the Fc for endosomal cleavage to separate the POI from the recycling TfRs, and (3) using an antibody binder instead of a native TF ligand to reduce trafficking to the recycling endosomes (REs). (C) Relative cell surface TfR expression levels across various non-tumorigenic and cancer cell lines characterized by flow cytometry. Cancer cell lines express higher levels of TfR compared to non-tumorigenic cell lines. Data are representative of 3 independent experiments. (D) Relative TFRC RNA expression levels in primary tumor compared to normal tissues based on the MERAV database. TFRC expression is significantly higher in most tumors than the corresponding normal tissues. T-test in (FIGS. 38F and G shows significance for comparing tumors to healthy tissue overall (p=3.98e−89), and for 14 out of 19 of the individual tumor/healthy tissue pairs. Female reproductive tissues are the endometrium, cervix, fallopian tubes, myometrium, ovary, placenta, and uterus. Central nervous system (CNS) tissues are the basal ganglia, brainstem, cerebral cortex, hippocampus, spinal cord, and vestibular nuclei superior. Brain tissues are hypothalamus, pituitary gland, thalamus, ganglia, and ganglion nodose). (E) Relative TFRC RNA expression levels in native T cells. TfR is upregulated by approximately 6-fold in activated CD4 and CD8 T cells compared to inactivated T cells with statistical significance (p=1.25e−68 for CD4 T cells and 4.81e−68 for CD8 T cells, FIG. 38H).

FIG. 39A-L shows example TransTAC degrader engineering. (A) Schematic of example CAR-TransTACs and control. TransTACv0.1 has a single CD19NT.1 domain, a single TF, and a knob-in-hole (KIH) Fc, v0.2 has two CD19NT.1s, two TFs, and a homodimeric Fc that connects the binders, v0.4 contains a cathepsin-sensitive linker between CD19NT.1 and Fc, v0.5 contains a H7 scFv for TfR binding, v1.0 contains both the H7 and the cathepsin sensitive linker. (B) Schematic of a myc-tagged anti-CD19 CAR receptor. (C) Flow cytometry measurements of cell-surface CAR expression levels in CAR-Jurkats treated with TransTACv0.1, v0.2, and control. TransTACv0.2 results in higher CAR clearance from cell surface than v0.1 and no hook effect. Data are representative of 2 independent experiments. (D) Characterization of whole-cell CAR levels by Western blot in CAR-Jurkats treated with TransTACs. TransTACv1.0 degrades approximately 80% of CAR; v0.2 didn't result in significant CAR degradation. (E-H) Schematics showing different TransTACs alter intracellular trafficking of the POI. Cleavage of the cathepsin sensitive-linker in v0.4 and v1.0 leads to separation of POI from TfR, hence enhanced LE/lysosomal trafficking of the POI and degradation; the H7 scFv in v0.5 and v1.0 reduces trafficking of the complex to the REs, hence increases the proportion of POI in EEs and subsequent proteolytic processing, when a cleavage linker is present. (I, J) Representative fluorescence images of Hela cells co-expressing CAR-GFP (green) and endosomal/lysosomal markers-mCherry (red) treated with various TransTAC molecules. Cell nucleus is stained with Hochest (blue). Untreated (UT) or control-treated cells had CAR-GFP localized at the cell membrane. v0.5 and v1.0 led to efficient degradation of CAR-GFP, manifested by the significantly lower GFP signals. v0.2-treated cells predominantly trafficked CAR to the REs, showing co-localization of CAR-GFP with mCherry-Rab11 (white arrows). v0.5-treated cells trafficked CAR to the EEs, showing co-localization of CAR-GFP with mCherry-Rab5 (white arrows). (K) Pearson correlation analysis of CAR-GFP colocalization with the Rab5 (EE), EEA1 (EE), and Rab11 (RE) markers. T-tests show Rab5, EEA1, Rab11 colocalization with CAR are statistically different for cells treated with v0.2 vs. v0.5. (L) Pearson correlation analysis of CAR-GFP colocalization with the Rab7 (LE) and Lamp1 (lysosome) markers. T-tests show Rab7 and Lamp1 colocalization with CAR are statistically significant for v0.2 vs. v0.4, and v0.5 vs. v1.0. For k and 1, number of cells used for each analysis are as follows: For v0.2, N=12, N=12, and N=13 for the EEA1, Rab5 and Rab 11 markers, respectively. For v0.5, N=10, N=22, and N=15 for the EEA1, Rab5 and Rab11 markers respectively. For v0.2, v0.4, v0.5, and v1.0 with the Lamp1 marker N=16, N=21, N=13, and N=13, respectively.

FIG. 40A-D shows examples of Developing TransTACs degraders for various membrane targets. (A) Schematic of membrane proteins targeted by TransTACs in the present study. These targets are either synthetic, or native, single- or multi-pass proteins expressed on cancer or immune cell surface. (B) PD-L1 degradation by TransTACs in MDA-MB-231 breast cancer cells analyzed by Western blot. A scFv or Fab format of atezolizumab is used as the PDL1 binding moiety. (C) EGFR degradation by TransTAC in A549 lung carcinoma cells. An affibody is used as the EGFR binding moiety. (D) CD20 degradation by TransTAC. A Fab format of rituximab is used as the CD20 binding moiety.

FIGS. 41A-H and 411 show example structure-activity relationship (SAR) studies of TransTACs, mechanisms, and in vivo characterizations. (A) Time-course measurement of cell surface CAR levels in CAR-Jurkats treated with TransTACs, revealing the fast kinetics of TransTAC-mediated CAR internalization. (B) Schematics of CAR-TransTAC variants consisting of one or two copies of anti-POI and anti-TfR binders in different protein geometries. (C) Cell surface CAR level measurements in CAR-Jurkats treated with CAR-TransTAC variants outlined in (B). The results highlight the impacts of having two vs. one TfR binders (v0.5 vs v0.7) and geometry (v0.8 vs. v0.9) in modulating protein internalization. Data are representative of 3 independent measurements. (D) Competition assay with a H7-Fc fusion protein. Concentration-dependent reduction of CAR internalization is observed with H7-Fc, proving internalization is mediated through TfR. Data are representative of 3 independent measurements. (E) Study of underlying degradation pathways with TransTACs. Intact lysosomal function is critical for degradation, as degradation is fully inhibited by bafilomycin in A549 cells treated with EGFR-TransTACs. (F) Whole-cell TfR level measurement with TransTAC treatment. TfR level stays consistent while PD-L1 is degraded in MDA-MB-231 cells treated with PDL1 TransTAC. (G) Schematic of mouse experiments to assess TransTAC safety and serum half-life via IP injection. (H) Weight monitoring of mice over time after TransTAC or control IgG injection. Results reveal no observable effects on mouse weight over time, showing molecules are well tolerated. N=2 per treatment group. (I) Western blot quantification of plasma levels of CD20-TransTAC and IgG control over time. N=2 per treatment group.

FIG. 42A-G shows example targeting of TKI-resistant lung cancer cells by EGFR-TransTACs. (A) Schematic representation of development of drug-resistant mutations in lung cancer cells and available treatment options. EGFR Del19 and L858R mutants can be targeted by first- and second-generation TKIs, T790M can be targeted by osimertinib, but cells that harbor the additional C797S mutation has no available targeted therapy options. (B) Schematic of EGFR TransTACs designed with different cleavable linkers and TfR binders. (C) Cell viability assay for PC9 WT cells treated with EGFR TransTAC variants illustrated in (B). v0.5 and v1.0 lead to potent cell inhibition; affibody-Fc control or v0.2 have no effect. Data are representative of 3 independent experiments. (D) Western blots showing efficient TransTAC1.0s-mediated EGFR degradation in PC9 WT cells and PC4 GR4 C797S cells. (E) Cell viability assay for lung cancer cells PC9 WT, PC9 GR4, PC9 GR4 C797S, and a normal fibroblast cell line HFF-1 treated with TransTACs and TKIs. PC9 WT cells responded to all three TKIs; PC9-GR4, which contains the T790M mutation, renders resistance to gefitinib; PC9 GR4 C797S renders resistance to osimertinib in addition to afatinib and gefitinib; all three PC9 cell lines were inhibited by EGFR-TransTACs. Neither TKIs nor TransTACs lead to significant toxicities in the HFF-1 cell line. Data are representative of 3 independent experiments. (F) Testing TransTAC efficacy and specificity in a co-culture assay of PC9 WT cancer cells and HFF-1 healthy cells, in comparison to TKIs and carboplatin/paclitaxel chemotherapy combination. PC9 WT cells and HFF-1 cells express GFP and mCherry, respectively. TransTACs and TKIs specifically inhibit PC9 WT cancer cells while sparing HFF-1 cells; chemotherapy inhibits both cell types. (G) Experiment of (F) with PC9 GR4 C797S cancer cell and HFF-1 healthy cell co-cultures. TransTACs and chemotherapy but not TKIs inhibit cancer cells; additionally, TransTACs and TKIs did not cause cytotoxicity to HFF-1 cells, but the chemotherapy treatments inhibit HFF-1s.

FIG. 43A-B shows example characterization of Fc fusions of wildtype (WT) CD19 ectodomain and the variants. CD19ecto-WT-Fc shows aggregations in SDS-PAGE gel while the variants derived from yeast display do not. Among the four variants, CD19NT.1 is selected for CAR-TransTAC engineering given its high expression level.

FIG. 44A-E shows example different CAR degradation efficiencies mediated by TransTAC variants. (A) Schematics of different generations of CAR-TransTACs and the CD19NT.1-Fc control. (B) Western blots showing neither the control nor v0.2 leads to CAR degradation. (C) Western blots showing v0.4-GFLG, which contains a cathepsin sensitive GFLG linker between the CD19NT.1 and Fc domains, leads to approximately 40-50% of CAR degradation. v0.3-GFLG, which contains the cleavable linker between the Fc and the TF domains, doesn't lead to significant CAR degradation, possibly due to Fc mediated CAR recycling. (D) Western blots showing different linker variants of v0.4 lead to varying CAR degradation efficiencies. (E) Western blots showing different linker variants of v1.0 lead to varying CAR degradation efficiencies. Among all variants, linker GFLG-VR and VR show highest degradation.

FIGS. 45A-D and 45E-F shows example colocalization analysis of internalized CAR with various endosomal/lysosomal markers. (A-C) Representative fluorescence images of Hela cells co-expressing CAR-GFP (green) and endosomal/lysosomal markers-mCherry (red), treated with various TransTACs or controls. EEA1: EE marker, Rab7: LE marker, Lamp1: lysosomal marker. Cell nucleus is stained with Hochest (blue). (D) Pearson correlation analysis of CAR-GFP colocalization with the five endosomal/lysosomal markers. T-tests show Rab5, EEA1, Rab11 colocalization with CAR are statistically different for cells treated with v0.2 vs. v0.5, and Rab7 and Lamp1 colocalization with CAR are statistically significant for v0.2 vs. v0.4, and v0.5 vs. v1.0. Cell numbers used for the analysis are as follows: For the control, UT, v0.2, v0.4, v0.5, and v1.0, N=5, N=4, N=12, N=15, N=10, and N=11, respectively for the EEA1 marker. For the control, UT, v0.2, v0.4, v0.5, and v1.0, N=12, N=11, N=12, N=15, N=22, and N=17, respectively for the Rab5 marker. For the control, UT, v0.2, v0.4, v0.5, and v1.0, N=9, N=7, N=8, N=12, N=26, and N=11, respectively for the Rab7 marker. For the control, UT, v0.2, v0.4, v0.5, and v1.0, N=9, N=6, N=13, N=13, N=15, and N=22, respectively for the Rabi 1 marker. For the control, UT, v0.2, v0.4, v0.5, and v1.0, N=17, N=12, N=16, N=21, N=13, and N=13, respectively for the Lamp1marker. (E-F) Incorporating a cathepsin sensitive linker into TransTAC enhances LE/lysosomal trafficking. Representative fluorescence images of Hela cells co-expressing CAR-GFP (green) and mCherry-Rab7 or Lamp1-mCherry (red), treated with TransTACv0.2 vs v0.4. Cell nucleus is stained with Hochest (blue). The v0.4 GFP images were collected with 1000× more exposure than the v0.2 images to get sufficient GFP signals for the Pearson coefficient analysis in FIG. 2i. v0.4-treated cells show colocalization of the internalized CAR-GFP with mCherry-Rab7 and Lamp1 (white arrows).

FIG. 46A-C shows example characterizing TransTAC degraders for various membrane proteins. Different linkers and geometry designs lead to varied degradation efficiencies. (A) Western blots showing controls or v0.2 and v0.4 PDL1 TransTAC variants do not lead to much target degradation in MDA-MB-231 cells. (B) Western blots showing EGFR TransTACv0.2 does not cause much target degradation in A549 cells, whereas v0.4 or v1.0 with different linkers cause varying degrees of degradation, with v1.0-EVR and GFLG-VR giving the highest degradation efficiency. (C) Western blots showing a rituximab-scFv-Fc control does not lead to significant CD20 degradation in Raji cells.

FIG. 47A-E shows examples of TransTAC regulating primary CAR-T cell activities. (A) Schematic of using CAR-TransTAC to reversibly control CAR-T cells. TransTAC-mediated removal of CAR from cell surface prevents CAR-T cells from engaging with CD19+ tumor cells, hence inhibits cytokine release and cytotoxicity. (B) Schematic of the setup of a primary CAR-T cell co-culture assay. Secreted IFN-γ levels are measured to determine CAR-T cell activation levels in the presence if CD19+A375 cells and TransTACs; live cell fluorescence microscopy is used to determine the antitumor effects. (C) Measurement of human primary CAR-T cell IFN-γ release in the co-culture assay described in (b) with an IFNγ split-luciferase assay (Promega). IFN-γ secretion is inhibited by TransTACv0.4 in a dose-dependent manner. TransTAC shows an IC50 Of approximately 0.4 nM. Data are representative of 2 independent experiments. (D) Fluorescence microscopy of mCherry-labeled A375 cells showing CAR-T cell-mediated A375 killing reversibly controlled with CAR-TransTACv4. (E) Overlay of bright field and mCherry channel images showing CAR-T cell-mediate A375 killing activity was resumed after TransTAC washout over time.

FIG. 48A-C shows example characterization of EGFR TransTACs. (A) Western blots showing EGFR-TransTAC leads to 40-50% target degradation in HEK293 cells overexpressing EGFR, a level significantly lower than A549 and PC9 cells, potentially due to lower levels of TfR expression. (B) IC50s of TransTACv1.0s and TKIs afatinib, gefitinib, and osimertinib in PC9 cells based on data presented elsewhere. (C) Flow cytometry analysis of the PC9 (GFP)/HFF-1(mCherry) cell ratios reflecting different sensitivities of the tumor/healthy cells to various treatments. Data are representative of 3 independent experiments.

FIG. 49A-B shows (A) Cleavage of the indicated linkers on yeast at pH 4.4 by recombinant cathepsin B as compared to cleavage of GFLGGVR (SEQ ID NO: 144). (B) Cleavage of the indicated linkers on yeast at pH 6.4 by recombinant cathepsin B as compared to GFLGGVR (SEQ ID NO: 144).

### DETAILED DESCRIPTION

Targeted protein degradation (TPD) is a rapidly growing field in drug discovery and pharmacology. Complementing traditional drug modalities, TPD molecules offer a novel therapeutic mechanism to tackle challenging targets, increase the therapeutic potential of currently used drugs, and the like. While many efforts in this field have focused on small molecules for intracellular targets, inducing targeted degradation of extracellular proteins is a new opportunity. Developing strategies to degrade extracellular proteins is of exceptional interest for both basic research and therapeutic intervention purposes.

Iron is an essential element for cells, and its transportation is facilitated by transferrin receptor (TfR). TfR undergoes rapid endocytosis as a recycling receptor, with an average internalization rate of 500 molecules per cell per second, making it one of the fastest internalizing receptors known. Furthermore, TfR is upregulated in cells that have a high demand for iron. This includes rapidly dividing cancer cells and activated T cells. TfR expression in these cells are higher than in non- or slowly-dividing normal tissues. TfR can be expressed on non-cancer cells at sufficient levels where the reagents and methods described herein can be used.

Herein, these features of TfR were leveraged and employed protein engineering strategies to develop a new technology for degrading membrane proteins. Herein, this technology is called receptor-mediated Targeting Chimeras (TransTACs). In some embodiments, TransTACs are heterobispecific antibodies that bring the protein of interest (POI) and TfR in close proximity at the cell surface and induce endocytosis of the POI/TfR complex and subsequent lysosomal-mediated POI degradation. TransTACs are effective in degrading various types of membrane proteins, including single-pass, multi-pass, native, and synthetic receptors, showing a degradation efficiency of over 80% for all targets in various cellular systems. A notable characteristic of TransTACs is its fast kinetics of targeted internalization, occurring on a timescale of minutes, making it a valuable molecular tool for rapidly knocking down cell-surface expression, offering temporal specificity for cell signaling studies that is not possible with genetic approaches. Moreover, TransTAC molecules are fully recombinant, modular, and cancer specific. These properties make TransTACs a versatile technology for manipulating cell surface targets in disease-specific manners.

TransTAC can have broad applicability in both basic research and translational applications. Herein is demonstrated non-limiting applications of TransTACs in reversible control of CAR-T cells, targeting of drug-resistant EGFR positive lung cancers, and others. TransTACs represent a new molecular archetype to control cell surface proteins.

TransTAC is the first bispecific antibody technology that repurposes a recycling ligand/receptor interaction for targeted protein internalization and degradation, which can significantly expand the scope of effectors at the cell surface amenable for such purposes.

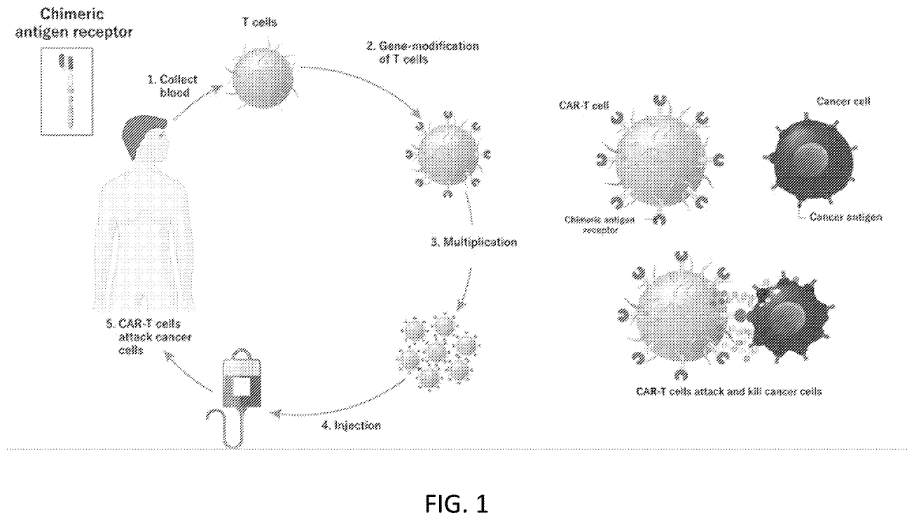

Chimeric antigen receptor (CAR) T cells have emerged as a promising therapy for patients with hematologic malignancies (FIG. 1). In some cases, however, CAR-T therapies can cause side effects in patients who receive these cells, including cytokine-release syndrome (CRS) and immune effector cell-associated neurotoxicity syndrome (ICANS).

Several regulatory mechanisms to control CAR-T cells in vivo have been developed to address these adverse events. However, these strategies have not satisfactorily met current needs since toxicities and fatalities continue to be reported in CAR-T clinical trials.

Herein are disclosed new, modular, and reversible strategies for modulating CAR-T cell activities. Generally, the methods do not require additional genetic engineering of CAR-T cells. In some embodiments, these strategies can modulate CAR-T toxicities. In some embodiments, these strategies can increase efficacy of CAR-T cell therapy. In some embodiments, these strategies are based on reversible internalization of CAR receptors.

In some embodiments, a bispecific modulator, including for example a transferrin receptor-mediated targeting chimera or TransTAC molecule, can colocalize a CAR receptor to an internalizing cell surface protein. In embodiments, bispecific modulators can downregulate cell surface levels of CAR. In embodiments, the bispecific modulators can inhibit CAR-T cell activation and/or function.

In embodiments, the bispecific modulators can have a first portion or moiety that is an antibody, antibody fragment or an alternative antibody scalfold that specifically binds to a target molecule on a cell (e.g., molecules of interest, such as proteins of interest, like a CAR, an EGFR, a CD20), and a second portion or moiety (e.g., transferrin or an antibody or antibody fragment) that can bind to an internalizing molecule on the cell surface (e.g., transferrin receptor). In embodiments, the bispecific modulators can have a first portion that is an antigen or ligand for a target protein on a cell (e.g., CD19 antigen or its variants for a CD19-specific CAR). In embodiments, the bispecific modulators can have a first portion that is an antibody or antibody fragment that can specifically bind a target molecule (e.g., molecule of interest) on a cell. The bispecific modulators can have a second portion that binds to an internalizing protein on the cell surface (e.g., transferrin receptor). Binding of a bispecific modulator to the target molecule and to the internalizing protein results in internalization of the target molecule.

In some embodiments, the bispecific modulators do not require engineering of the CAR-T receptor or CAR-T cells and can be applied to CAR-T therapies that are already approved or in clinical development.

In other embodiments, the bispecific modulators can be reversible. Reversibility can provide for fine tuning of CAR-T cell activities, for example for toxicity management and/or to rejuvenate the cells for continued treatment.

In some embodiments, bispecific modulators can be tailored to CAR-T cells that target different tumor antigens, for example, by replacing the components used in the designs (i.e., the traps and/or modulators can be modular).

Also disclosed are approaches for enhancing CAR-T efficacy. Temporal “rest” of CAR-T cells can reverse CAR-T exhaustion. In some embodiments, by alternating CAR-T cells between an “active” and a “resting” state, the disclosed reversible CAR modulators can increase efficacy of CAR-T cells.

Also disclosed are approaches for targeting cancer cells, including cancer cells with drug-resistant mutations. Cancers can evolve rapidly to evade therapy, often developing drug-resistant mutations that lead to treatment failure and disease recurrence. The C797S mutation of EGFR, for instance, poses a challenge in the treatment of non-small cell lung cancer (NSCLC). Emerging in roughly 10-26% of NSCLC patients following treatment with the third-generation EGFR tyrosine kinase inhibitor (TKI) Osimertinib, the C797S mutation affects a critical residue, C797, which forms covalent bonds with irreversible TKIs. Consequently, existing TKIs become ineffective against the disease. There is a need to develop drugs that target these drug-resistant oncogenes.

Disclosed are development of EGFR TransTAC degraders to target EGFR-driven lung cancers including patient populations with the C797S mutation. Herein is shown that (1) an EGFR TransTAC can effectively degrade drug resistant EGFR mutant proteins and hence inhibit cancer growth, and (2) EGFR TransTAC can specifically target cancer cells while sparing healthy cells because of the overexpression of TfR on cancer cells. The reagents and methods disclosed herein can be used on cells that are not cancer cells.

Detailed descriptions of one or more embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

As used herein, the term “about” can refer to approximately, roughly, around, or in the region of. When the term “about” is used in conjunction with a numerical range, it modifies that range by extending the boundaries above and below the numerical values set forth. The term “about” is used herein to modify a numerical value above and below the stated value by a variance of 20 percent up or down (higher or lower).

### CAR-T Cells, Toxicities and Controlling Toxicities

Chimeric antigen receptor (CAR) T cells have emerged as a promising treatment for patients with advanced B-cell cancers (FIG. 1). However, widespread application of the therapy can be limited by potentially life-threatening toxicities due to a lack of control of the transfused CAR-T cells. Toxicities are an obstacle for the development of CAR-T therapy for both blood cancers and solid tumors. Reported deaths from CAR-T therapies have recently been discussed in the literature (Neelapu, Sattva S., et al. “Toxicity management after chimeric antigen receptor T cell therapy: one size does not fit ‘ALL’.” Nature reviews Clinical oncology 15.4 (2018): 218-218).

Cytokine-release syndrome (CRS) and immune effector cell-associated neurotoxicity syndrome (ICANS) are the two most-common toxicities observed after CAR-T-cell therapy.

CRS can be characterized by high fever, hypoxia, hypotension, or multiorgan toxicity; it develops in 37%-93% patients with lymphoma and 77%-93% with leukemia. ICANS is characterized by confusion, delirium, seizures, or cerebral oedema; it develops in 23%-67% patients with lymphoma and 40%-62% with leukemia. Severe CRS and ICANS require monitoring and treatment in the intensive-care setting, and multiple fatalities have been reported due to unmanageable CRS or ICANS toxicities.

Three types of treatments for these toxicities are currently in use.

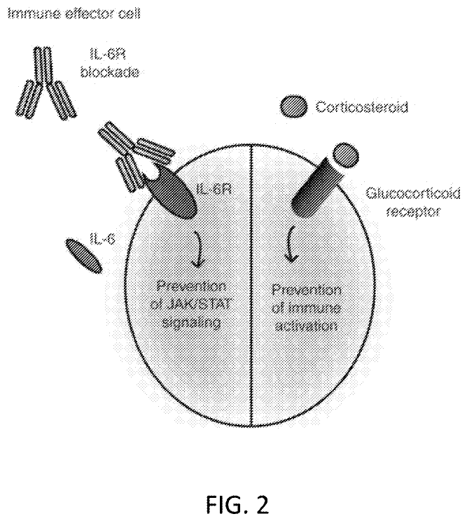

In some instances, patients are treated with immunosuppressive agents (FIG. 2), including systemic corticosteroids, IL-6 receptor antibody (e.g., tocilizumab), lymphocytotoxic anti-CD52 antibody (e.g., alemtuzumab), tyrosine kinase inhibitors (e.g., dasatinib) (LCK inhibitors do not inhibit already activated T cells) and the like. There are limitations to these treatments, however. For example, treatment with high-dose steroids can limit the time span over which CAR-T cells are functional and can induce hematological aplasia and toxicity. Anti-IL6 receptor antibody has a variety of biological activities and can non-specifically inhibit the immune system (Bonifant, Challice L., et al. “Toxicity and management in CAR T-cell therapy.” Molecular Therapy-Oncolytics 3 (2016): 16011).

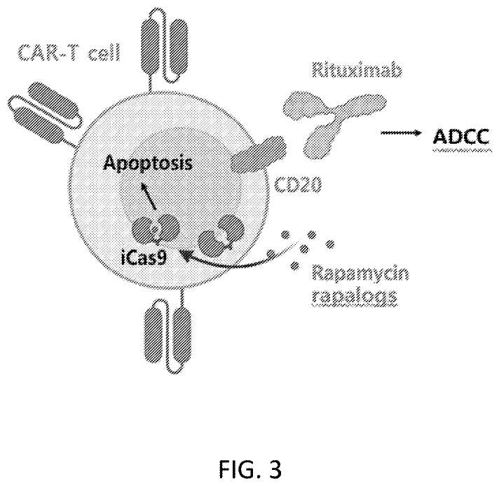

In some instances, patients are treated with suicide genes or elimination markers (FIG. 3), including iCasp9, anti-CD20 (e.g., rituximab), anti-EGFR (e.g., cetuximab) and the like. There are limitations to these treatments, however. For example, these treatments can irreversibly and/or permanently eliminate CAR-T cells from the body (Brandt, Lorke J B, et al. “Emerging approaches for regulation and control of CAR T cells: a mini review.” Frontiers in Immunology 11 (2020): 326).

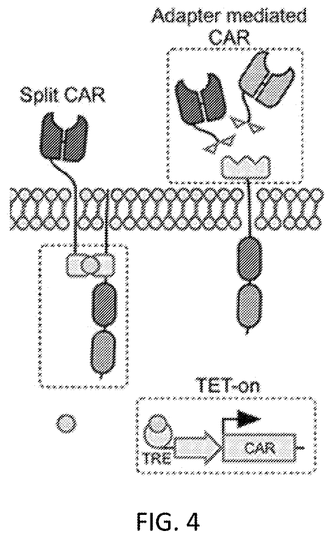

In some instances, CAR-T cells that have switchable CAR receptors can be used in patients (FIG. 4), including split-CAR, SMaSh-CAR, CAR PROTAC and the like. There are limitations to these treatments, however. For example, these treatments can compromise CAR-T activity, the switches can be leaky, and the switches can be immunogenic (Labanieh, Louai, et al. “Enhanced safety and efficacy of protease-regulated CAR-T cell receptors.” Cell 185.10 (2022): 1745-1763).

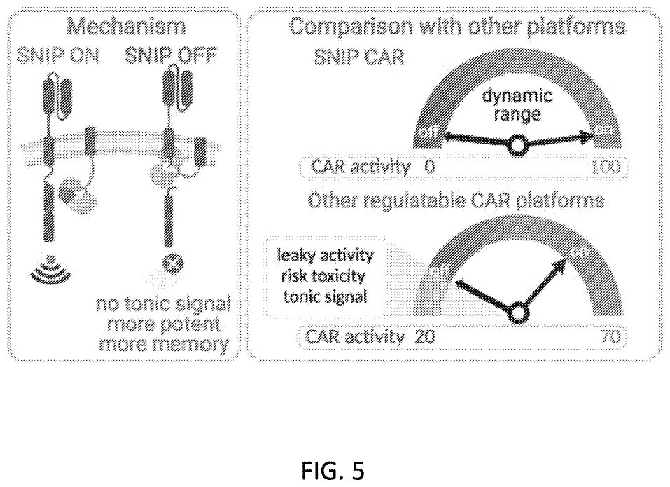

It is known, however, that reversible CAR-T regulatory mechanisms can be used to enhance CAR-T efficacy (FIG. 5). Constitutive CAR-T cells can manifest increased levels of exhaustion-associated proteins. In some embodiments, however, transient “rest” can reverse the exhaustion phenotype. In some embodiments, regulated CAR can be reversibly turned off and on to switch the CAR-T cells between an “Off” and “On” state (Weber, Evan W., et al. “Transient rest restores functionality in exhausted CAR-T cells through epigenetic remodeling.” Science 372.6537 (2021): eabal786; Labanieh, Louai, et al. “Enhanced safety and efficacy of protease-regulated CAR-T cell receptors.” Cell 185.10 (2022): 1745-1763).

In some embodiments of the invention disclosed herein, bispecific modulators (e.g., TransTAC molecules) are used to regulate CAR-T cells. In some embodiments, these bispecific modulators can reversibly modulate CAR-T cells.

### Bispecific Modulators

In some embodiments, strategies disclosed herein for regulating a molecule on a cell surface (e.g., molecules of interest, such as proteins of interest) and/or regulatory activity of such molecules can use a bispecific modulator approach. In some embodiments, a bispecific modulator molecule can have at least two moieties. A first moiety can be a ligand that the cell-surface molecule(s) can bind or an antibody or antibody fragment that can bind to the cell-surface molecule(s) (e.g., molecules of interest, such as proteins of interest). A second moiety can be a molecule that can bind to an internalizing receptor or membrane protein on a cell. In embodiments, a second molecule can be an antibody or antibody fragment that binds to an internalizing receptor or membrane protein on a cell. In embodiments, the bispecific modulator can be a bispecific antibody.

In some embodiments, the bispecific modulator approach can regulate molecules other than those on a cell surface. In some embodiments, the bispecific modulator can bind and internalize (and, optionally, degrade) proteins present in the extracellular/external environment. In some embodiments, these can be soluble proteins. In some embodiments, these proteins can include, as non-limiting examples, autoantibodies, cytokines, enzymes and the like.

In embodiments, a bispecific modulator having these two moieties can bind, or be bound by, a cell-surface or other molecule (e.g., molecules of interest, such as proteins of interest), and can bind to an internalizing receptor or membrane protein. After such bindings, the internalizing receptor or membrane protein can cause the cell-surface or other molecule to be internalized into the cell (e.g., endocytosis). In embodiments, the internalized cell-surface or other molecule can be degraded. In embodiments, this decreases the amount of the cell-surface molecule on the surface of a cell. In embodiments, the internalized cell-surface molecules are not functional. In some embodiments, the cell-surface molecule that is targeted by the first moiety of a bispecific modulator is different than the molecule targeted by the second moiety).

In some embodiments, administering the bispecific modulators to a subject can be used for targeted internalization of membrane or other proteins. In some embodiments, administering the bispecific modulators to a subject can be used for targeted degradation of membrane or other proteins.

In some embodiments, adding the bispecific modulators to cells or administering to a patient can cause targeted internalization and/or degradation of proteins on the surface of a cell or outside of a cell. In some embodiments, this internalization/degradation is reversible. For example, when a cell is no longer exposed to bispecific modulators, the membrane proteins to which the bispecific modulators are specific are no longer internalized/degraded. Generally, the membrane proteins are still synthesized and trafficked to the cell membrane. Therefore, when the bispecific modulators are removed or are no longer administered to a subject, there is not a stimulus to internalize/degrade the proteins. In some embodiments, a cellular membrane protein that can be internalized by a bispecific modulator, but not degraded, can be both internalized and degraded using a bispecific modulator that also contains a protease-sensitive linker. As discussed elsewhere, placement of a protease-sensitive linker within a bispecific modulator can provide release of a targeted cellular protein of interest from the bispecific modulator inside of a cell.

In some embodiments, internalization and degradation of the cell surface or other molecule (e.g., molecules of interest, such as proteins of interest) can kill the cell (e.g., in embodiments where the cell surface molecule is required for cell viability or cell division; EGFR in some embodiments). In some embodiments, internalization and degradation of the cell surface or other molecule does not kill the cell (e.g., in embodiments where the cell surface or other molecule is not required for cell viability or cell division: CAR in some embodiments).

In some embodiments, internalization of bispecific modulators or parts thereof can involve receptor-mediated endocytosis, also called clathrin-mediated endocytosis. In some embodiments, internalization of bispecific modulators can involve clathrin-independent endocytosis. In some embodiments, internalization of bispecific modulators can involve phagocytosis.

In embodiments, the cell-surface molecule, or molecule that is targeted by the first moiety (e.g., a molecule of interest, such as a protein of interest), can be a CAR molecule. In some embodiments, the CAR molecule can be on a CAR-T cell. In some embodiments, a strategy for regulating CAR-T activities includes internalizing CAR receptors with a bispecific modulator. In some embodiments, the molecule of interest targeted by the first moiety can be a cell regulator, like proteins that are part of immune checkpoint pathways (e.g., PD-L1) or other signal-transducing protein (e.g., EGFR). In some embodiments the molecule of interest can be a marker of a certain cell type (e.g., CD20 for B-cells).

In some embodiments, the molecule of interest targeted by the first moiety can be a protein. In some embodiments, the molecule of interest can be a membrane protein. The membrane protein can be an integral membrane protein. The membrane protein can be a transmembrane protein that has one or more transmembrane domains. In some embodiments, the molecule of interest can be a molecule external to a cell, for example, an autoantibody, cytokine, enzyme, and the like.

In some embodiments, the molecule of interest can bind a hormone, cytokine, growth factor, neurotransmitter, lipophilic signaling molecule (e.g., prostaglandin) or cell recognition molecule (e.g., integrin, selectin). The molecule of interest can be a receptor. The receptor can be a G-protein coupled receptor (GPCR), receptor tyrosine kinase (RTK) or transmembrane receptor (TMR).

In some embodiments, the molecule of interest (e.g., molecule of interest, such as a protein of interest) can be a ligand-gated ion channel-linked receptor or an enzyme-linked receptor. Non-limiting embodiments of a ligand-gated ion channel-linked receptor can Na⁺, K⁺, Ca²⁺, or Cl⁻ channels. Non-limiting embodiments of an enzyme-linked receptor can be a receptor tyrosine kinase, tyrosine-kinase-associated receptor (e.g., enzymes that associate with cytokines), receptor-like tyrosine phosphatase (e.g., that remove phosphate groups from tyrosines of intracellular proteins), receptor serine/threonine kinase, receptor guanylyl cyclase, or histidine-kinase-associated receptor. In some embodiments, the molecule of interest can be a tumor-specific antigen (TSA) or tumor-associated antigen (TAA).

In some embodiments, the molecule of interest can be a transporter. In some embodiments, the molecule of interest can be an ion transporter.

In some embodiments, the molecule that is targeted by the first moiety of a bispecific modulator (i.e., molecule of interest) can be a different molecule than the molecule targeted targeted by the second moiety (e.g., an internalizing receptor or membrane protein) of a bispecific modulator.

In some embodiments, the bispecific modulator is a single molecule. In some embodiments, the bispecific modulator can be a single polypeptide. In some embodiments, a single polypeptide can contain both the first moiety and the second moiety of a bispecific modulator.

In some embodiments, the first moiety can be an antigen or epitope to which the molecule of interest can bind. In some embodiments, the antigen or epitope can bind to a receptor on a cell. In some embodiments, the antigen or epitope can be a ligand for the receptor. In embodiments, the receptor can be a chimeric antigen receptor (CAR), T-cell receptor (TCR) or B-cell receptor (BCR).

In embodiments, the first moiety can be an antibody or antibody fragment that binds to an antigen or epitope from and/or specific to a tumor and/or cancer cell. In embodiments, the antigen or epitope that can be bound by a CAR, to which an antibody or antibody fragment can bind, can be CD19, B cell maturation antigen (BCMA), human epidermal growth factor 2 (HER2) and the like.

In some embodiments, the antibody that is the first moiety can be an scFv, Fab, single-domain antibody, nanobody, monobody, DARPin or affibody. Antibody fragments and other molecules that can be used are described in the section titled, “Antibodies” in this application.

In embodiments, the second moiety binds to a receptor or membrane protein on a cell. In some embodiments, the receptor or membrane protein bound by the second moiety is an internalizing receptor or membrane protein. In some embodiments, the internalizing receptor or membrane protein can mediate endocytosis. In some embodiments, the endocytosis can involve clathrin-coated pits. In some embodiments, the endocytosis may be clathrin independent. In some embodiments, the second moiety can bind a receptor that mediates phagocytosis. In embodiments, the second moiety can also be an antibody, antibody fragment, or other molecule.

In some embodiments, the first and/or second moieties can be any type of moiety that can bind to a cell surface molecule or extracellular molecule target. In some embodiments, the first and/or second moieties can be polypeptides, ligands, aptamers, nanoparticles, small molecules and the like.

In embodiments, a non-limiting list of internalizing receptors or membrane proteins that can be used in the bispecific modulators includes G-protein coupled receptors (GPCR), receptor tyrosine kinases (RTK) and transmembrane receptors (TMR) (Xu, Yanjie, et al. “Endocytosis and membrane receptor internalization: implication of F-BAR protein Carom.” Frontiers in bioscience (Landmark edition) 22 (2017): 1439). In some embodiments, GPCRs can include adrenoceptors, chemokine receptors, coagulation receptors and the like. In embodiments, RTKs can include colony stimulating factor receptors, epidermal growth factor receptors, tyrosine kinase receptors, fibroblast growth factor receptors, insulin-like growth factor receptors, platelet-derived growth factor receptors, transforming growth factor receptors and the like. In some embodiments, TMRs can include folate receptors, interleukin receptors (e.g., IL-2 receptors), low density lipoprotein receptors, transferrin receptors and the like.

In embodiments, a non-limiting list of internalizing receptors or membrane proteins that can be used in the bispecific modulators includes G-protein coupled receptors (GPCR), receptor tyrosine kinases (RTK) and transmembrane receptors (TMR) (Xu, Yanjie, et al. “Endocytosis and membrane receptor internalization: implication of F-BAR protein Carom.” Frontiers in bioscience (Landmark edition) 22 (2017): 1439). In some embodiments, GPCRs can include adrenoceptors, chemokine receptors, coagulation receptors and the like. In embodiments, RTKs can include colony stimulating factor receptors, epidermal growth factor receptors, tyrosine kinase receptors, fibroblast growth factor receptors, insulin-like growth factor receptors, platelet-derived growth factor receptors, transforming growth factor receptors and the like. In some embodiments, TMRs can include folate receptors, interleukin receptors (e.g., IL-2 receptors), low density lipoprotein receptors, transferrin receptors and the like.

In some embodiments, the internalizing receptor or membrane protein can be a transferrin receptor. In embodiments, a ligand (e.g., second moiety) that a transferrin receptor can bind (first moiety) can be transferrin or a fragment of transferrin. In some embodiments, the first moiety can be an antibody or antibody fragment that can bind transferrin receptor.

In some embodiments, the internalizing receptor or membrane protein can be a transferrin receptor (TfR). The transferrin receptor can be transferrin receptor I or transferrin receptor 2.

In some embodiments, transferrin receptor can have a high endocytosis rate of around 500 molecules per cell per second, making it good for inducing protein endocytosis. In some embodiments, transferrin receptor expression can be low in healthy tissues, but more highly expressed in various tumors and some activated immune cells, like brain, liver, breast, lung, colon and blood cancers.

In embodiments, a ligand (e.g., second moiety) that an internalizing receptor or membrane protein can bind can be at least a part of a naturally-occurring ligand. For example, the ligand can be at least a part of transferrin, cholesterol, low-density lipoprotein, and epidermal growth factor that can be bound by the cognate receptors.

In some embodiments, the ligand bound by a transferrin receptor can be transferrin or a fragment of transferrin. In some embodiments, the ligand that the transferrin receptor can bind can be approximately 80 kDa in size and have glycosylation modifications.

In some embodiments, the second moiety can be an antibody, antibody fragment, or other molecule that can bind the internalizing receptor or membrane protein. In some embodiments, the second moiety can be a scFv, Fab, single-domain antibody, nanobody, monobody, DARPin or affibody.

In some embodiments, an antibody or antibody fragment that can bind transferrin receptor can be an anti-TfR1 antagonistic scFv antibody identified through phage display. This antibody can be called “H7” (Goenaga, Anne-Laure, et al. “Identification and characterization of tumor antigens by using antibody phage display and intrabody strategies.” Molecular immunology 44.15, 2007: 3777-3788; Tillotson, Benjamin J., et al. “Engineering an anti-transferrin receptor ScFv for pH-sensitive binding leads to increased intracellular accumulation.” PLoS One 10.12, 2015: e0145820).

In one embodiment, an amino acid sequence of H7 molecules can include the molecules below, or can include molecules at least 60, 65, 70, 75, 80, 81, 82, 83, 84, 85, 86, 87, 88, 89, 90, 91, 92, 93, 94, 95, 96, 97, 98 or 99 percent identical to the amino acid sequences below:

H7 scFV-LC (SEQ ID NO: 1):
SELTQDPAVSVALGQTVRITCQGDSLRSYYASWYQQKPGQAPVLV
MYGRNERPSGVPDRFSGSKSGTSASLAISGLQPEDEANYYCAGWD
DSLTGPVFGGGTKLTVLG*
H7 scFV-HC (SEQ ID NO: 2):
QVQLQESGGGVVQPGRSLRLSCAASRFTFSSYAMHWVRQAPGKGL
EWVAVISYDGSNKYYADSVKGRFTISRDNSKNTLYLQMNSLRAED
TAVYYCARDLSGYGDYPDYWGQGTLVTVSS
H7-scFv (SEQ ID NO: 3)
SELTQDPAVSVALGQTVRITCQGDSLRSYYASWYQQKPGQAPVLV
MYGRNERPSGVPDRFSGSKSGTSASLAISGLQPEDEANYYCAGWD
DSLTGPVFGGGTKLTVLGGGGGSGGGGSGGGGSQVQLQESGGGVV
QPGRSLRLSCAASRFTFSSYAMHWVRQAPGKGLEWVAVISYDGSN
KYYADSVKGRFTISRDNSKNTLYLQMNSLRAEDTAVYYCARDLSG
YGDYPDYWGQGTLVTVSS
M16 (SEQ ID NO: 4):
SELTQDPAVSVALGQTVRITCQGDSLRSYYASWYQQKPGQAPVLV
MYGRNERPSGVPDRFSGSKSGTSASLAISGLQPEDEANYYCAGWD
DSLTGPVFGGGTKLTVLGGGGGSGGGGGGGGSQVQLQESGGGVVQ
PGRSLRLSCAASRYPFHHHDHHWVRQAPGKGLEWVAVISYDGSNK
YYADSVKGRFTISRDNSKNTLYLQMNSLRAEDTAVYYCARDLSGY
GDYPDYWGQGTLVTVSS

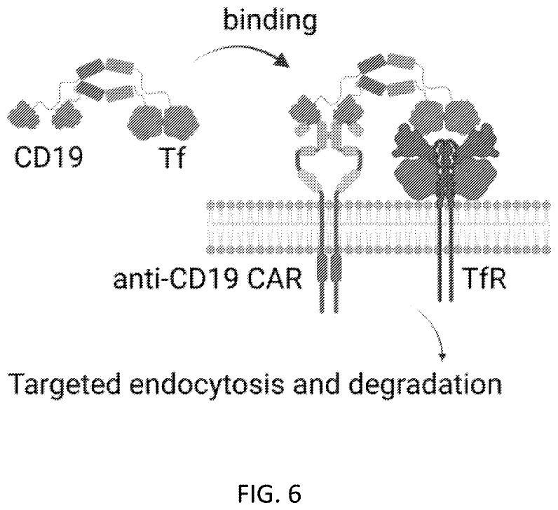

In some embodiments, a strategy for regulating CAR-T activities includes using a molecule (e.g., a bispecific modulator) to which a CAR on the surface of a CAR-T cell can bind (first moiety), and which can bind to an internalizing receptor or membrane protein (second moiety: e.g., transferrin) on the same or adjacent cell(s). The bispecific modulator can co-localize the CAR receptor to the internalizing cell surface receptor or membrane protein. For example, the internalized CARs cannot be activated or does not continue to function in an activated state. In some embodiments, the bispecific modulators can downregulate cell surface levels of CAR and inhibit CAR-T cell function (FIG. 6).

Herein, we disclose new mechanisms for modulating proteins at the cell membrane. Endocytosis is a common machinery of regulating membrane protein recycling and degradation. Among the various transmembrane proteins regulated by endocytosis, transferrin receptor (TfR) is a well-characterized recycling receptor with a rapid internalization rate (500 molecules/cell/s). TfR imports iron by binding to a plasma protein transferrin (Tf) in complex with iron. It is highly expressed in various cancers and affects cancer cells' proliferation, migration, invasion, apoptosis and metastasis.

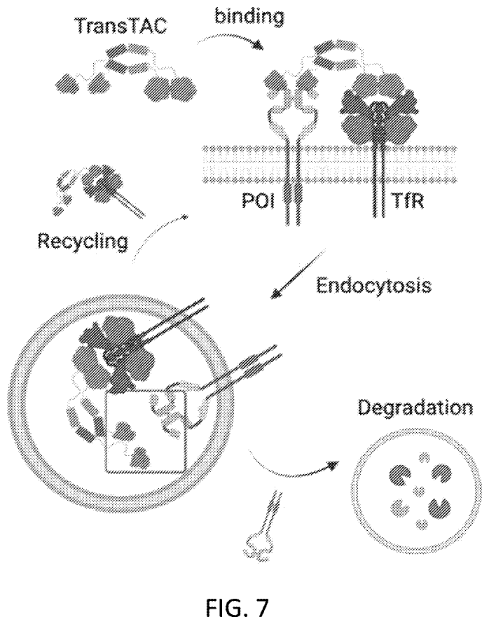

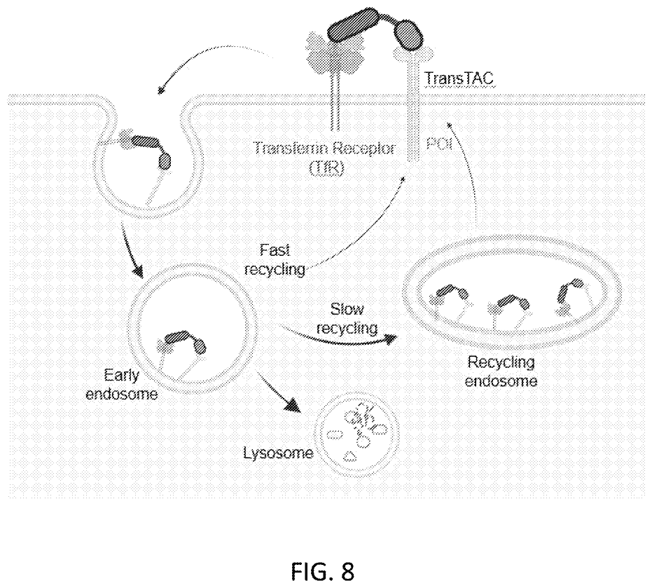

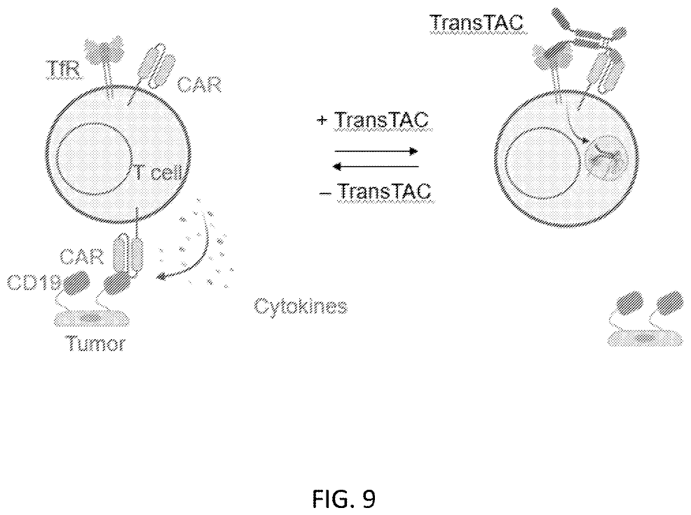

We showed that extracellular proteins, especially tumor-associated proteins of interest (POIs), could be selectively degraded by tethering the POI to a TfR with an antibody-Tf fusion protein (FIG. 7). We name this technology Transferrin receptor-mediated TArgeting_Chimera (TransTAC). Upon TfR/TransTAC mediated endocytosis, the receptor dissociates from the complex due to the different local environment of an endosome (FIG. 7, red square), and undergo lysosomal driven degradation.

The method is a new and generic archetype to degrade proteins with fully recombinant biological molecules. A universal approach for degrading membrane/extracellular proteins would open up unlimited possibilities to manipulate cell behaviors, thus serving as important research tools as well as expanding the PROTAC field's attempts to target challenging extracellular targets. The fully recombinant nature of TransTAC allows for simple generalization to broad range of targets and optimization of binding properties.

TfR-based degradation improves the specificity of tumor targeting. TfR is used because: (1) TfR is a recycling receptor, so the level of TfR on cells could remain consistent which is an important feature as a “carrier” protein; (2) Tf has been explored for iron or small molecule drug delivery and therefore has the necessary developability and stability as a therapeutic agent; and (3) TfR targeting provides additional tumor specificity; its expression is regulated by tumor-associated oxidation stress, inflammation, and hypoxia. Taken together, the modular nature, the genetic tractability, and the tumor specificity of TransTAC is a good approach for academic and translational applications.

In some embodiments, the bispecific modulators disclosed herein have an antigen to which a CAR can bind and a ligand for an internalizing receptor or membrane protein (e.g., transferrin receptor). In some embodiments, the bispecific modulators disclosed herein have an antibody that can bind to a CAR, or to another molecule on the cell surface such as EGFR, PD-L1, CD20, and a ligand for an internalizing receptor or membrane protein (e.g., molecules of interest, such as proteins of interest).

In some embodiments, the bispecific modulators can be fusion proteins of the formula R1-R2-R3. In some embodiments, the bispecific modulators can be fusion proteins of the formula R3-R2-R1. For example, R1 or R3 can be located at the C-terminus or N-terminus of fusion proteins disclosed herein. In some embodiments, the bispecific modulators can be dimers of R1-R2-R3 or R3-R2-R1 (homodimers).

In some embodiments, R1 can be a protein of interest binder (POIB) or a POIB means. In some embodiments, the POIB or means can be an antibody. In some embodiments, the POIB means can be a part of a molecule that the protein of interest can normally bind (e.g., the peptide that a CAR binds). The POIB or means can bind to a molecule of interest, such as a protein of interest, on a cell surface. In some embodiments, the POIB or means can bind to an extracellular domain of a transmembrane protein. In some embodiments, the POIB means can bind to an extracellular domain of a chimeric antigen receptor (CAR), a receptor tyrosine kinase, a checkpoint inhibitor binding molecule, a cell lineage-specific marker, and the like. In some embodiments, the POIB or means can bind to an extracellular domain of an epidermal growth factor receptor (EGFR), a programmed death-ligand (PD-L1) or CD20.

In some embodiments, the POIB means can bind to an extracellular domain of a B cell receptor (BCR), human leukocyte antigen (HLA), fibroblast growth factor receptor (FGFR), Notch proteins, or claudin-18.2.

In some embodiments, R3 can be a transferrin receptor binding means (TRB). The TRB binds to a transferrin receptor on the surface of cells. In some embodiments, the TRB can be an antibody that binds to the transferrin receptor (e.g., H7 or M16). In some embodiments, the TRB can be a polypeptide. In some embodiments, the TRB can be a ligand or part of a ligand to which the transferrin receptor can bind (e.g., transferrin). In some embodiments, a part of transferrin that can be used as a TRB is below (SEQ ID NO: 5):

(SEQ ID NO: 5)
VPDKTVRWCAVSEHEATKCQSFRDHMKSVIPSDGPSVACVKKASY
LDCIRAIAANEADAVTLDAGLVYDAYLAPNNLKPVVAEFYGSKED
PQTFYYAVAVVKKDSGFQMNQLRGKKSCHTGLGRSAGWNIPIGLL
YCDLPEPRKPLEKAVANFFSGSCAPCADGTDFPQLCQLCPGCGCS
TLNQYFGYSGAFKCLKDGAGDVAFVKHSTIFENLANKADRDQYEL
LCLDNTRKPVDEYKDCHLAQVPSHTVVARSMGGKEDLIWELLNQA
QEHFGKDKSKEFQLFSSPHGKDLLFKDSAHGFLKVPPRMDAKMYL
GYEYVTAIRNLREGTCPEAPTDECKPVKWCALSHHERLKCDEWSV
NSVGKIECVSAETTEDCIAKIMNGEADAMSLDGGFVYIAGKCGLV
PVLAENYNKSDNCEDTPEAGYFAIAVVKKSASDLTWDNLKGKKSC
HTAVGRTAGWNIPMGLLYNKINHCRFDEFFSEGCAPGSKKDSSLC
KLCMGSGLNLCEPNNKEGYYGYTGAFRCLVEKGDVAFVKHQTVPQ
NTGGKNPDPWAKNLNEKDYELLCLDGTRKPVEEYANCHLARAPNH
AVVTRKDKEACVHKILRQQQHLFGSNVTDCSGNFCLFRSETKDLL
FRDDTVCLAKLHDRNTYEKYLGEEYVKAVGNLRKCSTSSLLEACT
FRRP.

In some embodiments, R2 can be a linker of the formula R4-R5 or R5-R4. In some embodiments, R4 can be an Fc region from an antibody. In some embodiments, R4 can be an Fc region from IgG, IgM, IgA, IgE or IgD. In some embodiments, an Fc region can be (SEQ ID NO: 6):

DKTHTCPPCPAPELLGGPSVFLFPPKPKDTLMISRTPEVTCVVVD
VSHEDPEVKFNWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

In some embodiments, the Fc regions can dimerize, forming homodimers or heterodimer structures. In some embodiments, the Fc regions can have, or can be modified to have, cysteine amino acids that are capable of forming disulfide bonds. In some embodiments, dimers of the RT-R2-R3 fusion proteins can form through disulfide bonds (1 or more, such as 2 disulfide bonds) between cysteine residues in R2 regions of separate fusion protein molecules. In some embodiments, the disulfide bonds form between R4 in separate fusion molecules (e.g., Fc with disulfides can be a type of dimerization domain).

In certain embodiments, the Fc region can be a variant comprising an amino acid substitution which alters antigen-independent effector functions, like the circulating half-life of a molecule to which it is linked. Molecules linked to these Fe regions can exhibit either increased or decreased binding to FcRn compared to Fc regions lacking these substitutions, and can have an increased or decreased half-life in serum, respectively. Fc variants with improved affinity for FcRn are anticipated to have longer serum half-lives, and such molecules have useful applications in methods long half-life of the linked molecule is desired. In contrast, Fc variants with decreased FcRn binding affinity are expected to have shorter half-lives, and such molecules are also useful, for example, where a shortened circulation time can be advantageous. Fc variants with decreased FcRn binding affinity are also less likely to cross the placenta. In addition, other applications in which reduced FcRn binding affinity can be desired include those applications in which localization to the brain, kidney, and/or liver is desired. In one embodiment, the Fc variant-linked molecules can exhibit reduced transport across the epithelium of kidney glomeruli from the vasculature.

In another embodiment, the Fc variant-linked molecules can exhibit reduced transport across the blood brain barrier (BBB) from the brain, into the vascular space. In one embodiment, an Fc region with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions with altered FcRn binding activity are disclosed in PCT Publication No. WO05/047327 which is incorporated by reference herein. In certain exemplary embodiments, the bispecific modulators disclosed herein comprise an Fc domain having one or more of the following substitutions: V284E, H285E, N286D, K290E and S304D (EU numbering).

In some embodiments, a molecule disclosed herein can be linked to an Fc variant comprising an amino acid substitution which alters glycosylation. For example, the Fc variant can have reduced glycosylation (e.g., N- or O-linked glycosylation). In some embodiments, the Fc variant comprises reduced glycosylation of the N-linked glycan normally found at amino acid position 297 (EU numbering). In another embodiment, the molecules can have an amino acid substitution near or within a glycosylation motif, for example, an N-linked glycosylation motif that contains the amino acid sequence NXT or NXS. In a particular embodiment, the Fc variant can have amino acid substitution at amino acid position 228 or 299 (EU numbering). Exemplary amino acid substitutions which confer reduced or altered glycosylation are described in PCT Publication No, WO05/018572, which is incorporated by reference herein in its entirety.

In some embodiments, the molecules disclosed herein can be modified to eliminate glycosylation and can be referred to as “agly” molecules. Exemplary agly molecules, can have an aglycosylated Fc region of an IgG4 antibody which is devoid of Fc-effector function thereby eliminating the potential for Fc mediated toxicity to the normal vital tissues and cells. In yet other embodiments, the molecules disclosed herein can have an altered glycan. For example, there can be a reduced number of fucose residues on an N-glycan at Asn297 of the Fc region, i.e., is afucosylated. In some embodiments, the there can be an altered number of sialic acid residues on the N-glycan at Asn297 of the Fc region.

In some embodiments, the CH2 or CH3 region of the Fc antibody domain can be truncated or modified to adjust the half-life of the molecule. In some embodiments, an Fc truncation includes CH3 or CH2 (e.g., Gehlsen, Kurt R., et al. “Pharmacokinetics of engineered human monomeric and dimeric CH2 domains.” MAbs. Vol. 4. No. 4. Taylor & Francis, 2012; Ying, Tianlei, et al. “Engineered soluble monomeric IgG1 CH3 domain: generation, mechanisms of function, and implications for design of biological therapeutics.” Journal of Biological Chemistry 288.35 (2013): 25154-25164).

In some embodiments, R4 can be a dimerization domain. The dimerization domain can be any region that can associate with another dimerization domain, through covalent or non-covalent bonds, to form a dimer (e.g., a bispecific modulator that is a homodimer or heterodimer). In some embodiments, R4 is not an Fc region from an antibody.

There are many protein dimerization domains known in the art (e.g., see Dang, Dung Thanh. “Molecular Approaches to Protein Dimerization: Opportunities for Supramolecular Chemistry.” Frontiers in Chemistry 10 (2022): 829312). An example dimerization domain can include zipper motifs, like a leucine zipper.

In some embodiments, the dimerization can form between regions of the bispecific modulators that are not R4 regions.

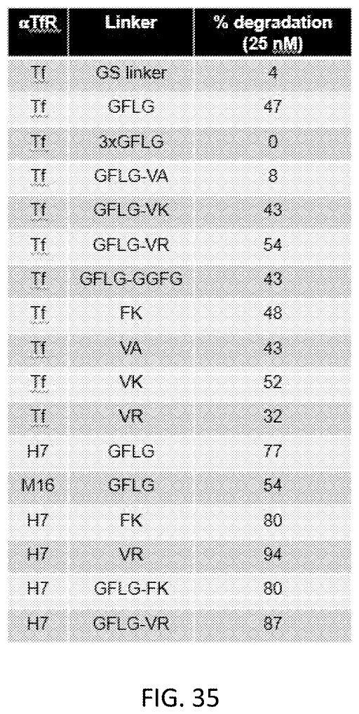

In some embodiments, R5 can be a protease-sensitive linking means. In some embodiments, the protease-sensitive linking means can be an amino acid sequence that can be cleaved by a protease. In some embodiments, the protease can be a protease in an endosome or lysosome. In some embodiments, the protease can be a cathepsin (e.g., cathepsin B) and the protease-sensitive linking means can be a cathepsin-cleavable peptide. Some example protease-sensitive linking means are shown in FIG. 35. In some embodiments, the protease-sensitive linking means can be GGFLGGVRGVDG (SEQ ID NO: 7) or GSGSGGEVRGVDG (SEQ ID NO: 8).

In some embodiments, a linkage (e.g., linker) can be located between various sections of the R1-R2-R3 fusion protein. In some embodiments, this linkage can be located between R2 and R3. In some embodiments, this linkage can be located between R1 and R2. In some embodiments, the linkage can be a glycine-rich linker (“GS linker). In some embodiments, a “GS” linker can be a combination of glycine and serine amino acids. In some embodiments, the GS linker can be GSSGGSGGSGGS (SEQ ID NO: 9). Other sequences are possible. In some embodiments, the GS linker can be SGGGG (SEQ ID NO: 10), SGGGSGGG (SEQ ID NO: 11), GSSGGSGGSGGS (SEQ ID NO: 12), GSGS (SEQ ID NO: 13), GSGGS (SEQ ID NO: 14), GSSGSS (SEQ ID NO: 15), GSSSSSS (SEQ ID NO: 16) and the like. In some embodiments, a GS linker can have at least 4 amino acids that are glycine and/or serine. In some embodiments, other amino acids can be part of a GS linker, as long as glycine and serine are in the majority.

In some embodiments, the bispecific modulators disclosed herein can include the following nucleotide and amino acid sequences, and molecules at least 60, 65, 70, 75, 80, 81, 82, 83, 84, 85, 86, 87, 88, 89, 90, 91, 92, 93, 94, 95, 96, 97, 98 or 99 percent identical to the nucleotide and amino acid sequences below. Specifically, the amino acid sequences of the bispecific modulators can be labeled as follows:
Times New Roman font underlined is signal peptide;

Times New Roman font bolded is anti-protein of interest
Fab-heavy chain, scFv, or affibody;
Times New Roman italicized font is linker encoded by restriction
enzyme site creation;
Times New Roman underlined and bolded font is GS linker;
Times New Roman underlined, italicized, and bolded font is
cleavable linker;
Courier New font is H7-scFv;
Courier New underlined font is Fc domain;
Courier New bolded font is TEV site;
Courier New italicized font is fragment from Transferrin,
Courier New underlined and bolded font is His-Tag;
Courier New bolded and italicized is light chain, and
Courier New underlined, italicized, and bolded font is Avi-Tag.
pDP14-CD19 ETD_Hole Fc
(SEQ ID NO: 17)
atgcgAatgcagctgctgctgctgattgcgctgagcctggcgctggtgaccaacagcactagtcc
cgaggaacctctagtggtgaaggtggaagagggagataacgctgtgctgcagtgcctcaagggga
cctcagatggccccactcagcagctgacctggtctcgggagtccccgcttaaacccttcttaaaa
ctcagcctggggctgccaggcctgggaatccacatgaggcccctggccatctggcttttcatctt
caacgtctctcaacagatggggggcttctacctgtgccagccggggcccccctctgagaaggcct
ggcagcctggctggacagtcaatgtggagggcagcggggagctgttccggtggaatgtttcggac
ctaggtggcctgggctgtggcctgaagaacaggtcctcagagggccccagctccccttccgggaa
gctcatgagccccaagctgtatgtgtgggccaaagaccgccctgagatctgggagggagagcctc
cgtgtctcccaccgagggacagcctgaaccagagcctcagccaggacctcaccatggcccctggc
tccacactctggctgtcctgtggggtaccccctgactctgtgtccaggggccccctctcctggac
ccatgtgcaccccaaggggcctaagtcattgctgagcctagagctgaaggacgatcgcccggcca
gagatatgtgggtaatggagacgggtctgttgttgccccgggccacagctcaagacgctggaaag
tattattgtcaccgtggcaacctgaccatgtcattccacctggagatcactgctcggccagtact
atggcactggctgctgaggactggtggctggaagactagtTCTGGTGGTGGTGGTGAGAATCTGT
ACTTTCAGAGCTCGGGCGGAGGATCgggtggaggcgagcccaaatcttgtgacaaaactcacaca
tgcCCCCCCTGCCCAGCGCCAGAATTGCTGGGCGGACCCAGCGTGTTCCTGTTCCCCCCCAAACC
TAAAGACACCCTGATGATCAGCCGAACCCCTGAGGTGACCTGCGTGGTGGTGGACGTGAGCCACG
AGGACCCCGAGGTGAAGTTCAACTGGTATGTGGACGGCGTGGAGGTCCACAATGCCAAAACGAAG
CCCAGGGAGGAGCAGTACAACAGCACCTACAGGGTAGTGAGCGTCTTGACCGTGCTGCACCAGGA
CTGGCTGAACGGCAAGGAATACAAATGCAAGGTCAGCAATAAGGCTCTGCCGGCTCCTATCGAGA
AGACAATCAGCAAGGCAAAGGGCCAGCCACGCGAACCGCAGGTGTATACTCTGCCCCCCAGCCGG
GACGAGCTGACCAAGAACCAGGTGTCCCTGAGCTGTGCCGTGAAAGGCTTCTACCCCAGCGACAT
CGCTGTGGAGTGGGAGAGTAACGGGCAGCCCGAGAACAACTACAAGACCACGCCTCCTGTGCTGG
ACAGCGACGGCAGCTTCTTCCTGGTGAGCAAGCTCACCGTGGACAAGAGCAGGTGGCAACAGGGC
AACGTGTTCAGCTGCTCTGTGATGCACGAGGCCCTGCACAACCATTACACCCAGAAGAGTCTCAG
TCTGAGCCCGGGAAAGGGTGGAGGCGGATCCGGCCTGAACGACATCTTCGAGGCTCAGAAAATCG
AATGGCACGAAGGCtaa
(SEQ ID NO: 18)
MRMQLLLLIALSLALVTNSTSPEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGWTVNVEGSGELFRWNVSD
LGGLGCGLKNRSSEGPSSPSGKLMSPKLYVWAKDRPEIWEGEPPCLPPRDSLNQSLSQDLTMAPG
STLWLSCGVPPDSVSRGPLSWTHVHPKGPKSLLSLELKDDRPARDMWVMETGLLLPRATAQDAGK
YYCHRGNLTMSFHLEITARPVLWHWLLRTGGWKTSSGGGGENLYFQSSGGGSGGGEPKSCDKTHT
CPPCPAPELLGGPSVFLFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSR
DELTKNQVSLSCAVKGFYPSDIAVEWESNGQPENNYKTTPPVLDSDGSFFLVSKLTVDKSRWQQG
NVFSCSVMHEALHNHYTQKSLSLSPGKGGGGSGLNDIFEAQKIEWHEG*
pDP16-EGFR-Affibody-FC-Tf
(SEQ ID NO: 19)
ATGTACAGGATGCAACTCCTGTCTTGCATTGCACTAAGTCTTGCACTTGTCACGAATTCGCTGCA
GGTAGATAACAAATTCAACAAAGAAATGTGGGCGGCGTGGGAAGAAATTCGCAACCTGCCGAACC
TGAACGGCTGGCAGATGACCGCGTTTATTGCGAGCCTGGTGGATGACCCAAGCCAAAGCGCTAAC
TTGCTAGCAGAAGCTAAAAAGCTAAATGATGCTCAGGCGCCGAAAGTAGACGGCAGCGGCAGCGA
CAAAACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTGTACACCCT
GCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGCACAACCACTACACGC
AGAAGAGCCTCTCCCTGTCTCCGGGTAAAggatcctctgggggaagtggaggtagcggtggttct
gtgcccgataagacagtgcggtggtgcgccgtgtctgagcacgaggccaccaagtgccagagctt
ccgggaccacatgaagtccgtgatccccagcgacggccctagcgtggcctgtgtgaagaaggcca
gctacctggactgcatccgggccattgccgccaatgaggccgacgccgtgacactggatgccggc
ctggtgtacgatgcctacctggcccccaacaacctgaagcccgtggtggccgagttctacggcag
caaagaggacccccagaccttctactacgccgtggccgtggtcaagaaggacagcggcttccaga
tgaaccagctgcggggcaagaagtcctgtcacaccggcctgggcagaagcgccggctggaacatc
cccatcggcctgctgtactgcgatctgcccgagccccggaagcctctggaaaaggccgtggccaa
cttcttcagcggcagctgcgccccttgtgctgacggaaccgacttcccccagctgtgtcagctgt
gccccggctgtggctgcagcaccctgaaccagtacttcggctacagcggcgccttcaagtgcctg
aaggacggcgctggcgacgtggccttcgtgaagcacagcaccatcttcgagaacctggccaacaa
ggccgaccgggaccagtacgagctgctgtgcctggacaacaccagaaagcccgtggacgagtaca
aggactgccacctcgcccaggtgccatctcacacagtggtggcccggtccatgggcggcaaagag
gatctgatctgggagctgctgaaccaggcccaggaacacttcggcaaggacaagagcaaagagtt
ccagctgttcagcagcccccacggcaaggatctgctgttcaaggacagcgcccacggctttctga
aggtgccccccagaatggacgccaagatgtacctgggctacgagtacgtgaccgccatccggaac
ctgagagagggcacctgtcccgaggcccccaccgatgagtgcaagcccgtgaagtggtgcgccct
gagccaccacgagcggctgaagtgcgacgagtggagcgtgaacagcgtgggcaagatcgagtgcg
tgagcgccgagacaaccgaggactgtatcgccaagatcatgaacggcgaggccgatgccatgagc
ctggacggcggcttcgtgtacattgccggcaagtgcggcctggtgcctgtgctggccgagaacta
caacaagagcgacaactgcgaggatacccccgaggccggctactttgccatcgcagtcgtgaaga
agtccgccagcgacctgacctgggacaatctgaagggcaagaaaagctgccacaccgccgtggga
aggaccgccgggtggaatattcctatggggctgctgtacaacaagatcaaccactgcagattcga
cgagttcttcagcgagggctgcgctcccggcagcaagaaagacagcagcctgtgcaagctgtgca
tgggcagcggcctgaacctgtgcgagcccaacaacaaagagggctactacggctacacaggggcc
ttccggtgtctggtggagaagggggacgtggcttttgtgaaacaccagaccgtgccccagaacac
cggcggcaagaaccccgacccctgggccaagaacctgaacgagaaggactacgaactgctgtgtc
tcgacggcacccggaagccagtggaggaatacgccaactgtcacctggccagagcccccaatcac
gccgtggtcacccggaaggacaaagaggcctgcgtccacaagatcctgcggcagcagcagcacct
gttcggcagcaacgtgaccgactgcagcggcaacttctgcctgttcagaagcgagacaaaggacc
tcctgttccgggacgacaccgtgtgtctggccaagctgcacgaccggaacacctacgagaagtac
ctgggcgaggaatatgtgaaggccgtgggcaatctgcggaagtgcagcacctctagcctgctgga
agcctgcacctttcgcagacctTAA
(SEQ ID NO: 20)
MYRMQLLSCIALSLALVTNSLQVDNKFNKEMWAAWEEIRNLPNLNGWQMTAFIASLVDDPSQSAN
LLAEAKKLNDAQAPKVDGSGSDKTHTCPPCPAPELLGGPSVFLFPPKPKDTLMISRTPEVTCVVV
DVSHEDPEVKENWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGKGSSGGSGGSGGS
VPDKTVRWCAVSEHEATKCQSFRDHMKSVIPSDGPSVACVKKASYLDCIRAIAANEADAVTLDAG
LVYDAYLAPNNLKPVVAEFYGSKEDPQTFYYAVAVVKKDSGFQMNQLRGKKSCHTGLGRSAGWNI
PIGLLYCDLPEPRKPLEKAVANFFSGSCAPCADGTDFPQLCQLCPGCGCSTLNQYFGYSGAFKCL
KDGAGDVAFVKHSTIFENLANKADRDQYELLCLDNTRKPVDEYKDCHLAQVPSHTVVARSMGGKE
DLIWELLNQAQEHFGKDKSKEFQLFSSPHGKDLLFKDSAHGFLKVPPRMDAKMYLGYEYVTAIRN
LREGTCPEAPTDECKPVKWCALSHHERLKCDEWSVNSVGKIECVSAETTEDCIAKIMNGEADAMS
LDGGFVYIAGKCGLVPVLAENYNKSDNCEDTPEAGYFAIAVVKKSASDLTWDNLKGKKSCHTAVG
RTAGWNIPMGLLYNKINHCRFDEFFSEGCAPGSKKDSSLCKLCMGSGLNLCEPNNKEGYYGYTGA
FRCLVEKGDVAFVKHQTVPQNTGGKNPDPWAKNLNEKDYELLCLDGTRKPVEEYANCHLARAPNH
AVVTRKDKEACVHKILRQQQHLFGSNVTDCSGNFCLFRSETKDLLFRDDTVCLAKLHDRNTYEKY
LGEEYVKAVGNLRKCSTSSLLEACTFRRP*
pDP18-EGFR-affibody
(SEQ ID NO: 21)
ATGTACAGGATGCAACTCCTGTCTTGCATTGCACTAAGTCTTGCACTTGTCACGAATTCGCTGCA
GGTAGATAACAAATTCAACAAAGAAATGTGGGCGGCGTGGGAAGAAATTCGCAACCTGCCGAACC
TGAACGGCTGGCAGATGACCGCGTTTATTGCGAGCCTGGTGGATGACCCAAGCCAAAGCGCTAAC
TTGCTAGCAGAAGCTAAAAAGCTAAATGATGCTCAGGCGCCGAAAGTAGACGGTGAGAATCTGTA
CTTTCAGAGCTCGGGCGGAGGATCGGGTGGAGGCCACCACCATCATCACCACCATCACGGATCCG
GCCTGAACGACATCTTCGAGGCTCAGAAAATCGAATGGCACGAAGGCggatcctctgggggaagt
ggaggtagcggtggttctgtgcccgataagacagtgcggtggtgcgccgtgtctgagcacgaggc
caccaagtgccagagcttccgggaccacatgaagtccgtgatccccagcgacggccctagcgtgg
cctgtgtgaagaaggccagctacctggactgcatccgggccattgccgccaatgaggccgacgcc
gtgacactggatgccggcctggtgtacgatgcctacctggcccccaacaacctgaagcccgtggt
ggccgagttctacggcagcaaagaggacccccagaccttctactacgccgtggccgtggtcaaga
aggacagcggcttccagatgaaccagctgcggggcaagaagtcctgtcacaccggcctgggcaga
agcgccggctggaacatccccatcggcctgctgtactgcgatctgcccgagccccggaagcctct
ggaaaaggccgtggccaacttcttcagcggcagctgcgccccttgtgctgacggaaccgacttcc
cccagctgtgtcagctgtgccccggctgtggctgcagcaccctgaaccagtacttcggctacagc
ggcgccttcaagtgcctgaaggacggcgctggcgacgtggccttcgtgaagcacagcaccatctt
cgagaacctggccaacaaggccgaccgggaccagtacgagctgctgtgcctggacaacaccagaa
agcccgtggacgagtacaaggactgccacctcgcccaggtgccatctcacacagtggtggcccgg
tccatgggggcaaagaggatctgatctgggagctgctgaaccaggcccaggaacacttcggcaag
gacaagagcaaagagttccagctgttcagcagcccccacggcaaggatctgctgttcaaggacag
cgcccacggctttctgaaggtgccccccagaatggacgccaagatgtacctgggctacgagtacg
tgaccgccatccggaacctgagagagggcacctgtcccgaggcccccaccgatgagtgcaagccc
gtgaagtggtgcgccctgagccaccacgagcggctgaagtgcgacgagtggagcgtgaacagcgt
gggcaagatcgagtgcgtgagcgccgagacaaccgaggactgtatcgccaagatcatgaacggcg
aggccgatgccatgagcctggacggcggcttcgtgtacattgccggcaagtgcggcctggtgcct
gtgctggccgagaactacaacaagagcgacaactgcgaggatacccccgaggccggctactttgc
catcgcagtcgtgaagaagtccgccagcgacctgacctgggacaatctgaagggcaagaaaagct
gccacaccgccgtgggaaggaccgccgggtggaatattcctatggggctgctgtacaacaagatc
aaccactgcagattcgacgagttcttcagcgagggctgcgctcccggcagcaagaaagacagcag
cctgtgcaagctgtgcatgggcagcggcctgaacctgtgcgagcccaacaacaaagagggctact
acggctacacaggggccttccggtgtctggtggagaagggggacgtggcttttgtgaaacaccag
accgtgccccagaacaccggcggcaagaaccccgacccctgggccaagaacctgaacgagaagga
ctacgaactgctgtgtctcgacggcacccggaagccagtggaggaatacgccaactgtcacctgg
ccagagcccccaatcacgccgtggtcacccggaaggacaaagaggcctgcgtccacaagatcctg
cggcagcagcagcacctgttcggcagcaacgtgaccgactgcagcggcaacttctgcctgttcag
aagcgagacaaaggacctcctgttccgggacgacaccgtgtgtctggccaagctgcacgaccgga
acacctacgagaagtacctgggcgaggaatatgtgaaggccgtgggcaatctgcggaagtgcagc
acctctagcctgctggaagcctgcacctttcgcagacctTAA
(SEQ ID NO: 22)
MYRMQLLSCIALSLALVTNSLQVDNKFNKEMWAAWEEIRNLPNLNGWQMTAFIASLVDDPSQSAN
LLAEAKKLNDAQAPKVDGENLYFQSSGGGSGGGHHHHHHHHGSGLNDIFEAQKIEWHEGGSSGGS
GGSGGSVPDKTVRWCAVSEHEATKCQSFRDHMKSVIPSDGPSVACVKKASYLDCIRAIAANEADA
VTLDAGLVYDAYLAPNNLKPVVAEFYGSKEDPQTFYYAVAVVKKDSGFQMNQLRGKKSCHTGLGR
SAGWNIPIGLLYCDLPEPRKPLEKAVANFFSGSCAPCADGTDFPQLCQLCPGCGCSTLNQYFGYS
GAFKCLKDGAGDVAFVKHSTIFENLANKADRDQYELLCLDNTRKPVDEYKDCHLAQVPSHTVVAR
SMGGKEDLIWELLNQAQEHFGKDKSKEFQLFSSPHGKDLLFKDSAHGFLKVPPRMDAKMYLGYEY
VTAIRNLREGTCPEAPTDECKPVKWCALSHHERLKCDEWSVNSVGKIECVSAETTEDCIAKIMNG
EADAMSLDGGFVYIAGKCGLVPVLAENYNKSDNCEDTPEAGYFAIAVVKKSASDLTWDNLKGKKS
CHTAVGRTAGWNIPMGLLYNKINHCRFDEFFSEGCAPGSKKDSSLCKLCMGSGLNLCEPNNKEGY
YGYTGAFRCLVEKGDVAFVKHQTVPQNTGGKNPDPWAKNLNEKDYELLCLDGTRKPVEEYANCHL
ARAPNHAVVTRKDKEACVHKILRQQQHLFGSNVTDCSGNFCLFRSETKDLLFRDDTVCLAKLHDR
NTYEKYLGEEYVKAVGNLRKCSTSSLLEACTFRRP*
pDP20-EGFR-Affibody-FC
(SEQ ID NO: 23)
ATGTACAGGATGCAACTCCTGTCTTGCATTGCACTAAGTCTTGCACTTGTCACGAATTCGCTGCA
GGTAGATAACAAATTCAACAAAGAAATGTGGGCGGCGTGGGAAGAAATTCGCAACCTGCCGAACC
TGAACGGCTGGCAGATGACCGCGTTTATTGCGAGCCTGGTGGATGACCCAAGCCAAAGCGCTAAC
TTGCTAGCAGAAGCTAAAAAGCTAAATGATGCTCAGGCGCCGAAAGTAGACGGCAGCGGCAGCGA
CAAAACTCACACATGCCCACCGTGCCCAGCACCTGAACTCCTGGGGGGACCGTCAGTCTTCCTCT
TCCCCCCAAAACCCAAGGACACCCTCATGATCTCCCGGACCCCTGAGGTCACATGCGTGGTGGTG
GACGTGAGCCACGAAGACCCTGAGGTCAAGTTCAACTGGTACGTGGACGGCGTGGAGGTGCATAA
TGCCAAGACAAAGCCGCGGGAGGAGCAGTACAACAGCACGTACCGTGTGGTCAGCGTCCTCACCG
TCCTGCACCAGGACTGGCTGAATGGCAAGGAGTACAAGTGCAAGGTCTCCAACAAAGCCCTCCCA
GCCCCCATCGAGAAAACCATCTCCAAAGCCAAAGGGCAGCCCCGAGAACCACAGGTGTACACCCT
GCCCCCATCCCGGGATGAGCTGACCAAGAACCAGGTCAGCCTGACCTGCCTGGTCAAAGGCTTCT
ATCCCAGCGACATCGCCGTGGAGTGGGAGAGCAATGGGCAGCCGGAGAACAACTACAAGACCACG
CCTCCCGTGCTGGACTCCGACGGCTCCTTCTTCCTCTACAGCAAGCTCACCGTGGACAAGAGCAG
GTGGCAGCAGGGGAACGTCTTCTCATGCTCCGTGATGCATGAGGCTCTGCACAACCACTACACGC
AGAAGAGCCTCTCCCTGTCTCCGGGTAAATAA
(SEQ ID NO: 24)
MYRMQLLSCIALSLALVTNSLQVDNKFNKEMWAAWEEIRNLPNLNGWQMTAFIASLVDDPSQSAN
LLAEAKKLNDAQAPKVDGSGSDKTHTCPPCPAPELLGGPSVELFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLSPGK*
pDP22-EGFR-affibody
(SEQ ID NO: 25)
ATGCGAATGCAGCTGCTGCTGCTGATTGCGCTGAGCCTGGCGCTGGTGACCAACAGCACTAGTCT
GCAGGTAGATAACAAATTCAACAAAGAAATGTGGGCGGCGTGGGAAGAAATTCGCAACCTGCCGA
ACCTGAACGGCTGGCAGATGACCGCGTTTATTGCGAGCCTGGTGGATGACCCAAGCCAAAGCGCT
AACTTGCTAGCAGAAGCTAAAAAGCTAAATGATGCTCAGGCGCCGAAAGTAGACGGCAGCGGCAG
CACTAGTTCTGGTGGTGGTGGTGAGAATCTGTACTTTCAGAGCTCGGGCGGAGGATCGGGTGGAG
GCCACCACCATCATCACCACCATCACGGATCCGGCCTGAACGACATCTTCGAGGCTCAGAAAATC
GAATGGCACGAAGGCTAA
(SEQ ID NO: 26)
MRMQLLLLIALSLALVTNSTSLQVDNKENKEMWAAWEEIRNLPNLNGWQMTAFIASLVDDPSQSA
NLLAEAKKLNDAQAPKVDGSGSTSSGGGGENLYFQSSGGGSGGGHHHHHHHHGSGLNDIFEAQKI
EWHEG*
pDP24-CD19-FC-Tf
(SEQ ID NO: 27)
ATGTACAGGATGCAACTCCTGTCTTGCATTGCACTAAGTCTTGCACTTGTCACGAATTCGcccga
ggaacctctagtggtgaaggtggaagagggagataacgctgtgctgcagtgcctcaaggggacct
cagatggccccactcagcagctgacctggtctcgggagtccccgcttaaacccttcttaaaactc
agcctggggctgccaggcctgggaatccacatgaggcccctggccatctggcttttcatcttcaa
cgtctctcaacagatggggggcttctacctgtgccagccggggcccccctctgagaaggcctggc
agcctggctggacagtcaatgtggagggcagcggggagctgttccggtggaatgtttcggaccta
ggtggcctgggctgtggcctgaagaacaggtcctcagagggccccagctccccttccgggaagct
catgagccccaagctgtatgtgtgggccaaagaccgccctgagatctgggagggagagcctccgt
gtctcccaccgagggacagcctgaaccagagcctcagccaggacctcaccatggcccctggctcc
acactctggctgtcctgtggggtaccccctgactctgtgtccaggggccccctctcctggaccca
tgtgcaccccaaggggcctaagtcattgctgagcctagagctgaaggacgatcgcccggccagag
atatgtgggtaatggagacgggtctgttgttgccccgggccacagctcaagacgctggaaagtat
tattgtcaccgtggcaacctgaccatgtcattccacctggagatcactgctcggccagtactatg
gcactggctgctgaggactggtggctggaagGTAGACGGCAGCGGCAGCGACAAAACTCACACAT
GCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCCTGCCCCCATCCCGGG
ATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGA
CTCCGACGGCTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCC
CTGTCTCCGGGTAAAggatcctctgggggaagtggaggtagcggtggttctgtgcccgataagac
agtgcggtggtgcgccgtgtctgagcacgaggccaccaagtgccagagcttccgggaccacatga
agtccgtgatccccagcgacggccctagcgtggcctgtgtgaagaaggccagctacctggactgc
atccgggccattgccgccaatgaggccgacgccgtgacactggatgccggcctggtgtacgatgc
ctacctggcccccaacaacctgaagcccgtggtggccgagttctacggcagcaaagaggaccccc
agaccttctactacgccgtggccgtggtcaagaaggacagcggcttccagatgaaccagctgcgg
ggcaagaagtcctgtcacaccggcctgggcagaagcgccggctggaacatccccatcggcctgct
gtactgcgatctgcccgagccccggaagcctctggaaaaggccgtggccaacttcttcagcggca
gctgcgccccttgtgctgacggaaccgacttcccccagctgtgtcagctgtgccccggctgtggc
tgcagcaccctgaaccagtacttcggctacagcggcgccttcaagtgcctgaaggacggcgctgg
cgacgtggccttcgtgaagcacagcaccatcttcgagaacctggccaacaaggccgaccgggacc
agtacgagctgctgtgcctggacaacaccagaaagcccgtggacgagtacaaggactgccacctc
gcccaggtgccatctcacacagtggtggcccggtccatgggcggcaaagaggatctgatctggga
gctgctgaaccaggcccaggaacacttcggcaaggacaagagcaaagagttccagctgttcagca
gcccccacggcaaggatctgctgttcaaggacagcgcccacggctttctgaaggtgccccccaga
atggacgccaagatgtacctgggctacgagtacgtgaccgccatccggaacctgagagagggcac
ctgtcccgaggcccccaccgatgagtgcaagcccgtgaagtggtgcgccctgagccaccacgagc
ggctgaagtgcgacgagtggagcgtgaacagcgtgggcaagatcgagtgcgtgagcgccgagaca
accgaggactgtatcgccaagatcatgaacggcgaggccgatgccatgagcctggacggcggctt
cgtgtacattgccggcaagtgcggcctggtgcctgtgctggccgagaactacaacaagagcgaca
actgcgaggatacccccgaggccggctactttgccatcgcagtcgtgaagaagtccgccagcgac
ctgacctgggacaatctgaagggcaagaaaagctgccacaccgccgtgggaaggaccgccgggtg
gaatattcctatggggctgctgtacaacaagatcaaccactgcagattcgacgagttcttcagcg
agggctgcgctcccggcagcaagaaagacagcagcctgtgcaagctgtgcatgggcagcggcctg
aacctgtgcgagcccaacaacaaagagggctactacggctacacaggggccttccggtgtctggt
ggagaagggggacgtggcttttgtgaaacaccagaccgtgccccagaacaccggcggcaagaacc
ccgacccctgggccaagaacctgaacgagaaggactacgaactgctgtgtctcgacggcacccgg
aagccagtggaggaatacgccaactgtcacctggccagagcccccaatcacgccgtggtcacccg
gaaggacaaagaggcctgcgtccacaagatcctgcggcagcagcagcacctgttcggcagcaacg
tgaccgactgcagcggcaacttctgcctgttcagaagcgagacaaaggacctcctgttccgggac
gacaccgtgtgtctggccaagctgcacgaccggaacacctacgagaagtacctgggcgaggaata
tgtgaaggccgtgggcaatctgcggaagtgcagcacctctagcctgctggaagcctgcacctttc
gcagacctTAA
(SEQ ID NO: 28)
MYRMQLLSCIALSLALVTNSPEEPLVVKVEEGDNAVLQCLKGTSDGPTQQLTWSRESPLKPFLKL
SLGLPGLGIHMRPLAIWLFIFNVSQQMGGFYLCQPGPPSEKAWQPGWTVNVEGSGELFRWNVSDL
GGLGCGLKNRSSEGPSSPSGKLMSPKLYVWAKDRPEIWEGEPPCLPPRDSLNQSLSQDLTMAPGS
TLWLSCGVPPDSVSRGPLSWTHVHPKGPKSLLSLELKDDRPARDMWVMETGLLLPRATAQDAGKY
YCHRGNLTMSFHLEITARPVLWHWLLRTGGWKVDGSGSDKTHTCPPCPAPELLGGPSVFLFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLS
LSPGKGSSGGSGGSGGSVPDKTVRWCAVSEHEATKCQSFRDHMKSVIPSDGPSVACVKKASYLDC
IRAIAANEADAVTLDAGLVYDAYLAPNNLKPVVAEFYGSKEDPQTFYYAVAVVKKDSGFQMNQLR
GKKSCHTGLGRSAGWNIPIGLLYCDLPEPRKPLEKAVANFFSGSCAPCADGTDFPQLCQLCPGCG
CSTLNQYFGYSGAFKCLKDGAGDVAFVKHSTIFENLANKADRDQYELLCLDNTRKPVDEYKDCHL
AQVPSHTVVARSMGGKEDLIWELLNQAQEHFGKDKSKEFQLFSSPHGKDLLFKDSAHGFLKVPPR
MDAKMYLGYEYVTAIRNLREGTCPEAPTDECKPVKWCALSHHERLKCDEWSVNSVGKIECVSAET
TEDCIAKIMNGEADAMSLDGGFVYIAGKCGLVPVLAENYNKSDNCEDTPEAGYFAIAVVKKSASD
LTWDNLKGKKSCHTAVGRTAGWNIPMGLLYNKINHCRFDEFFSEGCAPGSKKDSSLCKLCMGSGL
NLCEPNNKEGYYGYTGAFRCLVEKGDVAFVKHQTVPQNTGGKNPDPWAKNLNEKDYELLCLDGTR
KPVEEYANCHLARAPNHAVVTRKDKEACVHKILRQQQHLFGSNVTDCSGNFCLFRSETKDLLFRD
DTVCLAKLHDRNTYEKYLGEEYVKAVGNLRKCSTSSLLEACTFRRP*
pDP25-pFUSE-Tf-knob-Fc
(SEQ ID NO: 29)
ATGTACAGGATGCAACTCCTGTCTTGCATTGCACTAAGTCTTGCACTTGTCACGAATTCGgtgcc
cgataagacagtgcggtggtgcgccgtgtctgagcacgaggccaccaagtgccagagcttccggg
accacatgaagtccgtgatccccagcgacggccctagcgtggcctgtgtgaagaaggccagctac
ctggactgcatccgggccattgccgccaatgaggccgacgccgtgacactggatgccggcctggt
gtacgatgcctacctggcccccaacaacctgaagcccgtggtggccgagttctacggcagcaaag
aggacccccagaccttctactacgccgtggccgtggtcaagaaggacagcggcttccagatgaac
cagctgcggggcaagaagtcctgtcacaccggcctgggcagaagcgccggctggaacatccccat
cggcctgctgtactgcgatctgcccgagccccggaagcctctggaaaaggccgtggccaacttct
tcagcggcagctgcgccccttgtgctgacggaaccgacttcccccagctgtgtcagctgtgcccc
ggctgtggctgcagcaccctgaaccagtacttcggctacagcggcgccttcaagtgcctgaagga
cggcgctggcgacgtggccttcgtgaagcacagcaccatcttcgagaacctggccaacaaggccg
accgggaccagtacgagctgctgtgcctggacaacaccagaaagcccgtggacgagtacaaggac
tgccacctcgcccaggtgccatctcacacagtggtggcccggtccatgggcggcaaagaggatct
gatctgggagctgctgaaccaggcccaggaacacttcggcaaggacaagagcaaagagttccagc
tgttcagcagcccccacggcaaggatctgctgttcaaggacagcgcccacggctttctgaaggtg
ccccccagaatggacgccaagatgtacctgggctacgagtacgtgaccgccatccggaacctgag
agagggcacctgtcccgaggcccccaccgatgagtgcaagcccgtgaagtggtgcgccctgagcc
accacgagcggctgaagtgcgacgagtggagcgtgaacagcgtgggcaagatcgagtgcgtgagc
gccgagacaaccgaggactgtatcgccaagatcatgaacggcgaggccgatgccatgagcctgga
cggcggcttcgtgtacattgccggcaagtgcggcctggtgcctgtgctggccgagaactacaaca
agagcgacaactgcgaggatacccccgaggccggctactttgccatcgcagtcgtgaagaagtcc
gccagcgacctgacctgggacaatctgaagggcaagaaaagctgccacaccgccgtgggaaggac
cgccgggtggaatattcctatggggctgctgtacaacaagatcaaccactgcagattcgacgagt
tcttcagcgagggctgcgctcccggcagcaagaaagacagcagcctgtgcaagctgtgcatgggc
agcggcctgaacctgtgcgagcccaacaacaaagagggctactacggctacacaggggccttccg
gtgtctggtggagaagggggacgtggcttttgtgaaacaccagaccgtgccccagaacaccggcg
gcaagaaccccgacccctgggccaagaacctgaacgagaaggactacgaactgctgtgtctcgac
ggcacccggaagccagtggaggaatacgccaactgtcacctggccagagcccccaatcacgccgt
ggtcacccggaaggacaaagaggcctgcgtccacaagatcctgcggcagcagcagcacctgttcg
gcagcaacgtgaccgactgcagcggcaacttctgcctgttcagaagcgagacaaaggacctcctg
ttccgggacgacaccgtgtgtctggccaagctgcacgaccggaacacctacgagaagtacctggg
cgaggaatatgtgaaggccgtgggcaatctgcggaagtgcagcacctctagcctgctggaagcct
gcacctttcgcagacctCCATGGgagcccaaatcttgtgacaaaactcacacatgcCCCCCCTGC
CCAGCGCCAGAATTGCTGGGCGGACCCAGCGTGTTCCTGTTCCCCCCCAAACCTAAAGACACCCT
GATGATCAGCCGAACCCCTGAGGTGACCTGCGTGGTGGTGGACGTGAGCCACGAGGACCCCGAGG
TGAAGTTCAACTGGTATGTGGACGGCGTGGAGGTCCACAATGCCAAAACGAAGCCCAGGGAGGAG
CAGTACGGAAGCACCTACAGGGTAGTGAGCGTCTTGACCGTGCTGCACCAGGACTGGCTGAACGG
CAAGGAATACAAATGCAAGGTCAGCAATAAGGCTCTGCCGGCTCCTATCGAGAAGACAATCAGCA
AGGCAAAGGGCCAGCCACGCGAACCGCAGGTGTATACTCTGCCCCCCAGCCGGGACGAGCTGACC
AAGAACCAGGTGTCCCTGTGGTGTCTGGTGAAAGGCTTCTACCCCAGCGACATCGCTGTGGAGTG
GGAGAGTAACGGGCAGCCCGAGAACAACTACAAGACCACGCCTCCTGTGCTGGACAGCGACGGCA
GCTTCTTCCTGTATAGCAAGCTCACCGTGGACAAGAGCAGGTGGCAACAGGGCAACGTGTTCAGC
TGCTCTGTGATGCACGAGGCCCTGCACAACCATTACACCCAGAAGAGTCTCAGTCTGAGCCCGGG
AAAGggtggctctcatcatcaccatcaccactga
(SEQ ID NO: 30)
MYRMQLLSCIALSLALVTNSVPDKTVRWCAVSEHEATKCQSFRDHMKSVIPSDGPSVACVKKASY
LDCIRAIAANEADAVTLDAGLVYDAYLAPNNLKPVVAEFYGSKEDPQTFYYAVAVVKKDSGFQMN
QLRGKKSCHTGLGRSAGWNIPIGLLYCDLPEPRKPLEKAVANFFSGSCAPCADGTDFPQLCQLCP
GCGCSTLNQYFGYSGAFKCLKDGAGDVAFVKHSTIFENLANKADRDQYELLCLDNTRKPVDEYKD
CHLAQVPSHTVVARSMGGKEDLIWELLNQAQEHFGKDKSKEFQLFSSPHGKDLLFKDSAHGFLKV
PPRMDAKMYLGYEYVTAIRNLREGTCPEAPTDECKPVKWCALSHHERLKCDEWSVNSVGKIECVS
AETTEDCIAKIMNGEADAMSLDGGFVYIAGKCGLVPVLAENYNKSDNCEDTPEAGYFAIAVVKKS
ASDLTWDNLKGKKSCHTAVGRTAGWNIPMGLLYNKINHCRFDEFFSEGCAPGSKKDSSLCKLCMG
SGLNLCEPNNKEGYYGYTGAFRCLVEKGDVAFVKHQTVPQNTGGKNPDPWAKNLNEKDYELLCLD
GTRKPVEEYANCHLARAPNHAVVTRKDKEACVHKILRQQQHLFGSNVTDCSGNFCLFRSETKDLL
FRDDTVCLAKLHDRNTYEKYLGEEYVKAVGNLRKCSTSSLLEACTFRRPPWEPKSCDKTHTCPPC
PAPELLGGPSVFLFPPKPKDTLMISRTPEVTCVVVDVSHEDPEVKFNWYVDGVEVHNAKTKPREE
QYGSTYRVVSVLTVLHQDWLNGKEYKCKVSNKALPAPIEKTISKAKGQPREPQVYTLPPSRDELT
KNQVSLWCLVKGFYPSDIAVEWESNGQPENNYKTTPPVLDSDGSFFLYSKLTVDKSRWQQGNVFS
CSVMHEALHNHYTQKSLSLSPGKGGSHHHHHH*
pDP32-CD19-FC-CD19
(SEQ ID NO: 31)
atgcgAatgcagctgctgctgctgattgcgctgagcctggcgctggtgaccaacagcactagtcc
cgaggaacctctagtggtgaaggtggaagagggagataacgctgtgctgcagtgcctcaagggga
cctcagatggccccactcagcagctgacctggtctcgggagtccccgcttaaacccttcttaaaa
ctcagcctggggctgccaggcctgggaatccacatgaggcccctggccatctggcttttcatctt
caacgtctctcaacagatggggggcttctacctgtgccagccggggcccccctctgagaaggcct
ggcagcctggctggacagtcaatgtggagggcagcggggagctgttccggtggaatgtttcggac
ctaggtggcctgggctgtggcctgaagaacaggtcctcagagggccccagctccccttccgggaa
gctcatgagccccaagctgtatgtgtgggccaaagaccgccctgagatctgggagggagagcctc
cgtgtctcccaccgagggacagcctgaaccagagcctcagccaggacctcaccatggcccctggc
tccacactctggctgtcctgtggggtaccccctgactctgtgtccaggggccccctctcctggac
ccatgtgcaccccaaggggcctaagtcattgctgagcctagagctgaaggacgatcgcccggcca
gagatatgtgggtaatggagacgggtctgttgttgccccgggccacagctcaagacgctggaaag
tattattgtcaccgtggcaacctgaccatgtcattccacctggagatcactgctcggccagtact
atggcactggctgctgaggactggtggctggaagactagtTCTGGTGGTGGTGGTGAGAATCTGT
ACTTTCAGAGCTCGGGCGGAGGATCgggtggaggcgagcccaaatcttgtgacaaaactcacaca
tgcCCCCCCTGCCCAGCGCCAGAATTGCTGGGCGGACCCAGCGTGTTCCTGTTCCCCCCCAAACC
TAAAGACACCCTGATGATCAGCCGAACCCCTGAGGTGACCTGCGTGGTGGTGGACGTGAGCCACG
AGGACCCCGAGGTGAAGTTCAACTGGTATGTGGACGGCGTGGAGGTCCACAATGCCAAAACGAAG
CCCAGGGAGGAGCAGTACAACAGCACCTACAGGGTAGTGAGCGTCTTGACCGTGCTGCACCAGGA
CTGGCTGAACGGCAAGGAATACAAATGCAAGGTCAGCAATAAGGCTCTGCCGGCTCCTATCGAGA
AGACAATCAGCAAGGCAAAGGGCCAGCCACGCGAACCGCAGGTGTATACTCTGCCCCCCAGCCGG
GACGAGCTGACCAAGAACCAGGTGTCCCTGACCTGTCTGGTGAAAGGCTTCTACCCCAGCGACAT
CGCTGTGGAGTGGGAGAGTAACGGGCAGCCCGAGAACAACTACAAGACCACGCCTCCTGTGCTGG
ACAGCGACGGCAGCTTCTTCCTGTATAGCAAGCTCACCGTGGACAAGAGCAGGTGGCAACAGGGC
AACGTGTTCAGCTGCTCTGTGATGCACGAGGCCCTGCACAACCATTACACCCAGAAGAGTCTCAG
TCTGAGCCCGGGAAAGGGTGGAGGCGGATCCggaggtagcggtggttctGGAcccgaggaacctc
tagtggtgaaggtggaagagggagataacgctgtgctgcagtgcctcaaggggacctcagatggc
cccactcagcagctgacctggtctcgggagtccccgcttaaacccttcttaaaactcagcctggg
gctgccaggcctgggaatccacatgaggcccctggccatctggcttttcatcttcaacgtctctc
aacagatggggggcttctacctgtgccagccggggcccccctctgagaaggcctggcagcctggc
tggacagtcaatgtggagggcagcggggagctgttccggtggaatgtttcggacctaggtggcct
gggctgtggcctgaagaacaggtcctcagagggccccagctccccttccgggaagctcatgagcc
ccaagctgtatgtgtgggccaaagaccgccctgagatctgggagggagagcctccgtgtctccca
ccgagggacagcctgaaccagagcctcagccaggacctcaccatggcccctggctccacactctg
gctgtcctgtggggtaccccctgactctgtgtccaggggccccctctcctggacccatgtgcacc
ccaaggggcctaagtcattgctgagcctagagctgaaggacgatcgcccggccagagatatgtgg
gtaatggagacgggtctgttgttgccccgggccacagctcaagacgctggaaagtattattgtca
ccgtggcaacctgaccatgtcattccacctggagatcactgctcggccagtactatggcactggc
tgctgaggactggtggctggaagGGCCTGAACGACATCTTCGAGGCTCAGAAAATCGAATGGCAC
GAAGGCtaa
(SEQ ID NO: 32)
MRMQLLLLIALSLALVTNSTSPEEPLVVKVEEGDNAVLQCLKGTSDGPTQQLTWSRESPLKPFLK
LSLGLPGLGIHMRPLAIWLFIFNVSQQMGGFYLCQPGPPSEKAWQPGWTVNVEGSGELFRWNVSD
LGGLGCGLKNRSSEGPSSPSGKLMSPKLYVWAKDRPEIWEGEPPCLPPRDSLNQSLSQDLTMAPG
STLWLSCGVPPDSVSRGPLSWTHVHPKGPKSLLSLELKDDRPARDMWVMETGLLLPRATAQDAGK
YYCHRGNLTMSFHLEITARPVLWHWLLRTGGWKTSSGGGGENLYFQSSGGGSGGGEPKSCDKTHT
CPPCPAPELLGGPSVFLFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGSGGSGPEEPLVVKVEEGDNAVLQCLKGTSDG
PTQQLTWSRESPLKPFLKLSLGLPGLGIHMRPLAIWLFIFNVSQQMGGFYLCQPGPPSEKAWQPG
WTVNVEGSGELFRWNVSDLGGLGCGLKNRSSEGPSSPSGKLMSPKLYVWAKDRPEIWEGEPPCLP
PRDSLNQSLSQDLTMAPGSTLWLSCGVPPDSVSRGPLSWTHVHPKGPKSLLSLELKDDRPARDMW
VMETGLLLPRATAQDAGKYYCHRGNLTMSFHLEITARPVLWHWLLRTGGWKGLNDIFEAQKIEWH
EG*
pDP44-CD19 NT.1-FC-Tf
(SEQ ID NO: 33)
ATGTACAGGATGCAACTCCTGTCTTGCATTGCACTAAGTCTTGCACTTGTCACGAATTCGcccga
ggaacctctagtggtgaaggtggaagagggagataccgctgctctgtggtgcctcaaggggacct
cagatggccccactcagcagctgacctggtctcgggagtccccgcttaaacccttcttaaaatac
agcctgggggtgccaggcctgggagtccacgtcaggcccgatgccatctctgtcgtcatcaggaa
cgtctctcaacagatggggggcttctacctgtgccagccggggcccccctctgagaaggcctggc
agcctggctggacagtcaatgtggagggcagcggggagctgttccggtggaatgtttcggaccta
ggtggcctgggctgtggcctgaagaacaggtcctcagagggccccagctccccttccgggaagct
catgagccccaagctgtatgtgtgggccaaagaccgccctgagatctgggagggagagcctccgt
gtctcccaccgagggacagcctgaaccagagcctcagcagggacctcaccgtagcccctggctcc
acactctggctgtcctgtggggtaccccctgactctgtgtccaggggccccctctcctggaccca
tgtgcaccccaaggggcctaagtcattgctgagcctagagctgaaggacgatcgcccggccagag
atatgtgggtaatgggtacgtcactgatgttgccccgggccacagctcaagacgctggaaagtgg
tattgtcaccgtggcaacgtaaccacctcattccacctggaggtaatcgctcggccagtaaaggc
tcactcagacctgaggactggtggctggaagGTAGACGGCAGCGGCAGCGACAAAACTCACACAT
GCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCCTGCCCCCATCCCGGG
ATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGA
CTCCGACGGCTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCC
CTGTCTCCGGGTAAAggatcctctgggggaagtggaggtagcggtggttctgtgcccgataagac
agtgcggtggtgcgccgtgtctgagcacgaggccaccaagtgccagagcttccgggaccacatga
agtccgtgatccccagcgacggccctagcgtggcctgtgtgaagaaggccagctacctggactgc
atccgggccattgccgccaatgaggccgacgccgtgacactggatgccggcctggtgtacgatgc
ctacctggcccccaacaacctgaagcccgtggtggccgagttctacggcagcaaagaggaccccc
agaccttctactacgccgtggccgtggtcaagaaggacagcggcttccagatgaaccagctgcgg
ggcaagaagtcctgtcacaccggcctgggcagaagcgccggctggaacatccccatcggcctgct
gtactgcgatctgcccgagccccggaagcctctggaaaaggccgtggccaacttcttcagcggca
gctgcgccccttgtgctgacggaaccgacttcccccagctgtgtcagctgtgccccggctgtggc
tgcagcaccctgaaccagtacttcggctacagcggcgccttcaagtgcctgaaggacggcgctgg
cgacgtggccttcgtgaagcacagcaccatcttcgagaacctggccaacaaggccgaccgggacc
agtacgagctgctgtgcctggacaacaccagaaagcccgtggacgagtacaaggactgccacctc
gcccaggtgccatctcacacagtggtggcccggtccatgggcggcaaagaggatctgatctggga
gctgctgaaccaggcccaggaacacttcggcaaggacaagagcaaagagttccagctgttcagca
gcccccacggcaaggatctgctgttcaaggacagcgcccacggctttctgaaggtgccccccaga
atggacgccaagatgtacctgggctacgagtacgtgaccgccatccggaacctgagagagggcac
ctgtcccgaggcccccaccgatgagtgcaagcccgtgaagtggtgcgccctgagccaccacgagc
ggctgaagtgcgacgagtggagcgtgaacagcgtgggcaagatcgagtgcgtgagcgccgagaca
accgaggactgtatcgccaagatcatgaacggcgaggccgatgccatgagcctggacggcggctt
cgtgtacattgccggcaagtgcggcctggtgcctgtgctggccgagaactacaacaagagcgaca
actgcgaggatacccccgaggccggctactttgccatcgcagtcgtgaagaagtccgccagcgac
ctgacctgggacaatctgaagggcaagaaaagctgccacaccgccgtgggaaggaccgccgggtg
gaatattcctatggggctgctgtacaacaagatcaaccactgcagattcgacgagttcttcagcg
agggctgcgctcccggcagcaagaaagacagcagcctgtgcaagctgtgcatgggcagcggcctg
aacctgtgcgagcccaacaacaaagagggctactacggctacacaggggccttccggtgtctggt
ggagaagggggacgtggcttttgtgaaacaccagaccgtgccccagaacaccggcggcaagaacc
ccgacccctgggccaagaacctgaacgagaaggactacgaactgctgtgtctcgacggcacccgg
aagccagtggaggaatacgccaactgtcacctggccagagcccccaatcacgccgtggtcacccg
gaaggacaaagaggcctgcgtccacaagatcctgcggcagcagcagcacctgttcggcagcaacg
tgaccgactgcagcggcaacttctgcctgttcagaagcgagacaaaggacctcctgttccgggac
gacaccgtgtgtctggccaagctgcacgaccggaacacctacgagaagtacctgggcgaggaata
tgtgaaggccgtgggcaatctgcggaagtgcagcacctctagcctgctggaagcctgcacctttc
gcagacctTAA
(SEQ ID NO: 34)
MYRMQLLSCIALSLALVTNSPEEPLVVKVEEGDTAALWCLKGTSDGPTQQLTWSRESPLKPFLKY
SLGVPGLGVHVRPDAISVVIRNVSQQMGGFYLCQPGPPSEKAWQPGWTVNVEGSGELFRWNVSDL
GGLGCGLKNRSSEGPSSPSGKLMSPKLYVWAKDRPEIWEGEPPCLPPRDSLNQSLSRDLTVAPGS
TLWLSCGVPPDSVSRGPLSWTHVHPKGPKSLLSLELKDDRPARDMWVMGTSLMLPRATAQDAGKW
YCHRGNVTTSFHLEVIARPVKAHSDLRTGGWKVDGSGSDKTHTCPPCPAPELLGGPSVFLFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLS
LSPGKGSSGGSGGSGGSVPDKTVRWCAVSEHEATKCQSFRDHMKSVIPSDGPSVACVKKASYLDC
IRAIAANEADAVTLDAGLVYDAYLAPNNLKPVVAEFYGSKEDPQTFYYAVAVVKKDSGFQMNQLR
GKKSCHTGLGRSAGWNIPIGLLYCDLPEPRKPLEKAVANFFSGSCAPCADGTDFPQLCQLCPGCG
CSTLNQYFGYSGAFKCLKDGAGDVAFVKHSTIFENLANKADRDQYELLCLDNTRKPVDEYKDCHL
AQVPSHTVVARSMGGKEDLIWELLNQAQEHFGKDKSKEFQLFSSPHGKDLLFKDSAHGFLKVPPR
MDAKMYLGYEYVTAIRNLREGTCPEAPTDECKPVKWCALSHHERLKCDEWSVNSVGKIECVSAET
TEDCIAKIMNGEADAMSLDGGFVYIAGKCGLVPVLAENYNKSDNCEDTPEAGYFAIAVVKKSASD
LTWDNLKGKKSCHTAVGRTAGWNIPMGLLYNKINHCRFDEFFSEGCAPGSKKDSSLCKLCMGSGL
NLCEPNNKEGYYGYTGAFRCLVEKGDVAFVKHQTVPQNTGGKNPDPWAKNLNEKDYELLCLDGTR
KPVEEYANCHLARAPNHAVVTRKDKEACVHKILRQQQHLFGSNVTDCSGNFCLERSETKDLLFRD
DTVCLAKLHDRNTYEKYLGEEYVKAVGNLRKCSTSSLLEACTFRRP*
pDP49-CD19 NT.1-FC-GFLG-Tf
(SEQ ID NO: 35)
ATGTACAGGATGCAACTCCTGTCTTGCATTGCACTAAGTCTTGCACTTGTCACGAATTCGcccga
ggaacctctagtggtgaaggtggaagagggagataccgctgctctgtggtgcctcaaggggacct
cagatggccccactcagcagctgacctggtctcgggagtccccgcttaaacccttcttaaaatac
agcctgggggtgccaggcctgggagtccacgtcaggcccgatgccatctctgtcgtcatcaggaa
cgtctctcaacagatggggggcttctacctgtgccagccggggcccccctctgagaaggcctggc
agcctggctggacagtcaatgtggagggcagcggggagctgttccggtggaatgtttcggaccta
ggtggcctgggctgtggcctgaagaacaggtcctcagagggccccagctccccttccgggaagct
catgagccccaagctgtatgtgtgggccaaagaccgccctgagatctgggagggagagcctccgt
gtctcccaccgagggacagcctgaaccagagcctcagcagggacctcaccgtagcccctggctcc
acactctggctgtcctgtggggtaccccctgactctgtgtccaggggccccctctcctggaccca
tgtgcaccccaaggggcctaagtcattgctgagcctagagctgaaggacgatcgcccggccagag
atatgtgggtaatgggtacgtcactgatgttgccccgggccacagctcaagacgctggaaagtgg
tattgtcaccgtggcaacgtaaccacctcattccacctggaggtaatcgctcggccagtaaaggc
tcactcagacctgaggactggtggctggaagGTAGACGGCAGCGGCAGCGACAAAACTCACACAT
GCCCACCGTGCCCAGCACCTGAACTCCTGGGGGGACCGTCAGTCTTCCTCTTCCCCCCAAAACCC
AAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGC
CGCGGGAGGAGCAGTACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCCTGCCCCCATCCCGGG
ATGAGCTGACCAAGAACCAGGTCAGCCTGACCTGCCTGGTCAAAGGCTTCTATCCCAGCGACATC
GCCGTGGAGTGGGAGAGCAATGGGCAGCCGGAGAACAACTACAAGACCACGCCTCCCGTGCTGGA
CTCCGACGGCTCCTTCTTCCTCTACAGCAAGCTCACCGTGGACAAGAGCAGGTGGCAGCAGGGGA
ACGTCTTCTCATGCTCCGTGATGCATGAGGCTCTGCACAACCACTACACGCAGAAGAGCCTCTCC
CTGTCTCCGGGTAAAggatcctctggggggTTCCTGggaagcggtggttctgtgcccgataagac
agtgcggtggtgcgccgtgtctgagcacgaggccaccaagtgccagagcttccgggaccacatga
agtccgtgatccccagcgacggccctagcgtggcctgtgtgaagaaggccagctacctggactgc
atccgggccattgccgccaatgaggccgacgccgtgacactggatgccggcctggtgtacgatgc
ctacctggcccccaacaacctgaagcccgtggtggccgagttctacggcagcaaagaggaccccc
agaccttctactacgccgtggccgtggtcaagaaggacagcggcttccagatgaaccagctgcgg
ggcaagaagtcctgtcacaccggcctgggcagaagcgccggctggaacatccccatcggcctgct
gtactgcgatctgcccgagccccggaagcctctggaaaaggccgtggccaacttcttcagcggca
gctgcgccccttgtgctgacggaaccgacttcccccagctgtgtcagctgtgccccggctgtggc
tgcagcaccctgaaccagtacttcggctacagcggcgccttcaagtgcctgaaggacggcgctgg
cgacgtggccttcgtgaagcacagcaccatcttcgagaacctggccaacaaggccgaccgggacc
agtacgagctgctgtgcctggacaacaccagaaagcccgtggacgagtacaaggactgccacctc
gcccaggtgccatctcacacagtggtggcccggtccatgggcggcaaagaggatctgatctggga
gctgctgaaccaggcccaggaacacttcggcaaggacaagagcaaagagttccagctgttcagca
gcccccacggcaaggatctgctgttcaaggacagcgcccacggctttctgaaggtgccccccaga
atggacgccaagatgtacctgggctacgagtacgtgaccgccatccggaacctgagagagggcac
ctgtcccgaggcccccaccgatgagtgcaagcccgtgaagtggtgcgccctgagccaccacgagc
ggctgaagtgcgacgagtggagcgtgaacagcgtgggcaagatcgagtgcgtgagcgccgagaca
accgaggactgtatcgccaagatcatgaacggcgaggccgatgccatgagcctggacggcggctt
cgtgtacattgccggcaagtgcggcctggtgcctgtgctggccgagaactacaacaagagcgaca
actgcgaggatacccccgaggccggctactttgccatcgcagtcgtgaagaagtccgccagcgac
ctgacctgggacaatctgaagggcaagaaaagctgccacaccgccgtgggaaggaccgccgggtg
gaatattcctatggggctgctgtacaacaagatcaaccactgcagattcgacgagttcttcagcg
agggctgcgctcccggcagcaagaaagacagcagcctgtgcaagctgtgcatgggcagcggcctg
aacctgtgcgagcccaacaacaaagagggctactacggctacacaggggccttccggtgtctggt
ggagaagggggacgtggcttttgtgaaacaccagaccgtgccccagaacaccggggcaagaaccc
cgacccctgggccaagaacctgaacgagaaggactacgaactgctgtgtctcgacggcacccgga
agccagtggaggaatacgccaactgtcacctggccagagcccccaatcacgccgtggtcacccgg
aaggacaaagaggcctgcgtccacaagatcctgcggcagcagcagcacctgttcggcagcaacgt
gaccgactgcagcggcaacttctgcctgttcagaagcgagacaaaggacctcctgttccgggacg
acaccgtgtgtctggccaagctgcacgaccggaacacctacgagaagtacctgggcgaggaatat
gtgaaggccgtgggcaatctgcggaagtgcagcacctctagcctgctggaagcctgcacctttcg
cagacctaccggtCACCACCATCATCACCACCATCACTAA
(SEQ ID NO: 36)
MYRMQLLSCIALSLALVTNSPEEPLVVKVEEGDTAALWCLKGTSDGPTQQLTWSRESPLKPFLKY
SLGVPGLGVHVRPDAISVVIRNVSQQMGGFYLCQPGPPSEKAWQPGWTVNVEGSGELFRWNVSDL
GGLGCGLKNRSSEGPSSPSGKLMSPKLYVWAKDRPEIWEGEPPCLPPRDSLNQSLSRDLTVAPGS
TLWLSCGVPPDSVSRGPLSWTHVHPKGPKSLLSLELKDDRPARDMWVMGTSLMLPRATAQDAGKW
YCHRGNVTTSFHLEVIARPVKAHSDLRTGGWKVDGSGSDKTHTCPPCPAPELLGGPSVFLFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSVMHEALHNHYTQKSLS
LSPGKGSSGGFLGSGGSVPDKTVRWCAVSEHEATKCQSFRDHMKSVIPSDGPSVACVKKASYLDC
IRAIAANEADAVTLDAGLVYDAYLAPNNLKPVVAEFYGSKEDPQTFYYAVAVVKKDSGFQMNQLR
GKKSCHTGLGRSAGWNIPIGLLYCDLPEPRKPLEKAVANFFSGSCAPCADGTDFPQLCQLCPGCG
CSTLNQYFGYSGAFKCLKDGAGDVAFVKHSTIFENLANKADRDQYELLCLDNTRKPVDEYKDCHL
AQVPSHTVVARSMGGKEDLIWELLNQAQEHFGKDKSKEFQLFSSPHGKDLLFKDSAHGFLKVPPR
MDAKMYLGYEYVTAIRNLREGTCPEAPTDECKPVKWCALSHHERLKCDEWSVNSVGKIECVSAET
TEDCIAKIMNGEADAMSLDGGFVYIAGKCGLVPVLAENYNKSDNCEDTPEAGYFAIAVVKKSASD
LTWDNLKGKKSCHTAVGRTAGWNIPMGLLYNKINHCRFDEFFSEGCAPGSKKDSSLCKLCMGSGL
NLCEPNNKEGYYGYTGAFRCLVEKGDVAFVKHQTVPQNTGGKNPDPWAKNLNEKDYELLCLDGTR
KPVEEYANCHLARAPNHAVVTRKDKEACVHKILRQQQHLFGSNVTDCSGNFCLFRSETKDLLFRD
DTVCLAKLHDRNTYEKYLGEEYVKAVGNLRKCSTSSLLEACTFRRPTGHHHHHHHH*
pDP50-His8-CD19 NT.1-GFLG-FC-Tf
(SEQ ID NO: 37)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcGTAGACGGCGACAAAACTCACACATGCCCACCGTGCCCAGCACCTGAA
CTCCTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAACAGC
ACGTACCGTGTGGTCAGCGTCCTCACCGTCCTGCACCAGGACTGGCTGAATGGCAAGGAGTACAA
GTGCAAGGTCTCCAACAAAGCCCTCCCAGCCCCCATCGAGAAAACCATCTCCAAAGCCAAAGGGC
AGCCCCGAGAACCACAGGTGTACACCCTGCCCCCATCCCGGGATGAGCTGACCAAGAACCAGGTC
AGCCTGACCTGCCTGGTCAAAGGCTTCTATCCCAGCGACATCGCCGTGGAGTGGGAGAGCAATGG
GCAGCCGGAGAACAACTACAAGACCACGCCTCCCGTGCTGGACTCCGACGGCTCCTTCTTCCTCT
ACAGCAAGCTCACCGTGGACAAGAGCAGGTGGCAGCAGGGGAACGTCTTCTCATGCTCCGTGATG
CATGAGGCTCTGCACAACCACTACACGCAGAAGAGCCTCTCCCTGTCTCCGGGTAAAggatcctc
tgggggaagtggaggtagcggtggttctgtgcccgataagacagtgcggtggtgcgccgtgtctg
agcacgaggccaccaagtgccagagcttccgggaccacatgaagtccgtgatccccagcgacggc
cctagcgtggcctgtgtgaagaaggccagctacctggactgcatccgggccattgccgccaatga
ggccgacgccgtgacactggatgccggcctggtgtacgatgcctacctggcccccaacaacctga
agcccgtggtggccgagttctacggcagcaaagaggacccccagaccttctactacgccgtggcc
gtggtcaagaaggacagcggcttccagatgaaccagctgcggggcaagaagtcctgtcacaccgg
cctgggcagaagcgccggctggaacatccccatcggcctgctgtactgcgatctgcccgagcccc
ggaagcctctggaaaaggccgtggccaacttcttcagcggcagctgcgccccttgtgctgacgga
accgacttcccccagctgtgtcagctgtgccccggctgtggctgcagcaccctgaaccagtactt
cggctacagcggcgccttcaagtgcctgaaggacggcgctggcgacgtggccttcgtgaagcaca
gcaccatcttcgagaacctggccaacaaggccgaccgggaccagtacgagctgctgtgcctggac
aacaccagaaagcccgtggacgagtacaaggactgccacctcgcccaggtgccatctcacacagt
ggtggcccggtccatgggggcaaagaggatctgatctgggagctgctgaaccaggcccaggaaca
cttcggcaaggacaagagcaaagagttccagctgttcagcagcccccacggcaaggatctgctgt
tcaaggacagcgcccacggctttctgaaggtgccccccagaatggacgccaagatgtacctgggc
tacgagtacgtgaccgccatccggaacctgagagagggcacctgtcccgaggcccccaccgatga
gtgcaagcccgtgaagtggtgcgccctgagccaccacgagcggctgaagtgcgacgagtggagcg
tgaacagcgtgggcaagatcgagtgcgtgagcgccgagacaaccgaggactgtatcgccaagatc
atgaacggcgaggccgatgccatgagcctggacggcggcttcgtgtacattgccggcaagtgcgg
cctggtgcctgtgctggccgagaactacaacaagagcgacaactgcgaggatacccccgaggccg
gctactttgccatcgcagtcgtgaagaagtccgccagcgacctgacctgggacaatctgaagggc
aagaaaagctgccacaccgccgtgggaaggaccgccgggtggaatattcctatggggctgctgta
caacaagatcaaccactgcagattcgacgagttcttcagcgagggctgcgctcccggcagcaaga
aagacagcagcctgtgcaagctgtgcatgggcagcggcctgaacctgtgcgagcccaacaacaaa
gagggctactacggctacacaggggccttccggtgtctggtggagaagggggacgtggcttttgt
gaaacaccagaccgtgccccagaacaccggcggcaagaaccccgacccctgggccaagaacctga
acgagaaggactacgaactgctgtgtctcgacggcacccggaagccagtggaggaatacgccaac
tgtcacctggccagagcccccaatcacgccgtggtcacccggaaggacaaagaggcctgcgtcca
caagatcctgcggcagcagcagcacctgttcggcagcaacgtgaccgactgcagcggcaacttct
gcctgttcagaagcgagacaaaggacctcctgttccgggacgacaccgtgtgtctggccaagctg
cacgaccggaacacctacgagaagtacctgggcgaggaatatgtgaaggccgtgggcaatctgcg
gaagtgcagcacctctagcctgctggaagcctgcacctttcgcagacctTAA
(SEQ ID NO: 38)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVDGDKTHTCPPCPAPE
LLGGPSVFLFPPKPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVM
HEALHNHYTQKSLSLSPGKGSSGGSGGSGGSVPDKTVRWCAVSEHEATKCQSFRDHMKSVIPSDG
PSVACVKKASYLDCIRAIAANEADAVTLDAGLVYDAYLAPNNLKPVVAEFYGSKEDPQTFYYAVA
VVKKDSGFQMNQLRGKKSCHTGLGRSAGWNIPIGLLYCDLPEPRKPLEKAVANFFSGSCAPCADG
TDFPQLCQLCPGCGCSTLNQYFGYSGAFKCLKDGAGDVAFVKHSTIFENLANKADRDQYELLCLD
NTRKPVDEYKDCHLAQVPSHTVVARSMGGKEDLIWELLNQAQEHFGKDKSKEFQLESSPHGKDLL
FKDSAHGFLKVPPRMDAKMYLGYEYVTAIRNLREGTCPEAPTDECKPVKWCALSHHERLKCDEWS
VNSVGKIECVSAETTEDCIAKIMNGEADAMSLDGGFVYIAGKCGLVPVLAENYNKSDNCEDTPEA
GYFAIAVVKKSASDLTWDNLKGKKSCHTAVGRTAGWNIPMGLLYNKINHCRFDEFFSEGCAPGSK
KDSSLCKLCMGSGLNLCEPNNKEGYYGYTGAFRCLVEKGDVAFVKHQTVPQNTGGKNPDPWAKNL
NEKDYELLCLDGTRKPVEEYANCHLARAPNHAVVTRKDKEACVHKILRQQQHLFGSNVTDCSGNF
CLFRSETKDLLFRDDTVCLAKLHDRNTYEKYLGEEYVKAVGNLRKCSTSSLLEACTFRRP*
pDP85-cd20-scfv-GFLG-FC-Tf
(SEQ ID NO: 39)
ATGTACAGGATGCAACTCCTGTCTTGCATTGCACTAAGTCTTGCACTTGTCACGAATTCGCAGAT
TGTTCTGAGCCAGTCCCCGGCAATCCTCTCTGCCAGCCCAGGCGAAAAGGTGACAATGACTTGCC
GAGCGAGTTCCAGTGTCTCTTATATCCACTGGTTCCAGCAAAAGCCGGGAAGCAGCCCTAAACCA
TGGATATATGCAACGTCTAACCTGGCGAGCGGGGTCCCAGTGAGATTTTCCGGAAGCGGCAGCGG
AACTAGTTACTCTTTGACAATAAGCAGAGTGGAGGCTGAGGACGCTGCTACTTACTATTGCCAGC
AATGGACGAGTAACCCGCCGACGTTTGGAGGTGGAACGAAGCTGGAGATTAAAGGTGGAGGTGGT
TCTGGCGGAGGTGGTTCCGGTGGTGGTGGAAGTCAGGTGCAGCTCCAACAGCCTGGTGCCGAACT
TGTCAAACCTGGGGCTAGTGTGAAGATGAGTTGCAAAGCTTCAGGGTACACGTTTACGTCATACA
ACATGCATTGGGTAAAGCAAACACCAGGACGCGGCTTGGAATGGATCGGCGCGATATATCCAGGA
AACGGTGACACTTCTTATAACCAGAAGTTCAAGGGGAAAGCTACTCTCACAGCGGACAAATCTTC
TTCAACAGCGTATATGCAGTTGTCAAGCCTTACTAGCGAGGACAGTGCTGTTTATTACTGCGCCC
GGTCCACCTATTATGGGGGTGATTGGTACTTTAATGTTTGGGGCGCGGGTACTACCGTTACTGTG
TCCGCGGGTGGCAGCGGCAGCggtgggTTCCTGggaGGCGTAGACGGCGACAAAACTCACACATG
CCCACCGTGCCCAGCACCTGAACTCCTGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCC
TGTCTCCGGGTAAAggatcctctgggggaagtggaggtagcggtggttctgtgcccgataagaca
gtgcggtggtgcgccgtgtctgagcacgaggccaccaagtgccagagcttccgggaccacatgaa
gtccgtgatccccagcgacggccctagcgtggcctgtgtgaagaaggccagctacctggactgca
tccgggccattgccgccaatgaggccgacgccgtgacactggatgccggcctggtgtacgatgcc
tacctggcccccaacaacctgaagcccgtggtggccgagttctacggcagcaaagaggaccccca
gaccttctactacgccgtggccgtggtcaagaaggacagcggcttccagatgaaccagctgcggg
gcaagaagtcctgtcacaccggcctgggcagaagcgccggctggaacatccccatcggcctgctg
tactgcgatctgcccgagccccggaagcctctggaaaaggccgtggccaacttcttcagcggcag
ctgcgccccttgtgctgacggaaccgacttcccccagctgtgtcagctgtgccccggctgtggct
gcagcaccctgaaccagtacttcggctacagcggcgccttcaagtgcctgaaggacggcgctggc
gacgtggccttcgtgaagcacagcaccatcttcgagaacctggccaacaaggccgaccgggacca
gtacgagctgctgtgcctggacaacaccagaaagcccgtggacgagtacaaggactgccacctcg
cccaggtgccatctcacacagtggtggcccggtccatgggcggcaaagaggatctgatctgggag
ctgctgaaccaggcccaggaacacttcggcaaggacaagagcaaagagttccagctgttcagcag
cccccacggcaaggatctgctgttcaaggacagcgcccacggctttctgaaggtgccccccagaa
tggacgccaagatgtacctgggctacgagtacgtgaccgccatccggaacctgagagagggcacc
tgtcccgaggcccccaccgatgagtgcaagcccgtgaagtggtgcgccctgagccaccacgagcg
gctgaagtgcgacgagtggagcgtgaacagcgtgggcaagatcgagtgcgtgagcgccgagacaa
ccgaggactgtatcgccaagatcatgaacggcgaggccgatgccatgagcctggacggcggcttc
gtgtacattgccggcaagtgcggcctggtgcctgtgctggccgagaactacaacaagagcgacaa
ctgcgaggatacccccgaggccggctactttgccatcgcagtcgtgaagaagtccgccagcgacc
tgacctgggacaatctgaagggcaagaaaagctgccacaccgccgtgggaaggaccgccgggtgg
aatattcctatggggctgctgtacaacaagatcaaccactgcagattcgacgagttcttcagcga
gggctgcgctcccggcagcaagaaagacagcagcctgtgcaagctgtgcatgggcagcggcctga
acctgtgcgagcccaacaacaaagagggctactacggctacacaggggccttccggtgtctggtg
gagaagggggacgtggcttttgtgaaacaccagaccgtgccccagaacaccggcggcaagaaccc
cgacccctgggccaagaacctgaacgagaaggactacgaactgctgtgtctcgacggcacccgga
agccagtggaggaatacgccaactgtcacctggccagagcccccaatcacgccgtggtcacccgg
aaggacaaagaggcctgcgtccacaagatcctgcggcagcagcagcacctgttcggcagcaacgt
gaccgactgcagcggcaacttctgcctgttcagaagcgagacaaaggacctcctgttccgggacg
acaccgtgtgtctggccaagctgcacgaccggaacacctacgagaagtacctgggcgaggaatat
gtgaaggccgtgggcaatctgcggaagtgcagcacctctagcctgctggaagcctgcacctttcg
cagacctTAA
(SEQ ID NO: 40)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSSGGSGGSGGSVPDKT
VRWCAVSEHEATKCQSFRDHMKSVIPSDGPSVACVKKASYLDCIRAIAANEADAVTLDAGLVYDA
YLAPNNLKPVVAEFYGSKEDPQTFYYAVAVVKKDSGFQMNQLRGKKSCHTGLGRSAGWNIPIGLL
YCDLPEPRKPLEKAVANFFSGSCAPCADGTDFPQLCQLCPGCGCSTLNQYFGYSGAFKCLKDGAG
DVAFVKHSTIFENLANKADRDQYELLCLDNTRKPVDEYKDCHLAQVPSHTVVARSMGGKEDLIWE
LLNQAQEHFGKDKSKEFQLFSSPHGKDLLFKDSAHGFLKVPPRMDAKMYLGYEYVTAIRNLREGT
CPEAPTDECKPVKWCALSHHERLKCDEWSVNSVGKIECVSAETTEDCIAKIMNGEADAMSLDGGF
VYIAGKCGLVPVLAENYNKSDNCEDTPEAGYFAIAVVKKSASDLTWDNLKGKKSCHTAVGRTAGW
NIPMGLLYNKINHCRFDEFFSEGCAPGSKKDSSLCKLCMGSGLNLCEPNNKEGYYGYTGAFRCLV
EKGDVAFVKHQTVPQNTGGKNPDPWAKNLNEKDYELLCLDGTRKPVEEYANCHLARAPNHAVVTR
KDKEACVHKILRQQQHLFGSNVTDCSGNFCLFRSETKDLLFRDDTVCLAKLHDRNTYEKYLGEEY
VKAVGNLRKCSTSSLLEACTFRRP*
pDP95-Herceptin_HC_Fc-N297G, S427C
(SEQ ID NO: 41)
ATGTACAGGATGCAACTCCTGTCTTGCATTGCACTAAGTCTTGCACTTGTCACGAATTCGgaggt
tcagctggtggagtctggcggtggcctggtgcagccagggggctcactccgtttgtcctgtgcag
cttctggcttcaacATCAAGGACACCTATATCcactgggtgcgtcaggccccgggtaagggcctg
gaatgggttgcaCGCATCTACCCGACGAATGGCTACACGCGCTatgccgatAGCgtcaagggccg
tttcactataagcGCAGACACATCCAAAAACACAGCCtacctacaaatgaacagcttaagagctg
aggacactgccgtctattattgtTCACGCTGGGGGGGAGATGGGTTTTATGCAATGgactactgg
ggtcaaggaaccctggtcaccgtctcctcggcctccaccaagggtccatcggtcttccccctggc
accctcctccaagagcacctctgggggcacagcggccctgggctgcctggtcaaggactacttcc
ccgaaccggtgacggtgtcgtggaactcaggcgccctgaccagcggcgtgcacaccttcccggct
gtcctacagtcctcaggactctactccctcagcagcgtggtgaccgtgccctccagcagcttggg
cacccagacctacatctgcaacgtgaatcacaagcccagcaacaccaaggtcgacaaAAAGGTCg
agcccaaatcttgtgacaaaactcacacatgcCCCCCCTGCCCAGCGCCAGAATTGCTGGGCGGA
CCCAGCGTGTTCCTGTTCCCCCCCAAACCTAAAGACACCCTGATGATCAGCCGAACCCCTGAGGT
GACCTGCGTGGTGGTGGACGTGAGCCACGAGGACCCCGAGGTGAAGTTCAACTGGTATGTGGACG
GCGTGGAGGTCCACAATGCCAAAACGAAGCCCAGGGAGGAGCAGTACGGCAGCACCTACAGGGTA
GTGAGCGTCTTGACCGTGCTGCACCAGGACTGGCTGAACGGCAAGGAATACAAATGCAAGGTCAG
CAATAAGGCTCTGCCGGCTCCTATCGAGAAGACAATCAGCAAGGCAAAGGGCCAGCCACGCGAAC
CGCAGGTGTATACTCTGCCCCCCAGCCGGGACGAGCTGACCAAGAACCAGGTGTCCCTGACCTGT
CTGGTGAAAGGCTTCTACCCCAGCGACATCGCTGTGGAGTGGGAGAGTAACGGGCAGCCCGAGAA
CAACTACAAGACCACGCCTCCTGTGCTGGACAGCGACGGCAGCTTCTTCCTGTATAGCAAGCTCA
CCGTGGACAAGAGCAGGTGGCAACAGGGCAACGTGTTCTGCTGCTCTGTGATGCACGAGGCCCTG
CACAACCATTACACCCAGAAGAGTCTCAGTCTGAGCCCGGGAAAGtaa
(SEQ ID NO: 42)
MYRMQLLSCIALSLALVTNSEVQLVESGGGLVQPGGSLRLSCAASGFNIKDTYIHWVRQAPGKGL
EWVARIYPTNGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQYGSTYRV
VSVLTVLHQDWLNGKEYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRWQQGNVFCCSVMHEAL
HNHYTQKSLSLSPGK*
pDP69-His8-CD19 NT.1-2XGFLG-FC-Tf
(SEQ ID NO: 43)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtggaTTtCTGggcggcgggTTCCTGggaggcGTAGACGGCGACAAAACTCACACATGCCCACCG
TGCCCAGCACCTGAACTCCTGGGGGGACCGTCAGTCTTCCTCTTCCCCCCAAAACCCAAGGACAC
CCTCATGATCTCCCGGACCCCTGAGGTCACATGCGTGGTGGTGGACGTGAGCCACGAAGACCCTG
AGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAA
TGGCAAGGAGTACAAGTGCAAGGTCTCCAACAAAGCCCTCCCAGCCCCCATCGAGAAAACCATCT
CCAAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGACTCCGACG
GCTCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCTCC
GGGTAAAggatcctctgggggaagtggaggtagcggtggttctgtgcccgataagacagtgcggt
ggtgcgccgtgtctgagcacgaggccaccaagtgccagagcttccgggaccacatgaagtccgtg
atccccagcgacggccctagcgtggcctgtgtgaagaaggccagctacctggactgcatccgggc
cattgccgccaatgaggccgacgccgtgacactggatgccggcctggtgtacgatgcctacctgg
cccccaacaacctgaagcccgtggtggccgagttctacggcagcaaagaggacccccagaccttc
tactacgccgtggccgtggtcaagaaggacagcggcttccagatgaaccagctgcggggcaagaa
gtcctgtcacaccggcctgggcagaagcgccggctggaacatccccatcggcctgctgtactgcg
atctgcccgagccccggaagcctctggaaaaggccgtggccaacttcttcagcggcagctgcgcc
ccttgtgctgacggaaccgacttcccccagctgtgtcagctgtgccccggctgtggctgcagcac
cctgaaccagtacttcggctacagcggcgccttcaagtgcctgaaggacggcgctggcgacgtgg
ccttcgtgaagcacagcaccatcttcgagaacctggccaacaaggccgaccgggaccagtacgag
ctgctgtgcctggacaacaccagaaagcccgtggacgagtacaaggactgccacctcgcccaggt
gccatctcacacagtggtggcccggtccatgggcggcaaagaggatctgatctgggagctgctga
accaggcccaggaacacttcggcaaggacaagagcaaagagttccagctgttcagcagcccccac
ggcaaggatctgctgttcaaggacagcgcccacggctttctgaaggtgccccccagaatggacgc
caagatgtacctgggctacgagtacgtgaccgccatccggaacctgagagagggcacctgtcccg
aggcccccaccgatgagtgcaagcccgtgaagtggtgcgccctgagccaccacgagcggctgaag
tgcgacgagtggagcgtgaacagcgtgggcaagatcgagtgcgtgagcgccgagacaaccgagga
ctgtatcgccaagatcatgaacggcgaggccgatgccatgagcctggacggcggcttcgtgtaca
ttgccggcaagtgcggcctggtgcctgtgctggccgagaactacaacaagagcgacaactgcgag
gatacccccgaggccggctactttgccatcgcagtcgtgaagaagtccgccagcgacctgacctg
ggacaatctgaagggcaagaaaagctgccacaccgccgtgggaaggaccgccgggtggaatattc
ctatggggctgctgtacaacaagatcaaccactgcagattcgacgagttcttcagcgagggctgc
gctcccggcagcaagaaagacagcagcctgtgcaagctgtgcatgggcagcggcctgaacctgtg
cgagcccaacaacaaagagggctactacggctacacaggggccttccggtgtctggtggagaagg
gggacgtggcttttgtgaaacaccagaccgtgccccagaacaccggggcaagaaccccgacccct
gggccaagaacctgaacgagaaggactacgaactgctgtgtctcgacggcacccggaagccagtg
gaggaatacgccaactgtcacctggccagagcccccaatcacgccgtggtcacccggaaggacaa
agaggcctgcgtccacaagatcctgcggcagcagcagcacctgttcggcagcaacgtgaccgact
gcagcggcaacttctgcctgttcagaagcgagacaaaggacctcctgttccgggacgacaccgtg
tgtctggccaagctgcacgaccggaacacctacgagaagtacctgggcgaggaatatgtgaaggc
cgtgggcaatctgcggaagtgcagcacctctagcctgctggaagcctgcacctttcgcagacctT
AA
(SEQ ID NO: 44)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGFLGGVDGDKTHTCPP
CPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVF
SCSVMHEALHNHYTQKSLSLSPGKGSSGGSGGSGGSVPDKTVRWCAVSEHEATKCQSFRDHMKSV
IPSDGPSVACVKKASYLDCIRAIAANEADAVTLDAGLVYDAYLAPNNLKPVVAEFYGSKEDPQTF
YYAVAVVKKDSGFQMNQLRGKKSCHTGLGRSAGWNIPIGLLYCDLPEPRKPLEKAVANFFSGSCA
PCADGTDFPQLCQLCPGCGCSTLNQYFGYSGAFKCLKDGAGDVAFVKHSTIFENLANKADRDQYE
LLCLDNTRKPVDEYKDCHLAQVPSHTVVARSMGGKEDLIWELLNQAQEHFGKDKSKEFQLFSSPH
GKDLLFKDSAHGFLKVPPRMDAKMYLGYEYVTAIRNLREGTCPEAPTDECKPVKWCALSHHERLK
CDEWSVNSVGKIECVSAETTEDCIAKIMNGEADAMSLDGGFVYIAGKCGLVPVLAENYNKSDNCE
DTPEAGYFAIAVVKKSASDLTWDNLKGKKSCHTAVGRTAGWNIPMGLLYNKINHCRFDEFFSEGC
APGSKKDSSLCKLCMGSGLNLCEPNNKEGYYGYTGAFRCLVEKGDVAFVKHQTVPQNTGGKNPDP
WAKNLNEKDYELLCLDGTRKPVEEYANCHLARAPNHAVVTRKDKEACVHKILRQQQHLFGSNVTD
CSGNFCLFRSETKDLLFRDDTVCLAKLHDRNTYEKYLGEEYVKAVGNLRKCSTSSLLEACTFRRP
*
pDP70-His8-CD19 NT.1-3XGFLG-FC-Tf
(SEQ ID NO: 45)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtggaTTCCTGggcggcgggTTtCTGggaggcggaTTtCTGggaggcGTAGACGGCGACAAAACT
CACACATGCCCACCGTGCCCAGCACCTGAACTCCTGGGGGGACCGTCAGTCTTCCTCTTCCCCCC
AAAACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCCAAG
ACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCA
TCGAGAAAACCATCTCCAAAGCCAAAGGGCAGCCCCGAGAACCACAGGTGTACACCCTGCCCCCA
TCCCGGGATGAGCTGACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACAAGACCACGCCTCCCG
TGCTGGACTCCGACGGCTCCTTCTTCCTCTACAGCAAGCTCACCGTGGACAAGAGCAGGTGGCAG
CAGGGGAACGTCTTCTCATGCTCCGTGATGCATGAGGCTCTGCACAACCACTACACGCAGAAGAG
CCTCTCCCTGTCTCCGGGTAAAggatcctctgggggaagtggaggtagcggtggttctgtgcccg
ataagacagtgcggtggtgcgccgtgtctgagcacgaggccaccaagtgccagagcttccgggac
cacatgaagtccgtgatccccagcgacggccctagcgtggcctgtgtgaagaaggccagctacct
ggactgcatccgggccattgccgccaatgaggccgacgccgtgacactggatgccggcctggtgt
acgatgcctacctggcccccaacaacctgaagcccgtggtggccgagttctacggcagcaaagag
gacccccagaccttctactacgccgtggccgtggtcaagaaggacagcggcttccagatgaacca
gctgcggggcaagaagtcctgtcacaccggcctgggcagaagcgccggctggaacatccccatcg
gcctgctgtactgcgatctgcccgagccccggaagcctctggaaaaggccgtggccaacttcttc
agcggcagctgcgccccttgtgctgacggaaccgacttcccccagctgtgtcagctgtgccccgg
ctgtggctgcagcaccctgaaccagtacttcggctacagcggcgccttcaagtgcctgaaggacg
gcgctggcgacgtggccttcgtgaagcacagcaccatcttcgagaacctggccaacaaggccgac
cgggaccagtacgagctgctgtgcctggacaacaccagaaagcccgtggacgagtacaaggactg
ccacctcgcccaggtgccatctcacacagtggtggcccggtccatgggcggcaaagaggatctga
tctgggagctgctgaaccaggcccaggaacacttcggcaaggacaagagcaaagagttccagctg
ttcagcagcccccacggcaaggatctgctgttcaaggacagcgcccacggctttctgaaggtgcc
ccccagaatggacgccaagatgtacctgggctacgagtacgtgaccgccatccggaacctgagag
agggcacctgtcccgaggcccccaccgatgagtgcaagcccgtgaagtggtgcgccctgagccac
cacgagcggctgaagtgcgacgagtggagcgtgaacagcgtgggcaagatcgagtgcgtgagcgc
cgagacaaccgaggactgtatcgccaagatcatgaacggcgaggccgatgccatgagcctggacg
gcggcttcgtgtacattgccggcaagtgcggcctggtgcctgtgctggccgagaactacaacaag
agcgacaactgcgaggatacccccgaggccggctactttgccatcgcagtcgtgaagaagtccgc
cagcgacctgacctgggacaatctgaagggcaagaaaagctgccacaccgccgtgggaaggaccg
ccgggtggaatattcctatggggctgctgtacaacaagatcaaccactgcagattcgacgagttc
ttcagcgagggctgcgctcccggcagcaagaaagacagcagcctgtgcaagctgtgcatgggcag
cggcctgaacctgtgcgagcccaacaacaaagagggctactacggctacacaggggccttccggt
gtctggtggagaagggggacgtggcttttgtgaaacaccagaccgtgccccagaacaccggcggc
aagaaccccgacccctgggccaagaacctgaacgagaaggactacgaactgctgtgtctcgacgg
cacccggaagccagtggaggaatacgccaactgtcacctggccagagcccccaatcacgccgtgg
tcacccggaaggacaaagaggcctgcgtccacaagatcctgcggcagcagcagcacctgttcggc
agcaacgtgaccgactgcagcggcaacttctgcctgttcagaagcgagacaaaggacctcctgtt
ccgggacgacaccgtgtgtctggccaagctgcacgaccggaacacctacgagaagtacctgggcg
aggaatatgtgaaggccgtgggcaatctgcggaagtgcagcacctctagcctgctggaagcctgc
acctttcgcagacctTAA
(SEQ ID NO: 46)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGFLGGGFLGGVDGDKT
HTCPPCPAPELLGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGKGSSGGSGGSGGSVPDKTVRWCAVSEHEATKCQSFRD
HMKSVIPSDGPSVACVKKASYLDCIRAIAANEADAVTLDAGLVYDAYLAPNNLKPVVAEFYGSKE
DPQTFYYAVAVVKKDSGFQMNQLRGKKSCHTGLGRSAGWNIPIGLLYCDLPEPRKPLEKAVANFF
SGSCAPCADGTDFPQLCQLCPGCGCSTLNQYFGYSGAFKCLKDGAGDVAFVKHSTIFENLANKAD
RDQYELLCLDNTRKPVDEYKDCHLAQVPSHTVVARSMGGKEDLIWELLNQAQEHFGKDKSKEFQL
FSSPHGKDLLFKDSAHGFLKVPPRMDAKMYLGYEYVTAIRNLREGTCPEAPTDECKPVKWCALSH
HERLKCDEWSVNSVGKIECVSAETTEDCIAKIMNGEADAMSLDGGFVYIAGKCGLVPVLAENYNK
SDNCEDTPEAGYFAIAVVKKSASDLTWDNLKGKKSCHTAVGRTAGWNIPMGLLYNKINHCRFDEF
FSEGCAPGSKKDSSLCKLCMGSGLNLCEPNNKEGYYGYTGAFRCLVEKGDVAFVKHQTVPQNTGG
KNPDPWAKNLNEKDYELLCLDGTRKPVEEYANCHLARAPNHAVVTRKDKEACVHKILRQQQHLFG
SNVTDCSGNFCLFRSETKDLLFRDDTVCLAKLHDRNTYEKYLGEEYVKAVGNLRKCSTSSLLEAC
TERRP*
pDP71-His8-CD19 NT.1-GFLG-FK-FC-Tf
(SEQ ID NO: 47)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcTTCAAAggcGTAGACGGCGACAAAACTCACACATGCCCACCGTGCCCA
GCACCTGAACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAA
Aggatcctctgggggaagtggaggtagcggtggttctgtgcccgataagacagtgcggtggtgcg
ccgtgtctgagcacgaggccaccaagtgccagagcttccgggaccacatgaagtccgtgatcccc
agcgacggccctagcgtggcctgtgtgaagaaggccagctacctggactgcatccgggccattgc
cgccaatgaggccgacgccgtgacactggatgccggcctggtgtacgatgcctacctggccccca
acaacctgaagcccgtggtggccgagttctacggcagcaaagaggacccccagaccttctactac
gccgtggccgtggtcaagaaggacagcggcttccagatgaaccagctgcggggcaagaagtcctg
tcacaccggcctgggcagaagcgccggctggaacatccccatcggcctgctgtactgcgatctgc
ccgagccccggaagcctctggaaaaggccgtggccaacttcttcagcggcagctgcgccccttgt
gctgacggaaccgacttcccccagctgtgtcagctgtgccccggctgtggctgcagcaccctgaa
ccagtacttcggctacagcggcgccttcaagtgcctgaaggacggcgctggcgacgtggccttcg
tgaagcacagcaccatcttcgagaacctggccaacaaggccgaccgggaccagtacgagctgctg
tgcctggacaacaccagaaagcccgtggacgagtacaaggactgccacctcgcccaggtgccatc
tcacacagtggtggcccggtccatgggcggcaaagaggatctgatctgggagctgctgaaccagg
cccaggaacacttcggcaaggacaagagcaaagagttccagctgttcagcagcccccacggcaag
gatctgctgttcaaggacagcgcccacggctttctgaaggtgccccccagaatggacgccaagat
gtacctgggctacgagtacgtgaccgccatccggaacctgagagagggcacctgtcccgaggccc
ccaccgatgagtgcaagcccgtgaagtggtgcgccctgagccaccacgagcggctgaagtgcgac
gagtggagcgtgaacagcgtgggcaagatcgagtgcgtgagcgccgagacaaccgaggactgtat
cgccaagatcatgaacggcgaggccgatgccatgagcctggacggcggcttcgtgtacattgccg
gcaagtgcggcctggtgcctgtgctggccgagaactacaacaagagcgacaactgcgaggatacc
cccgaggccggctactttgccatcgcagtcgtgaagaagtccgccagcgacctgacctgggacaa
tctgaagggcaagaaaagctgccacaccgccgtgggaaggaccgccgggtggaatattcctatgg
ggctgctgtacaacaagatcaaccactgcagattcgacgagttcttcagcgagggctgcgctccc
ggcagcaagaaagacagcagcctgtgcaagctgtgcatgggcagcggcctgaacctgtgcgagcc
caacaacaaagagggctactacggctacacaggggccttccggtgtctggtggagaagggggacg
tggcttttgtgaaacaccagaccgtgccccagaacaccggcggcaagaaccccgacccctgggcc
aagaacctgaacgagaaggactacgaactgctgtgtctcgacggcacccggaagccagtggagga
atacgccaactgtcacctggccagagcccccaatcacgccgtggtcacccggaaggacaaagagg
cctgcgtccacaagatcctgcggcagcagcagcacctgttcggcagcaacgtgaccgactgcagc
ggcaacttctgcctgttcagaagcgagacaaaggacctcctgttccgggacgacaccgtgtgtct
ggccaagctgcacgaccggaacacctacgagaagtacctgggcgaggaatatgtgaaggccgtgg
gcaatctgcggaagtgcagcacctctagcctgctggaagcctgcacctttcgcagacctTAA
(SEQ ID NO: 48)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGFKGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSVPDKTVRWCAVSEHEATKCQSFRDHMKSVIP
SDGPSVACVKKASYLDCIRAIAANEADAVTLDAGLVYDAYLAPNNLKPVVAEFYGSKEDPQTFYY
AVAVVKKDSGFQMNQLRGKKSCHTGLGRSAGWNIPIGLLYCDLPEPRKPLEKAVANFFSGSCAPC
ADGTDFPQLCQLCPGCGCSTLNQYFGYSGAFKCLKDGAGDVAFVKHSTIFENLANKADRDQYELL
CLDNTRKPVDEYKDCHLAQVPSHTVVARSMGGKEDLIWELLNQAQEHFGKDKSKEFQLFSSPHGK
DLLFKDSAHGFLKVPPRMDAKMYLGYEYVTAIRNLREGTCPEAPTDECKPVKWCALSHHERLKCD
EWSVNSVGKIECVSAETTEDCIAKIMNGEADAMSLDGGFVYIAGKCGLVPVLAENYNKSDNCEDT
PEAGYFAIAVVKKSASDLTWDNLKGKKSCHTAVGRTAGWNIPMGLLYNKINHCRFDEFFSEGCAP
GSKKDSSLCKLCMGSGLNLCEPNNKEGYYGYTGAFRCLVEKGDVAFVKHQTVPQNTGGKNPDPWA
KNLNEKDYELLCLDGTRKPVEEYANCHLARAPNHAVVTRKDKEACVHKILRQQQHLFGSNVTDCS
GNFCLFRSETKDLLFRDDTVCLAKLHDRNTYEKYLGEEYVKAVGNLRKCSTSSLLEACTFRRP*
pDP72-His8-CD19 NT.1-GFLG-VA-FC-Tf
(SEQ ID NO: 49)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcGTAgctggcGTAGACGGCGACAAAACTCACACATGCCCACCGTGCCCA
GCACCTGAACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAA
Aggatcctctgggggaagtggaggtagcggtggttctgtgcccgataagacagtgcggtggtgcg
ccgtgtctgagcacgaggccaccaagtgccagagcttccgggaccacatgaagtccgtgatcccc
agcgacggccctagcgtggcctgtgtgaagaaggccagctacctggactgcatccgggccattgc
cgccaatgaggccgacgccgtgacactggatgccggcctggtgtacgatgcctacctggccccca
acaacctgaagcccgtggtggccgagttctacggcagcaaagaggacccccagaccttctactac
gccgtggccgtggtcaagaaggacagcggcttccagatgaaccagctgcggggcaagaagtcctg
tcacaccggcctgggcagaagcgccggctggaacatccccatcggcctgctgtactgcgatctgc
ccgagccccggaagcctctggaaaaggccgtggccaacttcttcagcggcagctgcgccccttgt
gctgacggaaccgacttcccccagctgtgtcagctgtgccccggctgtggctgcagcaccctgaa
ccagtacttcggctacagcggcgccttcaagtgcctgaaggacggcgctggcgacgtggccttcg
tgaagcacagcaccatcttcgagaacctggccaacaaggccgaccgggaccagtacgagctgctg
tgcctggacaacaccagaaagcccgtggacgagtacaaggactgccacctcgcccaggtgccatc
tcacacagtggtggcccggtccatgggcggcaaagaggatctgatctgggagctgctgaaccagg
cccaggaacacttcggcaaggacaagagcaaagagttccagctgttcagcagcccccacggcaag
gatctgctgttcaaggacagcgcccacggctttctgaaggtgccccccagaatggacgccaagat
gtacctgggctacgagtacgtgaccgccatccggaacctgagagagggcacctgtcccgaggccc
ccaccgatgagtgcaagcccgtgaagtggtgcgccctgagccaccacgagcggctgaagtgcgac
gagtggagcgtgaacagcgtgggcaagatcgagtgcgtgagcgccgagacaaccgaggactgtat
cgccaagatcatgaacggcgaggccgatgccatgagcctggacggcggcttcgtgtacattgccg
gcaagtgcggcctggtgcctgtgctggccgagaactacaacaagagcgacaactgcgaggatacc
cccgaggccggctactttgccatcgcagtcgtgaagaagtccgccagcgacctgacctgggacaa
tctgaagggcaagaaaagctgccacaccgccgtgggaaggaccgccgggtggaatattcctatgg
ggctgctgtacaacaagatcaaccactgcagattcgacgagttcttcagcgagggctgcgctccc
ggcagcaagaaagacagcagcctgtgcaagctgtgcatgggcagcggcctgaacctgtgcgagcc
caacaacaaagagggctactacggctacacaggggccttccggtgtctggtggagaagggggacg
tggcttttgtgaaacaccagaccgtgccccagaacaccggcggcaagaaccccgacccctgggcc
aagaacctgaacgagaaggactacgaactgctgtgtctcgacggcacccggaagccagtggagga
atacgccaactgtcacctggccagagcccccaatcacgccgtggtcacccggaaggacaaagagg
cctgcgtccacaagatcctgcggcagcagcagcacctgttcggcagcaacgtgaccgactgcagc
ggcaacttctgcctgttcagaagcgagacaaaggacctcctgttccgggacgacaccgtgtgtct
ggccaagctgcacgaccggaacacctacgagaagtacctgggcgaggaatatgtgaaggccgtgg
gcaatctgcggaagtgcagcacctctagcctgctggaagcctgcacctttcgcagacctTAA
(SEQ ID NO: 50)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVAGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSVPDKTVRWCAVSEHEATKCQSFRDHMKSVIP
SDGPSVACVKKASYLDCIRAIAANEADAVTLDAGLVYDAYLAPNNLKPVVAEFYGSKEDPQTFYY
AVAVVKKDSGFQMNQLRGKKSCHTGLGRSAGWNIPIGLLYCDLPEPRKPLEKAVANFFSGSCAPC
ADGTDFPQLCQLCPGCGCSTLNQYFGYSGAFKCLKDGAGDVAFVKHSTIFENLANKADRDQYELL
CLDNTRKPVDEYKDCHLAQVPSHTVVARSMGGKEDLIWELLNQAQEHFGKDKSKEFQLFSSPHGK
DLLFKDSAHGFLKVPPRMDAKMYLGYEYVTAIRNLREGTCPEAPTDECKPVKWCALSHHERLKCD
EWSVNSVGKIECVSAETTEDCIAKIMNGEADAMSLDGGFVYIAGKCGLVPVLAENYNKSDNCEDT
PEAGYFAIAVVKKSASDLTWDNLKGKKSCHTAVGRTAGWNIPMGLLYNKINHCRFDEFFSEGCAP
GSKKDSSLCKLCMGSGLNLCEPNNKEGYYGYTGAFRCLVEKGDVAFVKHQTVPQNTGGKNPDPWA
KNLNEKDYELLCLDGTRKPVEEYANCHLARAPNHAVVTRKDKEACVHKILRQQQHLFGSNVTDCS
GNFCLFRSETKDLLFRDDTVCLAKLHDRNTYEKYLGEEYVKAVGNLRKCSTSSLLEACTFRRP*
pDP73-His8-CD19 NT.1-GFLG-VK-FC-Tf
(SEQ ID NO: 51)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcGTAAAAggcGTAGACGGCGACAAAACTCACACATGCCCACCGTGCCCA
GCACCTGAACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAA
Aggatcctctgggggaagtggaggtagcggtggttctgtgcccgataagacagtgcggtggtgcg
ccgtgtctgagcacgaggccaccaagtgccagagcttccgggaccacatgaagtccgtgatcccc
agcgacggccctagcgtggcctgtgtgaagaaggccagctacctggactgcatccgggccattgc
cgccaatgaggccgacgccgtgacactggatgccggcctggtgtacgatgcctacctggccccca
acaacctgaagcccgtggtggccgagttctacggcagcaaagaggacccccagaccttctactac
gccgtggccgtggtcaagaaggacagcggcttccagatgaaccagctgcggggcaagaagtcctg
tcacaccggcctgggcagaagcgccggctggaacatccccatcggcctgctgtactgcgatctgc
ccgagccccggaagcctctggaaaaggccgtggccaacttcttcagcggcagctgcgccccttgt
gctgacggaaccgacttcccccagctgtgtcagctgtgccccggctgtggctgcagcaccctgaa
ccagtacttcggctacagcggcgccttcaagtgcctgaaggacggcgctggcgacgtggccttcg
tgaagcacagcaccatcttcgagaacctggccaacaaggccgaccgggaccagtacgagctgctg
tgcctggacaacaccagaaagcccgtggacgagtacaaggactgccacctcgcccaggtgccatc
tcacacagtggtggcccggtccatgggcggcaaagaggatctgatctgggagctgctgaaccagg
cccaggaacacttcggcaaggacaagagcaaagagttccagctgttcagcagcccccacggcaag
gatctgctgttcaaggacagcgcccacggctttctgaaggtgccccccagaatggacgccaagat
gtacctgggctacgagtacgtgaccgccatccggaacctgagagagggcacctgtcccgaggccc
ccaccgatgagtgcaagcccgtgaagtggtgcgccctgagccaccacgagcggctgaagtgcgac
gagtggagcgtgaacagcgtgggcaagatcgagtgcgtgagcgccgagacaaccgaggactgtat
cgccaagatcatgaacggcgaggccgatgccatgagcctggacggcggcttcgtgtacattgccg
gcaagtgcggcctggtgcctgtgctggccgagaactacaacaagagcgacaactgcgaggatacc
cccgaggccggctactttgccatcgcagtcgtgaagaagtccgccagcgacctgacctgggacaa
tctgaagggcaagaaaagctgccacaccgccgtgggaaggaccgccgggtggaatattcctatgg
ggctgctgtacaacaagatcaaccactgcagattcgacgagttcttcagcgagggctgcgctccc
ggcagcaagaaagacagcagcctgtgcaagctgtgcatgggcagcggcctgaacctgtgcgagcc
caacaacaaagagggctactacggctacacaggggccttccggtgtctggtggagaagggggacg
tggcttttgtgaaacaccagaccgtgccccagaacaccggcggcaagaaccccgacccctgggcc
aagaacctgaacgagaaggactacgaactgctgtgtctcgacggcacccggaagccagtggagga
atacgccaactgtcacctggccagagcccccaatcacgccgtggtcacccggaaggacaaagagg
cctgcgtccacaagatcctgcggcagcagcagcacctgttcggcagcaacgtgaccgactgcagc
ggcaacttctgcctgttcagaagcgagacaaaggacctcctgttccgggacgacaccgtgtgtct
ggccaagctgcacgaccggaacacctacgagaagtacctgggcgaggaatatgtgaaggccgtgg
gcaatctgcggaagtgcagcacctctagcctgctggaagcctgcacctttcgcagacctTAA
(SEQ ID NO: 52)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVKGVDGDKTHTCPPCP
APELLGGPSVFLFPPKPKDTLMISRTPEVTCVVVDVSHEDPEVKENWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSVPDKTVRWCAVSEHEATKCQSFRDHMKSVIP
SDGPSVACVKKASYLDCIRAIAANEADAVTLDAGLVYDAYLAPNNLKPVVAEFYGSKEDPQTFYY
AVAVVKKDSGFQMNQLRGKKSCHTGLGRSAGWNIPIGLLYCDLPEPRKPLEKAVANFFSGSCAPC
ADGTDFPQLCQLCPGCGCSTLNQYFGYSGAFKCLKDGAGDVAFVKHSTIFENLANKADRDQYELL
CLDNTRKPVDEYKDCHLAQVPSHTVVARSMGGKEDLIWELLNQAQEHFGKDKSKEFQLFSSPHGK
DLLFKDSAHGFLKVPPRMDAKMYLGYEYVTAIRNLREGTCPEAPTDECKPVKWCALSHHERLKCD
EWSVNSVGKIECVSAETTEDCIAKIMNGEADAMSLDGGFVYIAGKCGLVPVLAENYNKSDNCEDT
PEAGYFAIAVVKKSASDLTWDNLKGKKSCHTAVGRTAGWNIPMGLLYNKINHCREDEFFSEGCAP
GSKKDSSLCKLCMGSGLNLCEPNNKEGYYGYTGAFRCLVEKGDVAFVKHQTVPQNTGGKNPDPWA
KNLNEKDYELLCLDGTRKPVEEYANCHLARAPNHAVVTRKDKEACVHKILRQQQHLFGSNVTDCS
GNFCLFRSETKDLLFRDDTVCLAKLHDRNTYEKYLGEEYVKAVGNLRKCSTSSLLEACTFRRP*
pDP74-His8-CD19 NT.1-GFLG-VR-FC-Tf
(SEQ ID NO: 53)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcGTACGGggcGTAGACGGCGACAAAACTCACACATGCCCACCGTGCCCA
GCACCTGAACTCCTGGGGGGACCGTCAGTCTTCCTCTTCCCCCCAAAACCCAAGGACACCCTCAT
GATCTCCCGGACCCCTGAGGTCACATGCGTGGTGGTGGACGTGAGCCACGAAGACCCTGAGGTCA
AGTTCAACTGGTACGTGGACGGCGTGGAGGTGCATAATGCCAAGACAAAGCCGCGGGAGGAGCAG
TACAACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGGGA
GAGCAATGGGCAGCCGGAGAACAACTACAAGACCACGCCTCCCGTGCTGGACTCCGACGGCTCCT
TCTTCCTCTACAGCAAGCTCACCGTGGACAAGAGCAGGTGGCAGCAGGGGAACGTCTTCTCATGC
TCCGTGATGCATGAGGCTCTGCACAACCACTACACGCAGAAGAGCCTCTCCCTGTCTCCGGGTAA
Aggatcctctgggggaagtggaggtagcggtggttctgtgcccgataagacagtgcggtggtgcg
ccgtgtctgagcacgaggccaccaagtgccagagcttccgggaccacatgaagtccgtgatcccc
agcgacggccctagcgtggcctgtgtgaagaaggccagctacctggactgcatccgggccattgc
cgccaatgaggccgacgccgtgacactggatgccggcctggtgtacgatgcctacctggccccca
acaacctgaagcccgtggtggccgagttctacggcagcaaagaggacccccagaccttctactac
gccgtggccgtggtcaagaaggacagcggcttccagatgaaccagctgcggggcaagaagtcctg
tcacaccggcctgggcagaagcgccggctggaacatccccatcggcctgctgtactgcgatctgc
ccgagccccggaagcctctggaaaaggccgtggccaacttcttcagcggcagctgcgccccttgt
gctgacggaaccgacttcccccagctgtgtcagctgtgccccggctgtggctgcagcaccctgaa
ccagtacttcggctacagcggcgccttcaagtgcctgaaggacggcgctggcgacgtggccttcg
tgaagcacagcaccatcttcgagaacctggccaacaaggccgaccgggaccagtacgagctgctg
tgcctggacaacaccagaaagcccgtggacgagtacaaggactgccacctcgcccaggtgccatc
tcacacagtggtggcccggtccatgggcggcaaagaggatctgatctgggagctgctgaaccagg
cccaggaacacttcggcaaggacaagagcaaagagttccagctgttcagcagcccccacggcaag
gatctgctgttcaaggacagcgcccacggctttctgaaggtgccccccagaatggacgccaagat
gtacctgggctacgagtacgtgaccgccatccggaacctgagagagggcacctgtcccgaggccc
ccaccgatgagtgcaagcccgtgaagtggtgcgccctgagccaccacgagcggctgaagtgcgac
gagtggagcgtgaacagcgtgggcaagatcgagtgcgtgagcgccgagacaaccgaggactgtat
cgccaagatcatgaacggcgaggccgatgccatgagcctggacggcggcttcgtgtacattgccg
gcaagtgcggcctggtgcctgtgctggccgagaactacaacaagagcgacaactgcgaggatacc
cccgaggccggctactttgccatcgcagtcgtgaagaagtccgccagcgacctgacctgggacaa
tctgaagggcaagaaaagctgccacaccgccgtgggaaggaccgccgggtggaatattcctatgg
ggctgctgtacaacaagatcaaccactgcagattcgacgagttcttcagcgagggctgcgctccc
ggcagcaagaaagacagcagcctgtgcaagctgtgcatgggcagcggcctgaacctgtgcgagcc
caacaacaaagagggctactacggctacacaggggccttccggtgtctggtggagaagggggacg
tggcttttgtgaaacaccagaccgtgccccagaacaccggcggcaagaaccccgacccctgggcc
aagaacctgaacgagaaggactacgaactgctgtgtctcgacggcacccggaagccagtggagga
atacgccaactgtcacctggccagagcccccaatcacgccgtggtcacccggaaggacaaagagg
cctgcgtccacaagatcctgcggcagcagcagcacctgttcggcagcaacgtgaccgactgcagc
ggcaacttctgcctgttcagaagcgagacaaaggacctcctgttccgggacgacaccgtgtgtct
ggccaagctgcacgaccggaacacctacgagaagtacctgggcgaggaatatgtgaaggccgtgg
gcaatctgcggaagtgcagcacctctagcctgctggaagcctgcacctttcgcagacctTAA
(SEQ ID NO: 54)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVRGVDGDKTHTCPPCP
APELLGGPSVFLFPPKPKDTLMISRTPEVTCVVVDVSHEDPEVKENWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVESC
SVMHEALHNHYTQKSLSLSPGKGSSGGSGGSGGSVPDKTVRWCAVSEHEATKCQSFRDHMKSVIP
SDGPSVACVKKASYLDCIRAIAANEADAVTLDAGLVYDAYLAPNNLKPVVAEFYGSKEDPQTFYY
AVAVVKKDSGFQMNQLRGKKSCHTGLGRSAGWNIPIGLLYCDLPEPRKPLEKAVANFFSGSCAPC
ADGTDFPQLCQLCPGCGCSTLNQYFGYSGAFKCLKDGAGDVAFVKHSTIFENLANKADRDQYELL
CLDNTRKPVDEYKDCHLAQVPSHTVVARSMGGKEDLIWELLNQAQEHFGKDKSKEFQLFSSPHGK
DLLFKDSAHGFLKVPPRMDAKMYLGYEYVTAIRNLREGTCPEAPTDECKPVKWCALSHHERLKCD
EWSVNSVGKIECVSAETTEDCIAKIMNGEADAMSLDGGFVYIAGKCGLVPVLAENYNKSDNCEDT
PEAGYFAIAVVKKSASDLTWDNLKGKKSCHTAVGRTAGWNIPMGLLYNKINHCREDEFFSEGCAP
GSKKDSSLCKLCMGSGLNLCEPNNKEGYYGYTGAFRCLVEKGDVAFVKHQTVPQNTGGKNPDPWA
KNLNEKDYELLCLDGTRKPVEEYANCHLARAPNHAVVTRKDKEACVHKILRQQQHLFGSNVTDCS
GNFCLFRSETKDLLERDDTVCLAKLHDRNTYEKYLGEEYVKAVGNLRKCSTSSLLEACTFRRP*
pDP75-His8-CD19 NT.1-GFLG-GGFG-FC-Tf
(SEQ ID NO: 55)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCCTGggaggcggtgggTTCgggGTAGACGGCGACAAAACTCACACATGCCCACCGTGC
CCAGCACCTGAACTCCTGGGGGGACCGTCAGTCTTCCTCTTCCCCCCAAAACCCAAGGACACCCT
CATGATCTCCCGGACCCCTGAGGTCACATGCGTGGTGGTGGACGTGAGCCACGAAGACCCTGAGG
TCAAGTTCAACTGGTACGTGGACGGCGTGGAGGTGCATAATGCCAAGACAAAGCCGCGGGAGGAG
CAGTACAACAGCACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGATGAGCTGACC
AAGAACCAGGTCAGCCTGACCTGCCTGGTCAAAGGCTTCTATCCCAGCGACATCGCCGTGGAGTG
GGAGAGCAATGGGCAGCCGGAGAACAACTACAAGACCACGCCTCCCGTGCTGGACTCCGACGGCT
CCTTCTTCCTCTACAGCAAGCTCACCGTGGACAAGAGCAGGTGGCAGCAGGGGAACGTCTTCTCA
TGCTCCGTGATGCATGAGGCTCTGCACAACCACTACACGCAGAAGAGCCTCTCCCTGTCTCCGGG
TAAAggatcctctgggggaagtggaggtagcggtggttctgtgcccgataagacagtgcggtggt
gcgccgtgtctgagcacgaggccaccaagtgccagagcttccgggaccacatgaagtccgtgatc
cccagcgacggccctagcgtggcctgtgtgaagaaggccagctacctggactgcatccgggccat
tgccgccaatgaggccgacgccgtgacactggatgccggcctggtgtacgatgcctacctggccc
ccaacaacctgaagcccgtggtggccgagttctacggcagcaaagaggacccccagaccttctac
tacgccgtggccgtggtcaagaaggacagcggcttccagatgaaccagctgcggggcaagaagtc
ctgtcacaccggcctgggcagaagcgccggctggaacatccccatcggcctgctgtactgcgatc
tgcccgagccccggaagcctctggaaaaggccgtggccaacttcttcagcggcagctgcgcccct
tgtgctgacggaaccgacttcccccagctgtgtcagctgtgccccggctgtggctgcagcaccct
gaaccagtacttcggctacagcggcgccttcaagtgcctgaaggacggcgctggcgacgtggcct
tcgtgaagcacagcaccatcttcgagaacctggccaacaaggccgaccgggaccagtacgagctg
ctgtgcctggacaacaccagaaagcccgtggacgagtacaaggactgccacctcgcccaggtgcc
atctcacacagtggtggcccggtccatgggcggcaaagaggatctgatctgggagctgctgaacc
aggcccaggaacacttcggcaaggacaagagcaaagagttccagctgttcagcagcccccacggc
aaggatctgctgttcaaggacagcgcccacggctttctgaaggtgccccccagaatggacgccaa
gatgtacctgggctacgagtacgtgaccgccatccggaacctgagagagggcacctgtcccgagg
cccccaccgatgagtgcaagcccgtgaagtggtgcgccctgagccaccacgagcggctgaagtgc
gacgagtggagcgtgaacagcgtgggcaagatcgagtgcgtgagcgccgagacaaccgaggactg
tatcgccaagatcatgaacggcgaggccgatgccatgagcctggacggcggcttcgtgtacattg
ccggcaagtgcggcctggtgcctgtgctggccgagaactacaacaagagcgacaactgcgaggat
acccccgaggccggctactttgccatcgcagtcgtgaagaagtccgccagcgacctgacctggga
caatctgaagggcaagaaaagctgccacaccgccgtgggaaggaccgccgggtggaatattccta
tggggctgctgtacaacaagatcaaccactgcagattcgacgagttcttcagcgagggctgcgct
cccggcagcaagaaagacagcagcctgtgcaagctgtgcatgggcagcggcctgaacctgtgcga
gcccaacaacaaagagggctactacggctacacaggggccttccggtgtctggtggagaaggggg
acgtggcttttgtgaaacaccagaccgtgccccagaacaccggcggcaagaaccccgacccctgg
gccaagaacctgaacgagaaggactacgaactgctgtgtctcgacggcacccggaagccagtgga
ggaatacgccaactgtcacctggccagagcccccaatcacgccgtggtcacccggaaggacaaag
aggcctgcgtccacaagatcctgcggcagcagcagcacctgttcggcagcaacgtgaccgactgc
agcggcaacttctgcctgttcagaagcgagacaaaggacctcctgttccgggacgacaccgtgtg
tctggccaagctgcacgaccggaacacctacgagaagtacctgggcgaggaatatgtgaaggccg
tgggcaatctgcggaagtgcagcacctctagcctgctggaagcctgcacctttcgcagacctTAA
(SEQ ID NO: 56)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGGFGVDGDKTHTCPPC
PAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGNVES
CSVMHEALHNHYTQKSLSLSPGKGSSGGSGGSGGSVPDKTVRWCAVSEHEATKCQSFRDHMKSVI
PSDGPSVACVKKASYLDCIRAIAANEADAVTLDAGLVYDAYLAPNNLKPVVAEFYGSKEDPQTFY
YAVAVVKKDSGFQMNQLRGKKSCHTGLGRSAGWNIPIGLLYCDLPEPRKPLEKAVANFFSGSCAP
CADGTDFPQLCQLCPGCGCSTLNQYFGYSGAFKCLKDGAGDVAFVKHSTIFENLANKADRDQYEL
LCLDNTRKPVDEYKDCHLAQVPSHTVVARSMGGKEDLIWELLNQAQEHFGKDKSKEFQLFSSPHG
KDLLFKDSAHGFLKVPPRMDAKMYLGYEYVTAIRNLREGTCPEAPTDECKPVKWCALSHHERLKC
DEWSVNSVGKIECVSAETTEDCIAKIMNGEADAMSLDGGFVYIAGKCGLVPVLAENYNKSDNCED
TPEAGYFAIAVVKKSASDLTWDNLKGKKSCHTAVGRTAGWNIPMGLLYNKINHCRFDEFFSEGCA
PGSKKDSSLCKLCMGSGLNLCEPNNKEGYYGYTGAFRCLVEKGDVAFVKHQTVPQNTGGKNPDPW
AKNLNEKDYELLCLDGTRKPVEEYANCHLARAPNHAVVTRKDKEACVHKILRQQQHLFGSNVTDC
SGNFCLFRSETKDLLFRDDTVCLAKLHDRNTYEKYLGEEYVKAVGNLRKCSTSSLLEACTFRRP*
pDP76-His8-CD19 NT.1-FK-FC-Tf
(SEQ ID NO: 57)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gcTTCAAAggcGTAGACGGCGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACAGCA
AGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAAAggatcctctggggg
aagtggaggtagcggtggttctgtgcccgataagacagtgcggtggtgcgccgtgtctgagcacg
aggccaccaagtgccagagcttccgggaccacatgaagtccgtgatccccagcgacggccctagc
gtggcctgtgtgaagaaggccagctacctggactgcatccgggccattgccgccaatgaggccga
cgccgtgacactggatgccggcctggtgtacgatgcctacctggcccccaacaacctgaagcccg
tggtggccgagttctacggcagcaaagaggacccccagaccttctactacgccgtggccgtggtc
aagaaggacagcggcttccagatgaaccagctgcggggcaagaagtcctgtcacaccggcctggg
cagaagcgccggctggaacatccccatcggcctgctgtactgcgatctgcccgagccccggaagc
ctctggaaaaggccgtggccaacttcttcagcggcagctgcgccccttgtgctgacggaaccgac
ttcccccagctgtgtcagctgtgccccggctgtggctgcagcaccctgaaccagtacttcggcta
cagcggcgccttcaagtgcctgaaggacggcgctggcgacgtggccttcgtgaagcacagcacca
tcttcgagaacctggccaacaaggccgaccgggaccagtacgagctgctgtgcctggacaacacc
agaaagcccgtggacgagtacaaggactgccacctcgcccaggtgccatctcacacagtggtggc
ccggtccatgggcggcaaagaggatctgatctgggagctgctgaaccaggcccaggaacacttcg
gcaaggacaagagcaaagagttccagctgttcagcagcccccacggcaaggatctgctgttcaag
gacagcgcccacggctttctgaaggtgccccccagaatggacgccaagatgtacctgggctacga
gtacgtgaccgccatccggaacctgagagagggcacctgtcccgaggcccccaccgatgagtgca
agcccgtgaagtggtgcgccctgagccaccacgagcggctgaagtgcgacgagtggagcgtgaac
agcgtgggcaagatcgagtgcgtgagcgccgagacaaccgaggactgtatcgccaagatcatgaa
cggcgaggccgatgccatgagcctggacggcggcttcgtgtacattgccggcaagtgcggcctgg
tgcctgtgctggccgagaactacaacaagagcgacaactgcgaggatacccccgaggccggctac
tttgccatcgcagtcgtgaagaagtccgccagcgacctgacctgggacaatctgaagggcaagaa
aagctgccacaccgccgtgggaaggaccgccgggtggaatattcctatggggctgctgtacaaca
agatcaaccactgcagattcgacgagttcttcagcgagggctgcgctcccggcagcaagaaagac
agcagcctgtgcaagctgtgcatgggcagcggcctgaacctgtgcgagcccaacaacaaagaggg
ctactacggctacacaggggccttccggtgtctggtggagaagggggacgtggcttttgtgaaac
accagaccgtgccccagaacaccggcggcaagaaccccgacccctgggccaagaacctgaacgag
aaggactacgaactgctgtgtctcgacggcacccggaagccagtggaggaatacgccaactgtca
cctggccagagcccccaatcacgccgtggtcacccggaaggacaaagaggcctgcgtccacaaga
tcctgcggcagcagcagcacctgttcggcagcaacgtgaccgactgcagcggcaacttctgcctg
ttcagaagcgagacaaaggacctcctgttccgggacgacaccgtgtgtctggccaagctgcacga
ccggaacacctacgagaagtacctgggcgaggaatatgtgaaggccgtgggcaatctgcggaagt
gcagcacctctagcctgctggaagcctgcacctttcgcagacctTAA
(SEQ ID NO: 58)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGFKGVDGDKTHTCPPCPAPELL
GGPSVFLFPPKPKDTLMISRTPEVTCVVVDVSHEDPEVKENWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSVPDKTVRWCAVSEHEATKCQSFRDHMKSVIPSDGPS
VACVKKASYLDCIRAIAANEADAVTLDAGLVYDAYLAPNNLKPVVAEFYGSKEDPQTFYYAVAVV
KKDSGFQMNQLRGKKSCHTGLGRSAGWNIPIGLLYCDLPEPRKPLEKAVANFFSGSCAPCADGTD
FPQLCQLCPGCGCSTLNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKEFQLFSSPHGKDLLFK
DSAHGFLKVPPRMDAKMYLGYEYVTAIRNLREGTCPEAPTDECKPVKWCALSHHERLKCDEWSVN
SVGKIECVSAETTEDCIAKIMNGEADAMSLDGGFVYIAGKCGLVPVLAENYNKSDNCEDTPEAGY
FAIAVVKKSASDLTWDNLKGKKSCHTAVGRTAGWNIPMGLLYNKINHCRFDEFFSEGCAPGSKKD
SSLCKLCMGSGLNLCEPNNKEGYYGYTGAFRCLVEKGDVAFVKHQTVPQNTGGKNPDPWAKNLNE
KDYELLCLDGTRKPVEEYANCHLARAPNHAVVTRKDKEACVHKILRQQQHLFGSNVTDCSGNFCL
FRSETKDLLFRDDTVCLAKLHDRNTYEKYLGEEYVKAVGNLRKCSTSSLLEACTFRRP*
pDP77-His8-CD19 NT.1-VA-FC-Tf
(SEQ ID NO: 59)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gcGTAgctggcGTAGACGGCGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACAGCA
AGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAAAggatcctctggggg
aagtggaggtagcggtggttctgtgcccgataagacagtgcggtggtgcgccgtgtctgagcacg
aggccaccaagtgccagagcttccgggaccacatgaagtccgtgatccccagcgacggccctagc
gtggcctgtgtgaagaaggccagctacctggactgcatccgggccattgccgccaatgaggccga
cgccgtgacactggatgccggcctggtgtacgatgcctacctggcccccaacaacctgaagcccg
tggtggccgagttctacggcagcaaagaggacccccagaccttctactacgccgtggccgtggtc
aagaaggacagcggcttccagatgaaccagctgcggggcaagaagtcctgtcacaccggcctggg
cagaagcgccggctggaacatccccatcggcctgctgtactgcgatctgcccgagccccggaagc
ctctggaaaaggccgtggccaacttcttcagcggcagctgcgccccttgtgctgacggaaccgac
ttcccccagctgtgtcagctgtgccccggctgtggctgcagcaccctgaaccagtacttcggcta
cagcggcgccttcaagtgcctgaaggacggcgctggcgacgtggccttcgtgaagcacagcacca
tcttcgagaacctggccaacaaggccgaccgggaccagtacgagctgctgtgcctggacaacacc
agaaagcccgtggacgagtacaaggactgccacctcgcccaggtgccatctcacacagtggtggc
ccggtccatgggcggcaaagaggatctgatctgggagctgctgaaccaggcccaggaacacttcg
gcaaggacaagagcaaagagttccagctgttcagcagcccccacggcaaggatctgctgttcaag
gacagcgcccacggctttctgaaggtgccccccagaatggacgccaagatgtacctgggctacga
gtacgtgaccgccatccggaacctgagagagggcacctgtcccgaggcccccaccgatgagtgca
agcccgtgaagtggtgcgccctgagccaccacgagcggctgaagtgcgacgagtggagcgtgaac
agcgtgggcaagatcgagtgcgtgagcgccgagacaaccgaggactgtatcgccaagatcatgaa
cggcgaggccgatgccatgagcctggacggcggcttcgtgtacattgccggcaagtgcggcctgg
tgcctgtgctggccgagaactacaacaagagcgacaactgcgaggatacccccgaggccggctac
tttgccatcgcagtcgtgaagaagtccgccagcgacctgacctgggacaatctgaagggcaagaa
aagctgccacaccgccgtgggaaggaccgccgggtggaatattcctatggggctgctgtacaaca
agatcaaccactgcagattcgacgagttcttcagcgagggctgcgctcccggcagcaagaaagac
agcagcctgtgcaagctgtgcatgggcagcggcctgaacctgtgcgagcccaacaacaaagaggg
ctactacggctacacaggggccttccggtgtctggtggagaagggggacgtggcttttgtgaaac
accagaccgtgccccagaacaccggcggcaagaaccccgacccctgggccaagaacctgaacgag
aaggactacgaactgctgtgtctcgacggcacccggaagccagtggaggaatacgccaactgtca
cctggccagagcccccaatcacgccgtggtcacccggaaggacaaagaggcctgcgtccacaaga
tcctgcggcagcagcagcacctgttcggcagcaacgtgaccgactgcagcggcaacttctgcctg
ttcagaagcgagacaaaggacctcctgttccgggacgacaccgtgtgtctggccaagctgcacga
ccggaacacctacgagaagtacctgggcgaggaatatgtgaaggccgtgggcaatctgcggaagt
gcagcacctctagcctgctggaagcctgcacctttcgcagacctTAA
(SEQ ID NO: 60)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVAGVDGDKTHTCPPCPAPELL
GGPSVFLFPPKPKDTLMISRTPEVTCVVVDVSHEDPEVKENWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSVPDKTVRWCAVSEHEATKCQSFRDHMKSVIPSDGPS
VACVKKASYLDCIRAIAANEADAVTLDAGLVYDAYLAPNNLKPVVAEFYGSKEDPQTFYYAVAVV
KKDSGFQMNQLRGKKSCHTGLGRSAGWNIPIGLLYCDLPEPRKPLEKAVANFFSGSCAPCADGTD
FPQLCQLCPGCGCSTLNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKEFQLFSSPHGKDLLFK
DSAHGFLKVPPRMDAKMYLGYEYVTAIRNLREGTCPEAPTDECKPVKWCALSHHERLKCDEWSVN
SVGKIECVSAETTEDCIAKIMNGEADAMSLDGGFVYIAGKCGLVPVLAENYNKSDNCEDTPEAGY
FAIAVVKKSASDLTWDNLKGKKSCHTAVGRTAGWNIPMGLLYNKINHCRFDEFFSEGCAPGSKKD
SSLCKLCMGSGLNLCEPNNKEGYYGYTGAFRCLVEKGDVAFVKHQTVPQNTGGKNPDPWAKNLNE
KDYELLCLDGTRKPVEEYANCHLARAPNHAVVTRKDKEACVHKILRQQQHLFGSNVTDCSGNFCL
FRSETKDLLFRDDTVCLAKLHDRNTYEKYLGEEYVKAVGNLRKCSTSSLLEACTFRRP*
pDP78-His8-CD19 NT.1-VK-FC-Tf
(SEQ ID NO: 61)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtgtcatcaggaacgtctctcaacagatggggggcttctacctgtg
ccagccggggcccccctctgagaaggcctggcagcctggctggacagtcaatgtggagggcagcg
gggagctgttccggtggaatgtttcggacctaggtggcctgggctgtggcctgaagaacaggtcc
tcagagggccccagctccccttccgggaagctcatgagccccaagctgtatgtgtgggccaaaga
ccgccctgagatctgggagggagagcctccgtgtctcccaccgagggacagcctgaaccagagcc
tcagcagggacctcaccgtagcccctggctccacactctggctgtcctgtggggtaccccctgac
tctgtgtccaggggccccctctcctggacccatgtgcaccccaaggggcctaagtcattgctgag
cctagagctgaaggacgatcgcccggccagagatatgtgggtaatgggtacgtcactgatgttgc
cccgggccacagctcaagacgctggaaagtggtattgtcaccgtggcaacgtaaccacctcattc
cacctggaggtaatcgctcggccagtaaaggctcactcagacctgaggactggtggctggaaggg
cGTAAAAggcGTAGACGGCGACAAAACTCACACATGCCCACCGTGCCCAGCACCTGAACTCCTGG
GGGGACCGTCAGTCTTCCTCTTCCCCCCAAAACCCAAGGACACCCTCATGATCTCCCGGACCCCT
GAGGTCACATGCGTGGTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGTACAACAGCACGTACC
GTGTGGTCAGCGTCCTCACCGTCCTGCACCAGGACTGGCTGAATGGCAAGGAGTACAAGTGCAAG
GTCTCCAACAAAGCCCTCCCAGCCCCCATCGAGAAAACCATCTCCAAAGCCAAAGGGCAGCCCCG
AGAACCACAGGTGTACACCCTGCCCCCATCCCGGGATGAGCTGACCAAGAACCAGGTCAGCCTGA
CCTGCCTGGTCAAAGGCTTCTATCCCAGCGACATCGCCGTGGAGTGGGAGAGCAATGGGCAGCCG
GAGAACAACTACAAGACCACGCCTCCCGTGCTGGACTCCGACGGCTCCTTCTTCCTCTACAGCAA
GCTCACCGTGGACAAGAGCAGGTGGCAGCAGGGGAACGTCTTCTCATGCTCCGTGATGCATGAGG
CTCTGCACAACCACTACACGCAGAAGAGCCTCTCCCTGTCTCCGGGTAAAggatcctctggggga
agtggaggtagcggtggttctgtgcccgataagacagtgcggtggtgcgccgtgtctgagcacga
ggccaccaagtgccagagcttccgggaccacatgaagtccgtgatccccagcgacggccctagcg
tggcctgtgtgaagaaggccagctacctggactgcatccgggccattgccgccaatgaggccgac
gccgtgacactggatgccggcctggtgtacgatgcctacctggcccccaacaacctgaagcccgt
ggtggccgagttctacggcagcaaagaggacccccagaccttctactacgccgtggccgtggtca
agaaggacagcggcttccagatgaaccagctgcggggcaagaagtcctgtcacaccggcctgggc
agaagcgccggctggaacatccccatcggcctgctgtactgcgatctgcccgagccccggaagcc
tctggaaaaggccgtggccaacttcttcagcggcagctgcgccccttgtgctgacggaaccgact
tcccccagctgtgtcagctgtgccccggctgtggctgcagcaccctgaaccagtacttcggctac
agcggcgccttcaagtgcctgaaggacggcgctggcgacgtggccttcgtgaagcacagcaccat
cttcgagaacctggccaacaaggccgaccgggaccagtacgagctgctgtgcctggacaacacca
gaaagcccgtggacgagtacaaggactgccacctcgcccaggtgccatctcacacagtggtggcc
cggtccatgggcggcaaagaggatctgatctgggagctgctgaaccaggcccaggaacacttcgg
caaggacaagagcaaagagttccagctgttcagcagcccccacggcaaggatctgctgttcaagg
acagcgcccacggctttctgaaggtgccccccagaatggacgccaagatgtacctgggctacgag
tacgtgaccgccatccggaacctgagagagggcacctgtcccgaggcccccaccgatgagtgcaa
gcccgtgaagtggtgcgccctgagccaccacgagcggctgaagtgcgacgagtggagcgtgaaca
gcgtgggcaagatcgagtgcgtgagcgccgagacaaccgaggactgtatcgccaagatcatgaac
ggcgaggccgatgccatgagcctggacggcggcttcgtgtacattgccggcaagtgcggcctggt
gcctgtgctggccgagaactacaacaagagcgacaactgcgaggatacccccgaggccggctact
ttgccatcgcagtcgtgaagaagtccgccagcgacctgacctgggacaatctgaagggcaagaaa
agctgccacaccgccgtgggaaggaccgccgggtggaatattcctatggggctgctgtacaacaa
gatcaaccactgcagattcgacgagttcttcagcgagggctgcgctcccggcagcaagaaagaca
gcagcctgtgcaagctgtgcatgggcagcggcctgaacctgtgcgagcccaacaacaaagagggc
tactacggctacacaggggccttccggtgtctggtggagaagggggacgtggcttttgtgaaaca
ccagaccgtgccccagaacaccggcggcaagaaccccgacccctgggccaagaacctgaacgaga
aggactacgaactgctgtgtctcgacggcacccggaagccagtggaggaatacgccaactgtcac
ctggccagagcccccaatcacgccgtggtcacccggaaggacaaagaggcctgcgtccacaagat
cctgcggcagcagcagcacctgttcggcagcaacgtgaccgactgcagcggcaacttctgcctgt
tcagaagcgagacaaaggacctcctgttccgggacgacaccgtgtgtctggccaagctgcacgac
cggaacacctacgagaagtacctgggcgaggaatatgtgaaggccgtgggcaatctgcggaagtg
cagcacctctagcctgctggaagcctgcacctttcgcagacctTAA
(SEQ ID NO: 62)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVKGVDGDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSVPDKTVRWCAVSEHEATKCQSFRDHMKSVIPSDGPS
VACVKKASYLDCIRAIAANEADAVTLDAGLVYDAYLAPNNLKPVVAEFYGSKEDPQTFYYAVAVV
KKDSGFQMNQLRGKKSCHTGLGRSAGWNIPIGLLYCDLPEPRKPLEKAVANFFSGSCAPCADGTD
FPQLCQLCPGCGCSTLNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKEFQLFSSPHGKDLLFK
DSAHGFLKVPPRMDAKMYLGYEYVTAIRNLREGTCPEAPTDECKPVKWCALSHHERLKCDEWSVN
SVGKIECVSAETTEDCIAKIMNGEADAMSLDGGFVYIAGKCGLVPVLAENYNKSDNCEDTPEAGY
FAIAVVKKSASDLTWDNLKGKKSCHTAVGRTAGWNIPMGLLYNKINHCRFDEFFSEGCAPGSKKD
SSLCKLCMGSGLNLCEPNNKEGYYGYTGAFRCLVEKGDVAFVKHQTVPQNTGGKNPDPWAKNLNE
KDYELLCLDGTRKPVEEYANCHLARAPNHAVVTRKDKEACVHKILRQQQHLFGSNVTDCSGNFCL
FRSETKDLLFRDDTVCLAKLHDRNTYEKYLGEEYVKAVGNLRKCSTSSLLEACTFRRP*
pDP79-His8-CD19 NT.1-VR-FC-Tf
(SEQ ID NO: 63)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gcGTACGGggcGTAGACGGCGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGACCCC
TGAGGTCACATGCGTGGTGGTGGACGTGAGCCACGAAGACCCTGAGGTCAAGTTCAACTGGTACG
TGGACGGCGTGGAGGTGCATAATGCCAAGACAAAGCCGCGGGAGGAGCAGTACAACAGCACGTAC
CGTGTGGTCAGCGTCCTCACCGTCCTGCACCAGGACTGGCTGAATGGCAAGGAGTACAAGTGCAA
GGTCTCCAACAAAGCCCTCCCAGCCCCCATCGAGAAAACCATCTCCAAAGCCAAAGGGCAGCCCC
GAGAACCACAGGTGTACACCCTGCCCCCATCCCGGGATGAGCTGACCAAGAACCAGGTCAGCCTG
ACCTGCCTGGTCAAAGGCTTCTATCCCAGCGACATCGCCGTGGAGTGGGAGAGCAATGGGCAGCC
GGAGAACAACTACAAGACCACGCCTCCCGTGCTGGACTCCGACGGCTCCTTCTTCCTCTACAGCA
AGCTCACCGTGGACAAGAGCAGGTGGCAGCAGGGGAACGTCTTCTCATGCTCCGTGATGCATGAG
GCTCTGCACAACCACTACACGCAGAAGAGCCTCTCCCTGTCTCCGGGTAAAggatcctctggggg
aagtggaggtagcggtggttctgtgcccgataagacagtgcggtggtgcgccgtgtctgagcacg
aggccaccaagtgccagagcttccgggaccacatgaagtccgtgatccccagcgacggccctagc
gtggcctgtgtgaagaaggccagctacctggactgcatccgggccattgccgccaatgaggccga
cgccgtgacactggatgccggcctggtgtacgatgcctacctggcccccaacaacctgaagcccg
tggtggccgagttctacggcagcaaagaggacccccagaccttctactacgccgtggccgtggtc
aagaaggacagcggcttccagatgaaccagctgcggggcaagaagtcctgtcacaccggcctggg
cagaagcgccggctggaacatccccatcggcctgctgtactgcgatctgcccgagccccggaagc
ctctggaaaaggccgtggccaacttcttcagcggcagctgcgccccttgtgctgacggaaccgac
ttcccccagctgtgtcagctgtgccccggctgtggctgcagcaccctgaaccagtacttcggcta
cagcggcgccttcaagtgcctgaaggacggcgctggcgacgtggccttcgtgaagcacagcacca
tcttcgagaacctggccaacaaggccgaccgggaccagtacgagctgctgtgcctggacaacacc
agaaagcccgtggacgagtacaaggactgccacctcgcccaggtgccatctcacacagtggtggc
ccggtccatgggcggcaaagaggatctgatctgggagctgctgaaccaggcccaggaacacttcg
gcaaggacaagagcaaagagttccagctgttcagcagcccccacggcaaggatctgctgttcaag
gacagcgcccacggctttctgaaggtgccccccagaatggacgccaagatgtacctgggctacga
gtacgtgaccgccatccggaacctgagagagggcacctgtcccgaggcccccaccgatgagtgca
agcccgtgaagtggtgcgccctgagccaccacgagcggctgaagtgcgacgagtggagcgtgaac
agcgtgggcaagatcgagtgcgtgagcgccgagacaaccgaggactgtatcgccaagatcatgaa
cggcgaggccgatgccatgagcctggacggcggcttcgtgtacattgccggcaagtgcggcctgg
tgcctgtgctggccgagaactacaacaagagcgacaactgcgaggatacccccgaggccggctac
tttgccatcgcagtcgtgaagaagtccgccagcgacctgacctgggacaatctgaagggcaagaa
aagctgccacaccgccgtgggaaggaccgccgggtggaatattcctatggggctgctgtacaaca
agatcaaccactgcagattcgacgagttcttcagcgagggctgcgctcccggcagcaagaaagac
agcagcctgtgcaagctgtgcatgggcagcggcctgaacctgtgcgagcccaacaacaaagaggg
ctactacggctacacaggggccttccggtgtctggtggagaagggggacgtggcttttgtgaaac
accagaccgtgccccagaacaccggcggcaagaaccccgacccctgggccaagaacctgaacgag
aaggactacgaactgctgtgtctcgacggcacccggaagccagtggaggaatacgccaactgtca
cctggccagagcccccaatcacgccgtggtcacccggaaggacaaagaggcctgcgtccacaaga
tcctgcggcagcagcagcacctgttcggcagcaacgtgaccgactgcagcggcaacttctgcctg
ttcagaagcgagacaaaggacctcctgttccgggacgacaccgtgtgtctggccaagctgcacga
ccggaacacctacgagaagtacctgggcgaggaatatgtgaaggccgtgggcaatctgcggaagt
gcagcacctctagcctgctggaagcctgcacctttcgcagacctTAA
(SEQ ID NO: 64)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVRGVDGDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSVPDKTVRWCAVSEHEATKCQSFRDHMKSVIPSDGPS
VACVKKASYLDCIRAIAANEADAVTLDAGLVYDAYLAPNNLKPVVAEFYGSKEDPQTFYYAVAVV
KKDSGFQMNQLRGKKSCHTGLGRSAGWNIPIGLLYCDLPEPRKPLEKAVANFFSGSCAPCADGTD
FPQLCQLCPGCGCSTLNQYFGYSGAFKCLKDGAGDVAFVKHSTIFENLANKADRDQYELLCLDNT
RKPVDEYKDCHLAQVPSHTVVARSMGGKEDLIWELLNQAQEHFGKDKSKEFQLFSSPHGKDLLFK
DSAHGFLKVPPRMDAKMYLGYEYVTAIRNLREGTCPEAPTDECKPVKWCALSHHERLKCDEWSVN
SVGKIECVSAETTEDCIAKIMNGEADAMSLDGGFVYIAGKCGLVPVLAENYNKSDNCEDTPEAGY
FAIAVVKKSASDLTWDNLKGKKSCHTAVGRTAGWNIPMGLLYNKINHCRFDEFFSEGCAPGSKKD
SSLCKLCMGSGLNLCEPNNKEGYYGYTGAFRCLVEKGDVAFVKHQTVPQNTGGKNPDPWAKNLNE
KDYELLCLDGTRKPVEEYANCHLARAPNHAVVTRKDKEACVHKILRQQQHLFGSNVTDCSGNFCL
FRSETKDLLERDDTVCLAKLHDRNTYEKYLGEEYVKAVGNLRKCSTSSLLEACTFRRP*
pDP80-His8-CD19 NT.1-GGFG-FC-Tf
(SEQ ID NO: 65)
ATGTACAGGATGCAACTCCTGTCTTGCATTGCACTAAGTCTTGCACTTGTCACGAATTCGGGCCA
CCACCATCATCACCACCATCACaccggtcccgaggaacctctagtggtgaaggtggaagagggag
ataccgctgctctgtggtgcctcaaggggacctcagatggccccactcagcagctgacctggtct
cgggagtccccgcttaaacccttcttaaaatacagcctgggggtgccaggcctgggagtccacgt
caggcccgatgccatctctgtcgtcatcaggaacgtctctcaacagatggggggcttctacctgt
gccagccggggcccccctctgagaaggcctggcagcctggctggacagtcaatgtggagggcagc
ggggagctgttccggtggaatgtttcggacctaggtggcctgggctgtggcctgaagaacaggtc
ctcagagggccccagctccccttccgggaagctcatgagccccaagctgtatgtgtgggccaaag
accgccctgagatctgggagggagagcctccgtgtctcccaccgagggacagcctgaaccagagc
ctcagcagggacctcaccgtagcccctggctccacactctggctgtcctgtggggtaccccctga
ctctgtgtccaggggccccctctcctggacccatgtgcaccccaaggggcctaagtcattgctga
gcctagagctgaaggacgatcgcccggccagagatatgtgggtaatgggtacgtcactgatgttg
ccccgggccacagctcaagacgctggaaagtggtattgtcaccgtggcaacgtaaccacctcatt
ccacctggaggtaatcgctcggccagtaaaggctcactcagacctgaggactggtggctggaagg
gtgggTTCggaggcGTAGACGGCGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACTGGT
ACGTGGACGGCGTGGAGGTGCATAATGCCAAGACAAAGCCGCGGGAGGAGCAGTACAACAGCACG
TACCGTGTGGTCAGCGTCCTCACCGTCCTGCACCAGGACTGGCTGAATGGCAAGGAGTACAAGTG
CAAGGTCTCCAACAAAGCCCTCCCAGCCCCCATCGAGAAAACCATCTCCAAAGCCAAAGGGCAGC
CCCGAGAACCACAGGTGTACACCCTGCCCCCATCCCGGGATGAGCTGACCAAGAACCAGGTCAGC
CTGACCTGCCTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTTCCTCTACA
GCAAGCTCACCGTGGACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGGTAAAggatcctctgg
gggaagtggaggtagcggtggttctgtgcccgataagacagtgcggtggtgcgccgtgtctgagc
acgaggccaccaagtgccagagcttccgggaccacatgaagtccgtgatccccagcgacggccct
agcgtggcctgtgtgaagaaggccagctacctggactgcatccgggccattgccgccaatgaggc
cgacgccgtgacactggatgccggcctggtgtacgatgcctacctggcccccaacaacctgaagc
ccgtggtggccgagttctacggcagcaaagaggacccccagaccttctactacgccgtggccgtg
gtcaagaaggacagcggcttccagatgaaccagctgcggggcaagaagtcctgtcacaccggcct
gggcagaagcgccggctggaacatccccatcggcctgctgtactgcgatctgcccgagccccgga
agcctctggaaaaggccgtggccaacttcttcagcggcagctgcgccccttgtgctgacggaacc
gacttcccccagctgtgtcagctgtgccccggctgtggctgcagcaccctgaaccagtacttcgg
ctacagcggcgccttcaagtgcctgaaggacggcgctggcgacgtggccttcgtgaagcacagca
ccatcttcgagaacctggccaacaaggccgaccgggaccagtacgagctgctgtgcctggacaac
accagaaagcccgtggacgagtacaaggactgccacctcgcccaggtgccatctcacacagtggt
ggcccggtccatgggcggcaaagaggatctgatctgggagctgctgaaccaggcccaggaacact
tcggcaaggacaagagcaaagagttccagctgttcagcagcccccacggcaaggatctgctgttc
aaggacagcgcccacggctttctgaaggtgccccccagaatggacgccaagatgtacctgggcta
cgagtacgtgaccgccatccggaacctgagagagggcacctgtcccgaggcccccaccgatgagt
gcaagcccgtgaagtggtgcgccctgagccaccacgagcggctgaagtgcgacgagtggagcgtg
aacagcgtgggcaagatcgagtgcgtgagcgccgagacaaccgaggactgtatcgccaagatcat
gaacggcgaggccgatgccatgagcctggacggcggcttcgtgtacattgccggcaagtgcggcc
tggtgcctgtgctggccgagaactacaacaagagcgacaactgcgaggatacccccgaggccggc
tactttgccatcgcagtcgtgaagaagtccgccagcgacctgacctgggacaatctgaagggcaa
gaaaagctgccacaccgccgtgggaaggaccgccgggtggaatattcctatggggctgctgtaca
acaagatcaaccactgcagattcgacgagttcttcagcgagggctgcgctcccggcagcaagaaa
gacagcagcctgtgcaagctgtgcatgggcagcggcctgaacctgtgcgagcccaacaacaaaga
gggctactacggctacacaggggccttccggtgtctggtggagaagggggacgtggcttttgtga
aacaccagaccgtgccccagaacaccggcggcaagaaccccgacccctgggccaagaacctgaac
gagaaggactacgaactgctgtgtctcgacggcacccggaagccagtggaggaatacgccaactg
tcacctggccagagcccccaatcacgccgtggtcacccggaaggacaaagaggcctgcgtccaca
agatcctgcggcagcagcagcacctgttcggcagcaacgtgaccgactgcagcggcaacttctgc
ctgttcagaagcgagacaaaggacctcctgttccgggacgacaccgtgtgtctggccaagctgca
cgaccggaacacctacgagaagtacctgggcgaggaatatgtgaaggccgtgggcaatctgcgga
agtgcagcacctctagcctgctggaagcctgcacctttcgcagacctTAA
(SEQ ID NO: 66)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFGGVDGDKTHTCPPCPAPEL
LGGPSVFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMH
EALHNHYTQKSLSLSPGKGSSGGSGGSGGSVPDKTVRWCAVSEHEATKCQSFRDHMKSVIPSDGP
SVACVKKASYLDCIRAIAANEADAVTLDAGLVYDAYLAPNNLKPVVAEFYGSKEDPQTFYYAVAV
VKKDSGFQMNQLRGKKSCHTGLGRSAGWNIPIGLLYCDLPEPRKPLEKAVANFFSGSCAPCADGT
DFPQLCQLCPGCGCSTLNQYFGYSGAFKCLKDGAGDVAFVKHSTIFENLANKADRDQYELLCLDN
TRKPVDEYKDCHLAQVPSHTVVARSMGGKEDLIWELLNQAQEHFGKDKSKEFQLFSSPHGKDLLF
KDSAHGFLKVPPRMDAKMYLGYEYVTAIRNLREGTCPEAPTDECKPVKWCALSHHERLKCDEWSV
NSVGKIECVSAETTEDCIAKIMNGEADAMSLDGGFVYIAGKCGLVPVLAENYNKSDNCEDTPEAG
YFAIAVVKKSASDLTWDNLKGKKSCHTAVGRTAGWNIPMGLLYNKINHCRFDEFFSEGCAPGSKK
DSSLCKLCMGSGLNLCEPNNKEGYYGYTGAFRCLVEKGDVAFVKHQTVPQNTGGKNPDPWAKNLN
EKDYELLCLDGTRKPVEEYANCHLARAPNHAVVTRKDKEACVHKILRQQQHLFGSNVTDCSGNFC
LFRSETKDLLFRDDTVCLAKLHDRNTYEKYLGEEYVKAVGNLRKCSTSSLLEACTFRRP*
pDP86-PDL1-scfv-GFLG-fc-Tf
(SEQ ID NO: 67)
ATGTACAGGATGCAACTCCTGTCTTGCATTGCACTAAGTCTTGCACTTGTCACGAATTCGGATAT
ACAGATGACCCAATCCCCATCCAGCTTGTCCGCTAGCGTAGGCGATAGAGTAACTATTACATGCC
GCGCTAGTCAAGACGTGTCAACTGCAGTCGCGTGGTACCAACAAAAGCCTGGCAAAGCTCCGAAA
CTGCTGATTTACAGCGCGTCTTTCCTTTACTCTGGGGTACCTAGCCGATTTTCTGGGTCTGGTAG
CGGAACCGATTTCACGCTTACAATTTCTAGCCTCCAACCCGAAGATTTCGCGACGTACTACTGCC
AACAATACCTTTACCATCCAGCCACATTTGGACAGGGCACGAAGGTTGAAATAAAAGGCGGAGGT
GGATCTGGCGGAGGAGGAAGTGGGGGTGGAGGTTCAGAAGTTCAGCTGGTTGAATCAGGCGGCGG
ACTTGTTCAGCCGGGCGGAAGCCTTCGGCTTAGCTGTGCTGCCAGTGGCTTCACATTCAGTGATA
GCTGGATTCATTGGGTTCGCCAGGCACCAGGCAAAGGTTTGGAGTGGGTCGCCTGGATTAGTCCG
TATGGGGGCTCCACCTACTACGCTGACTCAGTGAAAGGGCGGTTTACCATTAGTGCTGATACGTC
CAAAAATACAGCTTACCTTCAGATGAACTCTCTGAGGGCCGAAGATACTGCTGTGTACTACTGCG
CTCGGAGACATTGGCCAGGAGGGTTCGATTACTGGGGGCAAGGCACTTTGGTGACAGTCAGTTCA
GGTGGTTCCGGCAGCGCAGGAggtgggTTCCTGggaGGCGTAGACGGCGACAAAACTCACACATG
CCCACCGTGCCCAGCACCTGAACTCCTGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAA
GACCCTGAGGTCAAGTTCAACTGGTACGTGGACGGCGTGGAGGTGCATAATGCCAAGACAAAGCC
GCGGGAGGAGCAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCTGCCCCCATCCCGGGA
TGAGCTGACCAAGAACCAGGTCAGCCTGACCTGCCTGGTCAAAGGCTTCTATCCCAGCGACATCG
CCGTGGAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGCAGGGGAA
CGTCTTCTCATGCTCCGTGATGCATGAGGCTCTGCACAACCACTACACGCAGAAGAGCCTCTCCC
TGTCTCCGGGTAAAggatcctctgggggaagtggaggtagcggtggttctgtgcccgataagaca
gtgcggtggtgcgccgtgtctgagcacgaggccaccaagtgccagagcttccgggaccacatgaa
gtccgtgatccccagcgacggccctagcgtggcctgtgtgaagaaggccagctacctggactgca
tccgggccattgccgccaatgaggccgacgccgtgacactggatgccggcctggtgtacgatgcc
tacctggcccccaacaacctgaagcccgtggtggccgagttctacggcagcaaagaggaccccca
gaccttctactacgccgtggccgtggtcaagaaggacagcggcttccagatgaaccagctgcggg
gcaagaagtcctgtcacaccggcctgggcagaagcgccggctggaacatccccatcggcctgctg
tactgcgatctgcccgagccccggaagcctctggaaaaggccgtggccaacttcttcagcggcag
ctgcgccccttgtgctgacggaaccgacttcccccagctgtgtcagctgtgccccggctgtggct
gcagcaccctgaaccagtacttcggctacagcggcgccttcaagtgcctgaaggacggcgctggc
gacgtggccttcgtgaagcacagcaccatcttcgagaacctggccaacaaggccgaccgggacca
gtacgagctgctgtgcctggacaacaccagaaagcccgtggacgagtacaaggactgccacctcg
cccaggtgccatctcacacagtggtggcccggtccatgggcggcaaagaggatctgatctgggag
ctgctgaaccaggcccaggaacacttcggcaaggacaagagcaaagagttccagctgttcagcag
cccccacggcaaggatctgctgttcaaggacagcgcccacggctttctgaaggtgccccccagaa
tggacgccaagatgtacctgggctacgagtacgtgaccgccatccggaacctgagagagggcacc
tgtcccgaggcccccaccgatgagtgcaagcccgtgaagtggtgcgccctgagccaccacgagcg
gctgaagtgcgacgagtggagcgtgaacagcgtgggcaagatcgagtgcgtgagcgccgagacaa
ccgaggactgtatcgccaagatcatgaacggcgaggccgatgccatgagcctggacggcggcttc
gtgtacattgccggcaagtgcggcctggtgcctgtgctggccgagaactacaacaagagcgacaa
ctgcgaggatacccccgaggccggctactttgccatcgcagtcgtgaagaagtccgccagcgacc
tgacctgggacaatctgaagggcaagaaaagctgccacaccgccgtgggaaggaccgccgggtgg
aatattcctatggggctgctgtacaacaagatcaaccactgcagattcgacgagttcttcagcga
gggctgcgctcccggcagcaagaaagacagcagcctgtgcaagctgtgcatgggcagcggcctga
acctgtgcgagcccaacaacaaagagggctactacggctacacaggggccttccggtgtctggtg
gagaagggggacgtggcttttgtgaaacaccagaccgtgccccagaacaccggcggcaagaaccc
cgacccctgggccaagaacctgaacgagaaggactacgaactgctgtgtctcgacggcacccgga
agccagtggaggaatacgccaactgtcacctggccagagcccccaatcacgccgtggtcacccgg
aaggacaaagaggcctgcgtccacaagatcctgcggcagcagcagcacctgttcggcagcaacgt
gaccgactgcagcggcaacttctgcctgttcagaagcgagacaaaggacctcctgttccgggacg
acaccgtgtgtctggccaagctgcacgaccggaacacctacgagaagtacctgggcgaggaatat
gtgaaggccgtgggcaatctgcggaagtgcagcacctctagcctgctggaagcctgcacctttcg
cagacctTAA
(SEQ ID NO: 68)
MYRMQLLSCIALSLALVTNSDIQMTQSPSSLSASVGDRVTITCRASQDVSTAVAWYQQKPGKAPK
LLIYSASFLYSGVPSRFSGSGSGTDFTLTISSLQPEDFATYYCQQYLYHPATFGQGTKVEIKGGG
GSGGGGSGGGGSEVQLVESGGGLVQPGGSLRLSCAASGFTFSDSWIHWVRQAPGKGLEWVAWISP
YGGSTYYADSVKGRFTISADTSKNTAYLQMNSLRAEDTAVYYCARRHWPGGFDYWGQGTLVTVSS
GGSGSAGGGFLGGVDGDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSSGGSGGSGGSVPDKT
VRWCAVSEHEATKCQSFRDHMKSVIPSDGPSVACVKKASYLDCIRAIAANEADAVTLDAGLVYDA
YLAPNNLKPVVAEFYGSKEDPQTFYYAVAVVKKDSGFQMNQLRGKKSCHTGLGRSAGWNIPIGLL
YCDLPEPRKPLEKAVANFFSGSCAPCADGTDFPQLCQLCPGCGCSTLNQYFGYSGAFKCLKDGAG
DVAFVKHSTIFENLANKADRDQYELLCLDNTRKPVDEYKDCHLAQVPSHTVVARSMGGKEDLIWE
LLNQAQEHFGKDKSKEFQLFSSPHGKDLLFKDSAHGFLKVPPRMDAKMYLGYEYVTAIRNLREGT
CPEAPTDECKPVKWCALSHHERLKCDEWSVNSVGKIECVSAETTEDCIAKIMNGEADAMSLDGGF
VYIAGKCGLVPVLAENYNKSDNCEDTPEAGYFAIAVVKKSASDLTWDNLKGKKSCHTAVGRTAGW
NIPMGLLYNKINHCRFDEFFSEGCAPGSKKDSSLCKLCMGSGLNLCEPNNKEGYYGYTGAFRCLV
EKGDVAFVKHQTVPQNTGGKNPDPWAKNLNEKDYELLCLDGTRKPVEEYANCHLARAPNHAVVTR
KDKEACVHKILRQQQHLFGSNVTDCSGNFCLFRSETKDLLFRDDTVCLAKLHDRNTYEKYLGEEY
VKAVGNLRKCSTSSLLEACTFRRP*
pDP96-CD20_HC_Fc-N297G, S427C-Tf
(SEQ ID NO: 69)
ATGTACAGGATGCAACTCCTGTCTTGCATTGCACTAAGTCTTGCACTTGTCACGAATTCGCAGAT
TGTTCTGAGCCAGTCCCCGGCAATCCTCTCTGCCAGCCCAGGCGAAAAGGTGACAATGACTTGCC
GAGCGAGTTCCAGTGTCTCTTATATCCACTGGTTCCAGCAAAAGCCGGGAAGCAGCCCTAAACCA
TGGATATATGCAACGTCTAACCTGGCGAGCGGGGTCCCAGTGAGATTTTCCGGAAGCGGCAGCGG
AACTAGTTACTCTTTGACAATAAGCAGAGTGGAGGCTGAGGACGCTGCTACTTACTATTGCCAGC
AATGGACGAGTAACCCGCCGACGTTTGGAGGTGGAACGAAGCTGGAGATTAAAGGTGGAGGTGGT
TCTGGCGGAGGTGGTTCCGGTGGTGGTGGAAGTCAGGTGCAGCTCCAACAGCCTGGTGCCGAACT
TGTCAAACCTGGGGCTAGTGTGAAGATGAGTTGCAAAGCTTCAGGGTACACGTTTACGTCATACA
ACATGCATTGGGTAAAGCAAACACCAGGACGCGGCTTGGAATGGATCGGCGCGATATATCCAGGA
AACGGTGACACTTCTTATAACCAGAAGTTCAAGGGGAAAGCTACTCTCACAGCGGACAAATCTTC
TTCAACAGCGTATATGCAGTTGTCAAGCCTTACTAGCGAGGACAGTGCTGTTTATTACTGCGCCC
GGTCCACCTATTATGGGGGTGATTGGTACTTTAATGTTTGGGGCGCGGGTACTACCGTTACTGTG
TCCGCGGGTGTAGACGGCAGCGGCAGCgacaaaactcacacatgcCCCCCCTGCCCAGCGCCAGA
ATTGCTGGGCGGACCCAGCGTGTTCCTGTTCCCCCCCAAACCTAAAGACACCCTGATGATCAGCC
GAACCCCTGAGGTGACCTGCGTGGTGGTGGACGTGAGCCACGAGGACCCCGAGGTGAAGTTCAAC
TGGTATGTGGACGGCGTGGAGGTCCACAATGCCAAAACGAAGCCCAGGGAGGAGCAGTACGGCAG
CACCTACAGGGTAGTGAGCGTCTTGACCGTGCTGCACCAGGACTGGCTGAACGGCAAGGAATACA
AATGCAAGGTCAGCAATAAGGCTCTGCCGGCTCCTATCGAGAAGACAATCAGCAAGGCAAAGGGC
CAGCCACGCGAACCGCAGGTGTATACTCTGCCCCCCAGCCGGGACGAGCTGACCAAGAACCAGGT
GTCCCTGACCTGTCTGGTGAAAGGCTTCTACCCCAGCGACATCGCTGTGGAGTGGGAGAGTAACG
GGCAGCCCGAGAACAACTACAAGACCACGCCTCCTGTGCTGGACAGCGACGGCAGCTTCTTCCTG
TATAGCAAGCTCACCGTGGACAAGAGCAGGTGGCAACAGGGCAACGTGTTCTGCTGCTCTGTGAT
GCACGAGGCCCTGCACAACCATTACACCCAGAAGAGTCTCAGTCTGAGCCCGGGAAAGggatcct
ctgggggaagtggaggtagcggtggttctgtgcccgataagacagtgcggtggtgcgccgtgtct
gagcacgaggccaccaagtgccagagcttccgggaccacatgaagtccgtgatccccagcgacgg
ccctagcgtggcctgtgtgaagaaggccagctacctggactgcatccgggccattgccgccaatg
aggccgacgccgtgacactggatgccggcctggtgtacgatgcctacctggcccccaacaacctg
aagcccgtggtggccgagttctacggcagcaaagaggacccccagaccttctactacgccgtggc
cgtggtcaagaaggacagcggcttccagatgaaccagctgcggggcaagaagtcctgtcacaccg
gcctgggcagaagcgccggctggaacatccccatcggcctgctgtactgcgatctgcccgagccc
cggaagcctctggaaaaggccgtggccaacttcttcagcggcagctgcgccccttgtgctgacgg
aaccgacttcccccagctgtgtcagctgtgccccggctgtggctgcagcaccctgaaccagtact
tcggctacagcggcgccttcaagtgcctgaaggacggcgctggcgacgtggccttcgtgaagcac
agcaccatcttcgagaacctggccaacaaggccgaccgggaccagtacgagctgctgtgcctgga
caacaccagaaagcccgtggacgagtacaaggactgccacctcgcccaggtgccatctcacacag
tggtggcccggtccatgggcggcaaagaggatctgatctgggagctgctgaaccaggcccaggaa
cacttcggcaaggacaagagcaaagagttccagctgttcagcagcccccacggcaaggatctgct
gttcaaggacagcgcccacggctttctgaaggtgccccccagaatggacgccaagatgtacctgg
gctacgagtacgtgaccgccatccggaacctgagagagggcacctgtcccgaggcccccaccgat
gagtgcaagcccgtgaagtggtgcgccctgagccaccacgagcggctgaagtgcgacgagtggag
cgtgaacagcgtgggcaagatcgagtgcgtgagcgccgagacaaccgaggactgtatcgccaaga
tcatgaacggcgaggccgatgccatgagcctggacggcggcttcgtgtacattgccggcaagtgc
ggcctggtgcctgtgctggccgagaactacaacaagagcgacaactgcgaggatacccccgaggc
cggctactttgccatcgcagtcgtgaagaagtccgccagcgacctgacctgggacaatctgaagg
gcaagaaaagctgccacaccgccgtgggaaggaccgccgggtggaatattcctatggggctgctg
tacaacaagatcaaccactgcagattcgacgagttcttcagcgagggctgcgctcccggcagcaa
gaaagacagcagcctgtgcaagctgtgcatgggcagcggcctgaacctgtgcgagcccaacaaca
aagagggctactacggctacacaggggccttccggtgtctggtggagaagggggacgtggctttt
gtgaaacaccagaccgtgccccagaacaccggcggcaagaaccccgacccctgggccaagaacct
gaacgagaaggactacgaactgctgtgtctcgacggcacccggaagccagtggaggaatacgcca
actgtcacctggccagagcccccaatcacgccgtggtcacccggaaggacaaagaggcctgcgtc
cacaagatcctgcggcagcagcagcacctgttcggcagcaacgtgaccgactgcagcggcaactt
ctgcctgttcagaagcgagacaaaggacctcctgttccgggacgacaccgtgtgtctggccaagc
tgcacgaccggaacacctacgagaagtacctgggcgaggaatatgtgaaggccgtgggcaatctg
cggaagtgcagcacctctagcctgctggaagcctgcacctttcgcagacctTAA
(SEQ ID NO: 70)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGVDGSGSDKTHTCPPCPAPELLGGPSVFLFPPKPKDTLMISRTPEVTCVVVDVSHEDPEVKEN
WYVDGVEVHNAKTKPREEQYGSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFL
YSKLTVDKSRWQQGNVFCCSVMHEALHNHYTQKSLSLSPGKGSSGGSGGSGGSVPDKTVRWCAVS
EHEATKCQSFRDHMKSVIPSDGPSVACVKKASYLDCIRAIAANEADAVTLDAGLVYDAYLAPNNL
KPVVAEFYGSKEDPQTFYYAVAVVKKDSGFQMNQLRGKKSCHTGLGRSAGWNIPIGLLYCDLPEP
RKPLEKAVANFFSGSCAPCADGTDFPQLCQLCPGCGCSTLNQYFGYSGAFKCLKDGAGDVAFVKH
STIFENLANKADRDQYELLCLDNTRKPVDEYKDCHLAQVPSHTVVARSMGGKEDLIWELLNQAQE
HFGKDKSKEFQLFSSPHGKDLLFKDSAHGFLKVPPRMDAKMYLGYEYVTAIRNLREGTCPEAPTD
ECKPVKWCALSHHERLKCDEWSVNSVGKIECVSAETTEDCIAKIMNGEADAMSLDGGFVYIAGKC
GLVPVLAENYNKSDNCEDTPEAGYFAIAVVKKSASDLTWDNLKGKKSCHTAVGRTAGWNIPMGLL
YNKINHCRFDEFFSEGCAPGSKKDSSLCKLCMGSGLNLCEPNNKEGYYGYTGAFRCLVEKGDVAF
VKHQTVPQNTGGKNPDPWAKNLNEKDYELLCLDGTRKPVEEYANCHLARAPNHAVVTRKDKEACV
HKILRQQQHLFGSNVTDCSGNFCLFRSETKDLLFRDDTVCLAKLHDRNTYEKYLGEEYVKAVGNL
RKCSTSSLLEACTFRRP*
pDP97-cd20-scfv-GFLG-FC-N297G, S427C-Tf
(SEQ ID NO: 71)
ATGTACAGGATGCAACTCCTGTCTTGCATTGCACTAAGTCTTGCACTTGTCACGAATTCGCAGAT
TGTTCTGAGCCAGTCCCCGGCAATCCTCTCTGCCAGCCCAGGCGAAAAGGTGACAATGACTTGCC
GAGCGAGTTCCAGTGTCTCTTATATCCACTGGTTCCAGCAAAAGCCGGGAAGCAGCCCTAAACCA
TGGATATATGCAACGTCTAACCTGGCGAGCGGGGTCCCAGTGAGATTTTCCGGAAGCGGCAGCGG
AACTAGTTACTCTTTGACAATAAGCAGAGTGGAGGCTGAGGACGCTGCTACTTACTATTGCCAGC
AATGGACGAGTAACCCGCCGACGTTTGGAGGTGGAACGAAGCTGGAGATTAAAGGTGGAGGTGGT
TCTGGCGGAGGTGGTTCCGGTGGTGGTGGAAGTCAGGTGCAGCTCCAACAGCCTGGTGCCGAACT
TGTCAAACCTGGGGCTAGTGTGAAGATGAGTTGCAAAGCTTCAGGGTACACGTTTACGTCATACA
ACATGCATTGGGTAAAGCAAACACCAGGACGCGGCTTGGAATGGATCGGCGCGATATATCCAGGA
AACGGTGACACTTCTTATAACCAGAAGTTCAAGGGGAAAGCTACTCTCACAGCGGACAAATCTTC
TTCAACAGCGTATATGCAGTTGTCAAGCCTTACTAGCGAGGACAGTGCTGTTTATTACTGCGCCC
GGTCCACCTATTATGGGGGTGATTGGTACTTTAATGTTTGGGGCGCGGGTACTACCGTTACTGTG
TCCGCGGGTGGCAGCGGCAGCggtgggTTCCTGggaGGCGTAGACGGCgacaaaactcacacatg
cCCCCCCTGCCCAGCGCCAGAATTGCTGGGCGGACCCAGCGTGTTCCTGTTCCCCCCCAAACCTA
AAGACACCCTGATGATCAGCCGAACCCCTGAGGTGACCTGCGTGGTGGTGGACGTGAGCCACGAG
GACCCCGAGGTGAAGTTCAACTGGTATGTGGACGGCGTGGAGGTCCACAATGCCAAAACGAAGCC
CAGGGAGGAGCAGTACGGCAGCACCTACAGGGTAGTGAGCGTCTTGACCGTGCTGCACCAGGACT
GGCTGAACGGCAAGGAATACAAATGCAAGGTCAGCAATAAGGCTCTGCCGGCTCCTATCGAGAAG
ACAATCAGCAAGGCAAAGGGCCAGCCACGCGAACCGCAGGTGTATACTCTGCCCCCCAGCCGGGA
CGAGCTGACCAAGAACCAGGTGTCCCTGACCTGTCTGGTGAAAGGCTTCTACCCCAGCGACATCG
CTGTGGAGTGGGAGAGTAACGGGCAGCCCGAGAACAACTACAAGACCACGCCTCCTGTGCTGGAC
AGCGACGGCAGCTTCTTCCTGTATAGCAAGCTCACCGTGGACAAGAGCAGGTGGCAACAGGGCAA
CGTGTTCTGCTGCTCTGTGATGCACGAGGCCCTGCACAACCATTACACCCAGAAGAGTCTCAGTC
TGAGCCCGGGAAAGggatcctctgggggaagtggaggtagcggtggttctgtgcccgataagaca
gtgcggtggtgcgccgtgtctgagcacgaggccaccaagtgccagagcttccgggaccacatgaa
gtccgtgatccccagcgacggccctagcgtggcctgtgtgaagaaggccagctacctggactgca
tccgggccattgccgccaatgaggccgacgccgtgacactggatgccggcctggtgtacgatgcc
tacctggcccccaacaacctgaagcccgtggtggccgagttctacggcagcaaagaggaccccca
gaccttctactacgccgtggccgtggtcaagaaggacagcggcttccagatgaaccagctgcggg
gcaagaagtcctgtcacaccggcctgggcagaagcgccggctggaacatccccatcggcctgctg
tactgcgatctgcccgagccccggaagcctctggaaaaggccgtggccaacttcttcagcggcag
ctgcgccccttgtgctgacggaaccgacttcccccagctgtgtcagctgtgccccggctgtggct
gcagcaccctgaaccagtacttcggctacagcggcgccttcaagtgcctgaaggacggcgctggc
gacgtggccttcgtgaagcacagcaccatcttcgagaacctggccaacaaggccgaccgggacca
gtacgagctgctgtgcctggacaacaccagaaagcccgtggacgagtacaaggactgccacctcg
cccaggtgccatctcacacagtggtggcccggtccatgggcggcaaagaggatctgatctgggag
ctgctgaaccaggcccaggaacacttcggcaaggacaagagcaaagagttccagctgttcagcag
cccccacggcaaggatctgctgttcaaggacagcgcccacggctttctgaaggtgccccccagaa
tggacgccaagatgtacctgggctacgagtacgtgaccgccatccggaacctgagagagggcacc
tgtcccgaggcccccaccgatgagtgcaagcccgtgaagtggtgcgccctgagccaccacgagcg
gctgaagtgcgacgagtggagcgtgaacagcgtgggcaagatcgagtgcgtgagcgccgagacaa
ccgaggactgtatcgccaagatcatgaacggcgaggccgatgccatgagcctggacggcggcttc
gtgtacattgccggcaagtgcggcctggtgcctgtgctggccgagaactacaacaagagcgacaa
ctgcgaggatacccccgaggccggctactttgccatcgcagtcgtgaagaagtccgccagcgacc
tgacctgggacaatctgaagggcaagaaaagctgccacaccgccgtgggaaggaccgccgggtgg
aatattcctatggggctgctgtacaacaagatcaaccactgcagattcgacgagttcttcagcga
gggctgcgctcccggcagcaagaaagacagcagcctgtgcaagctgtgcatgggcagcggcctga
acctgtgcgagcccaacaacaaagagggctactacggctacacaggggccttccggtgtctggtg
gagaagggggacgtggcttttgtgaaacaccagaccgtgccccagaacaccggcggcaagaaccc
cgacccctgggccaagaacctgaacgagaaggactacgaactgctgtgtctcgacggcacccgga
agccagtggaggaatacgccaactgtcacctggccagagcccccaatcacgccgtggtcacccgg
aaggacaaagaggcctgcgtccacaagatcctgcggcagcagcagcacctgttcggcagcaacgt
gaccgactgcagcggcaacttctgcctgttcagaagcgagacaaaggacctcctgttccgggacg
acaccgtgtgtctggccaagctgcacgaccggaacacctacgagaagtacctgggcgaggaatat
gtgaaggccgtgggcaatctgcggaagtgcagcacctctagcctgctggaagcctgcacctttcg
cagacctTAA
(SEQ ID NO: 72)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYGSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFCCSVMHEALHNHYTQKSLSLSPGKGSSGGSGGSGGSVPDKT
VRWCAVSEHEATKCQSFRDHMKSVIPSDGPSVACVKKASYLDCIRAIAANEADAVTLDAGLVYDA
YLAPNNLKPVVAEFYGSKEDPQTFYYAVAVVKKDSGFQMNQLRGKKSCHTGLGRSAGWNIPIGLL
YCDLPEPRKPLEKAVANFFSGSCAPCADGTDFPQLCQLCPGCGCSTLNQYFGYSGAFKCLKDGAG
DVAFVKHSTIFENLANKADRDQYELLCLDNTRKPVDEYKDCHLAQVPSHTVVARSMGGKEDLIWE
LLNQAQEHFGKDKSKEFQLFSSPHGKDLLFKDSAHGFLKVPPRMDAKMYLGYEYVTAIRNLREGT
CPEAPTDECKPVKWCALSHHERLKCDEWSVNSVGKIECVSAETTEDCIAKIMNGEADAMSLDGGF
VYIAGKCGLVPVLAENYNKSDNCEDTPEAGYFAIAVVKKSASDLTWDNLKGKKSCHTAVGRTAGW
NIPMGLLYNKINHCRFDEFFSEGCAPGSKKDSSLCKLCMGSGLNLCEPNNKEGYYGYTGAFRCLV
EKGDVAFVKHQTVPQNTGGKNPDPWAKNLNEKDYELLCLDGTRKPVEEYANCHLARAPNHAVVTR
KDKEACVHKILRQQQHLFGSNVTDCSGNFCLFRSETKDLLFRDDTVCLAKLHDRNTYEKYLGEEY
VKAVGNLRKCSTSSLLEACTERRP*
pDP98-CD20_HC Fc-N297G, S427C
(SEQ ID NO: 73)
ATGTACAGGATGCAACTCCTGTCTTGCATTGCACTAAGTCTTGCACTTGTCACGAATTCGCAGAT
TGTTCTGAGCCAGTCCCCGGCAATCCTCTCTGCCAGCCCAGGCGAAAAGGTGACAATGACTTGCC
GAGCGAGTTCCAGTGTCTCTTATATCCACTGGTTCCAGCAAAAGCCGGGAAGCAGCCCTAAACCA
TGGATATATGCAACGTCTAACCTGGCGAGCGGGGTCCCAGTGAGATTTTCCGGAAGCGGCAGCGG
AACTAGTTACTCTTTGACAATAAGCAGAGTGGAGGCTGAGGACGCTGCTACTTACTATTGCCAGC
AATGGACGAGTAACCCGCCGACGTTTGGAGGTGGAACGAAGCTGGAGATTAAAGGTGGAGGTGGT
TCTGGCGGAGGTGGTTCCGGTGGTGGTGGAAGTCAGGTGCAGCTCCAACAGCCTGGTGCCGAACT
TGTCAAACCTGGGGCTAGTGTGAAGATGAGTTGCAAAGCTTCAGGGTACACGTTTACGTCATACA
ACATGCATTGGGTAAAGCAAACACCAGGACGCGGCTTGGAATGGATCGGCGCGATATATCCAGGA
AACGGTGACACTTCTTATAACCAGAAGTTCAAGGGGAAAGCTACTCTCACAGCGGACAAATCTTC
TTCAACAGCGTATATGCAGTTGTCAAGCCTTACTAGCGAGGACAGTGCTGTTTATTACTGCGCCC
GGTCCACCTATTATGGGGGTGATTGGTACTTTAATGTTTGGGGCGCGGGTACTACCGTTACTGTG
TCCGCGGGTGTAGACGGCAGCGGCAGCgacaaaactcacacatgcCCCCCCTGCCCAGCGCCAGA
ATTGCTGGGCGGACCCAGCGTGTTCCTGTTCCCCCCCAAACCTAAAGACACCCTGATGATCAGCC
GAACCCCTGAGGTGACCTGCGTGGTGGTGGACGTGAGCCACGAGGACCCCGAGGTGAAGTTCAAC
TGGTATGTGGACGGCGTGGAGGTCCACAATGCCAAAACGAAGCCCAGGGAGGAGCAGTACGGCAG
CACCTACAGGGTAGTGAGCGTCTTGACCGTGCTGCACCAGGACTGGCTGAACGGCAAGGAATACA
AATGCAAGGTCAGCAATAAGGCTCTGCCGGCTCCTATCGAGAAGACAATCAGCAAGGCAAAGGGC
CAGCCACGCGAACCGCAGGTGTATACTCTGCCCCCCAGCCGGGACGAGCTGACCAAGAACCAGGT
GTCCCTGACCTGTCTGGTGAAAGGCTTCTACCCCAGCGACATCGCTGTGGAGTGGGAGAGTAACG
GGCAGCCCGAGAACAACTACAAGACCACGCCTCCTGTGCTGGACAGCGACGGCAGCTTCTTCCTG
TATAGCAAGCTCACCGTGGACAAGAGCAGGTGGCAACAGGGCAACGTGTTCTGCTGCTCTGTGAT
GCACGAGGCCCTGCACAACCATTACACCCAGAAGAGTCTCAGTCTGAGCCCGGGAAAGTAA
(SEQ ID NO: 74)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGGGGGSQVQLQQPGAELVKPGASVKMSCKASGYTFTSYNMHWVKQTPGRGLEWIGAIYPGN
GDTSYNQKFKGKATLTADKSSSTAYMQLSSLTSEDSAVYYCARSTYYGGDWYFNVWGAGTTVTVS
AGVDGSGSDKTHTCPPCPAPELLGGPSVFLFPPKPKDTLMISRTPEVTCVVVDVSHEDPEVKENW
YVDGVEVHNAKTKPREEQYGSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLY
SKLTVDKSRWQQGNVFCCSVMHEALHNHYTQKSLSLSPGK*
cd20-scFV-FC-Tf
(SEQ ID NO: 75)
ATGTACAGGATGCAACTCCTGTCTTGCATTGCACTAAGTCTTGCACTTGTCACGAATTCGCAGAT
TGTTCTGAGCCAGTCCCCGGCAATCCTCTCTGCCAGCCCAGGCGAAAAGGTGACAATGACTTGCC
GAGCGAGTTCCAGTGTCTCTTATATCCACTGGTTCCAGCAAAAGCCGGGAAGCAGCCCTAAACCA
TGGATATATGCAACGTCTAACCTGGCGAGCGGGGTCCCAGTGAGATTTTCCGGAAGCGGCAGCGG
AACTAGTTACTCTTTGACAATAAGCAGAGTGGAGGCTGAGGACGCTGCTACTTACTATTGCCAGC
AATGGACGAGTAACCCGCCGACGTTTGGAGGTGGAACGAAGCTGGAGATTAAAGGTGGAGGTGGT
TCTGGCGGAGGTGGTTCCGGTGGTGGTGGAAGTCAGGTGCAGCTCCAACAGCCTGGTGCCGAACT
TGTCAAACCTGGGGCTAGTGTGAAGATGAGTTGCAAAGCTTCAGGGTACACGTTTACGTCATACA
ACATGCATTGGGTAAAGCAAACACCAGGACGCGGCTTGGAATGGATCGGCGCGATATATCCAGGA
AACGGTGACACTTCTTATAACCAGAAGTTCAAGGGGAAAGCTACTCTCACAGCGGACAAATCTTC
TTCAACAGCGTATATGCAGTTGTCAAGCCTTACTAGCGAGGACAGTGCTGTTTATTACTGCGCCC
GGTCCACCTATTATGGGGGTGATTGGTACTTTAATGTTTGGGGCGCGGGTACTACCGTTACTGTG
TCCGCGGGTGTAGACGGCAGCGGCAGCGACAAAACTCACACATGCCCACCGTGCCCAGCACCTGA
ACTCCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAACAG
CACGTACCGTGTGGTCAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCTGACCAAGAACCAGGT
CAGCCTGACCTGCCTGGTCAAAGGCTTCTATCCCAGCGACATCGCCGTGGAGTGGGAGAGCAATG
GGCAGCCGGAGAACAACTACAAGACCACGCCTCCCGTGCTGGACTCCGACGGCTCCTTCTTCCTC
TACAGCAAGCTCACCGTGGACAAGAGCAGGTGGCAGCAGGGGAACGTCTTCTCATGCTCCGTGAT
GCATGAGGCTCTGCACAACCACTACACGCAGAAGAGCCTCTCCCTGTCTCCGGGTAAAggatcct
ctgggggaagtggaggtagcggtggttctgtgcccgataagacagtgcggtggtgcgccgtgtct
gagcacgaggccaccaagtgccagagcttccgggaccacatgaagtccgtgatccccagcgacgg
ccctagcgtggcctgtgtgaagaaggccagctacctggactgcatccgggccattgccgccaatg
aggccgacgccgtgacactggatgccggcctggtgtacgatgcctacctggcccccaacaacctg
aagcccgtggtggccgagttctacggcagcaaagaggacccccagaccttctactacgccgtggc
cgtggtcaagaaggacagcggcttccagatgaaccagctgcggggcaagaagtcctgtcacaccg
gcctgggcagaagcgccggctggaacatccccatcggcctgctgtactgcgatctgcccgagccc
cggaagcctctggaaaaggccgtggccaacttcttcagcggcagctgcgccccttgtgctgacgg
aaccgacttcccccagctgtgtcagctgtgccccggctgtggctgcagcaccctgaaccagtact
tcggctacagcggcgccttcaagtgcctgaaggacggcgctggcgacgtggccttcgtgaagcac
agcaccatcttcgagaacctggccaacaaggccgaccgggaccagtacgagctgctgtgcctgga
caacaccagaaagcccgtggacgagtacaaggactgccacctcgcccaggtgccatctcacacag
tggtggcccggtccatgggcggcaaagaggatctgatctgggagctgctgaaccaggcccaggaa
cacttcggcaaggacaagagcaaagagttccagctgttcagcagcccccacggcaaggatctgct
gttcaaggacagcgcccacggctttctgaaggtgccccccagaatggacgccaagatgtacctgg
gctacgagtacgtgaccgccatccggaacctgagagagggcacctgtcccgaggcccccaccgat
gagtgcaagcccgtgaagtggtgcgccctgagccaccacgagcggctgaagtgcgacgagtggag
cgtgaacagcgtgggcaagatcgagtgcgtgagcgccgagacaaccgaggactgtatcgccaaga
tcatgaacggcgaggccgatgccatgagcctggacggcggcttcgtgtacattgccggcaagtgc
ggcctggtgcctgtgctggccgagaactacaacaagagcgacaactgcgaggatacccccgaggc
cggctactttgccatcgcagtcgtgaagaagtccgccagcgacctgacctgggacaatctgaagg
gcaagaaaagctgccacaccgccgtgggaaggaccgccgggtggaatattcctatggggctgctg
tacaacaagatcaaccactgcagattcgacgagttcttcagcgagggctgcgctcccggcagcaa
gaaagacagcagcctgtgcaagctgtgcatgggcagcggcctgaacctgtgcgagcccaacaaca
aagagggctactacggctacacaggggccttccggtgtctggtggagaagggggacgtggctttt
gtgaaacaccagaccgtgccccagaacaccggcggcaagaaccccgacccctgggccaagaacct
gaacgagaaggactacgaactgctgtgtctcgacggcacccggaagccagtggaggaatacgcca
actgtcacctggccagagcccccaatcacgccgtggtcacccggaaggacaaagaggcctgcgtc
cacaagatcctgcggcagcagcagcacctgttcggcagcaacgtgaccgactgcagcggcaactt
ctgcctgttcagaagcgagacaaaggacctcctgttccgggacgacaccgtgtgtctggccaagc
tgcacgaccggaacacctacgagaagtacctgggcgaggaatatgtgaaggccgtgggcaatctg
cggaagtgcagcacctctagcctgctggaagcctgcacctttcgcagacctTAA
(SEQ ID NO: 76)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGVDGSGSDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGKGSSGGSGGSGGSVPDKTVRWCAVS
EHEATKCQSFRDHMKSVIPSDGPSVACVKKASYLDCIRAIAANEADAVTLDAGLVYDAYLAPNNL
KPVVAEFYGSKEDPQTFYYAVAVVKKDSGFQMNQLRGKKSCHTGLGRSAGWNIPIGLLYCDLPEP
RKPLEKAVANFFSGSCAPCADGTDFPQLCQLCPGCGCSTLNQYFGYSGAFKCLKDGAGDVAFVKH
STIFENLANKADRDQYELLCLDNTRKPVDEYKDCHLAQVPSHTVVARSMGGKEDLIWELLNQAQE
HFGKDKSKEFQLFSSPHGKDLLFKDSAHGFLKVPPRMDAKMYLGYEYVTAIRNLREGTCPEAPTD
ECKPVKWCALSHHERLKCDEWSVNSVGKIECVSAETTEDCIAKIMNGEADAMSLDGGFVYIAGKC
GLVPVLAENYNKSDNCEDTPEAGYFAIAVVKKSASDLTWDNLKGKKSCHTAVGRTAGWNIPMGLL
YNKINHCRFDEFFSEGCAPGSKKDSSLCKLCMGSGLNLCEPNNKEGYYGYTGAFRCLVEKGDVAF
VKHQTVPQNTGGKNPDPWAKNLNEKDYELLCLDGTRKPVEEYANCHLARAPNHAVVTRKDKEACV
HKILRQQQHLFGSNVTDCSGNFCLFRSETKDLLFRDDTVCLAKLHDRNTYEKYLGEEYVKAVGNL
RKCSTSSLLEACTFRRP*
PD-L1-scFV-FC-Tf
(SEQ ID NO: 77)
ATGTACAGGATGCAACTCCTGTCTTGCATTGCACTAAGTCTTGCACTTGTCACGAATTCGGATAT
ACAGATGACCCAATCCCCATCCAGCTTGTCCGCTAGCGTAGGCGATAGAGTAACTATTACATGCC
GCGCTAGTCAAGACGTGTCAACTGCAGTCGCGTGGTACCAACAAAAGCCTGGCAAAGCTCCGAAA
CTGCTGATTTACAGCGCGTCTTTCCTTTACTCTGGGGTACCTAGCCGATTTTCTGGGTCTGGTAG
CGGAACCGATTTCACGCTTACAATTTCTAGCCTCCAACCCGAAGATTTCGCGACGTACTACTGCC
AACAATACCTTTACCATCCAGCCACATTTGGACAGGGCACGAAGGTTGAAATAAAAGGCGGAGGT
GGATCTGGCGGAGGAGGAAGTGGGGGTGGAGGTTCAGAAGTTCAGCTGGTTGAATCAGGCGGCGG
ACTTGTTCAGCCGGGCGGAAGCCTTCGGCTTAGCTGTGCTGCCAGTGGCTTCACATTCAGTGATA
GCTGGATTCATTGGGTTCGCCAGGCACCAGGCAAAGGTTTGGAGTGGGTCGCCTGGATTAGTCCG
TATGGGGGCTCCACCTACTACGCTGACTCAGTGAAAGGGCGGTTTACCATTAGTGCTGATACGTC
CAAAAATACAGCTTACCTTCAGATGAACTCTCTGAGGGCCGAAGATACTGCTGTGTACTACTGCG
CTCGGAGACATTGGCCAGGAGGGTTCGATTACTGGGGGCAAGGCACTTTGGTGACAGTCAGTTCA
GGTGGTTCCGGCAGCGCAGGAGTAGACGGCAGCGGCAGCGACAAAACTCACACATGCCCACCGTG
CCCAGCACCTGAACTCCTGGGGGGACCGTCAGTCTTCCTCTTCCCCCCAAAACCCAAGGACACCC
TCATGATCTCCCGGACCCCTGAGGTCACATGCGTGGTGGTGGACGTGAGCCACGAAGACCCTGAG
GTCAAGTTCAACTGGTACGTGGACGGCGTGGAGGTGCATAATGCCAAGACAAAGCCGCGGGAGGA
GCAGTACAACAGCACGTACCGTGTGGTCAGCGTCCTCACCGTCCTGCACCAGGACTGGCTGAATG
GCAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGATGAGCTGAC
CAAGAACCAGGTCAGCCTGACCTGCCTGGTCAAAGGCTTCTATCCCAGCGACATCGCCGTGGAGT
GGGAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTCTC
ATGCTCCGTGATGCATGAGGCTCTGCACAACCACTACACGCAGAAGAGCCTCTCCCTGTCTCCGG
GTAAAggatcctctgggggaagtggaggtagcggtggttctgtgcccgataagacagtgcggtgg
tgcgccgtgtctgagcacgaggccaccaagtgccagagcttccgggaccacatgaagtccgtgat
ccccagcgacggccctagcgtggcctgtgtgaagaaggccagctacctggactgcatccgggcca
ttgccgccaatgaggccgacgccgtgacactggatgccggcctggtgtacgatgcctacctggcc
cccaacaacctgaagcccgtggtggccgagttctacggcagcaaagaggacccccagaccttcta
ctacgccgtggccgtggtcaagaaggacagcggcttccagatgaaccagctgcggggcaagaagt
cctgtcacaccggcctgggcagaagcgccggctggaacatccccatcggcctgctgtactgcgat
ctgcccgagccccggaagcctctggaaaaggccgtggccaacttcttcagcggcagctgcgcccc
ttgtgctgacggaaccgacttcccccagctgtgtcagctgtgccccggctgtggctgcagcaccc
tgaaccagtacttcggctacagcggcgccttcaagtgcctgaaggacggcgctggcgacgtggcc
ttcgtgaagcacagcaccatcttcgagaacctggccaacaaggccgaccgggaccagtacgagct
gctgtgcctggacaacaccagaaagcccgtggacgagtacaaggactgccacctcgcccaggtgc
catctcacacagtggtggcccggtccatgggcggcaaagaggatctgatctgggagctgctgaac
caggcccaggaacacttcggcaaggacaagagcaaagagttccagctgttcagcagcccccacgg
caaggatctgctgttcaaggacagcgcccacggctttctgaaggtgccccccagaatggacgcca
agatgtacctgggctacgagtacgtgaccgccatccggaacctgagagagggcacctgtcccgag
gcccccaccgatgagtgcaagcccgtgaagtggtgcgccctgagccaccacgagcggctgaagtg
cgacgagtggagcgtgaacagcgtgggcaagatcgagtgcgtgagcgccgagacaaccgaggact
gtatcgccaagatcatgaacggcgaggccgatgccatgagcctggacggcggcttcgtgtacatt
gccggcaagtgcggcctggtgcctgtgctggccgagaactacaacaagagcgacaactgcgagga
tacccccgaggccggctactttgccatcgcagtcgtgaagaagtccgccagcgacctgacctggg
acaatctgaagggcaagaaaagctgccacaccgccgtgggaaggaccgccgggtggaatattcct
atggggctgctgtacaacaagatcaaccactgcagattcgacgagttcttcagcgagggctgcgc
tcccggcagcaagaaagacagcagcctgtgcaagctgtgcatgggcagcggcctgaacctgtgcg
agcccaacaacaaagagggctactacggctacacaggggccttccggtgtctggtggagaagggg
gacgtggcttttgtgaaacaccagaccgtgccccagaacaccggcggcaagaaccccgacccctg
ggccaagaacctgaacgagaaggactacgaactgctgtgtctcgacggcacccggaagccagtgg
aggaatacgccaactgtcacctggccagagcccccaatcacgccgtggtcacccggaaggacaaa
gaggcctgcgtccacaagatcctgcggcagcagcagcacctgttcggcagcaacgtgaccgactg
cagcggcaacttctgcctgttcagaagcgagacaaaggacctcctgttccgggacgacaccgtgt
gtctggccaagctgcacgaccggaacacctacgagaagtacctgggcgaggaatatgtgaaggcc
gtgggcaatctgcggaagtgcagcacctctagcctgctggaagcctgcacctttcgcagacctTA
A
(SEQ ID NO: 78)
MYRMQLLSCIALSLALVTNSDIQMTQSPSSLSASVGDRVTITCRASQDVSTAVAWYQQKPGKAPK
LLIYSASFLYSGVPSRFSGSGSGTDFTLTISSLQPEDFATYYCQQYLYHPATFGQGTKVEIKGGG
GSGGGGSGGGGSEVQLVESGGGLVQPGGSLRLSCAASGFTFSDSWIHWVRQAPGKGLEWVAWISP
YGGSTYYADSVKGRFTISADTSKNTAYLQMNSLRAEDTAVYYCARRHWPGGFDYWGQGTLVTVSS
GGSGSAGVDGSGSDKTHTCPPCPAPELLGGPSVFLFPPKPKDTLMISRTPEVTCVVVDVSHEDPE
VKENWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKGSSGGSGGSGGSVPDKTVRW
CAVSEHEATKCQSFRDHMKSVIPSDGPSVACVKKASYLDCIRAIAANEADAVTLDAGLVYDAYLA
PNNLKPVVAEFYGSKEDPQTFYYAVAVVKKDSGFQMNQLRGKKSCHTGLGRSAGWNIPIGLLYCD
LPEPRKPLEKAVANFFSGSCAPCADGTDFPQLCQLCPGCGCSTLNQYFGYSGAFKCLKDGAGDVA
FVKHSTIFENLANKADRDQYELLCLDNTRKPVDEYKDCHLAQVPSHTVVARSMGGKEDLIWELLN
QAQEHFGKDKSKEFQLFSSPHGKDLLFKDSAHGFLKVPPRMDAKMYLGYEYVTAIRNLREGTCPE
APTDECKPVKWCALSHHERLKCDEWSVNSVGKIECVSAETTEDCIAKIMNGEADAMSLDGGFVYI
AGKCGLVPVLAENYNKSDNCEDTPEAGYFAIAVVKKSASDLTWDNLKGKKSCHTAVGRTAGWNIP
MGLLYNKINHCRFDEFFSEGCAPGSKKDSSLCKLCMGSGLNLCEPNNKEGYYGYTGAFRCLVEKG
DVAFVKHQTVPQNTGGKNPDPWAKNLNEKDYELLCLDGTRKPVEEYANCHLARAPNHAVVTRKDK
EACVHKILRQQQHLFGSNVTDCSGNFCLFRSETKDLLFRDDTVCLAKLHDRNTYEKYLGEEYVKA
VGNLRKCSTSSLLEACTERRP*
pDP124-cd20-scfv-FC-TfR-H7
(SEQ ID NO: 79)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGVDGSGSDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGKGSSGGSGGSGGSSELTQDPAVSVA
LGQTVRITCQGDSLRSYYASWYQQKPGQAPVLVMYGRNERPSGVPDRFSGSKSGTSASLAISGLQ
PEDEANYYCAGWDDSLTGPVFGGGTKLTVLGGGGGSGGGGSGGGGSQVQLQESGGGVVQPGRSLR
LSCAASRFTFSSYAMHWVRQAPGKGLEWVAVISYDGSNKYYADSVKGRFTISRDNSKNTLYLQMN
SLRAEDTAVYYCARDLSGYGDYPDYWGQGTLVTVSS*
pDP125-cd20-scfv-FC-TfR-M16
(SEQ ID NO: 80)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGVDGSGSDKTHTCPPCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGKGSSGGSGGSGGSSELTQDPAVSVA
LGQTVRITCQGDSLRSYYASWYQQKPGQAPVLVMYGRNERPSGVPDRESGSKSGTSASLAISGLQ
PEDEANYYCAGWDDSLTGPVFGGGTKLTVLGGGGGSGGGGSGGGGSQVQLQESGGGVVQPGRSLR
LSCAASRYPFHHHDHHWVRQAPGKGLEWVAVISYDGSNKYYADSVKGRFTISRDNSKNTLYLQMN
SLRAEDTAVYYCARDLSGYGDYPDYWGQGTLVTVSS*
pDP126-cd20-scfv-GFLG-FC-TfR-H7
(SEQ ID NO: 81)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSSGGSGGSGGSSELTQ
DPAVSVALGQTVRITCQGDSLRSYYASWYQQKPGQAPVLVMYGRNERPSGVPDRFSGSKSGTSAS
LAISGLQPEDEANYYCAGWDDSLTGPVFGGGTKLTVLGGGGGSGGGGSGGGGSQVQLQESGGGVV
QPGRSLRLSCAASRFTFSSYAMHWVRQAPGKGLEWVAVISYDGSNKYYADSVKGRFTISRDNSKN
TLYLQMNSLRAEDTAVYYCARDLSGYGDYPDYWGQGTLVTVSS*
pDP127-cd20-scfv-GFLG-FC-TfR-M16
(SEQ ID NO: 82)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSSGGSGGSGGSSELTQ
DPAVSVALGQTVRITCQGDSLRSYYASWYQQKPGQAPVLVMYGRNERPSGVPDRESGSKSGTSAS
LAISGLQPEDEANYYCAGWDDSLTGPVFGGGTKLTVLGGGGGSGGGGSGGGGSQVQLQESGGGVV
QPGRSLRLSCAASRYPFHHHDHHWVRQAPGKGLEWVAVISYDGSNKYYADSVKGRFTISRDNSKN
TLYLQMNSLRAEDTAVYYCARDLSGYGDYPDYWGQGTLVTVSS*
pDP155-His8-CD19 NT.1-2XGFLG-FC-TfR-H7
(SEQ ID NO: 83)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGFLGGVDGDKTHTCPP
CPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLYSKLTVDKSRWQQGNVF
SCSVMHEALHNHYTQKSLSLSPGKGSSGGSGGSGGSSELTQDPAVSVALGQTVRITCQGDSLRSY
YASWYQQKPGQAPVLVMYGRNERPSGVPDRESGSKSGTSASLAISGLQPEDEANYYCAGWDDSLT
GPVFGGGTKLTVLGGGGGSGGGGSGGGGSQVQLQESGGGVVQPGRSLRLSCAASRFTESSYAMHW
VRQAPGKGLEWVAVISYDGSNKYYADSVKGRFTISRDNSKNTLYLQMNSLRAEDTAVYYCARDLS
GYGDYPDYWGQGTLVTVSS*
pDP156-His8-CD19 NT.1-3XGFLG-FC-TfR-H7
(SEQ ID NO: 84)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGFLGGGFLGGVDGDKT
HTCPPCPAPELLGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQ
QGNVFSCSVMHEALHNHYTQKSLSLSPGKGSSGGSGGSGGSELTQDPAVSVALGQTVRITCQGDS
LRSYYASWYQQKPGQAPVLVMYGRNERPSGVPDRFSGSKSGTSASLAISGLQPEDEANYYCAGWD
DSLTGPVFGGGTKLTVLGGGGGSGGGGSGGGGSQVQLQESGGGVVQPGRSLRLSCAASRFTFSSY
AMHWVRQAPGKGLEWVAVISYDGSNKYYADSVKGRFTISRDNSKNTLYLQMNSLRAEDTAVYYCA
RDLSGYGDYPDYWGQGTLVTVSS*
pDP157-His8-CD19 NT.1-GFLG-FK-FC-TfR-H7
(SEQ ID NO: 85)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGFKGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSSELTQDPAVSVALGQTVRITCQGDSLRSYYA
SWYQQKPGQAPVLVMYGRNERPSGVPDRESGSKSGTSASLAISGLQPEDEANYYCAGWDDSLTGP
VFGGGTKLTVLGGGGGSGGGGSGGGGSQVQLQESGGGVVQPGRSLRLSCAASRFTFSSYAMHWVR
QAPGKGLEWVAVISYDGSNKYYADSVKGRFTISRDNSKNTLYLQMNSLRAEDTAVYYCARDLSGY
GDYPDYWGQGTLVTVSS*
pDP158-His8-CD19 NT.1-GFLG-VA-FC-TfR-H7
(SEQ ID NO: 86)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVAGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSSELTQDPAVSVALGQTVRITCQGDSLRSYYA
SWYQQKPGQAPVLVMYGRNERPSGVPDRESGSKSGTSASLAISGLQPEDEANYYCAGWDDSLTGP
VFGGGTKLTVLGGGGGSGGGGSGGGGSQVQLQESGGGVVQPGRSLRLSCAASRFTFSSYAMHWVR
QAPGKGLEWVAVISYDGSNKYYADSVKGRFTISRDNSKNTLYLQMNSLRAEDTAVYYCARDLSGY
GDYPDYWGQGTLVTVSS*
pDP159-His8-CD19 NT.1-GFLG-VK-FC-TfR-H7
(SEQ ID NO: 87)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVKGVDGDKTHTCPPCP
APELLGGPSVFLFPPKPKDTLMISRTPEVTCVVVDVSHEDPEVKENWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVESC
SVMHEALHNHYTQKSLSLSPGKGSSGGSGGSGGSELTQDPAVSVALGQTVRITCQGDSLRSYYAS
WYQQKPGQAPVLVMYGRNERPSGVPDRESGSKSGTSASLAISGLQPEDEANYYCAGWDDSLTGPV
FGGGTKLTVLGGGGGSGGGGSGGGGSQVQLQESGGGVVQPGRSLRLSCAASRFTFSSYAMHWVRQ
APGKGLEWVAVISYDGSNKYYADSVKGRFTISRDNSKNTLYLQMNSLRAEDTAVYYCARDLSGYG
DYPDYWGQGTLVTVSS*
pDP160-His8-CD19 NT.1-GFLG-VR-FC-TfR-H7
(SEQ ID NO: 88)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVRGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSSELTQDPAVSVALGQTVRITCQGDSLRSYYA
SWYQQKPGQAPVLVMYGRNERPSGVPDRESGSKSGTSASLAISGLQPEDEANYYCAGWDDSLTGP
VFGGGTKLTVLGGGGGSGGGGSGGGGSQVQLQESGGGVVQPGRSLRLSCAASRFTFSSYAMHWVR
QAPGKGLEWVAVISYDGSNKYYADSVKGRFTISRDNSKNTLYLQMNSLRAEDTAVYYCARDLSGY
GDYPDYWGQGTLVTVSS*
pDP161-His8-CD19 NT.1-GFLG-GGFG-FC-TfR-H7
(SEQ ID NO: 89)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGGGFGVDGDKTHTCPPC
PAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGNVES
CSVMHEALHNHYTQKSLSLSPGKGSSGGSGGSGGSSELTQDPAVSVALGQTVRITCQGDSLRSYY
ASWYQQKPGQAPVLVMYGRNERPSGVPDRESGSKSGTSASLAISGLQPEDEANYYCAGWDDSLTG
PVFGGGTKLTVLGGGGGSGGGGSGGGGSQVQLQESGGGVVQPGRSLRLSCAASRFTFSSYAMHWV
RQAPGKGLEWVAVISYDGSNKYYADSVKGRFTISRDNSKNTLYLQMNSLRAEDTAVYYCARDLSG
YGDYPDYWGQGTLVTVSS*
pDP162-His8-CD19 NT.1-FK-FC-TfR-H7
(SEQ ID NO: 90)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGFKGVDGDKTHTCPPCPAPELL
GGPSVFLFPPKPKDTLMISRTPEVTCVVVDVSHEDPEVKENWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGGSSGGSGGSGGSSELTQDPAVSVALGQTVRITCQGDSLRSYYASWYQ
QKPGQAPVLVMYGRNERPSGVPDRFSGSKSGTSASLAISGLQPEDEANYYCAGWDDSLTGPVFGG
GTKLTVLGGGGGSGGGGSGGGGSQVQLQESGGGVVQPGRSLRLSCAASRFTFSSYAMHWVRQAPG
KGLEWVAVISYDGSNKYYADSVKGRFTISRDNSKNTLYLQMNSLRAEDTAVYYCARDLSGYGDYP
DYWGQGTLVTVSS*
pDP163-His8-CD19 NT.1-VA-FC-TfR-H7
(SEQ ID NO: 91)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVAGVDGDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSSELTQDPAVSVALGQTVRITCQGDSLRSYYASWYQQ
KPGQAPVLVMYGRNERPSGVPDRFSGSKSGTSASLAISGLQPEDEANYYCAGWDDSLTGPVFGGG
TKLTVLGGGGGSGGGGSGGGGSQVQLQESGGGVVQPGRSLRLSCAASRFTFSSYAMHWVRQAPGK
GLEWVAVISYDGSNKYYADSVKGRFTISRDNSKNTLYLQMNSLRAEDTAVYYCARDLSGYGDYPD
YWGQGTLVTVSS*
pDP164-His8-CD19 NT.1-VK-FC-TfR-H7
(SEQ ID NO: 92)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVKGVDGDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSSELTQDPAVSVALGQTVRITCQGDSLRSYYASWYQQ
KPGQAPVLVMYGRNERPSGVPDRFSGSKSGTSASLAISGLQPEDEANYYCAGWDDSLTGPVFGGG
TKLTVLGGGGGSGGGGSGGGGSQVQLQESGGGVVQPGRSLRLSCAASRFTFSSYAMHWVRQAPGK
GLEWVAVISYDGSNKYYADSVKGRFTISRDNSKNTLYLQMNSLRAEDTAVYYCARDLSGYGDYPD
YWGQGTLVTVSS*
pDP165-His8-CD19 NT.1-VR-FC-TfR-H7
(SEQ ID NO: 93)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGVRGVDGDKTHTCPPCPAPELL
GGPSVFLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMHE
ALHNHYTQKSLSLSPGKGSSGGSGGSGGSSELTQDPAVSVALGQTVRITCQGDSLRSYYASWYQQ
KPGQAPVLVMYGRNERPSGVPDRESGSKSGTSASLAISGLQPEDEANYYCAGWDDSLTGPVFGGG
TKLTVLGGGGGSGGGGSGGGGSQVQLQESGGGVVQPGRSLRLSCAASRFTFSSYAMHWVRQAPGK
GLEWVAVISYDGSNKYYADSVKGRFTISRDNSKNTLYLQMNSLRAEDTAVYYCARDLSGYGDYPD
YWGQGTLVTVSS*
pDP166-His8-CD19 NT.1-GGFG-FC-TfR-H7
(SEQ ID NO: 94)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFGGVDGDKTHTCPPCPAPEL
LGGPSVFLFPPKPKDTLMISRTPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMH
EALHNHYTQKSLSLSPGKGSSGGSGGSGGSSELTQDPAVSVALGQTVRITCQGDSLRSYYASWYQ
QKPGQAPVLVMYGRNERPSGVPDRESGSKSGTSASLAISGLQPEDEANYYCAGWDDSLTGPVFGG
GTKLTVLGGGGGSGGGGSGGGGSQVQLQESGGGVVQPGRSLRLSCAASRFTFSSYAMHWVRQAPG
KGLEWVAVISYDGSNKYYADSVKGRFTISRDNSKNTLYLQMNSLRAEDTAVYYCARDLSGYGDYP
DYWGQGTLVTVSS*
pDP167-CD20_HC Fc-N297G,S427C-TfR-H7
(SEQ ID NO: 95)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGVDGSGSDKTHTCPPCPAPELLGGPSVFLFPPKPKDTLMISRTPEVTCVVVDVSHEDPEVKEN
WYVDGVEVHNAKTKPREEQYGSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFL
YSKLTVDKSRWQQGNVFCCSVMHEALHNHYTQKSLSLSPGKGSSGGSGGSGGSSELTQDPAVSVA
LGQTVRITCQGDSLRSYYASWYQQKPGQAPVLVMYGRNERPSGVPDRESGSKSGTSASLAISGLQ
PEDEANYYCAGWDDSLTGPVFGGGTKLTVLGGGGGSGGGGSGGGGSQVQLQESGGGVVQPGRSLR
LSCAASRFTFSSYAMHWVRQAPGKGLEWVAVISYDGSNKYYADSVKGRFTISRDNSKNTLYLQMN
SLRAEDTAVYYCARDLSGYGDYPDYWGQGTLVTVSS*
pDP168-CD20-scfv-GFLG-FC-N297G, S427C-TfR-H7
(SEQ ID NO: 96)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYGSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFCCSVMHEALHNHYTQKSLSLSPGKGSSGGSGGSGGSSELTQ
DPAVSVALGQTVRITCQGDSLRSYYASWYQQKPGQAPVLVMYGRNERPSGVPDRFSGSKSGTSAS
LAISGLQPEDEANYYCAGWDDSLTGPVFGGGTKLTVLGGGGGSGGGGSGGGGSQVQLQESGGGVV
QPGRSLRLSCAASRFTFSSYAMHWVRQAPGKGLEWVAVISYDGSNKYYADSVKGRFTISRDNSKN
TLYLQMNSLRAEDTAVYYCARDLSGYGDYPDYWGQGTLVTVSS*
pDP169-cd20-scfv-GFLG-FK-FC-(N297G, S427C)-TfR-H7
(SEQ ID NO: 97)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGFKGVDGDKTHTCPPCPAPELLGGPSVFLFPPKPKDTLMISRTPEVTCVVVDV
SHEDPEVKENWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGKGSSGGSGGSGGSSE
LTQDPAVSVALGQTVRITCQGDSLRSYYASWYQQKPGQAPVLVMYGRNERPSGVPDRESGSKSGT
SASLAISGLQPEDEANYYCAGWDDSLTGPVFGGGTKLTVLGGGGGSGGGGSGGGGSQVQLQESGG
GVVQPGRSLRLSCAASRFTFSSYAMHWVRQAPGKGLEWVAVISYDGSNKYYADSVKGRFTISRDN
SKNTLYLQMNSLRAEDTAVYYCARDLSGYGDYPDYWGQGTLVTVSS*
pDP170-cd20-scfv-GFLG-VR-FC-(N297G, S427C)-TfR-H7
(SEQ ID NO: 98)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVRGVDGDKTHTCPPCPAPELLGGPSVFLFPPKPKDTLMISRTPEVTCVVVDV
SHEDPEVKENWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGKGSSGGSGGSGGSSE
LTQDPAVSVALGQTVRITCQGDSLRSYYASWYQQKPGQAPVLVMYGRNERPSGVPDRESGSKSGT
SASLAISGLQPEDEANYYCAGWDDSLTGPVFGGGTKLTVLGGGGGSGGGGSGGGGSQVQLQESGG
GVVQPGRSLRLSCAASRFTFSSYAMHWVRQAPGKGLEWVAVISYDGSNKYYADSVKGRFTISRDN
SKNTLYLQMNSLRAEDTAVYYCARDLSGYGDYPDYWGQGTLVTVSS*
pDP171-His8-EGFR affi-FC-TfR-H7
(SEQ ID NO: 99)
MYRMQLLSCIALSLALVTNSGHHHHHHHHTGLQVDNKFNKEMWAAWEEIRNLPNLNGWQMTAFIA
SLVDDPSQSANLLAEAKKLNDAQAPKVDGSGSVDGDKTHTCPPCPAPELLGGPSVFLFPPKPKDT
LMISRTPEVTCVVVDVSHEDPEVKENWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GKGSSGGSGGSGGSSELTQDPAVSVALGQTVRITCQGDSLRSYYASWYQQKPGQAPVLVMYGRNE
RPSGVPDRFSGSKSGTSASLAISGLQPEDEANYYCAGWDDSLTGPVFGGGTKLTVLGGGGGSGGG
GSGGGGSQVQLQESGGGVVQPGRSLRLSCAASRFTFSSYAMHWVRQAPGKGLEWVAVISYDGSNK
YYADSVKGRFTISRDNSKNTLYLQMNSLRAEDTAVYYCARDLSGYGDYPDYWGQGTLVTVSS*
pDP172-His8-EGFR affi-GFLG-FC-TfR-H7
(SEQ ID NO: 100)
MYRMQLLSCIALSLALVTNSGHHHHHHHHTGLQVDNKFNKEMWAAWEEIRNLPNLNGWQMTAFIA
SLVDDPSQSANLLAEAKKLNDAQAPKVDGGFLGGVDGDKTHTCPPCPAPELLGGPSVFLFPPKPK
DTLMISRTPEVTCVVVDVSHEDPEVKENWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHNHYTQKSLSL
SPGKGSSGGSGGSGGSSELTQDPAVSVALGQTVRITCQGDSLRSYYASWYQQKPGQAPVLVMYGR
NERPSGVPDRESGSKSGTSASLAISGLQPEDEANYYCAGWDDSLTGPVFGGGTKLTVLGGGGGSG
GGGSGGGGSQVQLQESGGGVVQPGRSLRLSCAASRFTFSSYAMHWVRQAPGKGLEWVAVISYDGS
NKYYADSVKGRFTISRDNSKNTLYLQMNSLRAEDTAVYYCARDLSGYGDYPDYWGQGTLVTVSS*
pDP173-His8-EGFR affi-GFLG-FK-FC-TfR-H7
(SEQ ID NO: 101)
MYRMQLLSCIALSLALVTNSGHHHHHHHHTGLQVDNKFNKEMWAAWEEIRNLPNLNGWQMTAFIA
SLVDDPSQSANLLAEAKKLNDAQAPKVDGGFLGGFKGVDGDKTHTCPPCPAPELLGGPSVFLFPP
KPKDTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKS
LSLSPGKGSSGGSGGSGGSSELTQDPAVSVALGQTVRITCQGDSLRSYYASWYQQKPGQAPVLVM
YGRNERPSGVPDRESGSKSGTSASLAISGLQPEDEANYYCAGWDDSLTGPVFGGGTKLTVLGGGG
GSGGGGSGGGGSQVQLQESGGGVVQPGRSLRLSCAASRFTFSSYAMHWVRQAPGKGLEWVAVISY
DGSNKYYADSVKGRFTISRDNSKNTLYLQMNSLRAEDTAVYYCARDLSGYGDYPDYWGQGTLVTV
SS*
pDP174-His8-EGFR affi-GFLG-VR-FC-TfR-H7
(SEQ ID NO: 102)
MYRMQLLSCIALSLALVTNSGHHHHHHHHTGLQVDNKFNKEMWAAWEEIRNLPNLNGWQMTAFIA
SLVDDPSQSANLLAEAKKLNDAQAPKVDGGFLGGVRGVDGDKTHTCPPCPAPELLGGPSVFLFPP
KPKDTLMISRTPEVTCVVVDVSHEDPEVKENWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEALHNHYTQKS
LSLSPGKGSSGGSGGSGGSSELTQDPAVSVALGQTVRITCQGDSLRSYYASWYQQKPGQAPVLVM
YGRNERPSGVPDRFSGSKSGTSASLAISGLQPEDEANYYCAGWDDSLTGPVFGGGTKLTVLGGGG
GSGGGGSGGGGSQVQLQESGGGVVQPGRSLRLSCAASRFTFSSYAMHWVRQAPGKGLEWVAVISY
DGSNKYYADSVKGRFTISRDNSKNTLYLQMNSLRAEDTAVYYCARDLSGYGDYPDYWGQGTLVTV
SS*
pDP210-CD20-HC-(EVR)-FC (GRLR)-N297G-TfR-H7
(SEQ ID NO: 103)
MYRMQLLSCIALSLALVTNSQVQLQQPGAELVKPGASVKMSCKASGYTFTSYNMHWVKQTPGRGL
EWIGAIYPGNGDTSYNQKFKGKATLTADKSSSTAYMQLSSLTSEDSAVYYCARSTYYGGDWYFNV
WGAGTTVTVSAASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKAEPKSCGGSGSGGEVRGVDGDK
THTCPPCPAPELLRGPSVFLFPPKPKDTLMISRTPEVTCVVVDVSHEDPEVKENWYVDGVEVHNA
KTKPREEQYGSTYRVVSVLTVLHQDWLNGKEYKCKVSNKARPAPIEKTISKAKGQPREPQVYTLP
PSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKGSSGGSGGSGGSSELTQDPAVSVALGQTVRITCQG
DSLRSYYASWYQQKPGQAPVLVMYGRNERPSGVPDRESGSKSGTSASLAISGLQPEDEANYYCAG
WDDSLTGPVFGGGTKLTVLGGGGGSGGGGSGGGGSQVQLQESGGGVVQPGRSLRLSCAASRFTFS
SYAMHWVRQAPGKGLEWVAVISYDGSNKYYADSVKGRFTISRDNSKNTLYLQMNSLRAEDTAVYY
CARDLSGYGDYPDYWGQGTLVTVSS*
pDP213-CD20-HC-(EVR)-FC (GRLR)-N297G
(SEQ ID NO: 104)
MYRMQLLSCIALSLALVTNSQVQLQQPGAELVKPGASVKMSCKASGYTFTSYNMHWVKQTPGRGL
EWIGAIYPGNGDTSYNQKFKGKATLTADKSSSTAYMQLSSLTSEDSAVYYCARSTYYGGDWYFNV
WGAGTTVTVSAASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKAEPKSCGGSGSGGEVRGVDGDK
THTCPPCPAPELLRGPSVFLFPPKPKDTLMISRTPEVTCVVVDVSHEDPEVKENWYVDGVEVHNA
KTKPREEQYGSTYRVVSVLTVLHQDWLNGKEYKCKVSNKARPAPIEKTISKAKGQPREPQVYTLP
PSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK*
pDP219-FC-TfR-H7
(SEQ ID NO: 105)
MYRMQLLSCIALSLALVTNSGHHHHHHHHTGGGGGEVRGVDGSGSGFLGSGSDKTHTCPPCPAPE
LLGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVM
HEALHNHYTQKSLSLSPGKGSSGGSGGSGGSSELTQDPAVSVALGQTVRITCQGDSLRSYYASWY
QQKPGQAPVLVMYGRNERPSGVPDRESGSKSGTSASLAISGLQPEDEANYYCAGWDDSLTGPVFG
GGTKLTVLGGGGGSGGGGSGGGGSQVQLQESGGGVVQPGRSLRLSCAASRFTFSSYAMHWVRQAP
GKGLEWVAVISYDGSNKYYADSVKGRFTISRDNSKNTLYLQMNSLRAEDTAVYYCARDLSGYGDY
PDYWGQGTLVTVSS*
pDP223-pFUSE-H7 scFV-knob-Fc-His
(SEQ ID NO: 106)
MYRMQLLSCIALSLALVTNSSELTQDPAVSVALGQTVRITCQGDSLRSYYASWYQQKPGQAPVLV
MYGRNERPSGVPDRFSGSKSGTSASLAISGLQPEDEANYYCAGWDDSLTGPVFGGGTKLTVLGGG
GGSGGGGSGGGGSQVQLQESGGGVVQPGRSLRLSCAASRFTFSSYAMHWVRQAPGKGLEWVAVIS
YDGSNKYYADSVKGRFTISRDNSKNTLYLQMNSLRAEDTAVYYCARDLSGYGDYPDYWGQGTLVT
VSSPWEPKSCDKTHTCPPCPAPELLGGPSVFLFPPKPKDTLMISRTPEVTCVVVDVSHEDPEVKF
NWYVDGVEVHNAKTKPREEQYGSTYRVVSVLTVLHQDWLNGKEYKCKVSNKALPAPIEKTISKAK
GQPREPQVYTLPPSRDELTKNQVSLWCLVKGFYPSDIAVEWESNGQPENNYKTTPPVLDSDGSFF
LYSKLTVDKSRWQQGNVFSCSVMHEALHNHYTQKSLSLSPGKGGSHHHHHH*
pDP224-pFUSE-CD 19 NT.1-knob-Fc-H7 scFV-His
(SEQ ID NO: 107)
MYRMQLLSCIALSLALVTNSPEEPLVVKVEEGDTAALWCLKGTSDGPTQQLTWSRESPLKPFLKY
SLGVPGLGVHVRPDAISVVIRNVSQQMGGFYLCQPGPPSEKAWQPGWTVNVEGSGELFRWNVSDL
GGLGCGLKNRSSEGPSSPSGKLMSPKLYVWAKDRPEIWEGEPPCLPPRDSLNQSLSRDLTVAPGS
TLWLSCGVPPDSVSRGPLSWTHVHPKGPKSLLSLELKDDRPARDMWVMGTSLMLPRATAQDAGKW
YCHRGNVTTSFHLEVIARPVKAHSDLRTGGWKPWEPKSCDKTHTCPPCPAPELLGGPSVELFPPK
PKDTLMISRTPEVTCVVVDVSHEDPEVKFNWYVDGVEVHNAKTKPREEQYGSTYRVVSVLTVLHQ
DWLNGKEYKCKVSNKALPAPIEKTISKAKGQPREPQVYTLPPSRDELTKNQVSLWCLVKGFYPSD
IAVEWESNGQPENNYKTTPPVLDSDGSFFLYSKLTVDKSRWQQGNVFSCSVMHEALHNHYTQKSL
SLSPGKGSSGGSGGSGGSSELTQDPAVSVALGQTVRITCQGDSLRSYYASWYQQKPGQAPVLVMY
GRNERPSGVPDRFSGSKSGTSASLAISGLQPEDEANYYCAGWDDSLTGPVFGGGTKLTVLGGGGG
SGGGGSGGGGSQVQLQESGGGVVQPGRSLRLSCAASRFTFSSYAMHWVRQAPGKGLEWVAVISYD
GSNKYYADSVKGRFTISRDNSKNTLYLQMNSLRAEDTAVYYCARDLSGYGDYPDYWGQGTLVTVS
SGGSHHHHHH*
pDP225-2-Hole Fc_H7 scFV
(SEQ ID NO: 108)
MRMQLLLLIALSLALVTNSTSGEPKSCDKTHTCPPCPAPELLGGPSVFLFPPKPKDTLMISRTPE
VTCVVVDVSHEDPEVKENWYVDGVEVHNAKTKPREEQYNSTYRVVSVLTVLHQDWLNGKEYKCKV
SNKALPAPIEKTISKAKGQPREPQVYTLPPSRDELTKNQVSLSCAVKGFYPSDIAVEWESNGQPE
NNYKTTPPVLDSDGSFFLVSKLTVDKSRWQQGNVFSCSVMHEALHNHYTQKSLSLSPGKGSSGGS
GGSGGSSELTQDPAVSVALGQTVRITCQGDSLRSYYASWYQQKPGQAPVLVMYGRNERPSGVPDR
ESGSKSGTSASLAISGLQPEDEANYYCAGWDDSLTGPVFGGGTKLTVLGGGGGSGGGGSGGGGSQ
VQLQESGGGVVQPGRSLRLSCAASRFTESSYAMHWVRQAPGKGLEWVAVISYDGSNKYYADSVKG
RFTISRDNSKNTLYLQMNSLRAEDTAVYYCARDLSGYGDYPDYWGQGTLVTVSS*
pDP226-Hole Fc_-avitag
(SEQ ID NO: 109)
MRMQLLLLIALSLALVTNSTSGEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLSCAVKGFYPSDIAVEWESNGQPE
NNYKTTPPVLDSDGSFFLVSKLTVDKSRWQQGNVFSCSVMHEALHNHYTQKSLSLSPGKGGGGSG
LNDIFEAQKIEWHEG*
pDP227-Knob Fc_H7 scFV-His
(SEQ ID NO: 110)
MRMQLLLLIALSLALVTNSTSGEPKSCDKTHTCPPCPAPELLGGPSVFLFPPKPKDTLMISRTPE
VTCVVVDVSHEDPEVKENWYVDGVEVHNAKTKPREEQYGSTYRVVSVLTVLHQDWLNGKEYKCKV
SNKALPAPIEKTISKAKGQPREPQVYTLPPSRDELTKNQVSLWCLVKGFYPSDIAVEWESNGQPE
NNYKTTPPVLDSDGSFFLYSKLTVDKSRWQQGNVFSCSVMHEALHNHYTQKSLSLSPGKGSSGGS
GGSGGSSELTQDPAVSVALGQTVRITCQGDSLRSYYASWYQQKPGQAPVLVMYGRNERPSGVPDR
FSGSKSGTSASLAISGLQPEDEANYYCAGWDDSLTGPVFGGGTKLTVLGGGGGGGGGSGGGGSQV
QLQESGGGVVQPGRSLRLSCAASRFTFSSYAMHWVRQAPGKGLEWVAVISYDGSNKYYADSVKGR
FTISRDNSKNTLYLQMNSLRAEDTAVYYCARDLSGYGDYPDYWGQGTLVTVSSGGSHHHHHH*
PD-L1 TransTAC (pDP186)
(SEQ ID NO: 111)
MYRMQLLSCIALSLALVTNSDIQMTQSPSSLSASVGDRVTITCRASQDVSTAVAWYQQKPGKAPK
LLIYSASFLYSGVPSRFSGSGSGTDFTLTISSLQPEDFATYYCQQYLYHPATFGQGTKVEIKGGG
GSGGGGSGGGGSEVQLVESGGGLVQPGGSLRLSCAASGFTFSDSWIHWVRQAPGKGLEWVAWISP
YGGSTYYADSVKGRFTISADTSKNTAYLQMNSLRAEDTAVYYCARRHWPGGFDYWGQGTLVTVSS
GGSGSAGGGFLGGVRGVDGDKTHTCPPCPAPELLGGPSVFLFPPKPKDTLMISRTPEVTCVVVDV
SHEDPEVKENWYVDGVEVHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGKGSSGGSGGSGGSSE
LTQDPAVSVALGQTVRITCQGDSLRSYYASWYQQKPGQAPVLVMYGRNERPSGVPDRESGSKSGT
SASLAISGLQPEDEANYYCAGWDDSLTGPVFGGGTKLTVLGGGGGSGGGGSGGGGSQVQLQESGG
GVVQPGRSLRLSCAASRFTFSSYAMHWVRQAPGKGLEWVAVISYDGSNKYYADSVKGRFTISRDN
SKNTLYLQMNSLRAEDTAVYYCARDLSGYGDYPDYWGQGTLVTVSS
CD20 TransTAC:
Heavy Chain (pDP210)
(SEQ ID NO: 112)
MYRMQLLSCIALSLALVTNSQVQLQQPGAELVKPGASVKMSCKASGYTFTSYNMHWVKQTPGRGL
EWIGAIYPGNGDTSYNQKFKGKATLTADKSSSTAYMQLSSLTSEDSAVYYCARSTYYGGDWYFNV
WGAGTTVTVSAASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKAEPKSCGGSGSGGEVRGVDGDK
THTCPPCPAPELLRGPSVFLFPPKPKDTLMISRTPEVTCVVVDVSHEDPEVKFNWYVDGVEVHNA
KTKPREEQYGSTYRVVSVLTVLHQDWLNGKEYKCKVSNKARPAPIEKTISKAKGQPREPQVYTLP
PSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGKGSSGGSGGSGGSSELTQDPAVSVALGQTVRITCQG
DSLRSYYASWYQQKPGQAPVLVMYGRNERPSGVPDRESGSKSGTSASLAISGLQPEDEANYYCAG
WDDSLTGPVEGGGTKLTVLGGGGGSGGGGSGGGGSQVQLQESGGGVVQPGRSLRLSCAASRFTFS
SYAMHWVRQAPGKGLEWVAVISYDGSNKYYADSVKGRFTISRDNSKNTLYLQMNSLRAEDTAVYY
CARDLSGYGDYPDYWGQGTLVTVSS
Light Chain (pDP118)
(SEQ ID NO: 113)
MYRMQLLSCIALSLALVTNSQIVLSQSPAILSASPGEKVIMTCRASSSVSYIHWFQQKPGSSPKP
WIYATSNLASGVPVRFSGSGSGTSYSLTISRVEAEDAATYYCQQWTSNPPTFGGGTKLEIKRTVA
APSVFIFPPSDEQLKSGTASVVCLLNNFYPREAKVQWCVDNALQSGNSQESVTEQDSKDSTYSLS
STLTLSKADYEKHKVYACEVTHQGLSSPVTKSENRGEC
CD20 TransTAC-M16 version (pDP127):
Heavy chain
(the light chain is as immediately above)
(SEQ ID NO: 114)
MYRMQLLSCIALSLALVTNSQIVLSQSPAILSASPGEKVTMTCRASSSVSYIHWFQQKPGSSPKP
WIYATSNLASGVPVRFSGSGSGTSYSLTISRVEAEDAATYYCQQWTSNPPTFGGGTKLEIKGGGG
SGGGGSGGGGSQVQLQQPGAELVKPGASVKMSCKASGYTFTSYNMHWVKQTPGRGLEWIGAIYPG
NGDTSYNQKFKGKATLTADKSSSTAYMQLSSLTSEDSAVYYCARSTYYGGDWYFNVWGAGTTVTV
SAGGSGSGGFLGGVDGDKTHTCPPCPAPELLGGPSVFLFPPKPKDTLMISRTPEVTCVVVDVSHE
DPEVKENWYVDGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSSGGSGGSGGSSELTQ
DPAVSVALGQTVRITCQGDSLRSYYASWYQQKPGQAPVLVMYGRNERPSGVPDRFSGSKSGTSAS
LAISGLQPEDEANYYCAGWDDSLTGPVFGGGTKLTVLGGGGGSGGGGSGGGGSQVQLQESGGGVV
QPGRSLRLSCAASRYPFHHHDHHWVRQAPGKGLEWVAVISYDGSNKYYADSVKGRFTISRDNSKN
TLYLQMNSLRAEDTAVYYCARDLSGYGDYPDYWGQGTLVTVSS*
EGFR TransTAC (pDP211)
(SEQ ID NO: 115)
MYRMQLLSCIALSLALVTNSLQVDNKFNKEMWAAWEEIRNLPNLNGWQMTAFIASLVDDPSQSAN
LLAEAKKLNDAQAPKVDGGGGEVRGVDGDKTHTCPPCPAPELLGGPSVFLFPPKPKDTLMISRTP
EVTCVVVDVSHEDPEVKENWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSLSPGKGSSGG
SGGSGGSSELTQDPAVSVALGQTVRITCQGDSLRSYYASWYQQKPGQAPVLVMYGRNERPSGVPD
RFSGSKSGTSASLAISGLQPEDEANYYCAGWDDSLTGPVFGGGTKLTVLGGGGGSGGGGSGGGGS
QVQLQESGGGVVQPGRSLRLSCAASRFTFSSYAMHWVRQAPGKGLEWVAVISYDGSNKYYADSVK
GRFTISRDNSKNTLYLQMNSLRAEDTAVYYCARDLSGYGDYPDYWGQGTLVTVSS
CD19 CAR TransTAC (pDP160)
(SEQ ID NO: 116)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVRGVDGDKTHTCPPCP
APELLGGPSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKLTVDKSRWQQGNVFSC
SVMHEALHNHYTQKSLSLSPGKGSSGGSGGSGGSSELTQDPAVSVALGQTVRITCQGDSLRSYYA
SWYQQKPGQAPVLVMYGRNERPSGVPDRESGSKSGTSASLAISGLQPEDEANYYCAGWDDSLTGP
VFGGGTKLTVLGGGGGSGGGGSGGGGSQVQLQESGGGVVQPGRSLRLSCAASRFTESSYAMHWVR
QAPGKGLEWVAVISYDGSNKYYADSVKGRFTISRDNSKNTLYLQMNSLRAEDTAVYYCARDLSGY
GDYPDYWGQGTLVTVSS
CD19 CAR TransTAC earlier version (pDP50)
(SEQ ID NO: 117)
MYRMQLLSCIALSLALVTNSGHHHHHHHHTGPEEPLVVKVEEGDTAALWCLKGTSDGPTQQLTWS
RESPLKPFLKYSLGVPGLGVHVRPDAISVVIRNVSQQMGGFYLCQPGPPSEKAWQPGWTVNVEGS
GELFRWNVSDLGGLGCGLKNRSSEGPSSPSGKLMSPKLYVWAKDRPEIWEGEPPCLPPRDSLNQS
LSRDLTVAPGSTLWLSCGVPPDSVSRGPLSWTHVHPKGPKSLLSLELKDDRPARDMWVMGTSLML
PRATAQDAGKWYCHRGNVTTSFHLEVIARPVKAHSDLRTGGWKGGFLGGVDGDKTHTCPPCPAPE
LLGGPSVFLFPPKPKDTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSCSVM
HEALHNHYTQKSLSLSPGKGSSGGSGGSGGSVPDKTVRWCAVSEHEATKCQSFRDHMKSVIPSDG
PSVACVKKASYLDCIRAIAANEADAVTLDAGLVYDAYLAPNNLKPVVAEFYGSKEDPQTFYYAVA
VVKKDSGFQMNQLRGKKSCHTGLGRSAGWNIPIGLLYCDLPEPRKPLEKAVANFFSGSCAPCADG
TDFPQLCQLCPGCGCSTLNQYFGYSGAFKCLKDGAGDVAFVKHSTIFENLANKADRDQYELLCLD
NTRKPVDEYKDCHLAQVPSHTVVARSMGGKEDLIWELLNQAQEHFGKDKSKEFQLFSSPHGKDLL
FKDSAHGFLKVPPRMDAKMYLGYEYVTAIRNLREGTCPEAPTDECKPVKWCALSHHERLKCDEWS
VNSVGKIECVSAETTEDCIAKIMNGEADAMSLDGGFVYIAGKCGLVPVLAENYNKSDNCEDTPEA
GYFAIAVVKKSASDLTWDNLKGKKSCHTAVGRTAGWNIPMGLLYNKINHCRFDEFFSEGCAPGSK
KDSSLCKLCMGSGLNLCEPNNKEGYYGYTGAFRCLVEKGDVAFVKHQTVPQNTGGKNPDPWAKNL
NEKDYELLCLDGTRKPVEEYANCHLARAPNHAVVTRKDKEACVHKILRQQQHLFGSNVTDCSGNF
CLFRSETKDLLFRDDTVCLAKLHDRNTYEKYLGEEYVKAVGNLRKCSTSSLLEACTFRRP

In some embodiments, a bispecific modulator can be a heterodimer between a fusion protein R1-R2-R3 (R1 is POIB; R2 is R4-R5 or R5-R4 with R4 as antibody Fc region, and R5 as protease-sensitive linker; R3 is TRB, as above) and a fusion protein R3-R4 or R4-R3, where R3 is a TRB and R4 is an antibody Fc region. In some embodiments, optionally, there can be a protease-sensitive linking means between R3 and R4, or R3-R4 or R4-R3.

In some embodiments, a bispecific modulator disclosed herein can have the formula R1-R6-R3, where R1 is a protein of interest binder (POIB), R6 is a dimerization means, and R3 is a transferrin receptor binding (TRB) means. In some embodiments, R6 can be a moiety that links R1 and R3 (e.g., a linking means that connects R1 and R3). In some embodiments, R6 can be an amino acid linker. In some embodiments, the amino acid linker can be protease sensitive. In some embodiments, R6 can be a dimerization domain that can form a dimer with another copy of R6 through a covalent (e.g., cysteine-containing Fc antibody region or other dimerization domain) or non-covalent bond. In some embodiments, R6 can be a combination of a linking molecule as above (e.g., that can be protease sensitive) and a dimerization domain.

In some embodiments, a bispecific modulator disclosed herein can have the formula R1-R6-R3, where R1 is a protein of interest binder (POIB), R6 is a dimerization means, and R3 is a transferrin receptor binding (TRB) means. In some embodiments, R6 can be a moiety that links R1 and R3, and contains a dimerization domain. The dimerization domain can form a dimer with another copy of R6 through a covalent (e.g., cysteine-containing Fc antibody region) or non-covalent bond. In some embodiments, the dimerization domain can be an Fc antibody region that has one or more cysteines. In some embodiments, or optionally, a linkage between R1 and R6, or between R6 and R3, can be a protease-sensitive linking means.

In some embodiments, a first component of a bispecific modulator can be R1-R6 or R6-R1, where R1 can be a POIB, and R6 can be a dimerization means as above and, optionally, also a protease sensitive amino acid linker. A second component of the bispecific modulator can be R3-R7, where R3 can be a TRB means, and R7 can be a multimerization domain as above and, optionally, also a protease sensitive amino acid linker. A bispecific modulator can be formed when the multimerization domain (e.g., dimerization domain) of the first component forms covalent or non-covalent bonding with the multimerization domain of the second component.

In some embodiments, the bispecific modulators disclosed herein are designed to target cell-surface molecules (e.g., molecules of interest, such as proteins of interest) on tumor cells. In some embodiments, these cell-surface molecules can regulate growth of the cells. In some embodiments, targeting of these cell-surface molecules can kill the tumor cells. In some embodiments, the cell-surface molecule targeted by the bispecific modulators can be epidermal growth factor receptor (EGFR). In some embodiments, these bispecific modulators have 10-50-fold better IC₅₀ for tumor/cancer cells than other treatments.

In some embodiments, the bispecific modulators disclosed herein are used to internalize and degrade multi-pass transmembrane proteins (i.e., transmembrane proteins that span the membrane multiple times and create multiple extracellular domains). In some embodiments, CD20 is a protein targeted using these bispecific modulators.

In some embodiments, a bispecific modulator can be one polypeptide chain. In some embodiments, a bispecific modulator can be two polypeptide chains. In some embodiments of a two-polypeptide configuration, two binders are encoded in two polypeptide chains. In some embodiments, the two polypeptide chains can be linked or connected by a dimerizing domain. In some embodiments, the two polypeptide chains can be linked or connected by a knob-in-hole Fc.

### Targeted Receptor Degradation by TransTAC

In some embodiments, cell-surface molecules (i.e., molecules of interest, such as proteins of interest) that a bispecific modulator targets can be internalized by the bispecific modulator. In some embodiments, the internalized molecule may not be degraded or may be minimally degraded inside the cell. In some embodiments, the bispecific modulators disclosed herein are modified to more efficiently degrade targeted proteins. In some embodiments, the bispecific modulators are modified to contain amino acid sequences sensitive to proteases (e.g., see FIG. 35). The proteases can be endosomal or lysosomal proteases. In some embodiments, a peptide linker that is a target for cathepsin proteases can be used. When the linker is cleaved by the protease, the molecule of interest is released from the bispecific modulator.

In some embodiments, the linkers are sensitive to cleavage by cathepsins. In some embodiments, the cathepsins can be cathepsin A, B, C, D, E, F, G, H, K, L1, L2, O, S, W or Z.

In some embodiments, the molecule of interest can be released from bispecific modulators other than by inclusion of a protease-sensitive linker (e.g., pH-dependent binding of TRB).

Cleavage of the linker inside the cell (e.g., in endosomes) can release the molecule of interest from the internalizing receptor or membrane protein and increase the likelihood that the molecule of interest will be degraded. In some embodiments, the protease-sensitive peptide linker can be positioned such that cleavage of the bispecific modulators by the protease releases or dissociates the targeted protein from the bispecific modulator, allowing the targeted protein to be more completely degraded.

In some embodiments, the molecule of interest can be released from bispecific modulators other than by inclusion of a protease-sensitive linker (e.g., pH-dependent binding of TRB).

In some embodiments, bispecific modulator is R1-R2-R3, as described earlier, and where R2 can be R4-R5 or R5-R4 (R5 is protease-sensitive linking means), the protease-sensitive linking means can be located between a POIB (R1) and an Fc region from an antibody (R4), as in R1-R5-R4-R3. In some embodiments, the protease-sensitive linking means can be located between an Fc region from an antibody (R4) and a TRB (R3), as in R1-R4-R5-R3. In some embodiments, release of the targeted protein from the bispecific modulator can be accomplished by incorporating low pH-sensitive amino acid regions into the bispecific modulators. In some embodiments, when the bispecific modulator is inside an endosome, the low pH environment can release/dissociate the targeted protein from the bispecific modulator such that the targeted protein is more efficiently degraded.

In some embodiments, a linker can be sensitive to the low pH present in endosomes. In some embodiments, the low pH can cause cleavage of the linker.

In some embodiments, the transferrin receptor binding means (TRB) can bind to the transferrin dependent on pH. For example, the TRB may have less affinity for transferrin receptor at lower pH found in endosomes. The lower affinity may result in the TRB releasing the transferrin receptor. This release can facilitate degradation of the protein of interest bound to the POIB. Such a TRB can be “M16” as shown in FIG. 35.

In some embodiments, the protease-sensitive linker can be located between the first moiety (targets a molecule of interest) and the second moiety (binds to an internalizing receptor or membrane protein). In some embodiments, the linker can be localized closer to the first moiety than to the second moiety.

In some embodiments, the protease sensitive linking means can include Gly-Phe-Leu-Gly (GFLG; SEQ ID NO: 118). In some embodiments, the peptide linker can include a Valine-Arginine (VR) and/or Phenylalanine-Lysine (FK) sequence. In some embodiments, the peptide linker can be a GFLG (SEQ ID NO: 118), 3×GFLG (GFLGGFLGGFLG; SEQ ID NO: 119), GFLGVA (SEQ ID NO: 120), GFLGVK (SEQ ID NO: 121), GFLGVR (SEQ ID NO: 122), GFLGGFLG (SEQ ID NO: 123), FK, VA, EVA, or VK linker (FIG. 35). In some embodiments, the peptide linker can be GGFLGGVRGVDG (SEQ ID NO: 7) or GSGSGGEVRGVDG (SEQ ID NO: 8). In some embodiments, the peptide linker can be GFLGGVR (SEQ ID NO: 144) or GGGEVRG (SEQ ID NO: 145).

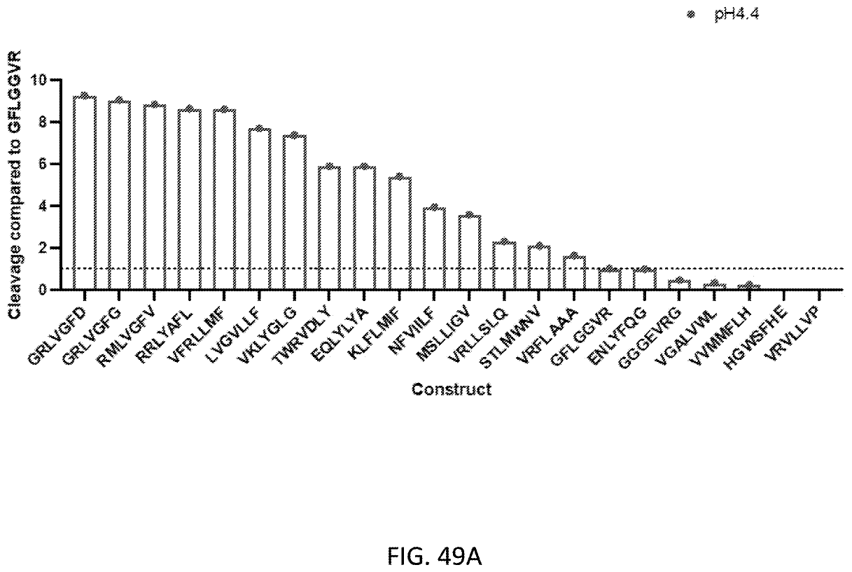

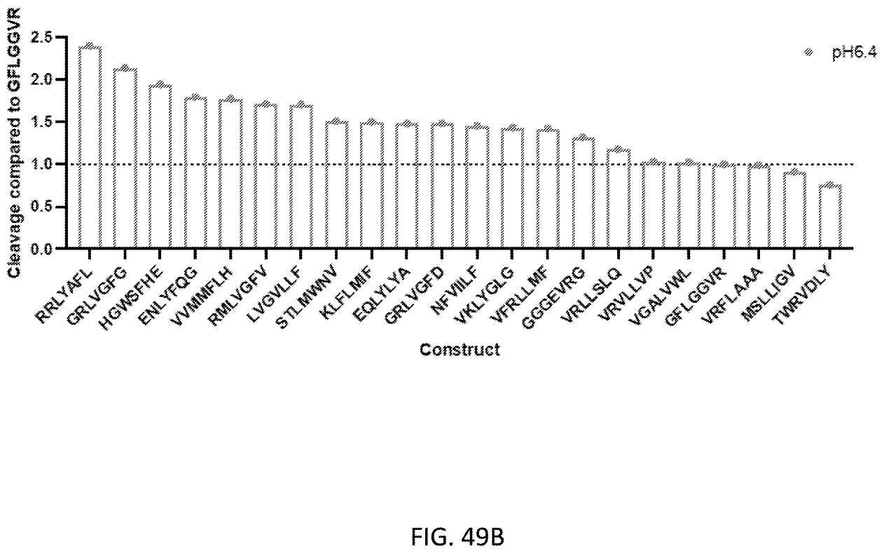

In experiments, (FIG. 49A-B) a yeast-displayed peptide library was used to identify peptides not known to be sensitive to cathepsin cleavage. These peptides can be from a combination of small motifs found in SEQ ID NOs: 144 and 145. In some embodiments, these peptides can be GRLVGFD (SEQ ID NO: 124), GRLVGFG (SEQ ID NO: 125), RMLVGFV (SEQ ID NO: 126), RRLYAFL (SEQ ID NO: 127), VFRLLMF (SEQ ID NO: 128), LVGVLLF (SEQ ID NO: 129), VKLYGLG (SEQ ID NO: 130), TWRVDLY (SEQ ID NO: 131), EQLYLYA (SEQ ID NO: 132), KLFLMIF (SEQ ID NO:133), NFVIILF (SEQ ID NO: 134), MSLLIGV (SEQ ID NO: 135), VRLLSLQ (SEQ ID NO: 136), STLMWNV (SEQ ID NO: 137), VRFLAAA (SEQ ID NO: 138), HGWSFHE (SEQ ID NO: 139), ENLYFQG (SEQ ID NO: 140), VVMMFLH (SEQ ID NO: 141), VFRLLMF (SEQ ID NO:142), or VGALVWL (SEQ ID NO: 143).

Other sequences can be used.

In some embodiments, any combination of these peptide linkers and/or valine-citrulline (VC) linker and/or glutamate-valine-arginine (EVR) linker can be used.

### Antibodies

Unique recombinant monoclonal antibodies are disclosed which can be part of the bispecific modulators disclosed herein. In embodiments, an antibody may be used in the first and/or second moiety of the bispecific modulators discloses herein. [00396]“Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together. “Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the antibodies disclosed herein can exhibit increased cross-reactivity in comparison to an unmodified antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

Some embodiments also feature antibodies that have a specified percentage identity or similarity to the amino acid or nucleotide sequences of the antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e., BLAST or BLAST 2.0), manual alignment or visual inspection can be utilized to determine percent sequence identity or similarity for the nucleic acids and proteins of the present invention.

Aspects of the invention provide isolated antibodies. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. “Specifically binds” or “immunoreacts with” can refer to the antibody reacting with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, Fab′, Fab, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,5 13; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight approximately 53,000-70,000. The four chains are joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgAQ1 and IgA2) or subclass of an immunoglobulin molecule.

Light chains are classified as either kappa or lambda (x, X). Each heavy chain class can be bound with either a kappa or lambda light chain. For example, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a β-sheet conformation and the CDRs form loops which connect, and in some cases form part of, the 3-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and can have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ allows the cancellation of all parameters not related to affinity, and is equal to the equilibrium binding constant, K_{D}. (See, generally, Davies et al. (1990) Annual Rev Biochem 59:439-473). An antibody of the invention can specifically bind to an epitope when the equilibrium binding constant (K_{D}) is ≤1 μM, ≤10 μM, ≤10 nM, ≤10 μM, or ≤100 μM to about 1 μM, as measured by kinetic assays such as radioligand binding assays or similar assays known to those skilled in the art, such as BIAcore or Octet (BLI). For example, in some embodiments, the K_{D} is between about 1E−12 M and a K_{D} about 1E−11 M. In some embodiments, the K_{D} is between about 1E−11 M and a K_{D} about 1E−10 M. In some embodiments, the K_{D} is between about 1E−10 M and a K_{D} about 1E−9 M. In some embodiments, the K_{D} is between about 1E−9 M and a K_{D} about 1E−8 M. In some embodiments, the K_{D} is between about 1E−8 M and a K_{D} about 1E−7 M. In some embodiments, the K_{D} is between about 1E−7 M and a K_{D} about 1E−6 M. For example, in some embodiments, the K_{D} is about 1E−12 M while in other embodiments the K_{D} is about 1E−11 M. In some embodiments, the K_{D} is about 1E−10 M while in other embodiments the K_{D} is about 1E−9 M. In some embodiments, the K_{D} is about 1E−8 M while in other embodiments the K_{D} is about 1E−7 M. In some embodiments, the K_{D} is about 1E−6 M while in other embodiments the K_{D} is about 1E−5 M. In some embodiments, for example, the K_{D} is about 3 E−11 M, while in other embodiments the K_{D} is about 3E−12 M. In some embodiments, the K_{D} is about 6E−11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the antibody can be monovalent or bivalent, and can comprise a single or double chain. Functionally, the binding affinity of the antibody is within the range of 10⁻⁵ M to 10⁻¹²M. For example, the binding affinity of the antibody is from 10⁻⁶ M to 10⁻²M, from 10⁻⁷ M to 10⁻¹²M, from 10⁻⁸ M to 10⁻¹M, from 10⁻⁹ M to 10⁻²M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹⁰ M, from 10−M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

Those skilled in the art will recognize that one can determine, without undue experimentation, if a human monoclonal antibody has the same specificity as a human monoclonal antibody of the invention by ascertaining whether the former prevents the latter from specifically binding. For example, if the human monoclonal antibody being tested competes with the human monoclonal antibody of the invention, as shown by a decrease in binding by the human monoclonal antibody of the invention, then the two monoclonal antibodies bind to the same, or to a closely related, epitope.

Another way to determine whether a human monoclonal antibody has the specificity of a human monoclonal antibody of the invention is to pre-incubate the human monoclonal antibody of the invention with an epitope, with which it is normally reactive, and then add the human monoclonal antibody being tested to determine if the human monoclonal antibody being tested is inhibited in its ability to bind the epitope. If the human monoclonal antibody being tested is inhibited then, it has the same, or functionally equivalent, epitopic specificity as the monoclonal antibody of the invention. Screening of human monoclonal antibodies of the invention can be also carried out by utilizing epitopes and determining whether the test monoclonal antibody is able to neutralize polypeptides containing the epitope.

Various procedures known within the art can be used for the production of polyclonal or monoclonal antibodies directed against a protein of the invention, or against derivatives, fragments, analogs homologs or orthologs thereof. (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site that can immunoreact with a specific epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or are capable of producing antibodies that will specifically bind to the immunizing agent. Alternatively, the lymphocytes can be immunized in vitro.

### Nucleic Acids, Vectors and Cells Expressing Bispecific Modulators

Also disclosed are nucleic acids encoding all or part of the bispecific modulators described herein. Also disclosed are various vectors (e.g., plasmids, viral, and the like) that include the nucleic acids. Also disclosed are various cells (e.g., prokaryotic, eukaryotic) that contain nucleic acids or vectors and can express the fusion proteins.

### Methods

Disclosed herein are methods for administrating the bispecific modulators described herein to a subject. In various embodiments, the bispecific modulators can internalize membrane proteins of interest (e.g., cellular receptors or other membrane proteins) and selectively degrade and/or modulate these proteins. In some embodiments, the bispecific modulators can target CAR receptors on CAR-T cells. In some embodiments, the methods are used to treat toxicities in subjects who have received CAR-T cell infusion for treatment of cancer (e.g., toxicities due to cytokine release). In some embodiments, the methods are used to improve efficacy of CAR-T cells that have been administered to a subject to treat cancer.

In some embodiments, membrane proteins on cancer cells can be targeted to degrade and/or modulate the proteins (e.g., epidermal growth factor receptor or EGFR, Programmed death-ligand or PD-L1). In some examples, bispecific modulators can be used to improve anti-tumor responses in this way.

In some embodiments, the reagents and methods disclosed herein can be used with cells that are not cancer cells.

### Therapeutic Preparations

Aspects of the invention are drawn towards therapeutic preparations. As used herein, the term “therapeutic preparation” can refer to any compound or composition that can be used or administered for therapeutic effects (e.g., bispecific modulators). As used herein, the term “therapeutic effects” can refer to effects sufficient to result in amelioration of symptoms, e.g., treatment, healing, prevention or amelioration of the relevant medical condition, or an increase in rate of treatment, healing, prevention or amelioration of such conditions.

Embodiments as described herein can be administered to a subject in the form of a pharmaceutical composition or therapeutic preparation prepared for the intended route of administration. Such compositions and preparations can comprise, for example, the active ingredient(s) and a pharmaceutically acceptable carrier. Such compositions and preparations can be in a form adapted to oral, subcutaneous, parenteral (such as, intravenous, intraperitoneal), intramuscular, rectal, epidural, intratracheal, intranasal, dermal, vaginal, buccal, ocularly, or pulmonary administration, such as in a form adapted for administration by a peripheral route or is suitable for oral administration or suitable for parenteral administration. Other routes of administration are subcutaneous, intraperitoneal and intravenous, and such compositions can be prepared in a manner well-known to the person skilled in the art, e.g., as described in “Remington's Pharmaceutical Sciences”, 17. Ed. Alfonso R. Gennaro (Ed.), Mark Publishing Company, Easton, Pa., U.S.A., 1985 and more recent editions and in the monographs in the “Drugs and the Pharmaceutical Sciences” series, Marcel Dekker. The compositions and preparations can appear in conventional forms, for example, solutions and suspensions for injection, capsules and tablets, in the form of enteric formulations, e.g., as disclosed in U.S. Pat. No. 5,350,741, and for oral administration.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EM™ (BASF, Parsippany, N.J.) or phosphate buffered saline (PBS). In all cases, the composition can be sterile and can be fluid to the extent that easy syringeability exists. In embodiments, it can be stable under the conditions of manufacture and storage and can be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by using a coating such as lecithin, by the maintenance of the required particle size in the case of dispersion and by using surfactants. Prevention of the action of microorganisms can be achieved by various antibacterial and antifungal agents, for example, parabens, chlorobutanol, phenol, ascorbic acid, and thimerosal. In many cases, it can be useful to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the compound in the required amount in an appropriate solvent with one or a combination of ingredients enumerated herein, as required, followed by filtered sterilization. Dispersions are prepared by incorporating the active compound into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Oral formula of the drug can be administered once a day, twice a day, three times a day, or four times a day, for example, depending on the half-life of the drug.

Pharmaceutically compatible binding agents, and/or adjuvant materials can be included as part of the composition administered to a subject. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel® (sodium starch glycolate), or corn starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as known in the art.

In embodiments, administering can comprise the placement of a pharmaceutical composition into a subject by a method or route which results in at least partial localization of the composition at a desired site such that desired effect is produced.

For example, the pharmaceutical composition can be administered by bolus injection or by infusion. A bolus injection can refer to a route of administration in which a syringe is connected to the IV access device and the medication is injected directly into the subject. The term “infusion” can refer to an intravascular injection.

Embodiments as described herein can be administered to a subject one time (e.g., as a single injection, bolus, or deposition). Alternatively, administration can be once or twice daily to a subject for a period of time, such as from about 2 weeks to about 28 days. Administration can continue for up to one year. In embodiments, administration can continue for the life of the subject. It can also be administered once or twice daily to a subject for period of 1, 2, 3, 4, 5, 6, 7, 8, 9, 10, 11, 12 times per year, or a combination thereof.

In embodiments, compositions as described herein can be administered to a subject chronically. “Chronic administration” can refer to administration in a continuous manner, such as to maintain the therapeutic effect (activity) over a prolonged period of time.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of a reagent or therapeutic composition of the invention can be the amount needed to achieve a therapeutic objective. As noted herein, this can be a binding interaction between the reagent or therapeutic composition and its target that, in certain cases, interferes with the functioning of the target. The amount required to be administered will furthermore depend on the binding affinity of the reagent or therapeutic composition for its specific target and will also depend on the rate at which an administered reagent or therapeutic composition is depleted from the free volume other subject to which it is administered. The dosage administered to a subject (e.g., a patient) of the binding polypeptides described herein is about 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration is often possible. Further, the dosage and frequency of administration of reagent or therapeutic composition of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where fragments (e.g., antibody fragments) are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the specific indication being treated, for example, those with complementary activities that do not adversely affect each other. Alternatively, or in addition, the composition can comprise an agent that enhances its function, such as, for example, a cytotoxic agent, cytokine (e.g., IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions. Sustained-released preparations can be prepared.

The pharmaceutical or therapeutic carrier or diluent employed can be a conventional solid or liquid carrier. Nonlimiting examples of solid carriers are lactose, terra alba, sucrose, cyclodextrin, talc, gelatin, agar, pectin, acacia, magnesium stearate, stearic acid or lower alkyl ethers of cellulose. Nonlimiting examples of liquid carriers are syrup, peanut oil, olive oil, phospholipids, fatty acids, fatty acid amines, polyoxyethylene and water. Similarly, the carrier or diluent can include any sustained release material known in the art, such as glyceryl monostearate or glyceryl distearate, alone or mixed with a wax.

When a solid carrier is used for oral administration, the preparation can be tableted, placed in a hard gelatin capsule in powder or pellet form or it can be in the form of a troche or lozenge. The amount of solid carrier will vary widely but can be from about 25 mg to about 1 g.

When a liquid carrier is used, the preparation can be in the form of a syrup, emulsion, soft gelatin capsule or sterile injectable liquid such as an aqueous or non-aqueous liquid suspension or solution.

The composition and/or preparation can also be in a form suited for local or systemic injection or infusion and can, as such, be formulated with sterile water or an isotonic saline or glucose solution. The compositions can be in a form adapted for peripheral administration only, except for centrally administrable forms. The compositions and/or preparations can be in a form adapted for central administration.

The compositions and/or preparations can be sterilized by conventional sterilization techniques which are well known in the art. The resulting aqueous solutions can be packaged for use or filtered under aseptic conditions and lyophilized, the lyophilized preparation being combined with the sterile aqueous solution prior to administration. The compositions and/or preparations can contain pharmaceutically and/or therapeutically acceptable auxiliary substances as required to approximate physiological conditions, such as buffering agents, tonicity adjusting agents and the like, for instance sodium acetate, sodium lactate, sodium chloride, potassium chloride, calcium chloride, etc.

### Embodiments

Disclosed below in numbered paragraphs are example embodiments disclosed herein.

1. A bispecific modulator as disclosed herein.

2. The bispecific modulator of embodiment 1, comprising:
a. a first moiety comprising an antigen or epitope that can be bound by a chimeric antigen receptor (CAR); and
b. a second moiety that can bind to an internalizing receptor or membrane protein on a cell.

3. The bispecific modulator of embodiment 1, comprising:
a. a first moiety comprising an antibody that can bind to a CAR; and
b. a second moiety that can bind to an internalizing receptor or membrane protein on a cell.

4. The bispecific modulator of one of embodiments 2 or 3, wherein the second moiety comprises an antibody.

5. The bispecific modulator of one of embodiments 2 or 3, wherein binding of the first moiety by the CAR and binding of the second moiety by the internalizing receptor or membrane protein on the cell causes internalization of the CAR into the cell.

6. The bispecific modulator of embodiment 5, wherein binding of the first moiety by the CAR and binding of the second moiety by the internalizing receptor or membrane protein on the cell causes internalization of the CAR into the cell.

7. The bispecific modulator or embodiment 5, wherein binding of the first moiety by the CAR and binding of the second moiety by the internalizing receptor or membrane protein on the cell causes internalization of CAR into the cell and degradation of the CAR.

8. A molecule with at least two moieties, comprising:
a. a first moiety that can bind to a molecule of interest on a cell; and
b. a second moiety that can bind to an internalizing molecule on a cell.

9. The molecule of embodiment 29, wherein:
a. the first moiety comprises an antibody, antibody fragment, ligand, peptide, small molecule, or apatamer; and
b. the second moiety comprises an antibody, antibody fragment, ligand, peptide, a small molecule, or an apatamer.

10. The molecule of embodiment 8, wherein the molecule of interest on the cell is not the same as the internalizing molecule on the cell.

11. The molecule of embodiment 8, wherein the first moiety and the second moiety comprise one polypeptide.

12. The molecule of embodiment 9, wherein the internalizing molecule on the cell comprises an internalizing receptor.

13. The molecule of embodiment 12, wherein the internalizing molecule can be internalized by clathrin-mediated endocytosis.

14. The molecule of embodiment 12, wherein the internalizing molecule can be internalized by clathrin-independent endocytosis.

15. The molecule of embodiment 12, wherein the internalizing molecule comprises transferrin receptor.

16. The molecule of embodiment 12, wherein the internalizing molecule comprises a G-protein coupled receptor (GPCR), receptor tyrosine kinase (RTK) or transmembrane receptor (TMR).

17. The molecule of embodiment 16, wherein the GPCR comprises an adrenoceptor, chemokine receptor, or coagulation receptor.

18. The molecule of embodiment 16, wherein the RTK comprises a colony stimulating factor receptor, epidermal growth factor receptor, tyrosine kinase receptor, fibroblast growth factor receptor, insulin-like growth factor receptor, platelet-derived growth factor receptor, or transforming growth factor receptor.

19. The molecule of embodiment 16, wherein the TMR comprises a folate receptor, interleukin receptor (e.g., IL-2 receptors), low density lipoprotein receptor, or transferrin receptor.

20. The molecule of embodiment 12, wherein the internalizing molecule comprises a transferrin receptor (TfR).

21. The molecule of embodiment 20, wherein the transferrin receptor comprises transferrin receptor 1 (TfR1) or transferrin receptor 2 (TfR2).

22. The molecule of embodiment 9, wherein the ligand for the internalizing molecule comprises at least a part of a natural-occurring ligand that can be bound by the receptor.

23. The molecule of embodiment 22, wherein the ligand for the internalizing molecule comprises at least a part of transferrin, cholesterol, low-density lipoprotein, and epidermal growth factor.

24. The molecule of embodiment 9, wherein the ligand for the internalizing molecule can be bound by a G-protein coupled receptor (GPCR), receptor tyrosine kinase (RTK) or transmembrane receptor (TMR).

25. The molecule of embodiment 22, wherein the ligand for the internalizing molecule can be bound by a transferrin receptor.

26. The molecule of embodiment 22, wherein the ligand for the internalizing molecule comprises transferrin protein or a portion of a transferrin protein.

27. The molecule of embodiment 9, wherein the second moiety comprises a Fab, scFv, single-domain antibody, nanobody, monobody, DARPin or affibody.

28. The molecule of embodiment 27, wherein the second moiety comprises an H7 scFv.

29. The molecule of embodiment 27, wherein the second moiety comprises H7 Fab or an engineered H7 antibody variant.

30. The molecule of embodiment 9, wherein the second moiety can bind to a transferrin receptor, cholesterol receptor, low-density lipoprotein receptor, or epidermal growth factor receptor.

31. The molecule of embodiment 9, wherein the second moiety can bind to a G-protein coupled receptor (GPCR), receptor tyrosine kinase (RTK), or transmembrane receptor (TMR).

32. The molecule of embodiment 9, wherein the second moiety can bind to transferrin receptor (TfR).

33. The molecule of embodiment 8, wherein the molecule of interest comprises a protein.

34. The molecule of embodiment 33, wherein the protein comprises a membrane protein.

35. The molecule of embodiment 33, wherein the protein comprises an integral membrane protein.

36. The molecule of embodiment 33, wherein the protein comprises an extracellular protein.

37. The molecule of embodiment 36, wherein the extracellular protein is found in an external environment.

38. The molecule of embodiment 36, wherein the extracellular protein is selected from the group consisting of an autoantibody, a cytokine, an enzyme, and combinations thereof.

39. The molecule of embodiment 33, wherein the protein comprises a transmembrane protein.

40. The molecule of embodiment 39, wherein the transmembrane protein has one (1) or more transmembrane domains.

41. The molecule of embodiment 8, wherein the molecule of interest can bind a hormone, cytokine, growth factor, neurotransmitter, lipophilic signaling molecule (e.g., prostaglandin) or cell recognition molecule (e.g., integrin, selectin).

42. The molecule of embodiment 8 wherein the molecule of interest comprises a receptor.

43. The molecule of embodiment 42, wherein the receptor comprises a G-protein coupled receptor (GPCR).

44. The molecule of embodiment 42, wherein the receptor comprises a receptor tyrosine kinase (RTK) or transmembrane receptor (TMR).

45. The molecule of embodiment 39, wherein the receptor comprises a ligand-gated ion channel-linked molecule, a transporter, an enzyme-linked molecule, or a G-protein-linked receptor.

46. The molecule of embodiment 45, wherein the ligand-gated ion channel linked molecule provides for movement of Na+, K+, Ca2+, or Cl− to move across a plasma membrane of a cell.

47. The molecule of embodiment 45, wherein the enzyme-linked molecule comprises a receptor tyrosine kinase, tyrosine-kinase-associated receptor (e.g., enzymes that associate with cytokines), receptor-like tyrosine phosphatase (e.g., that remove phosphate groups from tyrosines of intracellular proteins), receptor serine/threonine kinase, receptor guanylyl cyclase, or histidine-kinase-associated receptor.

48. The molecule of embodiment 8, wherein the molecule of interest comprises a chimeric antigen receptor (CAR), receptor tyrosine kinase (e.g., EGFR), a molecule to which a checkpoint inhibitor can bind (e.g., PD-L1), or lineage-specific marker (e.g., CD20).

49. The molecule of embodiment 8, wherein the molecule of interest comprises a CAR, EGFR, CD20 or PD-L1.

50. The molecule of embodiment 9, wherein the first moiety comprises an amino acid sequence to which a receptor can bind.

51. The molecule of embodiment 50, wherein the first moiety comprises a ligand for the receptor.

52. The molecule of embodiment 51, wherein the ligand comprises an ectodomain of CD19 and the receptor comprises a CAR specific for CD19.

53. The molecule of embodiment 50, wherein the receptor comprises a chimeric antigen receptor (CAR), T-cell receptor (TCR) or B-cell receptor (BCR).

54. The molecule of embodiment 9, wherein the first moiety comprises an scFv, Fab, single-domain antibody, nanobody, monobody, DARPin or affibody.

55. The molecule of embodiment 8 or 9, additionally comprising a peptide linker that can be cleaved by a protease.

56. The molecule of embodiment 55, wherein the protease comprises an endosomal/lysosomal protease.

57. The molecule of embodiment 56, wherein the protease comprises cathepsin.

58. The molecule of embodiment 55, wherein the peptide linker is located between the first moiety and the second moiety on a polypeptide that comprises the first moiety and the second moiety.

59. The molecule of embodiment 58, wherein the peptide linker is closer to the first moiety than to the second moiety.

60. The molecule of embodiment 55, wherein the peptide linker comprises Gly-Phe-Leu-Gly (GFLG).

61. The molecule of embodiment 55, wherein the peptide linker comprises Valine-Arginine (VR) and/or Phenylalanine-Ly sine (FK).

62. The molecule of embodiment 55, wherein the peptide linker comprises a GS, GFLG, 3×GFLG, GFLG-VA, GFLG-VK, GFLG-VR, GFLG-GFLG, FK, VA, EVR, VK linker or combinations thereof (FIG. 35).

63. The molecule of embodiment 55, wherein the second moiety comprises an scFv, Fab, single-domain antibody, nanobody, monobody, DARPin or affibody.

64. The molecule of embodiment 63, wherein the scFV comprises H7.

65. The molecule of embodiment 55, wherein cleavage of the peptide linker by the protease can provide for trapping and/or degradation of the molecule of interest inside a cell into which the molecule with at least two moieties is internalized.

66. The molecule of embodiment 8 or 9, comprising an amino acid sequence SEQ ID NO: 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, or 79-117 or an amino acid sequence at least 85, 86, 87, 88, 89, 90, 91, 92, 93, 94, 95, 96, 97, 98 or 99 percent identical thereto.

67. The molecule of embodiment 8 or 9, wherein a nucleotide sequence encoding the molecule comprises SEQ ID NO: 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, or 77 or a nucleotide sequence at least 85, 86, 87, 88, 89, 90, 91, 92, 93, 94, 95, 96, 97, 98 or 99 percent identical thereto.

68. A bispecific modulator, comprising:
a. a first antibody or antibody fragment that binds to a transferrin receptor (TfR) on a cell surface; and
b. a second antibody or antibody fragment that binds to a transmembrane protein on the cell surface that is not TfR.

69. A bispecific modulator, comprising:
a. a transferrin protein or part of a transferrin protein that binds to a TfR on a cell surface; and
b. an antibody or antibody fragment that binds to a transmembrane protein on the cell surface that is not TfR.

70. The bispecific modulator of embodiment 68 or 69, wherein the first antibody or antibody fragment and second antibody or antibody fragment (embodiment 68), or transferrin/part of transferrin and the antibody or antibody fragment (embodiment 69), are part of one polypeptide.

71. The bispecific modulator of embodiment 68 or 69, wherein the first antibody or antibody fragment and second antibody or antibody fragment, or transferrin/part of transferrin and the antibody or antibody fragment, are more than one polypeptide.

72. The bispecific modulator of embodiment 71, wherein the more than one polypeptide comprises two polypeptide chains connected by a dimerizing domain.

73. The bispecific modulator of embodiment 72, wherein the dimerizing domain comprises a knob-in-hole Fc.

74. The bispecific modulator of embodiment 68 or 69, additionally comprising a peptide linker that can be cleaved by an endosomal/lysosomal protease.

75. A nucleic acid(s) encoding the molecule of any one of embodiments 8-67, or bispecific modulator of any one of embodiments 68-74.

76. A vector comprising the nucleic acid of embodiment 75.

77. A cell comprising the vector of embodiment 76.

78. A method for treating a toxicity associated with CAR-T therapy, or for increasing the efficacy of immune checkpoint and targeted cancer therapy, comprising administering the molecule of any one of embodiments 8-67, or bispecific modulator of any one of embodiments 68-74 to a subject.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—Constructing TransTACs (Bispecific Modulators) for Downregulating EGFR

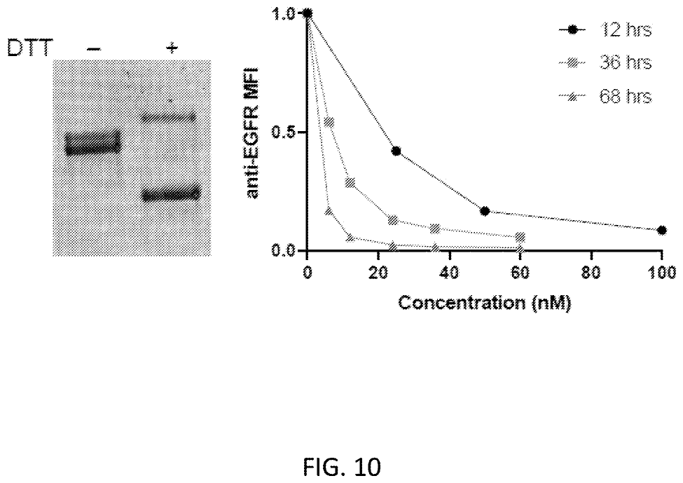

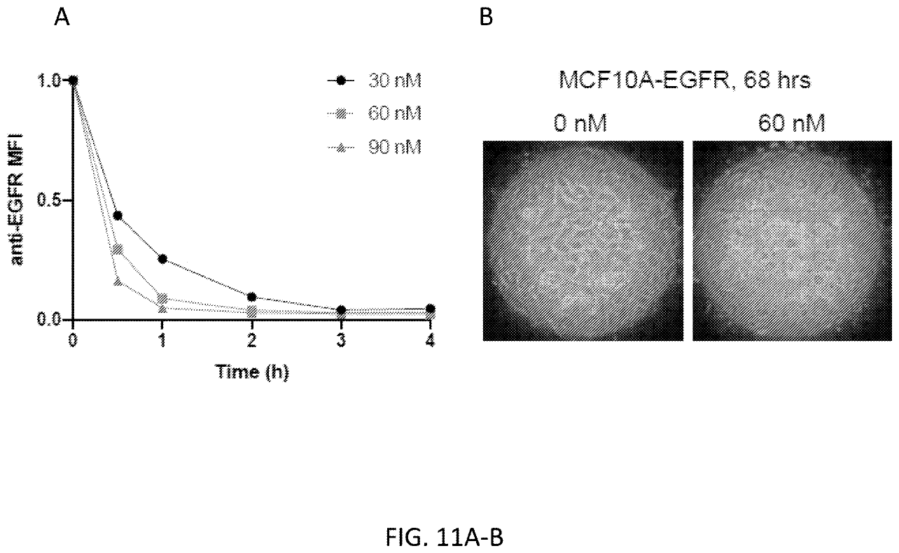

An anti-EGFR affibody-Fc-Tf TransTAC was expressed and validated by SDS-PAGE (FIG. 10, Left). Zero to 60 nM of TransTAC was incubated with a MCF10A EGFR overexpression cell line for 12, 36 or 68 hrs. A drastic dose-dependent decrease of EGFR level was observed by flow cytometry with an IC₅₀<6 nM at 68 hrs (FIG. 10, Right). We observed the same dose-dependent decrease of EGFR with A549, an adenocarcinomic lung cancer cell line (FIG. 11A). A time course experiment was then performed with 30, 60, or 90 nM of TransTAC on A549 cells, which showed EGFR was effectively downregulated with a half-life of <30 min at 90 nM TransTAC. Additionally, we observed TransTAC-mediated killing of the MCF10A-EGFR cells (FIG. 11B). These data demonstrate the effectiveness of the construct.

Notably, TransTAC downregulated more than 99% of cell surface EGFR at equilibrium, while recently reported LYTACs only reached 70-80% downregulation. In addition, the kinetics are also different. TransTAC-driven EGFR internalization has a half-life of <30 min while LYTACs' are around 10-20 hrs. These distinctions are due to the different endocytosis kinetics of the carrier proteins used in the studies.

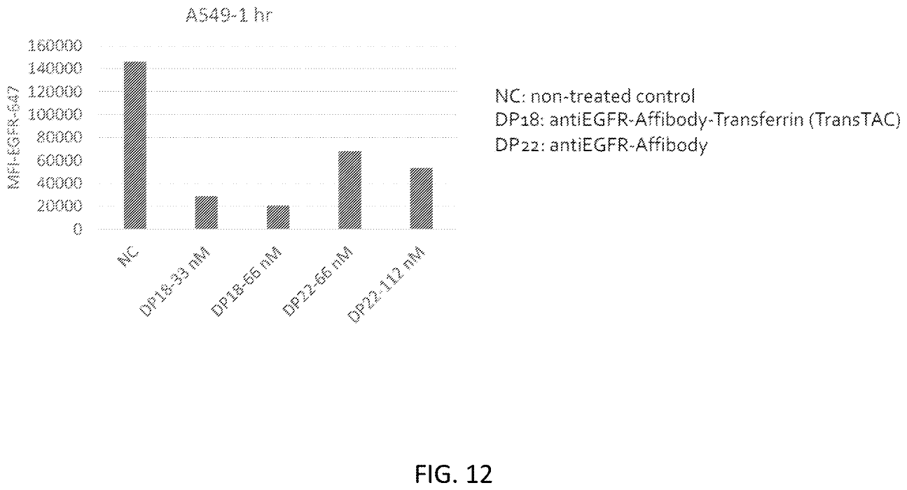

Additional data (FIG. 12) show that using A549 cells that express EGF receptor (EGFR) on the cell surface, TransTAC targeting EGFR internalized the receptor to a greater degree than an EGFR-specific antibody alone.

### Example 2—Constructing TransTACs (Bispecific Modulators) for Downregulating Anti-CD19 CAR

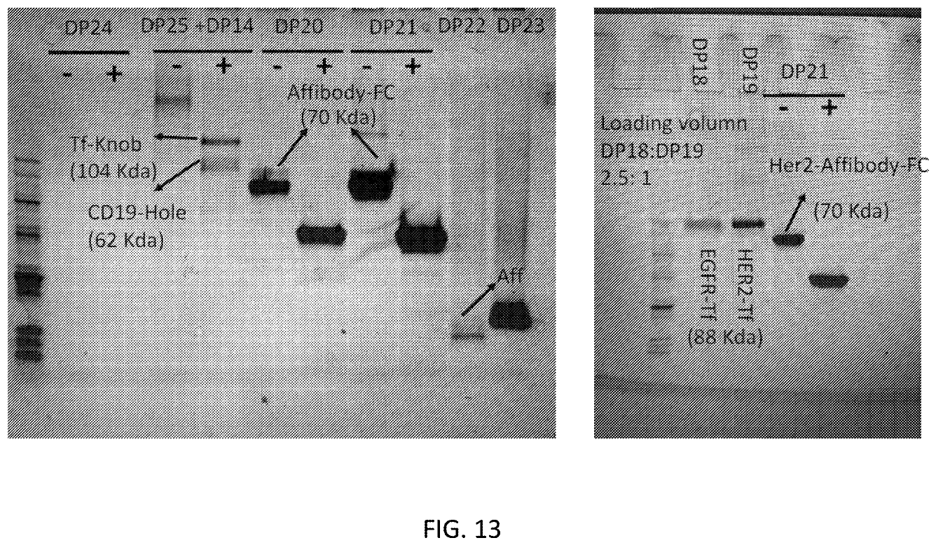

Data show that transferrin fusion proteins with antibodies specific for CD19, EGFR and HER2 express well in expi293 cells (FIG. 13).

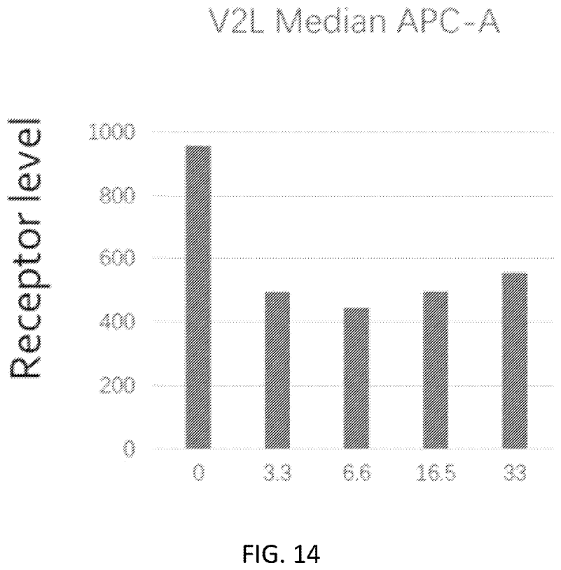

FIG. 14 shows data indicating that a knob-in-hole version of TransTAC targeting of an anti-CD19 chimeric antigen receptor internalized the receptor.

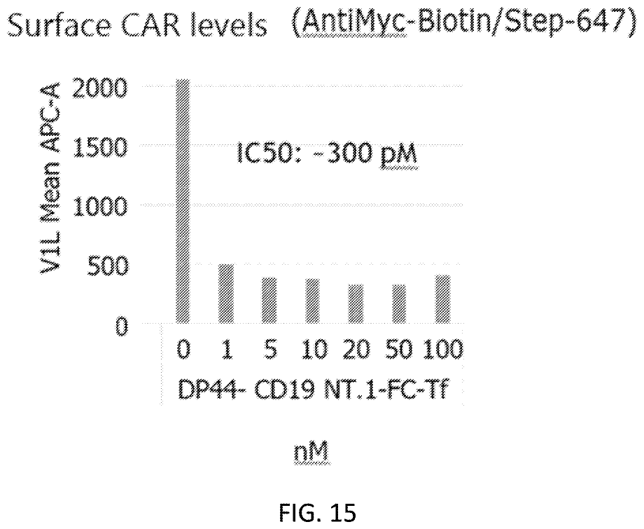

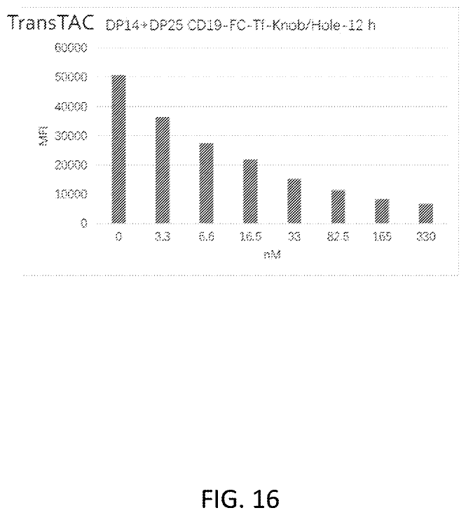

In FIG. 15 are shown data measuring surface CAR levels in cells using anti-myc-biotin/Streptavidin 647, similar to the study shown in FIG. 14. The data in FIG. 15 show that a homodimeric TransTAC targeting of the CAR effectively internalized the CAR. In studies as shown in FIG. 16, cells expressing an anti-CD19 CAR were incubated with K562 cells (expressing CD19) with a TransTAC bispecific modulator. Cell activation was measured. The data showed that the TransTAC bispecific modulator began to show inhibitory activity at concentrations below 10 nm. These data indicate that the TransTAC molecules effectively internalized CAR and downregulated CAR-T cell activity.

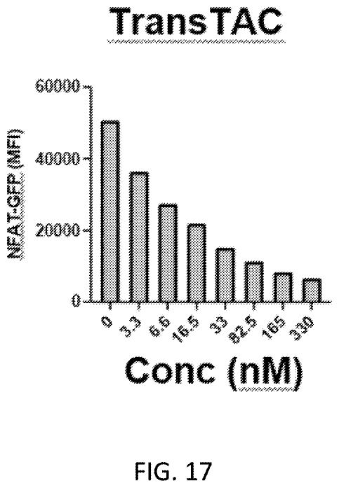

The data in FIG. 17 are similar to those shown in FIG. 16. The data in FIG. 12 show that TransTAC improved the IC₅₀ for inhibiting cell activation to about 10-20 nM.

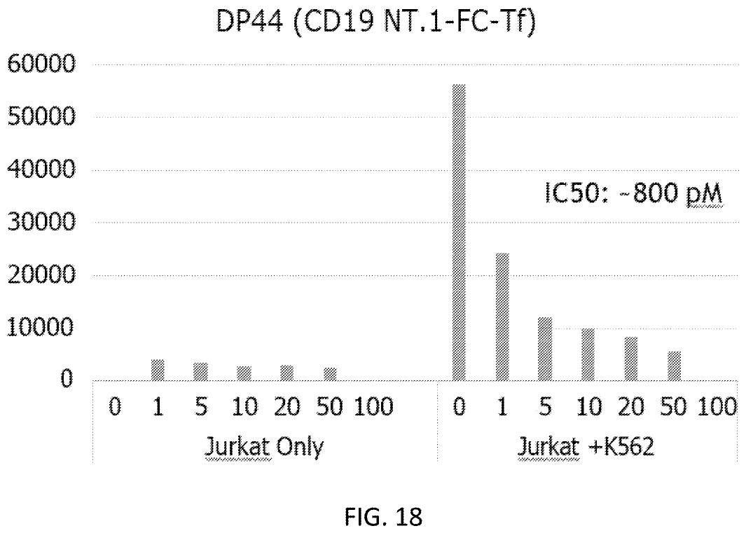

The data in FIG. 18 show that a TransTAC molecule with a CD19NT.1 variant ectodomain blocks CAR-T activation with an IC₅₀ of about 800 μM. The data show that, in absence of the K562 cells which express CD19, the TransTAC molecule has no effect on the Jurkat cells.

### Example 3—TransTAC Molecules Containing Protease Sites to Increase Target Degradation

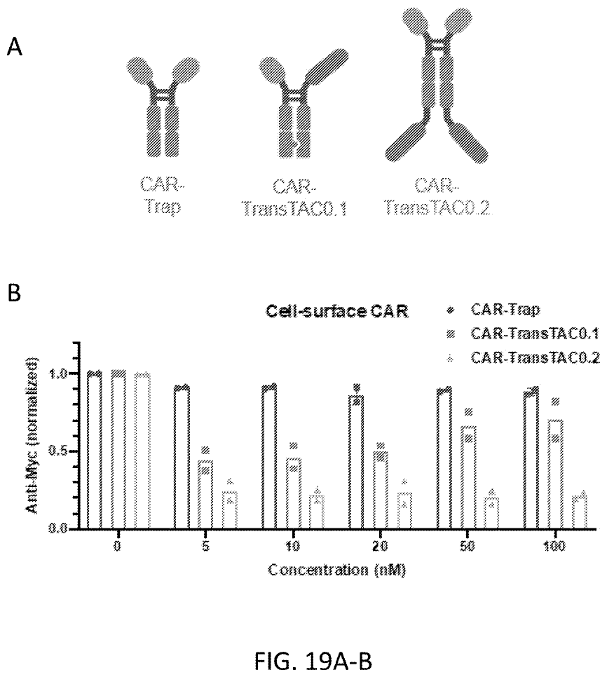

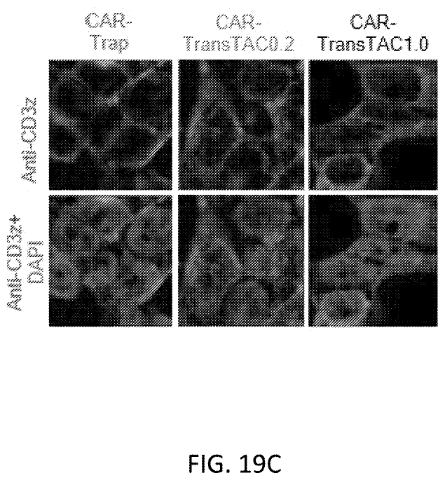

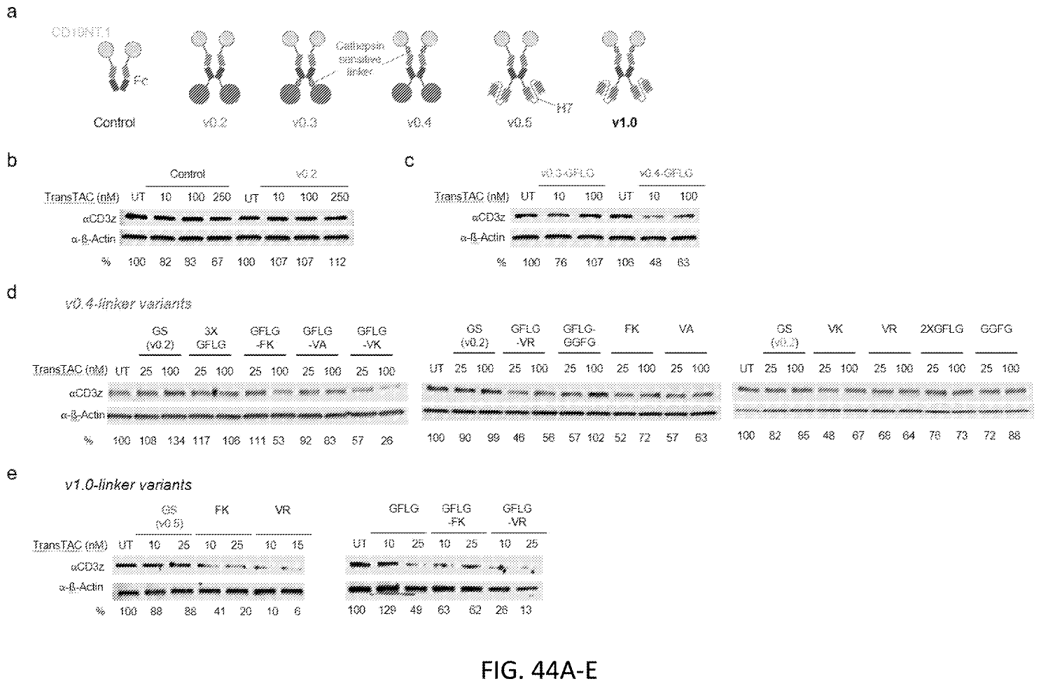

FIG. 19A-B is a schematic of molecules used in these studies (A) and results obtained with the molecules (B). TransTAC1.0 for the figures in this Example is TransTAC0.4 as shown in FIG. 44A.

FIG. 19C illustrates fluorescence microscopy results of the targeted CAR on the cells in FIG. 19B.

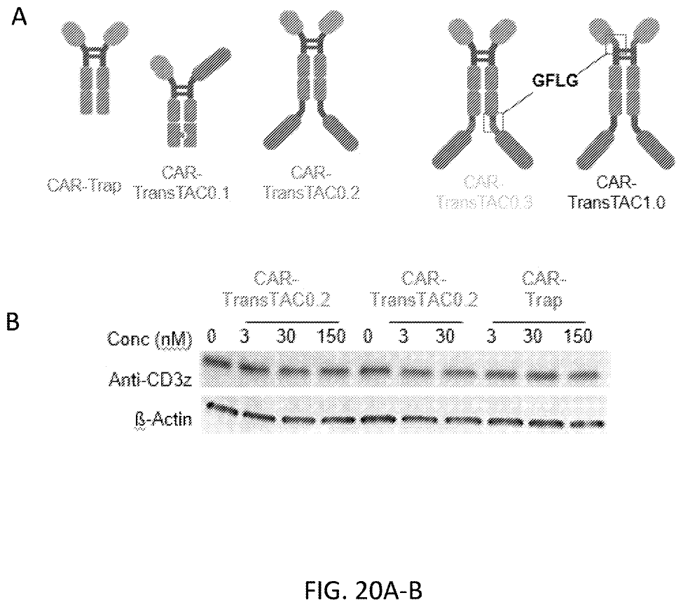

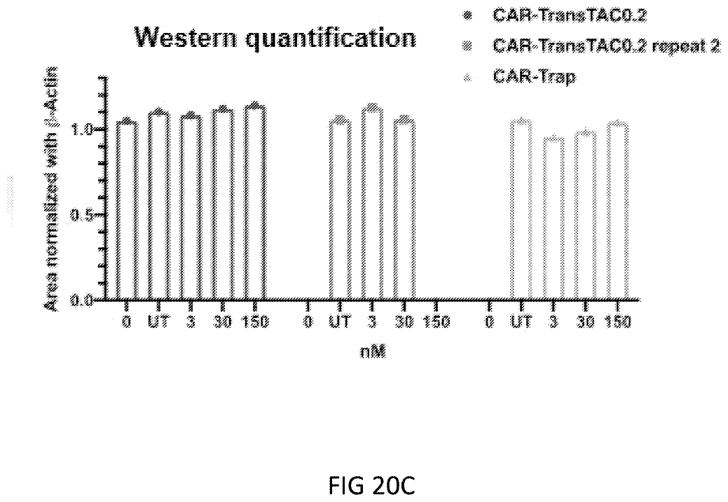

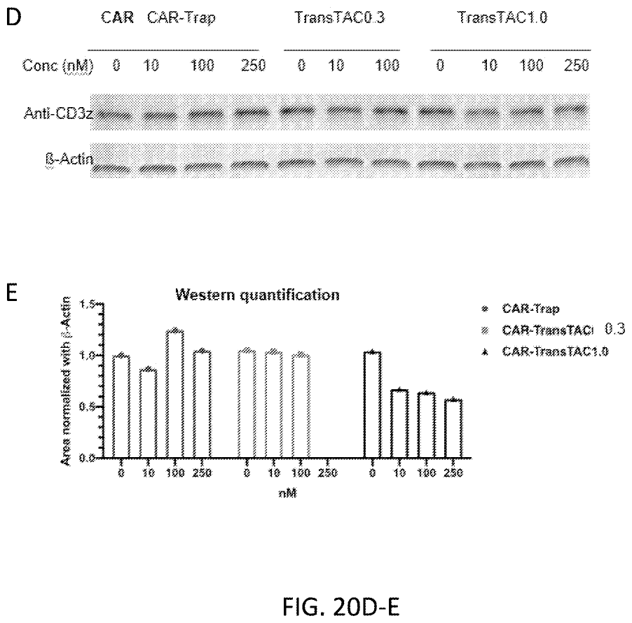

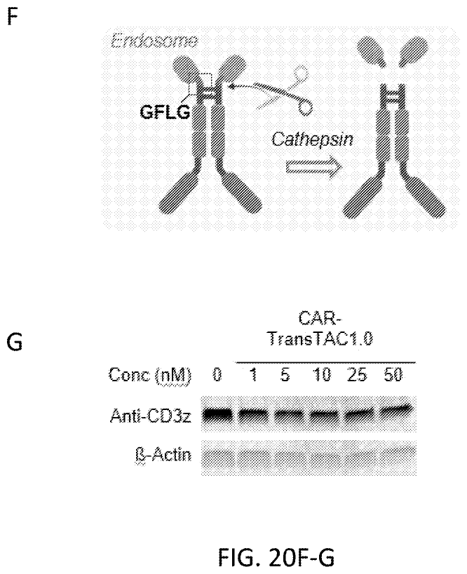

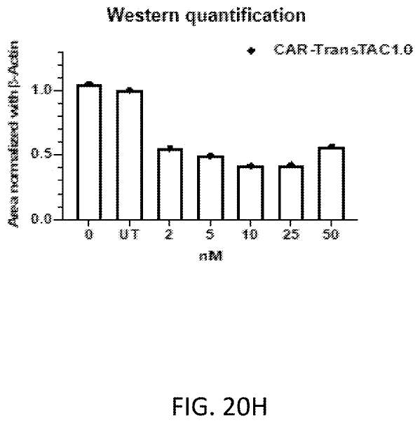

FIG. 20A-B is a schematic of molecules used in these studies (A) and Western blot results of the targeted CAR and actin control (B).

FIG. 20C shows a graph of the data from FIG. 20B.

FIG. 20D-E shows Western blot results of of additional molecules used in these studies (D) and a graph of the data (E).

FIG. 20F-G shows a TransTAC molecule that contains GFLG linkers (F) and Western blot data using the molecule (G).

FIG. 20H shows a graph of the data from FIG. 20G.

FIG. 20I shows results from other cathepsin-sensitive TransTAC molecules.

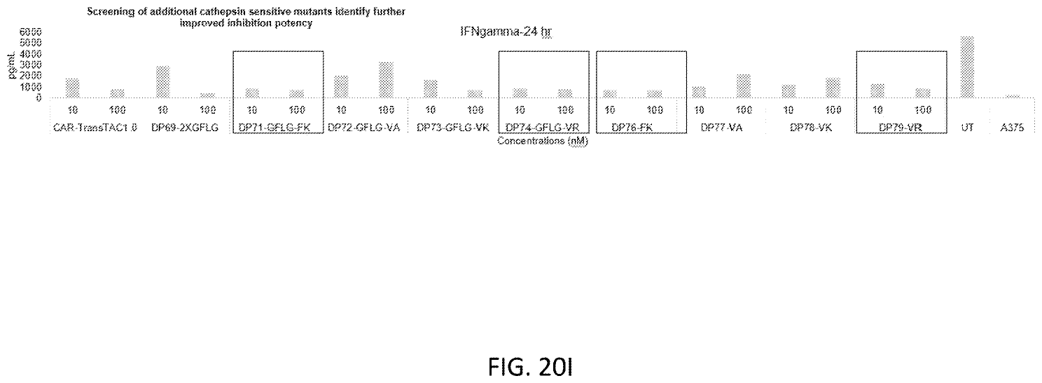

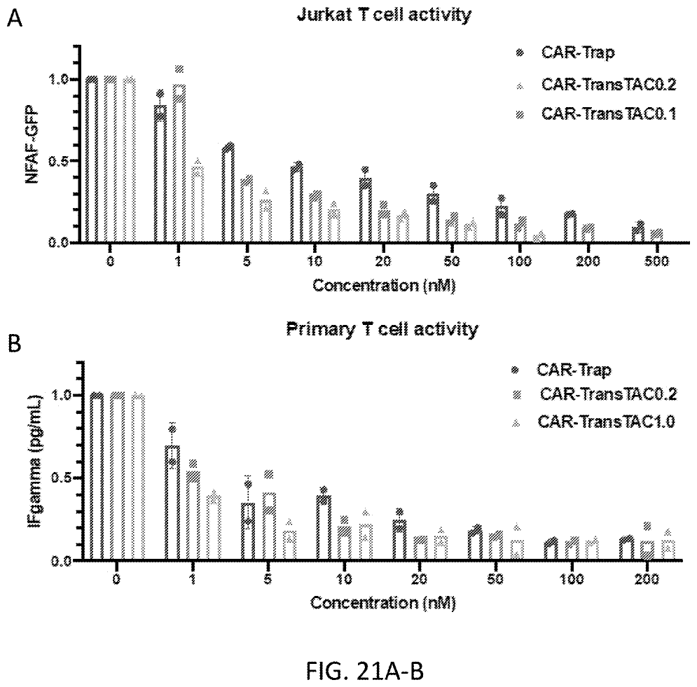

FIG. 21A-B shows results indicating that the molecules shown in FIG. 20A inhibited activation in Jukat cells (A) and inhibited interferon gamma (IFN-γ) release from primary T cells (B) in an experiment in which cells expressing an anti-CD19 CAR receptor were incubated with CD19-positive A375 cells.

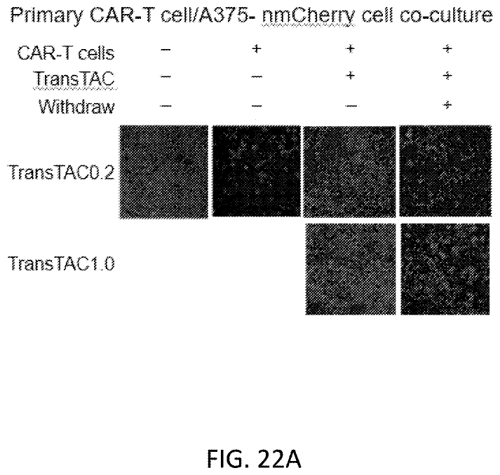

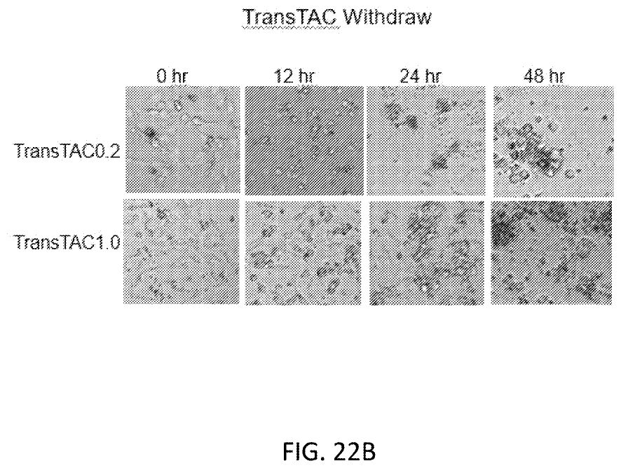

FIG. 22A shows results indicating that adding the indicated molecules shown in FIG. 20A stopped human primary anti-CD19 CAR-T cells from killing CD19-positive A375 target cells. The A375 cells express a nucleus mCherry for fluorescence microscopy. The results show that removing the molecules led to reactivation of the CAR-T cells and killing of the CD19-positive A375 target cells. The photographs show the red fluorescence channel of the fluorescence microscope.

FIG. 22B shows results indicating that removal of the indicated molecules let to reactivation of the CAR-T cells and killing of the CD19-positive A375 target cells. The photographs show an overlay of the red fluorescence channel and the white-field channel.

### Example 4—TransTAC Molecules Targeting Epidermal Growth Factor Receptor (EGFR)

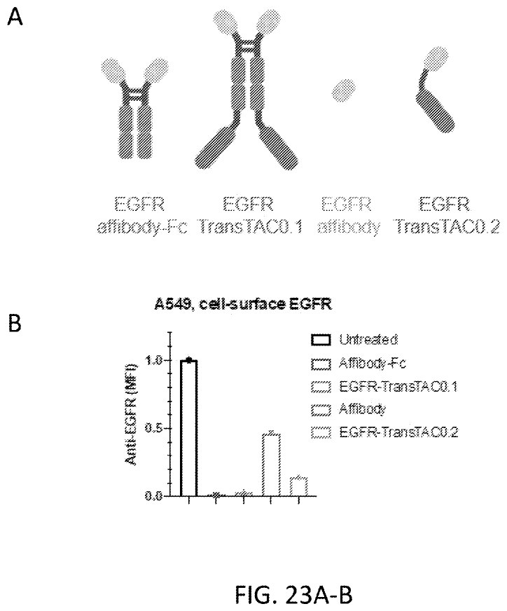

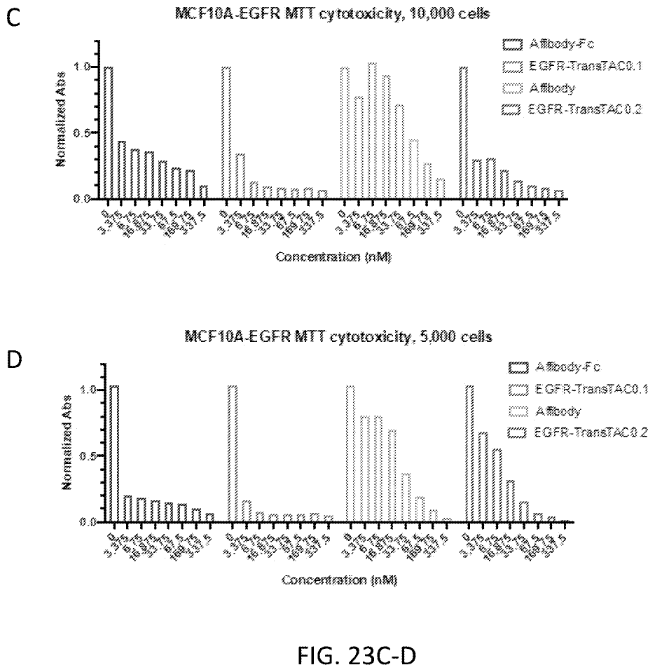

FIG. 23A-B shows a schematic diagram of molecules used in these studies (A). The molecules contain antibodies, affibodies and TransTAC molecules specific for EGFR. Also shown are results showing reduction of EGFR levels on the surface of A549 cells using the molecules (B).

FIG. 23C-D show results on inhibition of cell proliferation using the molecules of FIG. 53A.

### Example 5—TransTAC Molecules Targeting CD20

These data show an example approach using a TransTAC molecule having an antibody specific for CD20 and a molecule that binds to the transferrin receptor (transferrin or antibody).

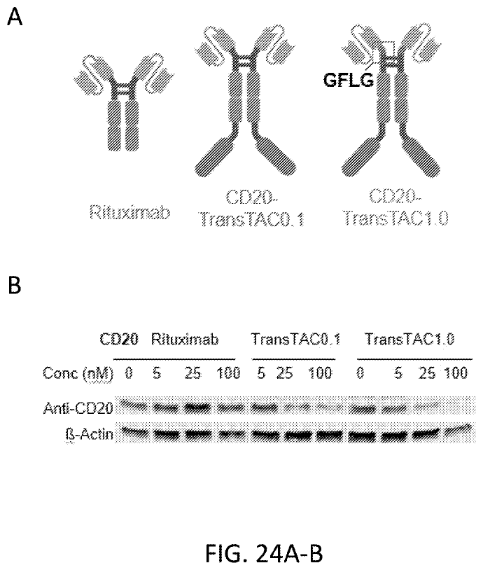

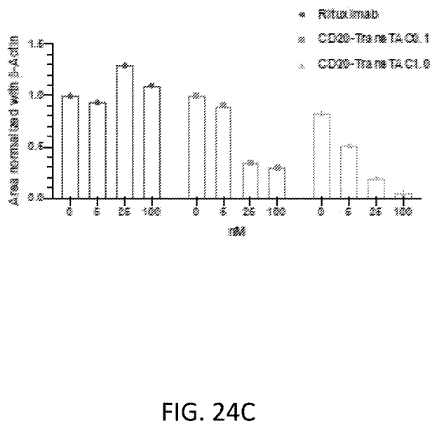

FIG. 24B-C shows a schematic diagram of molecules used in these studies (B) and results obtained from their use (C).

FIG. 24D shows a graph of the normalized data from FIG. 24C.

The data show that the TransTAC molecules are more rapidly internalized/degraded than the anti-CD20 Rituximab antibody, that targets CD20 alone.

### Example 6—Reversible Control of Receptor Functions

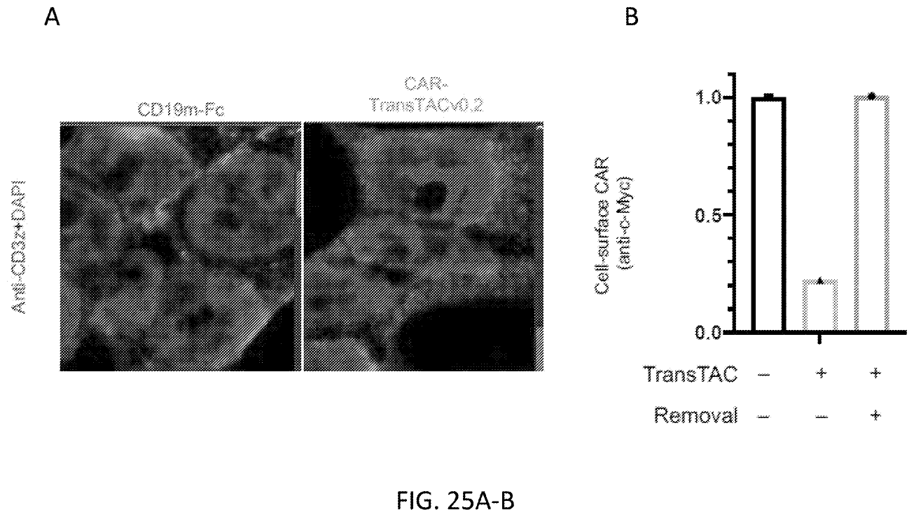

FIG. 25A shows cells cells expressing a CAR. The cells in the left panel have been contacted with an CD19m-Fc antibody (the CD19NT.1 variant). The cells in the right panel have been contacted with a TransTAC molecule that binds to the CAR. Nuclei of the cells in both panels have been stained with DAPI. The cells have also been stained with an anti-CD3z antibody that stains the CAR. Immunofluoresce from the anti-CD3z antibody is localized to the surface of cells in the left panel. Anti-CD3z antibody fluorescence is localized to the cell cytoplasm in cells of the right panel. The data show that the TransTAC molecule caused internalization of the CAR.

FIG. 25B shows reversibility of CAR internalization by TransTAC. In the first bar in the graph, cell-surface CAR-specific immunofluorescence for cells that have not been contacted by TransTAC is relatively high (about 1.0). In the second bar of the graph, the cells have been contacted by TransTAC and cell-surface CAR-specific immunofluorescence is low (about 0.2). In the third bar of the graph, the cells have been contacted by TransTAC, but TransTAC then was removed. After 24 hours, cell-surface CAR-specific immunofluorescence has increased to similar levels as cells not exposed to TransTAC (about 1.0).

These data show that TransTAC-enabled internalization of a CAR receptor serves as a reversible CAR-T cell off-switch. This can be used to moderate any toxicities associated with CAR-T therapy.

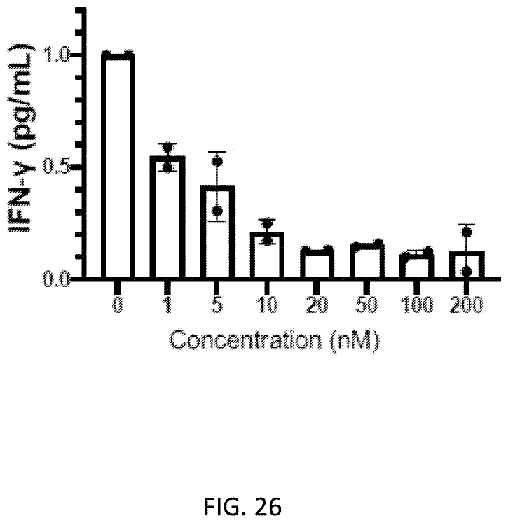

FIG. 26 shows that TransTAC inhibited/stopped interferon gamma production.

As discussed in Example 8, FIG. 22A demonstrates that TransTAC stopped primary human anti-CD19 CAR-T cells from killing CD19-positive A375 target cells. The data show that removing the TransTAC led to reactivation of the CAR-T cells and killing of the CD19-positive A375 target cells. FIG. 22B shows that removal of the TransTAC molecules led to reactivation of CAR-T cells and killing of the targets.

### Example 7—Targeted Membrane Protein Degradation

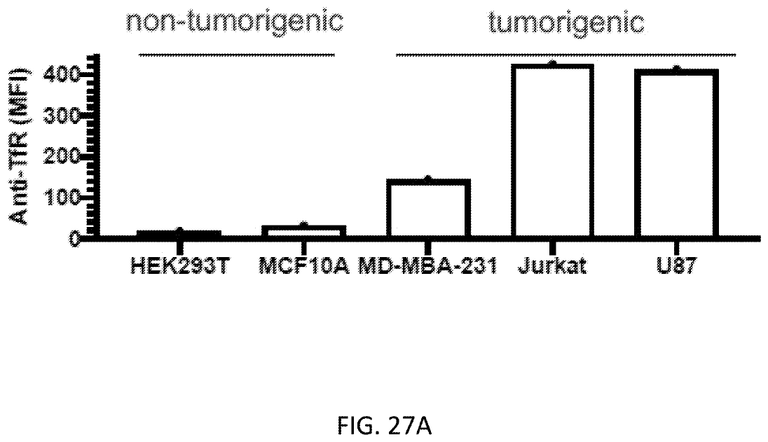

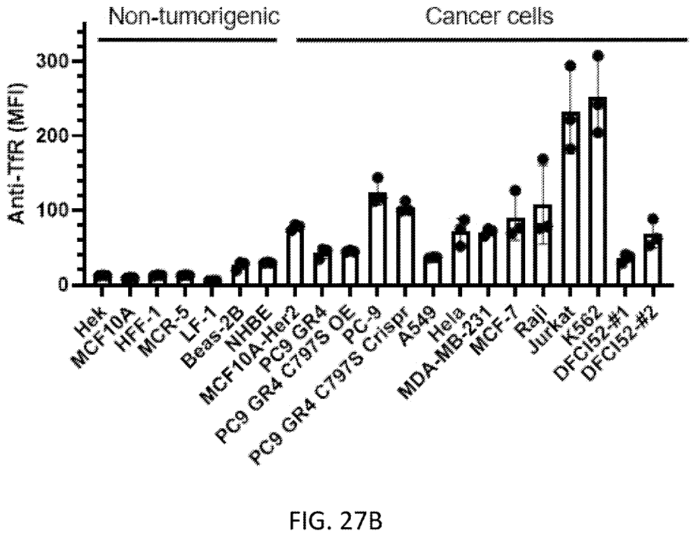

FIG. 27A-B show expression of the transferrin receptor on various cells, as indicated by fluorescent antibodies. The data indicate that transferrin receptor can be expressed at higher levels on the surface of tumor cells as compared to non-tumor cells.

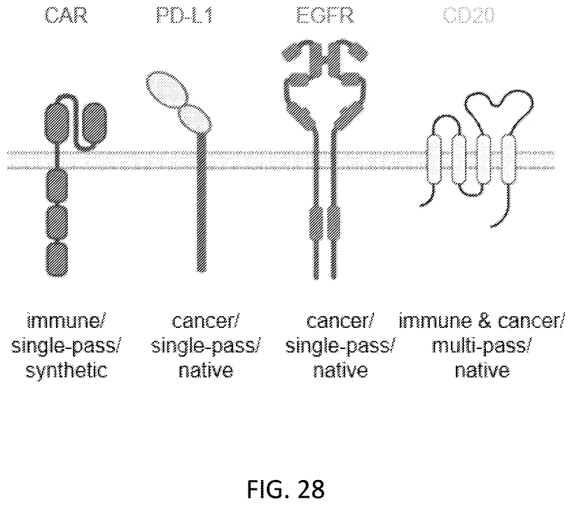

FIG. 28 shows examples of cell-surface proteins that can be targeted by TransTAC.

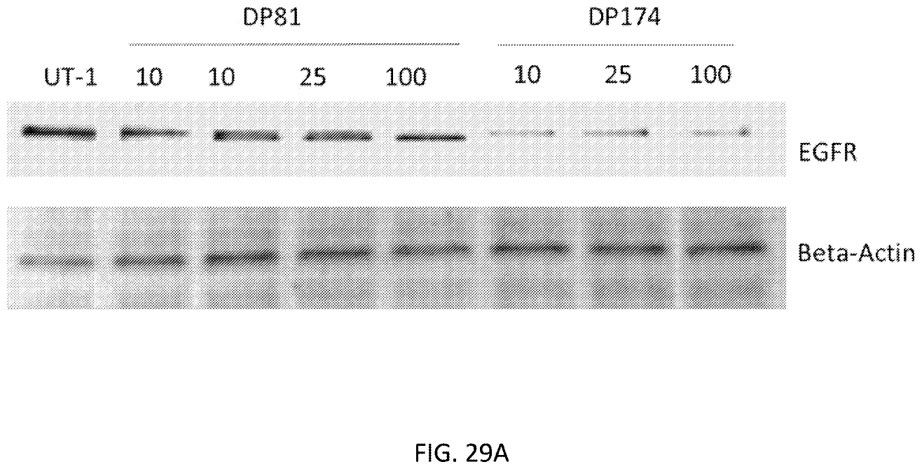

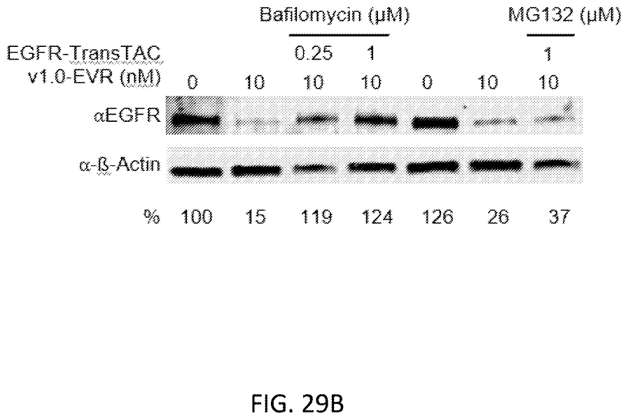

FIG. 29A shows that TransTAC molecules (DP81 and DP174) decrease the amount of EGFR in the cells.

FIG. 29B shows that a TransTAC molecule can degrade EGFR. The data show that TransTAC-mediated degradation of EGFR was sensitive to bafilomycin (inhibitor of autophagosome-lysosome fusion) and to MG132 (proteosome inhibitor). These data show that TransTAC-induced EGFR degradation was mediated by the lysosomal pathway.

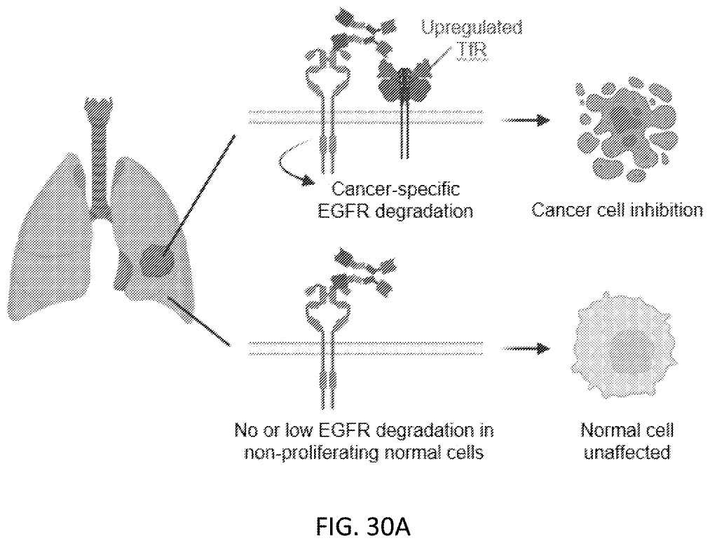

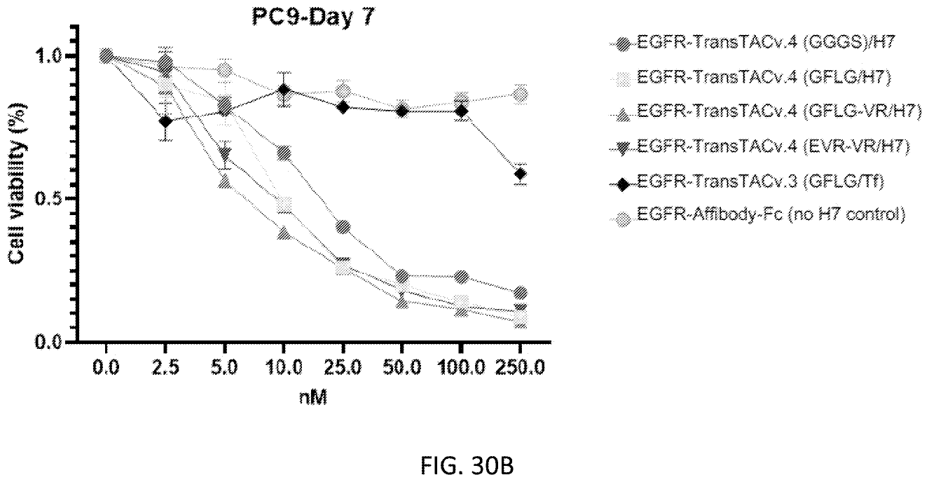

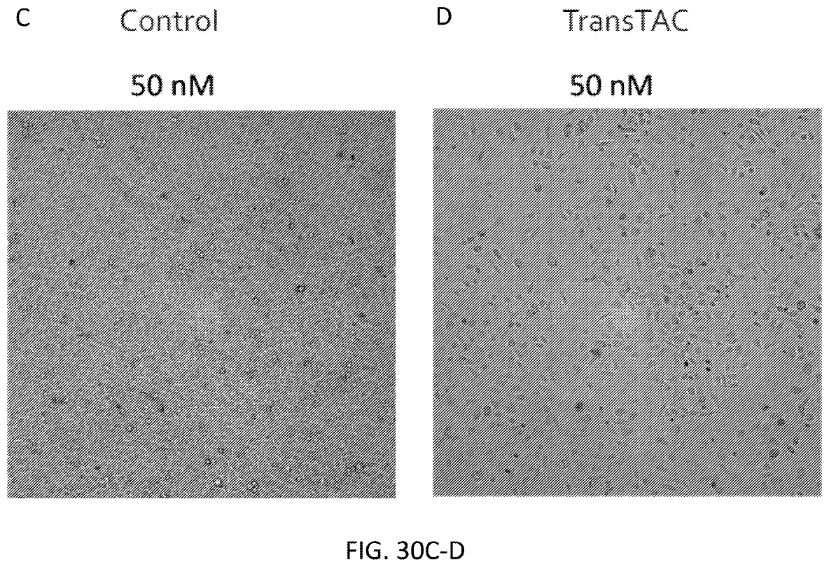

FIG. 30A shows an approach to treating lung cancer using TransTAC. The high expression of TfR in cancer cells enables targeting specificity.

FIG. 30B shows that an EGFR TransTAC molecule can inhibit PC9 cancer cells (lung adenocarcinoma).

FIG. 30C-D show that an EGFR TransTAC molecule could inhibit PC9 cancer cells.

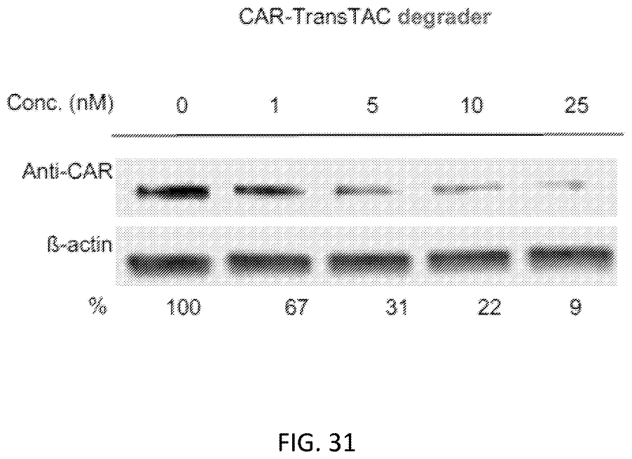

FIG. 31 shows that an anti-CAR TransTAC molecule can decrease the amount of CAR in these cells.

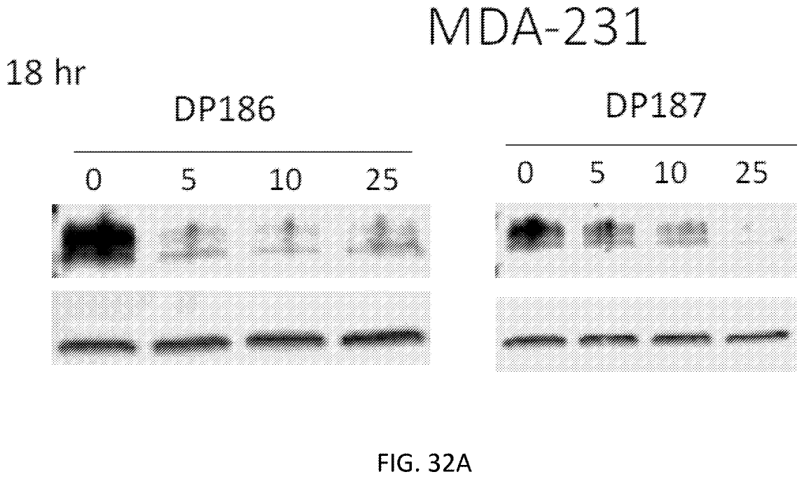

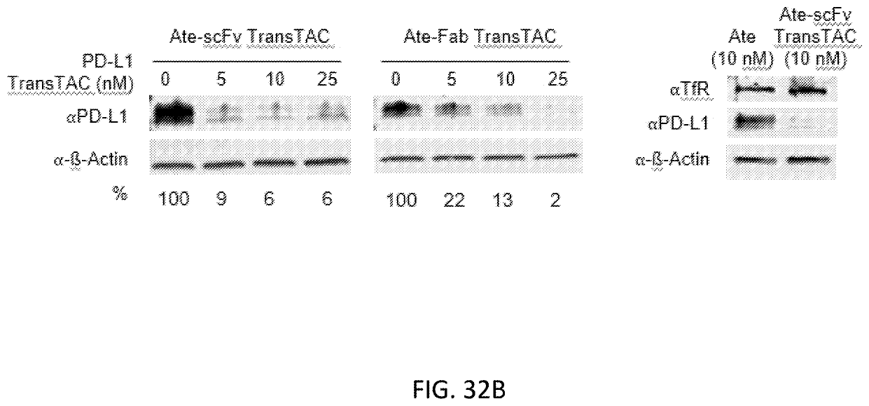

FIG. 32A shows that anti-PD-L1 TransTAC molecules (DP186, DP187) can decrease the amount of PD-L1 in these cells.

FIG. 32B illustrates data demonstrating that anti-PD-L1 TransTAC molecules can decrease the amount of PD-L1 in cells.

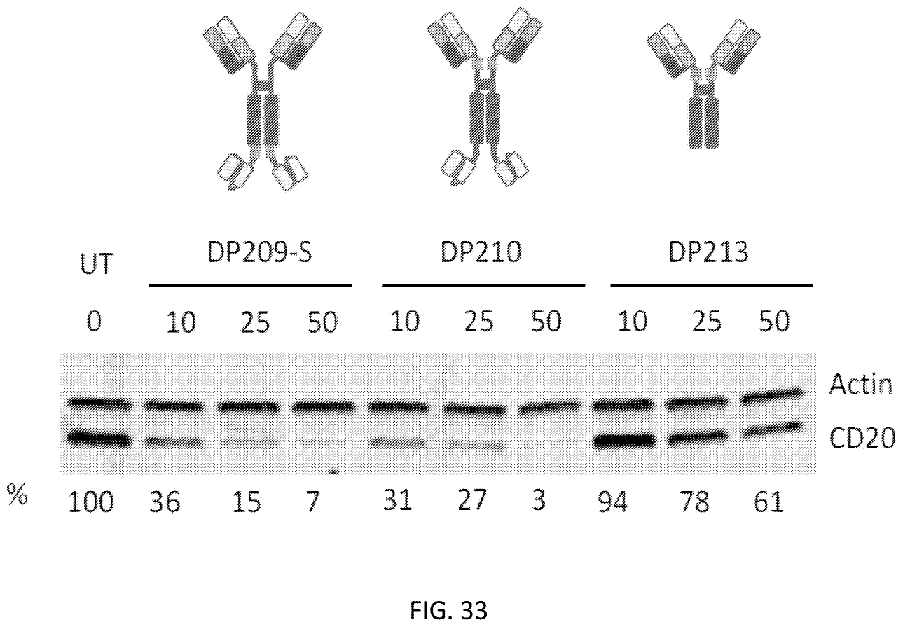

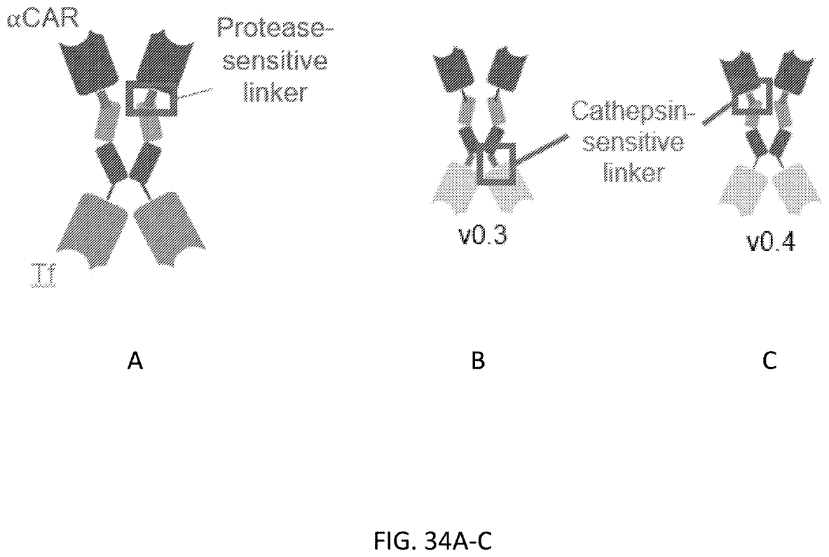

FIG. 33 shows that anti-CD20 TransTAC molecules (DP209S, DP210, DP213) can decrease the amount of CD20 in cells.

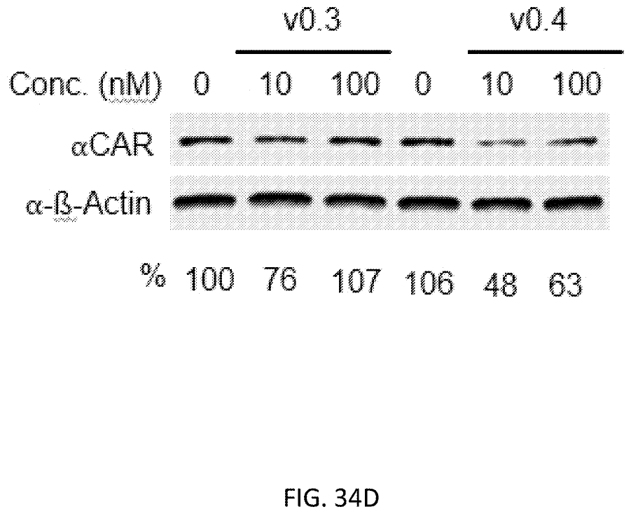

FIGS. 34A-C and 34D show example TransTAC molecules that include protease-sensitive linkers and example data obtained with the molecules.

FIG. 35 shows example data obtained with TransTAC molecules containing various protease-sensitive linkers.

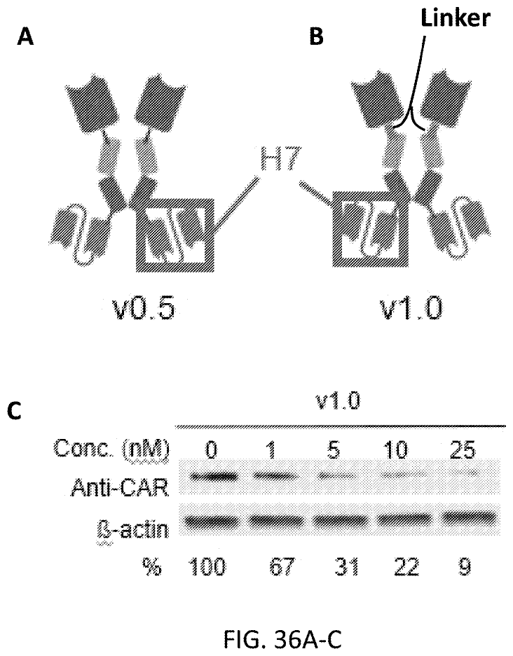

FIG. 36A-C show example TransTAC molecules that include an antibody fragment specific for transferrin binding and example data obtained with the molecules.

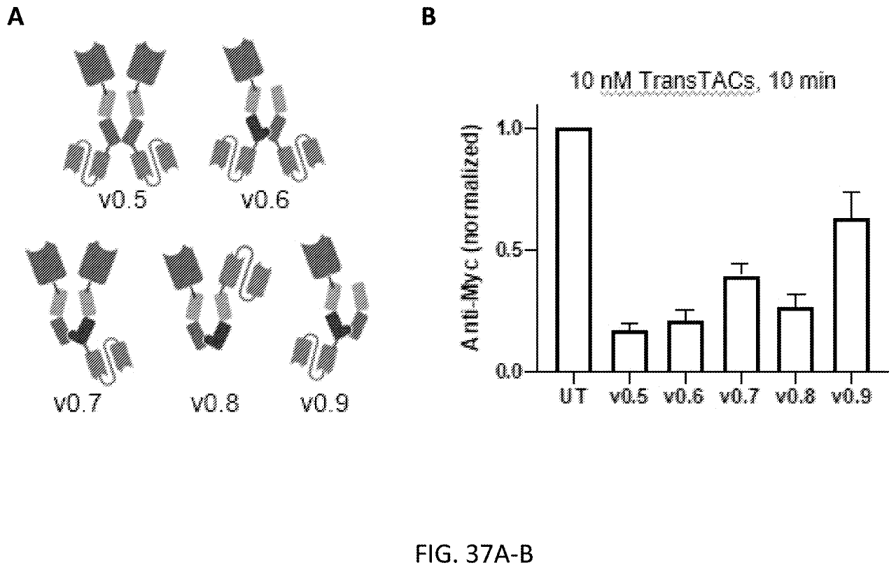

FIG. 37A-B show examples of TransTAC molecules and example data obtained with the molecules.

### Example 8—TransTAC for Cancer

Targeted therapy with tyrosine kinase inhibitors is a standard treatment for lung cancer with EGFR mutations. We reasoned that co-targeting an EGFR and a TfR receptor on lung cancer cells could lead to EGFR inhibition while maintaining high tumor specificity (FIG. 30A). We generated an anti-EGFR affibody*H7*GFLG-VR TransTAC. Incubation of the molecule with a human lung adenocarcinoma A549 cell line led to >90% EGFR degradation (FIG. 30B). Notably, treatment of a non-tumorigenic HEK cell line engineered to overexpress EGFR with the TransTAC molecules resulted in much less EGFR degradation, highlighting the tumor-specificity of the technology. This specificity results from higher TfR expression in tumor cells (FIG. 27A-C).

### Example 9—Transferrin Receptor Upregulation in Cancer Cell Lines, Primary Tumors and Activated T Cells

Increased expression of TfR on cancer cells and some immune cells was illustrated in Example 7.

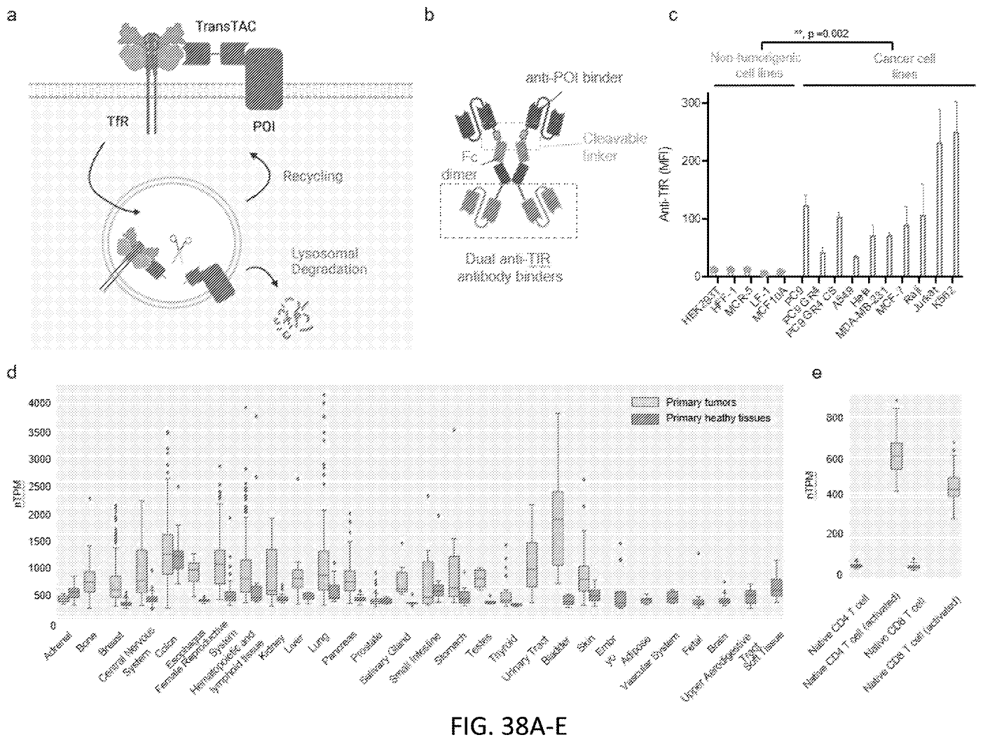

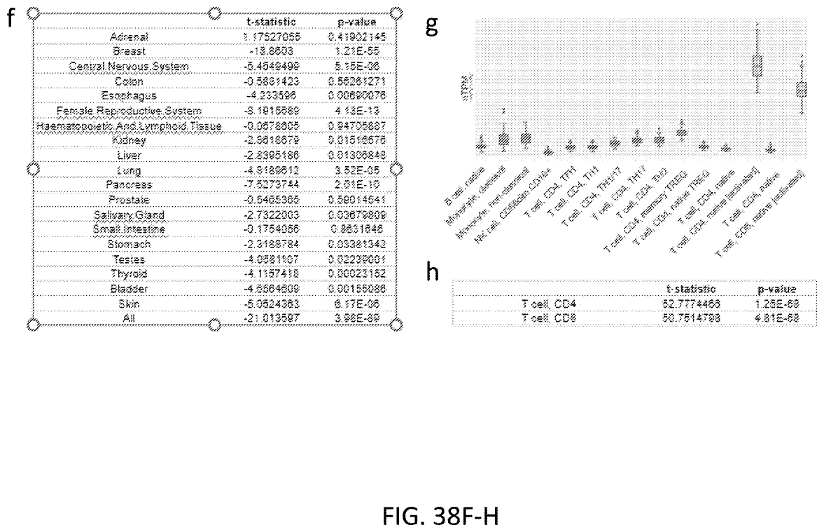

In additional studies, we measured cell-surface TfR levels using flow cytometry on five non-tumorigenic cell lines, including HEK293T (embryonic kidney), MCF10A (mammary epithelial), HFF-1 (foreskin fibroblast), MCR-5 (lung fibroblast), and LF-1 (fetal lung fibroblast), and 10 cancer cell lines, including Hela (cervical cancer); Raji (lymphoma); Jurkat and K562 (leukemias); MDA-MB-231 and MCF-7 (breast cancers); PC9 and A549 (lung cancers), and PC9 cells with EGFR drug-resistant mutants. We observed that cancer cell lines expressed 2-26 fold more TfR at the cell surface compared to non-tumorigenic cell lines (FIG. 38C). Among the cancer cell lines, leukemia cell lines Jurkat and K562 exhibited the highest TfR expression. Our findings demonstrate upregulation of TfR in cancer cell lines.

Since cell lines have been modified to be immortalized, there is a possibility of changes in protein expression. Therefore, we further showed TfR expression is upregulated in primary tumors compared to primary healthy tissues, by performing a transcriptomics analysis of TFRC, the gene for TfR1. Microarray transcriptomics data of TFRC in primary healthy tissues and tumors was obtained from the MERAV database. Paired tissue analysis for healthy vs. tumor samples was performed using a custom python script. TFRC expression is statistically significantly increased in cancers overall (p=3.98e−89) and in 14 out of 19 specific tissues, including breast, lung, pancreas, liver, bladder, skin, esophagus, thyroid, testes, stomach, salivary gland, kidney, central nervous system, and female reproductive system tumors (FIG. 38D). These findings provide further evidence to support TfR as a cancer-upregulated target.

To investigate whether TfR could also be a potential target for immune cell modulation, the DICE dataset was analyzed, which contains gene expression profiles of human immune cells isolated from blood samples of healthy donors. While most immune cells express low level of TfRs, an approximately 6-fold higher TfR expression in activated CD4 and CD8 T cells was observed compared to inactivated T cells, a level comparable to the TfR levels in some malignant tissues, indicating TfR can be a target for modulating activated T cells (FIG. 38E). These findings indicate that TfR is not only upregulated in tumors but also in activated T cells, highlighting its value as a cell-surface receptor for both cancer and immune modulation. Our transcriptomic analysis provides a detailed comparison of TfR expression in specific tissues, serving as a roadmap for future selection of disease indications for our technology and beyond.

### Example 10—Targeted Protein Endosomal Trapping with Early Versions of TransTAC Designs

Experiments on TransTAC molecules specific for CD19-specific CARs are also described in Example 2 but more detailed in this Example.

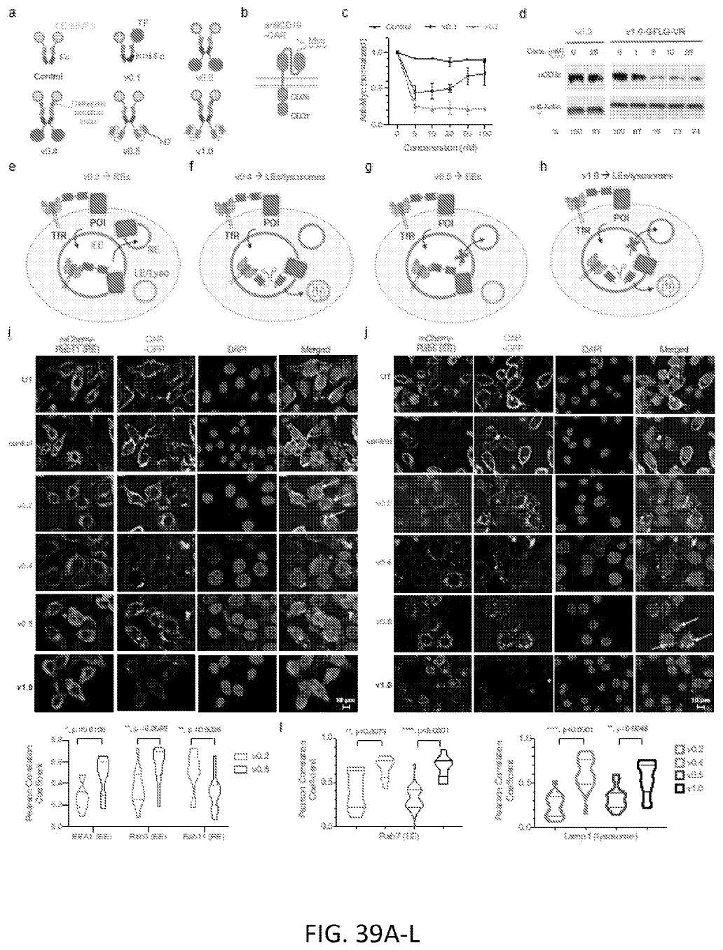

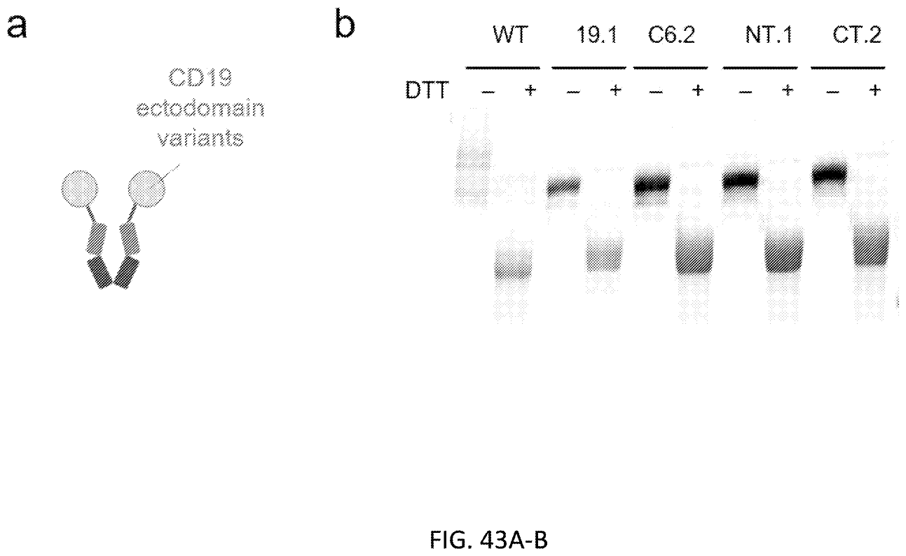

In these experiments, we used Jurkat cells expressing an N-terminal myc epitope tag CAR for measuring cell-surface CAR levels (FIG. 39B). Initially, we attempted to use the ectodomain of native CD19 as the CAR binding component but observed significant protein aggregation in SDS-PAGE gel (FIG. 43B, lane 1-2). We then evaluated a small panel of CD19 ectodomain variants that were developed earlier using yeast surface display (Klesmith, Justin R., et al. “Retargeting CD19 chimeric antigen receptor T cells via engineered CD19-fusion proteins.” Molecular pharmaceutics 16.8, 2019: 3544-3558) and showed they expressed and behaved well (FIG. 43A-B). Ultimately, we selected mutant CD19NT.1, which exhibited better expression, to be used in CAR-TransTAC.

We tested the concept that a molecule containing two TfR ligands would be more effective than one ligand in driving targeted CAR internalization, as TfR is a homodimeric receptor and requires binding of two transferrins (TF) to fully prime the TfR dimer for its physiological functions. Thus, we created two versions of TransTACs: v0.1, a knob-in-hole Fc construct with a single TF for binding TfR and one CD19NT.1 for CAR, and v0.2, a Fc fusion with two TFs and two CD19NT.1s (FIG. 39A). We also designed a control molecule lacking the TF ligand. These CAR-TransTACs were recombinantly expressed in 293expi cells, purified by protein A resin, and then incubated with myc-CAR-Jurkat cells. After 18-24 hours, we measured the cell surface CAR levels using an anti-myc antibody. We found that treatment with both v0.1 and v0.2 significantly decreased the cell-surface CAR levels, with v0.1 exhibiting a Dₘₐₓ of 60% and v0.2 exhibiting a Dₘₐₓ of 80% (FIG. 39C). Interestingly, a hook effect was observed with v0.1, but not v0.2. In contrast, treatment with the control CD19NT.1-Fc protein did not result in a decrease in cell surface CAR levels. These findings showed that CAR-TransTACs can effectively internalize CAR from the cell surface via a TF-dependent mechanism, and that in some embodiments a dimeric TransTAC is more effective than a monomer.

However, despite the ability to efficiently remove CAR from the cell surface, TransTACv0.2 did not result in CAR degradation, shown by whole-cell lysate western blots (FIG. 39D, 44A-B), indicating that the internalized receptor was trapped inside cells and was not degraded.

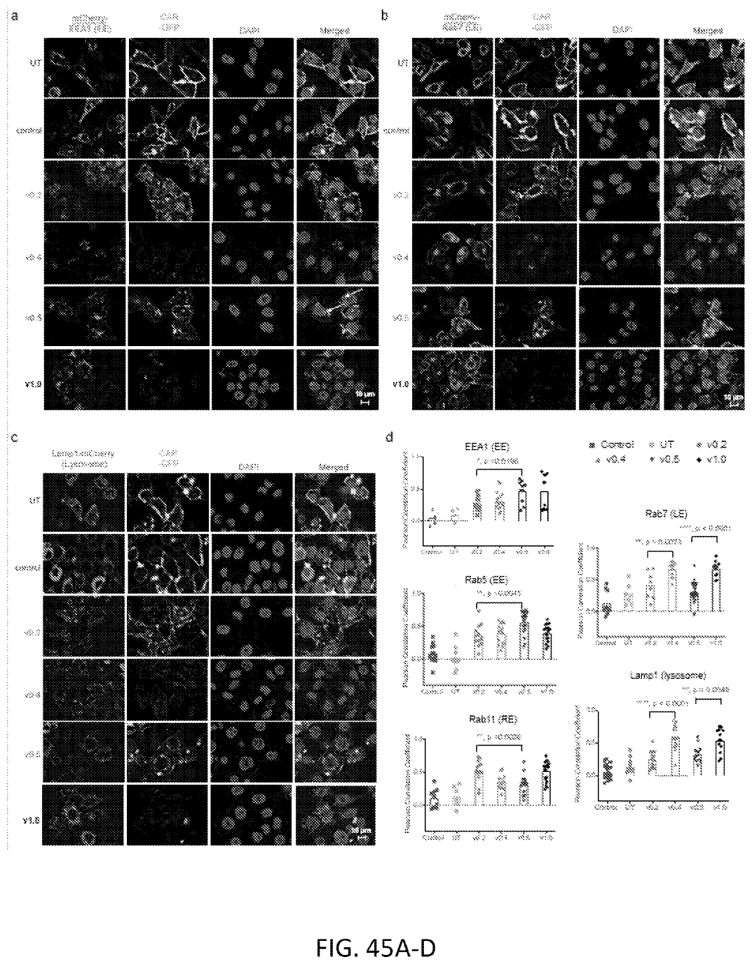

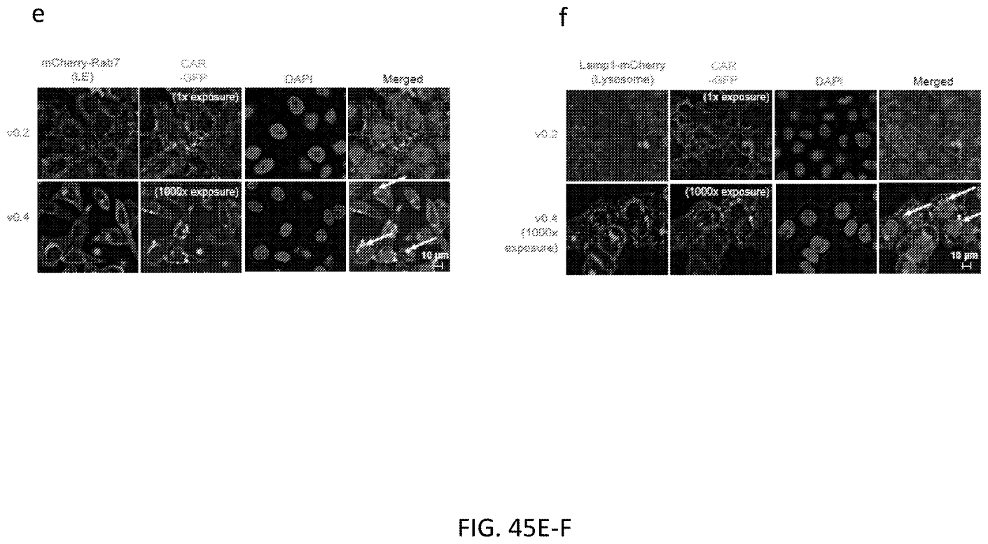

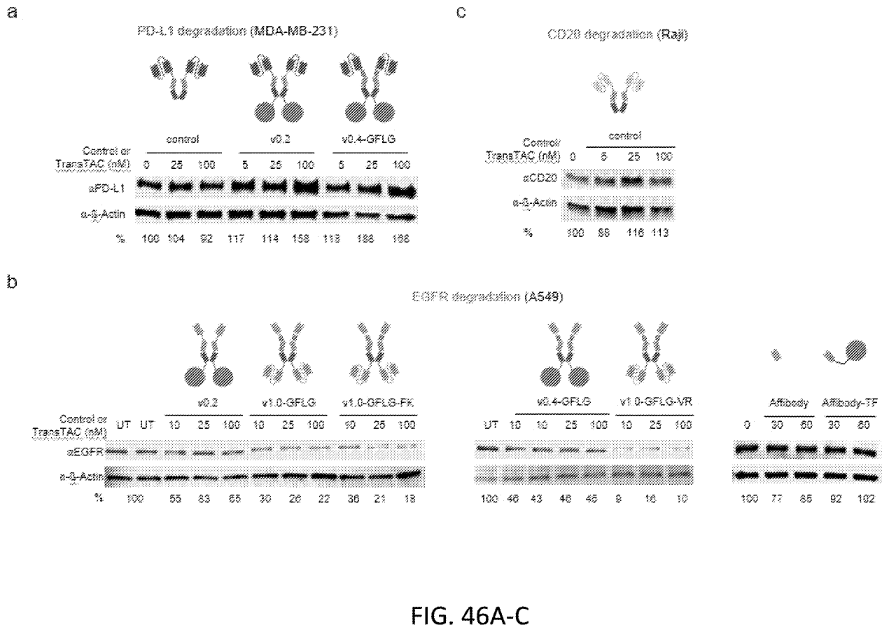

To understand the subcellular destination of the internalized CAR by v0.2, we stably expressed CAR-GFP and mCherry tagged to different endosomal and lysosomal markers, including Rab5+ or EEA+(EEs), Rab7+(LEs), Rab11+(REs), and Lamp1+(lysosomes), in a Hela cell line (FIG. 391, J, FIG. 45A-D). Fluorescence microscopy imaging of v0.2-treated cells showed co-localization of CAR-GFP with Rab11, indicating that the internalized CAR trafficked to the REs (FIG. 391, white arrow). Therefore, TransTACv0.2 effectively removes POIs from the cell membrane by trapping them in the recycling endosomal compartments in a target cell.

### Example 11—Degrader Engineering by Rationally Rewiring Intracellular Trafficking Pathways of the Internalized Protein Complex

Experiments related to intracellular trafficking are described in and are described with more details in this example.

Additional studies were to develop a next-generation TransTAC that not only traps the POI, but also leads to its degradation. This can be particularly beneficial for cancer-associated targets, as degradation can allow more sustained inhibition of protein functions.

To direct the target protein to degradation, we tested whether the POI (protein of interest) needs to disengage from the recycling Tf/TfR complex in the EE, which is where sorting into degradative or recycling pathways occurs (FIG. 38A, 39E, F). Proteases localized in the endosomes, such as the cysteine protease cathepsins, can be utilized to separate the POI from the Tf/TfR complex. Therefore, we incorporated a cathepsin B-sensitive Gly-Phe-Leu-Gly (GFLG) linker into TransTACs, either between the Fc domain and the Tf ligand (v0.3), or between CD19NT.1 and the Fc domain (v0.4) (FIG. 39A, 44A). Indeed, this linker modification altered the intracellular trafficking of CAR-GFP. With TransTACv0.4, a significant portion of the receptor now co-localizing with Rab7 (late endosome) and Lamp1 (lysosomes) (FIG. 39L, 45D-F). Furthermore, using western blotting, we observed that approximately 50% of CAR was degraded (FIG. 44C). Comparing to v0.4, the degradation with v0.3 was lower (FIG. 44C), possibly because CD19NT.1 remained linked to the Fc domain after separating from Tf/TfR, which could mediate recycling via the FcRn pathways. Overall, our studies indicated that the incorporation of a protease-cleavable linker in TransTACs leads to the degradation of POIs.

Our next goal was to improve efficiency of degradation and expand our understanding of proteolysis in endosomes. Traditionally, protein degradation was believed to occur primarily in the acidic lysosomes or LEs and little was known about proteolytic activity in the EE. To look for optimal protease substrates in EEs, we conducted a small-scale linker screening, thinking that protease activity in EEs would correlate with degradation efficiency. We screened a panel of 14 linkers containing single or combined cathepsin B cleavage motifs (Poreba, Marcin. “Protease-activated prodrugs: strategies, challenges, and future directions.” The FEBS Journal 287.10, 2020: 1936-1969), such as GFLG, Gly-Gly-Phe-Gly (GGFG), Phe-Lys (FK), Val-Ala (VA), Val-Lys (VK), and Val-Arg (VR), in both TransTACv0.4 and v1.0, using western assays (FIG. 44D-E). Overall, we found that incorporating dipeptide motifs VK, VR, and FK helped to improve cleavage activity, comparing to a GFLG sequence. Based on these results, we selected a GFLG-VR linker and an EVR linker for developing later generations of TransTACs. We used the EVR linker instead of VR to prepare for in vivo studies since previous research has found that including a glutamic acid improves the stability of linker in mouse serum.

Subsequently, we took another step in optimizing TransTACs by substituting the TF ligand with an anti-TfR single-chain Fv (scFv) called H7, a TF-competitive antibody identified by phage display (FIG. 39A) (Goenaga, Anne-Laure, et al. “Identification and characterization of tumor antigens by using antibody phage display and intrabody strategies.” Molecular immunology 44.15, 2007: 3777-3788; Tillotson, Benjamin J., et al. “Engineering an anti-transferrin receptor ScFv for pH-sensitive binding leads to increased intracellular accumulation.” PLoS One 10.12, 2015: e0145820). This substitution aimed to reduce RE sorting, a step that we felt would be important in increasing degradation efficiency, as proteins sorted to the RE cannot be trafficked to LE/lysosomes for degradation (FIG. 39G, H). The logic was that certain molecular features of the TF/TfR complex are involved in RE sorting and that using a synthetic antibody binder like H7 could alter this sorting decision and redirect the complex's intracellular trafficking after iron release in the EE.

Based on this, we generated two versions of TransTACs: v0.5 containing the H7 binder but no cleavable linker, and v1.0 containing both the H7 and the cleavable linker (FIG. 39A). Cells treated with v0.5 showed that CAR-GFP predominantly colocalized with EE markers Rab5 and EEA (FIG. 39G, J, 45A,D). Pearson colocalization coefficients of the corresponding markers are statistically different from cells treated with TransTACv0.2 that contains the TF as the anti-TfR ligand (FIG. 39K). This result validated that replacing TF with an anti-TfR antibody can reduce RE trafficking. Moreover, re-trafficking led to a significant improvement in degradation efficiency. With TransTACv1.0, we observed over 80% degradation of CAR in both western and fluorescence microscopy assays (FIG. 39D, I, J; FIG. 45A-C). In addition to improving degradation, the H7 substitution also increased the yield of the protein by approximately sevenfold, making the expression level of TransTACs similar to that of conventional antibodies. Taken together, our rational protein engineering efforts have successfully developed a novel protein design, TransTACv1.0, as a potent molecular degrader for CAR, which is fully recombinant and expresses robustly. CAR-TransTACv1.0 represents the first recombinant protein degrader made to target a synthetic receptor.

### Example 12—Reversible Control of Primary CAR-T Cell Functions with CAR-TransTAC

Experiments related to efficacy and reversibility of TransTAC molecules to control CAR-T cells are described in Examples 2 and 6.

In additional studies, we investigated the use of TransTAC as OFF switches to fine tune CAR-T cell activity and to manage associated toxicities, such as those that can manifest as cytokine release syndrome (CRS) caused by overactivation of CAR-T cells (FIG. 47A).

As a proof of concept, we showed CAR-TransTACv0.4 can effectively inhibit human primary CAR-T cells.

We isolated primary CD8+ T cells from human PBMCs and generated anti-CD19 CAR-T cells through lenti-viral transduction. For the tumor cells, we used an adherent melanoma cell line, A375, which has been engineered to express CD19 and a nuclear mCherry to facilitate live cell imaging. We observed CAR-TransTAC v0.4 potently inhibited IFN-γ secretion, with an IC50 of approximately 0.4 nM and a Dₘₐₓ of 88% (FIG. 47B, C). The molecules also effectively blocked the tumor-killing activities (FIG. 47B, D). Furthermore, the inhibition was reversible, as the removal of the TransTAC resumed tumor killing activities of primary CAR-T cells (FIG. 47B, E).

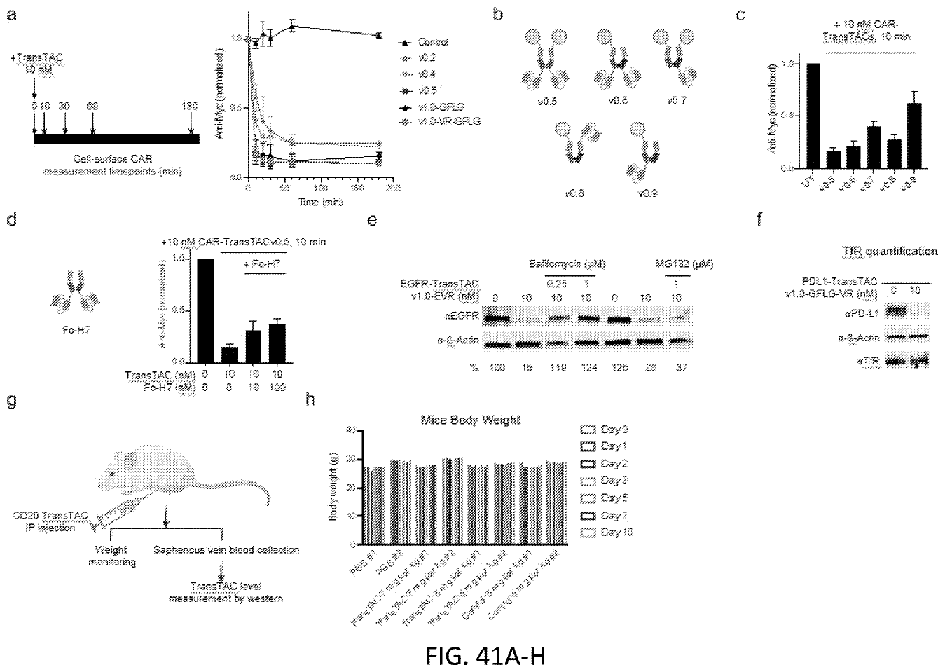

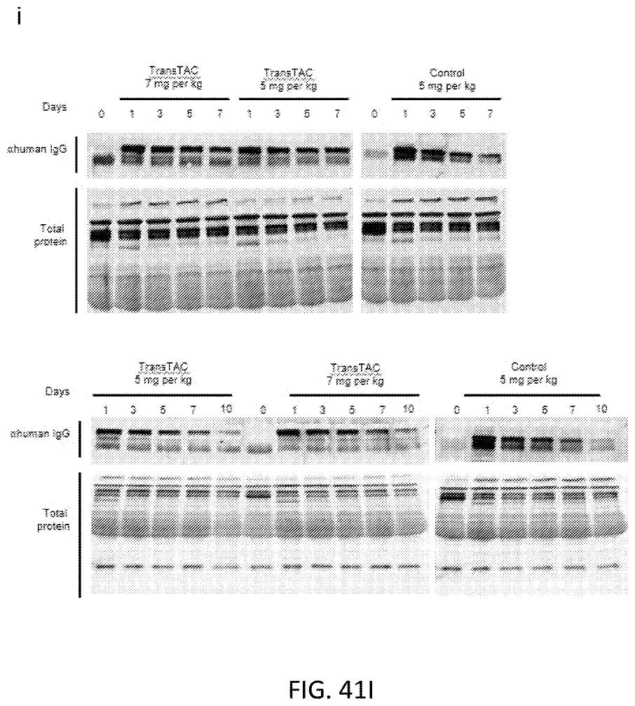

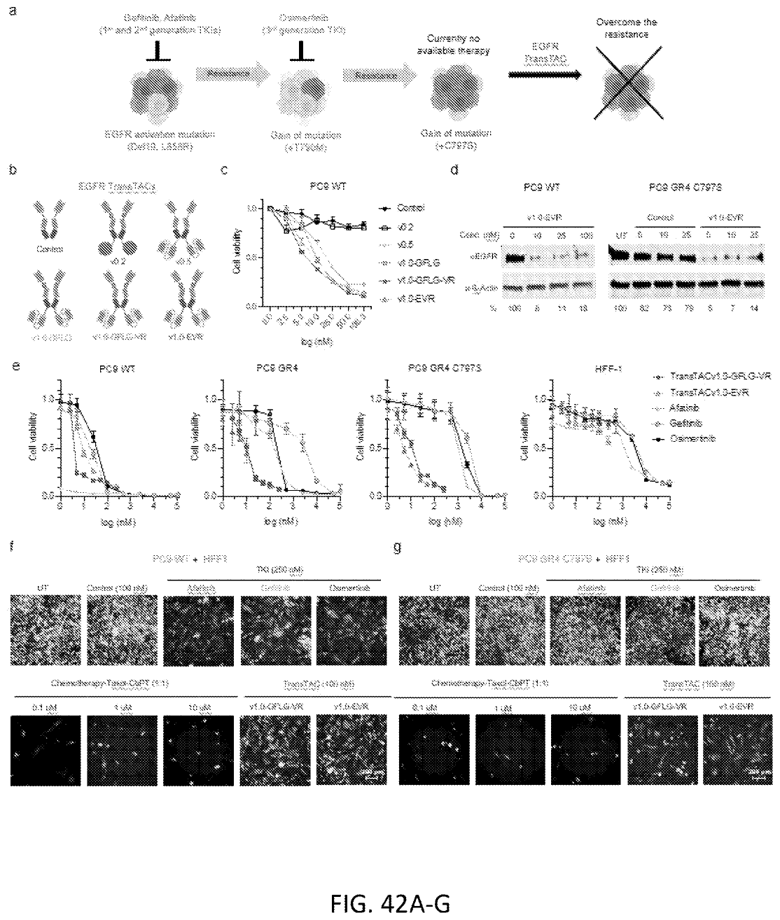

To gain a better understanding of the factors that influence the varying performances of the CAR-TransTACs, and to determine the general structure-function activity (SAR) relationships of TransTACs, we generated and tested four TransTACv0.5 variants, v0.6-v0.9, each containing one or two copies of CD19NT.1 or H7 in different geometries (FIG. 41B). Our findings revealed that having two H7s was better than having two CD19NT.1s in enhancing CAR internalization (FIG. 41C, D). This highlights the importance of dual binding to a dimeric TfR, rather than having two anti-POI binders, in creating a potent TransTAC. It also demonstrated that TransTAC-mediated CAR internalization was not the result of CAR crosslinking. Additionally, we observed significant differences in the internalization efficiency for different geometries of the molecules (FIG. 41C), indicating that the tertiary complex structure plays a role in influencing TransTAC efficiency. Furthermore, we generated an Fc-H7 molecule as a competitor for TfR binding and observed a dose-dependent decrease of CAR internalization in the presence of the competitor in solution with TransTACv0.5 treatment (FIG. 47D). This observation further indicates that TransTACs function through a TfR-dependent mechanism.

As CAR clustering can lead to low-level spontaneous CAR-T cell activation, we hypothesized that our dimeric CAR OFF-switch molecules, by inducing CAR clustering, may have influenced their inhibition effects on CAR-T cells. Therefore, we developed CAR OFF switches containing only one CD19NT.1 domain and compared them to the dimeric variants. The study indicated CD19NT.1 monomer had a significant 100-fold decrease in potency compared to the CD19NT.1-Fc dimers. This finding highlights the importance of avidity for CD19NT.1-Fc in achieving effective CAR-T cell inhibition. As CAR-T/tumor interactions involve multiple CAR/antigen interactions at the immunological synapse, a molecule with multiple copies appears better to effectively compete with tumor antigens for CAR binding. To further understand the role of multivalency, we also generated a tetrameric variant, which showed similar potency in CAR-T cell inhibition compared to the dimer.

CAR-TransTACs did not rely on competition with tumor CD19 for its effects and therefore did not require a dimeric format to be effective. We observed that a monomeric CD19NT.1-based TransTAC (a domain to which CAR can bind fused to an Fc region)exhibited robust performance and even outperformed the dimeric variant for blocking CAR-Jurkat cells.

### Example 13—Expansion of TransTAC-Addressable Targets

Experiments on TransTAC molecules specific for additional cell-surface molecules are described in Examples 1, 4, 5 and 7.

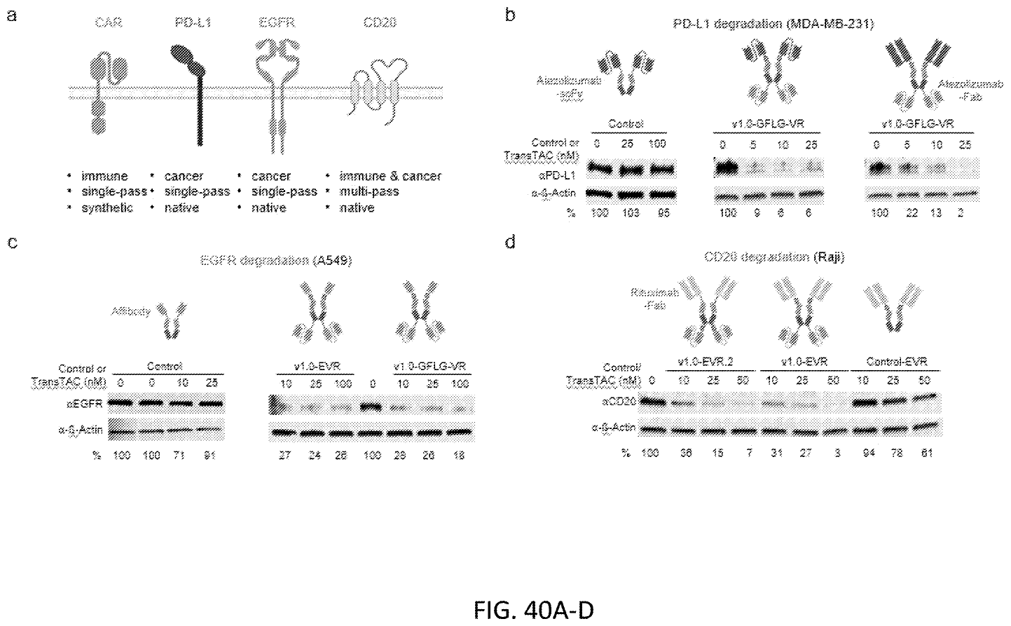

In additional studies, we interrogated the generalizability of TransTACs. To date, all biologics-based degraders have been developed to target single-pass membrane proteins. We sought to expand the scope of targets by including both single-pass membrane targets such as epidermal growth factor receptor (EGFR) and programmed death-ligand 1 (PDL1), as well as a multi-pass membrane protein, cluster of differentiate 20 (CD20) (FIG. 40A). These targets have diverse functions and regulatory pathways and are found on a wide range of cancer and immune cells.

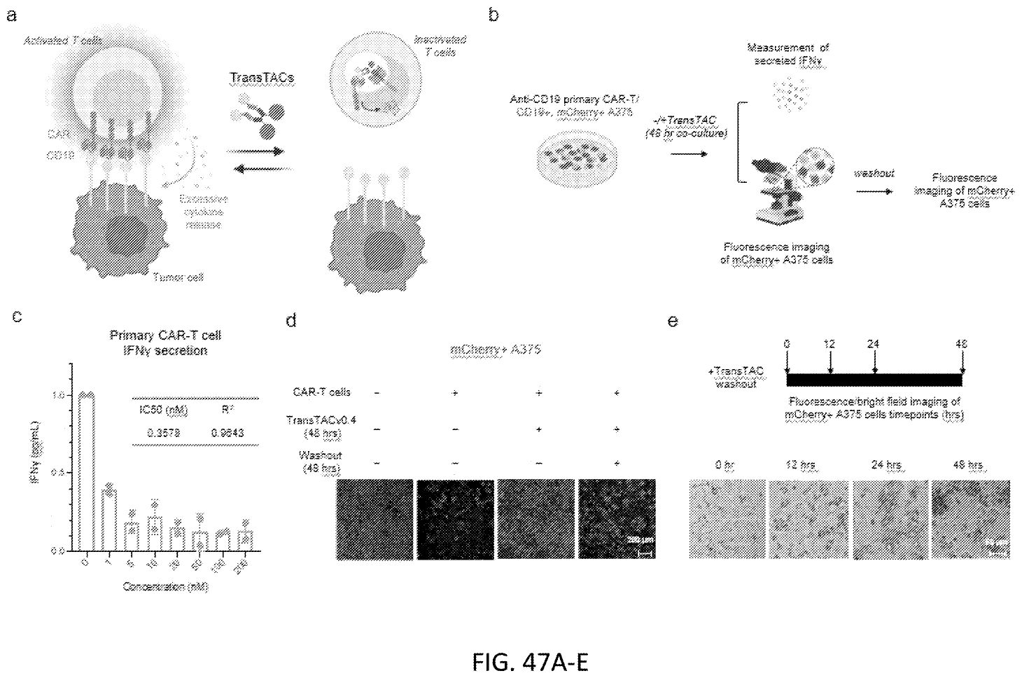

Our first target is programmed death-ligand 1 (PD-L1), an immune checkpoint receptor ligand, downregulation of which can enhance anti-tumor T cell activity. PD-L1 targeting using monoclonal antibodies have seen moderate success in the clinic, thus new mechanisms to target this protein could be highly valuable. A PD-L1-TransTAC was created using a fragment antigen binding (Fab) or single-chain variable fragment (scFv) of atezolizumab as the PDL1 binding domain. Up to 98% PD-L1 degradation was observed in MDA-MB-231 breast cancer cells treated with PD-L1-TransTACs, while control groups lacking H7 or containing TransTACv0.2 and v0.4 with a TF ligand showed no or little PD-L1 degradation (FIG. 40B; FIG. 46A).

Next, we aimed to target epidermal growth factor receptor (EGFR), a receptor tyrosine kinase that plays an important role in the development and progression of various types of cancers such as lung and brain. An affibody (Friedman, Mikaela, et al. “Directed evolution to low nanomolar affinity of a tumor-targeting epidermal growth factor receptor-binding affibody molecule.” Journal of molecular biology 376.5, 2008: 1388-1402) was used to bind EGFR and create an EGFR-TransTAC. A549 lung carcinoma cells treated with EGFR-TransTACv1.0s containing GFLG-VR or EVR linkers showed up to 80-90% reduction of EGFR, whereas control groups exhibited little to no degradation (FIG. 40C, FIG. 46B). Different linkers in v1.0 resulted in varying degrees of EGFR degradation, but all were lower than TransTACs with GFLG-VR or EVR, while v0.2 had no effect (FIG. 46B). These results were consistent with the observations with the CAR-TransTAC variants, validating the importance of those modifications made to improve TransTAC.

Cluster of differentiate 20 (CD20) is a B cell-specific surface marker with four transmembrane domains and an unknown function. Knocking down cell surface CD20s with a degrader can be valuable. A CD20-TransTAC was created using a Fab format of Rituximab, the first clinically approved CD20 antibody, to bind CD20. Treatment of Raji cells, a human B lymphoblastoid cell line, with the resulting CD20-TransTAC resulted in up to 97% reduction of CD20, while control groups led to no or significantly less degradation (FIG. 40D, FIG. 46C).

Together, the successful generation of degraders against all four targets demonstrates the modularity and generality of the TransTAC design. Importantly, high potencies were observed for all four targets studied, all reaching >80% in various cellular systems, which demonstrates the efficiency of targeted degradation using the TransTAC degrader designs.

### Example 14—Kinetics, Structure-Activity Relationship (SAR), Mechanism, and In Vivo Characterization of TransTACs

We conducted further characterizations of the degraders to understand their underlying mechanisms and SARs.

First, we studied the kinetics of TransTAC-mediated protein internalization by measuring the time-course change of cell-surface CAR levels (FIG. 41A). We observed a rapid elimination of CAR from the cell surface, with only 17% remaining after 10 minutes and 13% after 20 minutes of treatment with TransTACv1.0-GFLG-VR. Furthermore, this response was long-lasting, with 10% of CAR observed at the cell surface after 3 hours with v1.0. This fast and sustained protein downregulation highlights TransTACs as a promising research tool for knocking down cell surface proteins as an alternative to genetic methods, offering temporal resolution of membrane protein regulation.

To further understand how the number of binders and geometry of TransTACs influence its behavior, we generated and tested four CAR-TransTACv0.5 variants, v0.6-v0.9, each containing one or two copies of CD19NT.1 or H7 (FIG. 41B). Our findings revealed that having two H7s was more critical than having two CD19NT.1s in enhancing CAR internalization (v0.6 vs. v0.7, FIG. 41C). This highlights the importance of dual binding to a dimeric TfR, rather than having two anti-POI binders, in creating a potent TransTAC. It also indicated that TransTAC-mediated CAR internalization was not the result of CAR crosslinking. Additionally, we observed significant differences in the internalization efficiency for molecules in different geometries, indicating that the tertiary complex structure plays a role in influencing TransTAC efficiency (v0.8 vs. v0.9, FIG. 41C). Furthermore, we generated an Fc-H7 molecule as a competitor for TfR binding and observed a dose-dependent decrease of CAR internalization in the presence of the competitor in solution with TransTACv0.5 treatment (FIG. 41D) This observation further validates that TransTACs function through a TfR-dependent mechanism. These SAR analyses offer valuable insights to guide future TransTAC designs.

We next investigated the cellular mechanism underlying TransTAC-mediated protein degradation. Two primary pathways involved in the degradation of cellular proteins were tested: the lysosomal pathway and the proteosome pathway. A549 cells were treated either with bafilomycin, a vacuolar proton pump inhibitor that inhibits lysosomal acidification, or MG132, a proteasomal inhibitor. We observed 1 μM bafilomycin prevented TransTAC-mediated EGFR degradation, whereas 1 μM MG132 had a much less significant effect (FIG. 41E). These results show that intact lysosomal function was essential for TransTAC-mediated protein degradation.

To determine whether TfR level remains consistent or reduced with TransTAC treatment, we characterized whole-cell TfR expression using western blotting assay with a PD-L1-TransTAC. No change in TfR level was observed, which is in clear contrast to the loss of PD-L1 in the same assay (FIG. 41F). This result validates our hypothesis that the POI was separated from TfR before being routed to degradation, while TfR is recycled.

Lastly, we asked whether TransTACs would be well tolerated and have similar antibody clearance to IgGs in vivo. We intraperitoneally injected 5 or 7 mg/kg (body weight) CD20 TransTAC or 5 mg/kg IgG control into nude mice (FIG. 41G). No significant weight changes were observed with either the TransTACs or the control (FIG. 41H). Western blotting analysis of plasma antibody levels revealed that the TransTAC remained in plasma up to 10 d after injection with a half-life of approximately 10 d, which is longer than the tested control IgG and comparable to the reported half-life of IgGs in mice (FIG. 41I). It was known that the scFv-H7 antibody cross-reactive with mouse TfR. Together, these results demonstrate that TransTACs are well-tolerated and have favorable pharmacokinetics and are not being rapidly cleared despite cross-reactivity with mouse cells.

### Example 15—Targeting Drug Resistant Small Cell Lung Cancer with EGFR-TransTACs

Experiments related to TransTAC molecules for treating cancer are described in Example 8

Additionally, cancers evolve rapidly to evade therapy, often developing drug-resistant mutations that lead to treatment failure and disease recurrence. The C797S mutation of EGFR, in particular, poses a substantial challenge in the treatment of non-small cell lung cancer (NSCLC), which accounts for 85% of all lung cancer cases. Emerging in roughly 10-26% of NSCLC patients following treatment with the third-generation EGFR tyrosine kinase inhibitors (TKI) Osimertinib, the C797S mutation affects a critical residue, C797, which forms covalent bonds with irreversible TKIs. Consequently, existing TKI therapies become ineffective against the disease.

EGFR-TransTACs, which can induce targeted degradation of EGFR in TfR-upregulated cancer cells, can target EGFR driven lung cancer patients including the C797S-mutant population (FIG. 42A). Three lung cancer cell lines were used: PC9-wildtype (WT), PC9 GR4, and PC9 GR4 C797S. PC9-WT cell is a lung adenocarcinoma cell line with a deletion in exon 19 (Del 19) of the EGFR gene, sensitive to all three-generations of TKIs. PC9-GR4 is a gefitinib-resistant, osimertinib sensitive cell line carrying the T790M mutation (Del 19/T790M), generated through a previously established drug selection protocol. Finally, PC9 GR4 C797S (Del 19/T790M/C797S) is a CRISPR-engineered cell line harboring an additional C797S mutation, making it further resistant to Osimertinib.

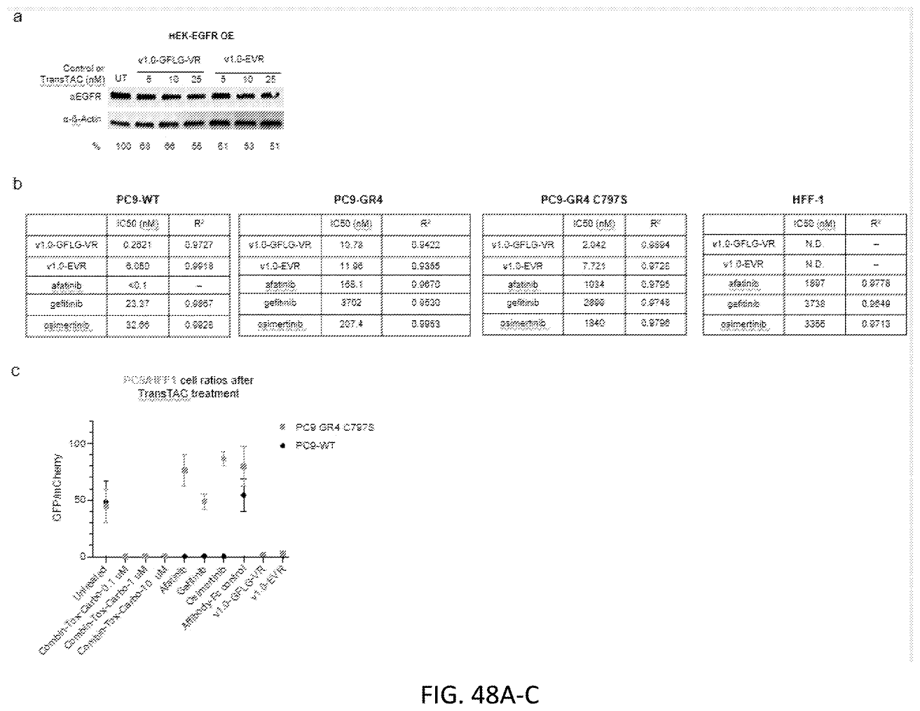

We generated several EGFR-affibody-based TransTAC variants (FIG. 42B) and evaluated their dose-dependent inhibition efficiency first on PC9-WT cells using a 3-(4,5-dimethylthiazol-2-yl)-2,5-diphenyltetrazolium bromide MTT cell viability assay. TransTACv1.0 and v0.5 variants led to dose-dependent inhibition of the cells, with IC50s in the low-nM range, whereas the control affibody-Fc fusion and v0.2 showed no or little response (FIG. 42C). Consistently, more than 90% maximal EGFR degradation was observed in PC9-WT cells and PC9 GR4 C797S cells with TransTACv1.0 treatments, whereas the affibody-Fc control led to no degradation (FIG. 42D). The same TransTAC molecules only induced approximately 40-50% EGFR degradation in an engineered EGFR-overexpressing HEK293 cell line, which expresses 3-10× less TfRs compared to the three PC9 cell lines (FIG. 48A). This result indicates that the efficiency of TransTAC-mediated protein degradation is positively correlated with TfR expression levels, highlighting the potential advantage of TransTAC technology's cancer-specificity.

We next compared EGFR-TransTACs with first-, second-, and third-generation EGFR TKIs gefitinib, afatinib, and osimertinib. The sensitivity of the three cell lines to these TKIs was consistent with previous reports (FIG. 42E, 48B). PC9-WT cells showed sensitivity to all three TKIs, with IC50s ranging from <0.1 to 33 nM. PC9-GR4 cells were less sensitive to afatinib and osimertinib, with IC50s of 168 and 207 nM, respectively, and showed full resistance to gefitinib. PC9-GR4-C797S cells did not respond to any of the three inhibitors.

Different from the TKIs, TransTACv1.0s effectively inhibited all the three cell lines, exhibiting IC50s in the low or sub-nM range (FIG. 42E, 48B). In particular, TransTACv1.0-GFLG-VR had an IC50 of 2 nM and TransTACv1.0-EVR had an IC50 of 8 nM against the PC9 GR4 C797S cells. To evaluate the off-tumor toxicities, a healthy cell human fibroblast cell line (HFF-1) was included in the assay. Neither the TransTACs or TKIs showed significant inhibition until the concentration of the molecules reached the high nM or M range (FIG. 42E, 48B).

To further compare the efficacy and specificity of TransTACs with standard care therapies, we performed a co-culture assay of normal and cancer cells and monitored the effects of drugs using live cell fluorescence imaging (FIG. 42F-G). PC9 and PC9 GR4 C797S cells were engineered to express GFP and the HFF-1 cells expressed mCherry. The cells were mixed in a 1:10 ratio and treated with TransTACs or TKIs. Consistent with the MTT assay results, TransTACs demonstrated high efficacy against both PC9-WT and PC9 GR4 C797S cells. In contrast, the TKIs inhibited PC9 WT cancer cells but not the PC9 GR4 C797S cells. The UT or affibody-Fc control molecule showed no effect on either cells.

Additionally, TransTACs were also compared with chemotherapy drugs. Unlike TransTACs, which didn't kill the HFF1 healthy cells, a combination of carboplatin and paclitaxel chemotherapy were cytotoxic to both cancer and normal cells (FIG. 42F-G). This is consistent with previous notions that chemotherapy often has high off-tumor toxicities, despite it being the first-line therapy for many cancer types.

Furthermore, the results of live cell imaging were validated by conducting flow cytometry analysis to determine the ratio of GFP/mCherry positive cells after treatments, which reflects the relative drug cytotoxicity to cancer cells versus healthy cells (FIG. 48C). Our analysis indicated that TransTACs inhibited both PC9 WT and the GR4 CS cells, showing a near-zero cancer/healthy cell ratio, indicating high cancer targeting potency and specificity. In contrast, the TKIs were much less effective against the GR4 CS cells, and the cancer cells dominated the entire population of the cell mixtures.

Taken together, these findings demonstrated that the EGFR TransTAC molecules can target lung cancer cells harboring the EGFR C797S mutation. Furthermore, the comparison with current standard of cares demonstrates superior on-tumor efficacy and specificity of TransTACs.

### Example 16—a Schematic Illustration of the Design Principles we have Discovered for Enhancing Degradation Efficiency of TransTACs

FIG. 38b is an illustration of an example TransTAC degrader. Generally, TransTACs are recombinant proteins consisting of anti-POI binders and anti-TfR binders for bridging POI and TfR in close proximity on the cell surface. We found many formats for TransTACs can efficiently eliminate the target proteins from the cell surface as outlined in FIGS. 39A and 44A. Therefore, all confer effectiveness as modulators of membrane proteins.

However, we discovered at least three example design principles to make TransTACs efficient internalizers and degraders: (1) a dimeric TransTAC drives more efficient protein internalization than a monomeric heterobispecific TransTAC; (2) a cathepsin B-sensitive linker is important for allowing lysosomal trafficking of the POI; and (3) an antibody binder for targeting TfR, rather than a native transferrin (TF) ligand, could reduce trafficking of the POI to the recycling endosomes (REs) and hence enhance degradation efficiency

Using specific variants of the molecules, we could choose to induce endosomal trapping or lysosomal degradation of the targets, offering customizable possibilities for modular manipulation of membrane proteins.

### Example 17—Identification of Peptides not Known to be Sensitive to Cathepsin Cleavage

A yeast-displayed peptide library was used to identify peptides not known to be sensitive to cathepsin cleavage. These peptides can be from a combination of small motifs found in SEQ ID NOs: 144 and 145. The studies were performed at pH 4.4 (FIG. 49A) and at pH 6.4 (FIG. 49B). These peptides include GRLVGFD (SEQ ID NO: 124), GRLVGFG (SEQ ID NO: 125), RMLVGFV (SEQ ID NO: 126), RRLYAFL (SEQ ID NO: 127), VFRLLMF (SEQ ID NO: 128), LVGVLLF (SEQ ID NO: 129), VKLYGLG (SEQ TD NO: 130), TWRVDLY (SEQ ID NO: 131), EQLYLYA (SEQ ID NO: 132), KLFLMIF (SEQ ID NO:133), NFVIILF (SEQ ID NO: 134), MSLLIGV (SEQ ID NO: 135), VRLLSLQ (SEQ ID NO: 136), STLMWNV (SEQ ID NO: 137), VRFLAAA (SEQ ID NO: 138), HGWSFHE (SEQ ID NO: 139), ENLYFQG (SEQ ID NO: 140), VVMMFLH (SEQ ID NO: 141), VFRLLMF (SEQ ID NO: 142), or VGALVWL (SEQ ID NO: 143).

### EQUIVALENTS

Those skilled in the art will recognize, or be able to ascertain, using no more than routine experimentation, numerous equivalents to the specific substances and procedures described herein. Such equivalents are considered to be within the scope of this invention.

What is claimed:

1. A fusion protein of Formula I:
R1-R2-R3 (I):
wherein:
R1 is at least one protein of interest (POI) binder (POIB);
R2 is a linker of the formula R4-R5 or R5-R4, wherein:
R4 is IgG Fc region; and
R5 is a protease-sensitive linking means; and
R3 is a transferrin receptor binding (TRB) means, and
optionally, wherein a linkage between R2 and R3 is a glycine-rich linker.

2. The fusion protein of claim 1, wherein the protease-sensitive linking means comprises a cathepsin-cleavable peptide.

3. The fusion protein of claim 2, wherein the cathepsin-cleavable peptide linker is selected from the group consisting of FK, VA, VK, SEQ ID NO: 7, 8, 118, 119, 120, 121, 122, 123, 124, 125, 126, 127, 128, 129, 130, 131, 132, 133, 134, 135, 136, 137, 138, 139, 140, 141, 142, 143, 144 and 145.

4. The fusion protein of claim 1, wherein the TRB binding means is H7 or M16.

5. The fusion protein of any one of claims 1-4, wherein the fusion protein of Formula I is a homodimer.

6. The fusion protein of any one of claims 1-5, wherein the fusion protein of Formula I is a heterodimer linked to a fusion protein of Formula II:
R4′-R3′ (II):
wherein:
R4′ is IgG Fc region; and
R3′ is a transferrin receptor binding (TRB) means; and
optionally, wherein a linkage between R3′ and R4′ is a protease-sensitive linking means.

7. The fusion protein of any one of claims 1-6, wherein the TRB comprises an antibody or polypeptide.

8. The fusion protein of any one of claims 1-7, wherein the TRB is selected from the group consisting of SEQ ID NOs: 3, 4, and 5.

9. The fusion protein of any one of claims 1-8, wherein the POIB comprises an antibody.

10. The fusion protein of any one of claims 1-9, wherein the POIB binds to an extracellular domain of a transmembrane protein.

11. The fusion protein of claim 10, wherein the extracellular region of the membrane protein comprises a chimeric antigen receptor (CAR), a receptor tyrosine kinase, a checkpoint inhibitor binding molecule, or a cell lineage-specific marker.

12. The fusion protein of any one of claims 1-11, wherein the POIB binds to an extracellular domain of an epidermal growth factor receptor (EGFR), a programmed death-ligand 1 (PD-L1), or CD20.

13. The fusion protein of any one of claims 1-12, wherein a linkage between R2 and R3 is a glycine-rich linker selected from the group consisting of of SEQ ID NO: 9, 10, 11, 12, 13, 14, 15 and 16.

14. A nucleic acid sequence encoding the fusion protein of any one of claims 1-13.

15. A method for treating a subject that has cancer, comprising administering the fusion protein of any one of claims 1-13 to the subject.

16. The fusion protein of any one of claims 1-13 for use in treating a cancer in a patient.

17. A homodimer of a fusion protein of Formula I:
R1-R2-R3 (III):
wherein:
R1 is at least one protein of interest (POI) binder (POIB);
R2 is a linker of the formula R4-R5 or R5-R4, wherein:
R4 is IgG Fc region; and
R5 is a protease-sensitive linking means; and
R3 is a transferrin receptor binding (TRB) means, and
optionally, wherein a linkage between R2 and R3 is a glycine-rich linker.

18. The homodimer of claim 17, additionally comprising a disulfide bond between cysteine amino acids in R4 of separate fusion proteins.

19. A homodimer of a fusion protein of Formula I:
R1-R6-R3 (I):
wherein:
R1 is at least one protein of interest (POI) binder (POIB);
R6 is a dimerization means; and
R3 is a transferrin receptor binding (TRB) means;
wherein the homodimer optionally has a protease-sensitive linking means between R1 and R6.

20. A pharmaceutical composition, comprising the fusion protein or homodimer of a fusion protein of any one of claims 1-13 or 17-19.
